SEQUENCE LISTING (133 sequences)

SEQ ID NO: 1 - DNA, 834 bp, unidentified
  source 1..834: mol_type = genomic DNA; note = Escherichia coli K-12
atgcagcgcttatttcttttggttgccgtcatgttactgagcggatgcttaaccgccccgcctaaagaagccgccagaccgacattaatgcctcgtgctcagagctacaaagatttgacccatctgccagcgccgacgggtaaaatctttgtttcggtatacaacattcaggacgaaaccgggcaatttaaaccctacccggcaagtaacttctccactgctgttccgcaaagcgccacggcaatgctggtcacggcactgaaagattctcgctggtttataccgctggagcgccagggcttacaaaacctgcttaacgagcgcaagattattcgtgcggcacaagaaaacggcacggttgccattaataaccgaatcccgctgcaatctttaacggcggcaaatatcatggttgaaggttcgattatcggttatgaaagcaacgtcaaatctggcggggttggggcaagatattttggcatcggtgccgacacgcaataccagctcgatcagattgccgtgaacctgcgcgtcgtcaatgtgagtaccggcgagatcctttcttcggtgaacaccagtaagacgatactttcctatgaagttcaggccggggttttccgctttattgactaccagcgcttgcttgaaggggaagtgggttacacctcgaacgaacctgttatgctgtgcctgatgtcggctatcgaaacaggggtcattttcctgattaatgatggtatcgaccgtggtctgtgggatttgcaaaataaagcagaacggcagaatgacattctggtgaaataccgccatatgtcggttccaccggaatcctga

SEQ ID NO: 2 - protein, 277 aa, unidentified
  source 1..277: note = Escherichia coli K-12
MQRLFLLVAVMLLSGCLTAPPKEAARPTLMPRAQSYKDLTHLPAPTGKIFVSVYNIQDETGQFKPYPASNFSTAVPQSATAMLVTALKDSRWFIPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEVGYTSNEPVMLCLMSAIETGVIFLINDGIDRGLWDLQNKAERQNDILVKYRHMSVPPES

SEQ ID NO: 3 - protein, 262 aa, unidentified
  source 1..262: note = Escherichia coli K-12
CLTAPPKEAARPTLMPRAQSYKDLTHLPAPTGKIFVSVYNIQDETGQFKPYPASNFSTAVPQSATAMLVTALKDSRWFIPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEVGYTSNEPVMLCLMSAIETGVIFLINDGIDRGLWDLQNKAERQNDILVKYRHMSVPPES

SEQ ID NO: 4 - DNA, 414 bp, unidentified
  source 1..414: mol_type = genomic DNA; note = Escherichia coli K-12
atgcgtgtcaaacatgcagtagttctactcatgcttatttcgccattaagttgggctggaaccatgactttccagttccgtaatccaaactttggtggtaacccaaataatggcgcttttttattaaatagcgctcaggcccaaaactcttataaagatccgagctataacgatgactttggtattgaaacaccctcagcgttagataactttactcaggccatccagtcacaaattttaggtgggctactgtcgaatattaataccggtaaaccgggccgcatggtgaccaacgattatattgtcgatattgccaaccgcgatggtcaattgcagttgaacgtgacagatcgtaaaaccggacaaacctcgaccatccaggtttcgggtttacaaaataactcaaccgatttt

SEQ ID NO: 5 - protein, 138 aa, unidentified
  source 1..138: note = Escherichia coli K-12
MRVKHAVVLLMLISPLSWAGTMTFQFRNPNFGGNPNNGAFLLNSAQAQNSYKDPSYNDDFGIETPSALDNFTQAIQSQILGGLLSNINTGKPGRMVTNDYIVDIANRDGQLQLNVTDRKTGQTSTIQVSGLQNNSTDF

SEQ ID NO: 6 - protein, 119 aa, unidentified
  source 1..119: note = Escherichia coli K-12
GTMTFQFRNPNFGGNPNNGAFLLNSAQAQNSYKDPSYNDDFGIETPSALDNFTQAIQSQILGGLLSNINTGKPGRMVTNDYIVDIANRDGQLQLNVTDRKTGQTSTIQVSGLQNNSTDF

SEQ ID NO: 7 - DNA, 106 bp, synthetic construct
  source 1..106: mol_type = other DNA
atgcgtgtcaaacatgcagtagttctactcatgcttatttcgccattaagttgggctggaaccatgactttccagttccgtcatcaccatcaccatcactaagccc

SEQ ID NO: 8 - protein, 33 aa, synthetic construct
MRVKHAVVLLMLISPLSWAGTMTFQFRHHHHHH

SEQ ID NO: 9 - DNA, 139 bp, synthetic construct
  source 1..139: mol_type = other DNA
atgcgtgtcaaacatgcagtagttctactcatgcttatttcgccattaagttgggctggaaccatgactttccagttccgtaatccaaactttggtggtaacccaaataatggccatcaccatcaccatcactaagccc

SEQ ID NO: 10 - protein, 44 aa, synthetic construct
MRVKHAVVLLMLISPLSWAGTMTFQFRNPNFGGNPNNGHHHHHH

SEQ ID NO: 11 - DNA, 169 bp, synthetic construct
  source 1..169: mol_type = other DNA
atgcgtgtcaaacatgcagtagttctactcatgcttatttcgccattaagttgggctggaaccatgactttccagttccgtaatccaaactttggtggtaacccaaataatggcgcttttttattaaatagcgctcaggcccaacatcaccatcaccatcactaagccc

SEQ ID NO: 12 - protein, 54 aa, synthetic construct
MRVKHAVVLLMLISPLSWAGTMTFQFRNPNFGGNPNNGAFLLNSAQAQHHHHHH

SEQ ID NO: 13 - DNA, 217 bp, synthetic construct
  source 1..217: mol_type = other DNA
atgcgtgtcaaacatgcagtagttctactcatgcttatttcgccattaagttgggctggaaccatgactttccagttccgtaatccaaactttggtggtaacccaaataatggcgcttttttattaaatagcgctcaggcccaaaactcttataaagatccgagctataacgatgactttggtattgaaacacatcaccatcaccatcactaagccc

SEQ ID NO: 14 - protein, 70 aa, synthetic construct
MRVKHAVVLLMLISPLSWAGTMTFQFRNPNFGGNPNNGAFLLNSAQAQNSYKDPSYNDDFGIETHHHHHH

SEQ ID NO: 15 - protein, 34 aa, synthetic construct
GTMTFQFRNPNFGGNPNNGAFLLNSAQAQNSYKD

SEQ ID NO: 16 - protein, 25 aa, synthetic construct
GTMTFQFRNPNFGGNPNNGAFLLKD

SEQ ID NO: 17 - protein, 32 aa, synthetic construct
TELVYTPVNPAFGGNPLNGTWLLNNAQAQNDY

SEQ ID NO: 18 - protein, 32 aa, synthetic construct
TELIYEPVNPNFGGNPLNGSYLLNNAQAQDRH

SEQ ID NO: 19 - protein, 32 aa, synthetic construct
SELVYTPVNPNFGGNPLNTSHLFGGANAINDY

SEQ ID NO: 20 - protein, 32 aa, synthetic construct
TQLVYTPVNPAFGGSYLNGSYLLANASAQNEH

SEQ ID NO: 21 - protein, 32 aa, synthetic construct
SSLVYEPVNPTFGGNPLNTTHLFSRAEAINDY

SEQ ID NO: 22 - protein, 32 aa, synthetic construct
TELVYEPINPSFGGNPLNGSFLLSKANSQNAH

SEQ ID NO: 23 - protein, 32 aa, synthetic construct
TEIVYQPINPSFGGNPMNGSFLLQKAQSQNAH

SEQ ID NO: 24 - protein, 33 aa, synthetic construct
SSLVYQPVNPAFGGPQLNGSWLQAEANAQNIPQ

SEQ ID NO: 25 - protein, 31 aa, synthetic construct
GSLVYTPTNPAFGGSPLNGSWQMQQATAGNH

SEQ ID NO: 26 - protein, 32 aa, synthetic construct
QQLIYQPTNPSFGGYAANTTHLFATANAQKTA

SEQ ID NO: 27 - protein, 32 aa, synthetic construct
GDLVYTPVNPSFGGSPLNSAHLLSIAGAQKNA

SEQ ID NO: 28 - protein, 32 aa, synthetic construct
AELGYTPVNPSFGGSPLNGSTLLSEASAQKPN

SEQ ID NO: 29 - protein, 31 aa, synthetic construct
TELVFSFTNPSFGGDPMIGNFLLNKADSQKR

SEQ ID NO: 30 - protein, 32 aa, synthetic construct
QQLVYKSINPFFGGGDSFAYQQLLASANAQND

SEQ ID NO: 31 - protein, 31 aa, synthetic construct
QALVYHPNNPAFGGNTFNYQWMLSSAQAQDR

SEQ ID NO: 32 - protein, 32 aa, synthetic construct
TELVYTPKNPAFGGSPLNGSYLLGNAQAQNDY

SEQ ID NO: 33 - protein, 32 aa, synthetic construct
GQLIYQPINPSFGGDPLLGNHLLNKAQAQDTK

SEQ ID NO: 34 - protein, 32 aa, synthetic construct
TQLIYTPVNPNFGGSYLNGSYLLANASVQNDH

SEQ ID NO: 35 - protein, 32 aa, synthetic construct
QAFVYHPNNPNFGGNTFNYSWMLSSAQAQDRT

SEQ ID NO: 36 - protein, 31 aa, synthetic construct
QGLIYKPKNPAFGGDTFNYQWLASSAESQNK

SEQ ID NO: 37 - protein, 8 aa, synthetic construct
GTMTFQFR

SEQ ID NO: 38 - protein, 19 aa, synthetic construct
GTMTFQFRNPNFGGNPNNG

SEQ ID NO: 39 - protein, 29 aa, synthetic construct
GTMTFQFRNPNFGGNPNNGAFLLNSAQAQ

SEQ ID NO: 40 - protein, 45 aa, synthetic construct
GTMTFQFRNPNFGGNPNNGAFLLNSAQAQNSYKDPSYNDDFGIET

SEQ ID NO: 41 - DNA, 24 bp, synthetic construct
  source 1..24: mol_type = other DNA
acggaactggaaagtcatggttcc

SEQ ID NO: 42 - DNA, 30 bp, synthetic construct
  source 1..30: mol_type = other DNA
gccattatttgggttaccaccaaagtttgg

SEQ ID NO: 43 - DNA, 30 bp, synthetic construct
  source 1..30: mol_type = other DNA
ttgggcctgagcgctatttaataaaaaagc

SEQ ID NO: 44 - DNA, 33 bp, synthetic construct
  source 1..33: mol_type = other DNA
tgtttcaataccaaagtcatcgttatagctcgg

SEQ ID NO: 45 - DNA, 25 bp, synthetic construct
  source 1..25: mol_type = other DNA
catcaccatcaccatcactaagccc

SEQ ID NO: 46 - DNA, 32 bp, synthetic construct
  source 1..32: mol_type = other DNA
cccccatatgggaaccatgactttccagttcc

SEQ ID NO: 47 - DNA, 55 bp, synthetic construct
  source 1..55: mol_type = other DNA
ccccgaattcctaatggtgatggtgatggtggtaaaaatcggttgagttattttg

SEQ ID NO: 48 - DNA, 58 bp, synthetic construct
  source 1..58: mol_type = other DNA
ggggacaagtttgtacaaaaaagcaggctacctcaggcgataaagccatgaaacgtta

SEQ ID NO: 49 - DNA, 72 bp, synthetic construct
  source 1..72: mol_type = other DNA
ggggaccactttgtacaagaaagctgggtgtttaaactcatttttcgaactgcgggtggctccaagcgctgg

SEQ ID NO: 50 - DNA, 59 bp, synthetic construct
  source 1..59: mol_type = other DNA
caaaataactcaaccgattttcatcaccatcaccatcactaagccccagcttcataagg

SEQ ID NO: 51 - DNA, 59 bp, synthetic construct
  source 1..59: mol_type = other DNA
ccttatgaagctggggcttagtgatggtgatggtgatgaaaatcggttgagttattttg

SEQ ID NO: 52 - DNA, 21 bp, synthetic construct
  source 1..21: mol_type = other DNA
agcctgcttttttgtacaaac

SEQ ID NO: 53 - DNA, 23 bp, synthetic construct
  source 1..23: mol_type = other DNA
ataaaaaattgttcggaggctgc

SEQ ID NO: 54 - protein, 30 aa, synthetic construct
GTMTFQFRNPNFGGNPNNGAFLLNSAQAQN

SEQ ID NO: 55 - protein, 35 aa, synthetic construct
GTMTFQFRNPNFGGNPNNGAFLLNSAQAQNSYKDP

SEQ ID NO: 56 - protein, 155 aa, synthetic construct
MRVKHAVVLLMLISPLSWAGTMCFQFRNPNFGGNPSNGAFLLNSAQAQNSYKDPENLYFQSSYNDDFGIETPSALDNFTQAIQSQILGGLLSNINTGKPGRMVTNDYIVDIANRDGQLQLNVTDRKTGQTSTIQVSGLQNNSTDFSAWSHPQFEK

SEQ ID NO: 57 - protein, 155 aa, synthetic construct
MRVKHAVVLLMLISPLSWAGTMTFQFRNPNFGGNPSNGAFLLNSAQAQNSYKDPENLYFQSSYNDDFGIETPSALDNFTQAIQSQILGGLLSNINTGKPGRMVTNDYIVDIANRDGQLQLNVTDRKTGQTSTIQVSGLQNNSTDFSAWSHPQFEK

SEQ ID NO: 58 - protein, 155 aa, synthetic construct
MRVKHAVVLLMLISPLSWACTMTFQFRNPNFGGNPSNGAFLLNSAQAQNSYKDPENLYFQSSYNDDFGIETPSALDNFTQAIQSQILGGLLSNINTGKPGRMVTNDYIVDIANRDGQLQLNVTDRKTGQTSTIQVSGLQNNSTDFSAWSHPQFEK

SEQ ID NO: 59 - protein, 155 aa, synthetic construct
MRVKHAVVLLMLISPLSWACTMTFQFRNPNFGGNPNNGAFLLNSAQAQNSYKDPENLYFQSSYNDDFGIETPSALDNFTQAIQSQILGGLLSNINTGKPGRMVTNDYIVDIANRDGQLQLNVTDRKTGQTSTIQVSGLQNNSTDFSAWSHPQFEK

SEQ ID NO: 60 - protein, 155 aa, synthetic construct
MRVKHAVVLLMLISPLSWAGTMTFQFRNPNFGGNPNNGAFLLNSAQAQNSYKDPSYNDDFGIETENLYFQSPSALDNFTQAIQSQILGGLLSNINTGKPGRMVTNDYIVDIANRDGQLQLNVTDRKTGQTSTIQVSGLQNNSTDFHHHHHHHHHH

SEQ ID NO: 61 - protein, 155 aa, synthetic construct
MRVKHAVVLLMLISPLSWAGTMTFQFRNPNFGGNPNNGAFLLNSAQAQNSYKDPENLYFQSSYNDDFGIETPSALDNFTQAIQSQILGGLLSNINTGKPGRMVTNDYIVDIANRDGQLQLNVTDRKTGQTSTIQVSGLQNNSTDFHHHHHHHHHH

SEQ ID NO: 62 - protein, 155 aa, synthetic construct
MRVKHAVVLLMLISPLSWAGTMTFQFRNPNFGGNPNNGAFLLNSAQAQNENLYFQSSYKDPSYNDDFGIETPSALDNFTQAIQSQILGGLLSNINTGKPGRMVTNDYIVDIANRDGQLQLNVTDRKTGQTSTIQVSGLQNNSTDFHHHHHHHHHH

SEQ ID NO: 63 - protein, 149 aa, synthetic construct
MRVKHAVVLLMLISPLSWAGTMTFQFRNPNFGGNPNNGAFLLNSAQAQNSYKDPSYNDDFGIETENLYFQSFTQAIQSQILGGLLSNINTGKPGRMVTNDYIVDIANRDGQLQLNVTDRKTGQTSTIQVSGLQNNSTDFHHHHHHHHHH

SEQ ID NO: 64 - protein, 149 aa, synthetic construct
MRVKHAVVLLMLISPLSWAGTMTFQFRNPNFGGNPNNGAFLLNSAQAQNENLYFQSYNDDFGIETPSALDNFTQAIQSQILGGLLSNINTGKPGRMVTNDYIVDIANRDGQLQLNVTDRKTGQTSTIQVSGLQNNSTDFHHHHHHHHHH

SEQ ID NO: 65 - protein, 155 aa, synthetic construct
MRVKHAVVLLMLISPLSWACTMTFQFRNPNFGGNPNNGAFLLNSAQAQNSYKDLEVLFQGPSYNDDFGIETPSALDNFTQAIQSQILGGLLSNINTGKPGRMVTNDYIVDIANRDGQLQLNVTDRKTGQTSTIQVSGLQNNSTDFSAWSHPQFEK

SEQ ID NO: 66 - protein, 156 aa, synthetic construct
MRVKHAVVLLMLISPLSWACTMTFQFRNPNFGGNPNNGAFLLNSAQAQNSYKDPSYNDDFGILEVLFQGPETPSALDNFTQAIQSQILGGLLSNINTGKPGRMVTNDYIVDIANRDGQLQLNVTDRKTGQTSTIQVSGLQNNSTDFSAWSHPQFEK

SEQ ID NO: 67 - protein, 148 aa, synthetic construct
MRVKHAVVLLMLISPLSWACTMTFQFRNPNFGGNPNNGAFLLNSAQAQNSYKDPSYNLEVLFQGPSALDNFTQAIQSQILGGLLSNINTGKPGRMVTNDYIVDIANRDGQLQLNVTDRKTGQTSTIQVSGLQNNSTDFSAWSHPQFEK

SEQ ID NO: 68 - protein, 248 aa, Citrobacter koseri
MPRAQSYKDLTHLPMPTGKIFVSVYNIQDETGQFKPYPASNFSTAVPQSATAMLVTALKDSRWFIPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEIGYTSNEPVMLCLMSAIETGVIFLINDGIDRGLWDLQNKAERQNDILVKYRHMSVPPES

SEQ ID NO: 69 - protein, 223 aa, Salmonella enterica
CLTAPPKQAAKPTLMPRAQSYKDLTHLPAPTGKIFVSVYNIQDETGQFKPYPASNFSTAVPQSATAMLVTALKDSRWFIPLERQGLQNLLNERKIIRAAQENGTVAMNNRIPLQSLTAANIMVEGSIIGYESNVKSGGVGARYFGIGADTQYQLDQIAVNLRVVNVSTGEILSSVNTSKTILSYEVQAGVFRFIDYQRLLEGEIGYTSNEPVMLCLMSAIETG

SEQ ID NO: 70 - protein, 262 aa, Citrobacter amalonaticus
CLTAPPKEAAKPTLMPRAQSYKDLTHLPIPTGKIFVSVYNIQDETGQFKPYPASNFSTAVPQSATAMLVTALKDSRWFVPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEIGYTSNEPVMLCLMSAIETGVIFLINDGIDRGLWDLQNKADRQNDILVKYRHMSVPPES

SEQ ID NO: 71 - protein, 262 aa, Citrobacter rodentium
CLTTPPKEAAKPTLMPRAQSYKDLTHLPVPTGKIFVSVYNIQDETGQFKPYPASNFSTAVPQSATAMLVTALKDSRWFIPLERQGLQNLLNERKIIRAAQENGTVAINNRIPLPSLTAANIMVEGSIIGYESNVKSGGAGARYFGIGADTQYQLDQIAVNLRVVNVSTGEILSSVNTSKTILSYEVQAGVFRFIDYQRLLEGEIGYTSNEPVMLCLMSAIETGVIFLINDGIDRGLWDLQNKADRQNDILVKYRQMSVPPES

SEQ ID NO: 72 - protein, 262 aa, Enterobacter asburiae
CLTAPPKEAAKPTLMPRAQSYRDLTHLPAPTGKIFVSVYNIQDETGQFKPYPASNFSTAVPQSATAMLVTALKDSHWFIPLERQGLQNLLNERKIIRAAQENGTVANNNRMPLQSLAAANVMIEGSIIGYESNVKSGGVGARYFGIGADTQYQLDQIAVNLRVVNVSTGEVLSSVNTSKTILSYEVQAGVFRFIDYQRLLEGEIGYTSNEPVMMCLMSAIETGVIFLINDGIDRGLWDLQNKADAQNPVLVKYRDMSVPPES

SEQ ID NO: 73 - protein, 262 aa, Yokenella regensburgei
CLTAPPKEAAKPTLMPRAQSYRDLTHLPLPSGKVFVSVYNIQDETGQFKPYPASNFSTAVPQSATAMLVTALKDSRWFVPLERQGLQNLLNERKIIRAAQENGTVADNNRIPLQSLTAANVMIEGSIIGYESNVKSGGVGARYFGIGADTQYQLDQIAVNLRVVNVSTGEVLSSVNTSKTILSYEVQAGVFRFVDYQRLLEGEIGYTSNEPVMLCLMSAIETGVIYLINDGIERGLWDLQQKADVDNPILARYRNMSAPPES

SEQ ID NO: 74 - protein, 262 aa, unidentified
  source 1..262: note = Cronobacter pulveris
CLTAPPKEAAKPTLMPRAQSYRDLTNLPDPKGKLFVSVYNIQDETGQFKPYPASNFSTAVPQSATSMLVTALKDSRWFIPLERQGLQNLLNERKIIRAAQENGTVAENNRMPLQSLVAANVMIEGSIIGYESNVKSGGVGARYFGIGGDTQYQLDQIAVNLRVVNVSTGEVLSSVNTSKTILSYEVQAGVFRFIDYQRLLEGEIGYTANEPVMLCLMSAIETGVIHLINDGINRGLWELKNKGDAKNTILAKYRSMAVPPES

SEQ ID NO: 75 - protein, 262 aa, Rahnella aquatilis
CLTAAPKEAARPTLLPRAPSYTDLTHLPSPQGRIFVSVYNIQDETGQFKPYPACNFSTAVPQSATAMLVSALKDSKWFIPLERQGLQNLLNERKIIRAAQENGSVAINNQRPLSSLVAANILIEGSIIGYESNVKSGGVGARYFGIGASTQYQLDQIAVNLRAVDVNTGEVLSSVNTSKTILSYEVQAGVFRFIDYQRLLEGELGYTTNEPVMLCLMSAIESGVIYLVNDGIERNLWQLQNPSEINSPILQRYKNNIVPAES

SEQ ID NO: 76 - protein, 259 aa, Kluyvera ascorbata
CITSPPKQAAKPTLLPRSQSYQDLTHLPEPQGRLFVSVYNISDETGQFKPYPASNFSTSVPQSATAMLVSALKDSNWFIPLERQGLQNLLNERKIIRAAQENGTVAVNNRTQLPSLVAANILIEGSIIGYESNVKSGGAGARYFGIGASTQYQLDQIAVNLRVVNVSTGEVLSSVNTSKTILSYEFQAGVFRYIDYQRLLEGEVGYTVNEPVMLCLMSAIETGVIYLVNDGISRNLWQLKNASDINSPVLEKYKSIIVP

SEQ ID NO: 77 - protein, 259 aa, Hafnia alvei
CLTAPPKQAAKPTLMPRAQSYQDLTHLPEPAGKLFVSVYNIQDETGQFKPYPASNFSTAVPQSATAMLVSALKDSGWFIPLERQGLQNLLNERKIIRAAQENGTAAVNNQHQLSSLVAANVLVEGSIIGYESNVKSGGAGARFFGIGASTQYQLDQIAVNLRVVDVNTGQVLSSVNTSKTILSYEVQAGVFRYIDYQRLLEGEIGYTTNEPVMLCVMSAIETGVIYLVNDGINRNLWTLKNPQDAKSSVLERYKSTIVP

SEQ ID NO: 78 - protein, 255 aa, unidentified
  source 1..255: note = Enterobacteriaceae bacterium strain FGI 57
CITTPPQEAAKPTLLPRDATYKDLVSLPQPRGKIYVAVYNIQDETGQFQPYPASNFSTSVPQSATAMLVSSLKDSRWFVPLERQGLNNLLNERKIIRAAQQNGTVGDNNASPLPSLYSANVIVEGSIIGYASNVKTGGFGARYFGIGGSTQYQLDQVAVNLRIVNVHTGEVLSSVNTSKTILSYEIQAGVFRFIDYQRLLEGEAGFTTNEPVMTCLMSAIEEGVIHLINDGINKKLWALSNAADINSEVLTRYRK

SEQ ID NO: 79 - protein, 258 aa, Plesiomonas shigelloides
ITEVPKEAAKPTLMPRASTYKDLVALPKPNGKIIVSVYSVQDETGQFKPLPASNFSTAVPQSGNAMLTSALKDSGWFVPLEREGLQNLLNERKIIRAAQENGTVAANNQQPLPSLLSANVVIEGAIIGYDSDIKTGGAGARYFGIGADGKYRVDQVAVNLRAVDVRTGEVLLSVNTSKTILSSELSAGVFRFIEYQRLLELEAGYTTNEPVMMCMMSALEAGVAHLIVEGIRQNLWSLQNPSDINNPIIQRYMKEDVP

SEQ ID NO: 80 - protein, 248 aa, unidentified
  source 1..248: note = Vibrio fischeri ES114
PETSESPTLMQRGANYIDLISLPKPQGKIFVSVYDFRDQTGQYKPQPNSNFSTAVPQGGTALLTMALLDSEWFYPLERQGLQNLLTERKIIRAAQKKQESISNHGSTLPSLLSANVMIEGGIVAYDSNIKTGGAGARYLGIGGSGQYRADQVTVNIRAVDVRSGKILTSVTTSKTILSYEVSAGAFRFVDYKELLEVELGYTNNEPVNIALMSAIDSAVIHLIVKGVQQGLWRPANLDTRNNPIFKKY

SEQ ID NO: 81 - protein, 248 aa, Aliivibrio logei
PDASESPTLMQRGATYLDLISLPKPQGKIYVSVYDFRDQTGQYKPQPNSNFSTAVPQGGTALLTMALLDSEWFYPLERQGLQNLLTERKIIRAAQKKQESISNHGSTLPSLLSANVMIEGGIVAYDSNIKTGGAGARYLGIGGSGQYRADQVTVNIRAVDVRSGKILTSVTTSKTILSYELSAGAFRFVDYKELLEVELGYTNNEPVNIALMSAIDSAVIHLIVKGIEEGLWRPENQNGKENPIFRKY

SEQ ID NO: 82 - protein, 254 aa, unidentified
  source 1..254: note = Photobacterium sp. AK15
PETSKEPTLMARGTAYQDLVSLPLPKGKVYVSVYDFRDQTGQYKPQPNSNFSTAVPQGGAALLTTALLDSRWFMPLEREGLQNLLTERKIIRAAQKKDEIPTNHGVHLPSLASANIMVEGGIVAYDTNIQTGGAGARYLGVGASGQYRTDQVTVNIRAVDVRTGRILLSVTTSKTILSKELQTGVFKFVDYKDLLEAELGYTTNEPVNLAVMSAIDAAVVHVIVDGIKTGLWEPLRGEDLQHPIIQEYMNRSKP

SEQ ID NO: 83 - protein, 261 aa, Aeromonas veronii
CATHIGSPVADEKATLMPRSVSYKELISLPKPKGKIVAAVYDFRDQTGQYLPAPASNFSTAVTQGGVAMLSTALWDSQWFVPLEREGLQNLLTERKIVRAAQNKPNVPGNNANQLPSLVAANILIEGGIVAYDSNVRTGGAGAKYFGIGASGEYRVDQVTVNLRAVDIRSGRILNSVTTSKTVMSQQVQAGVFRFVEYKRLLEAEAGFSTNEPVQMCVMSAIESGVIRLIANGVRDNLWQLADQRDIDNPILQEYLQDNAP

SEQ ID NO: 84 - protein, 239 aa, unidentified
  source 1..239: note = Shewanella sp. ECSMB14101
ASSSLMPKGESYYDLINLPAPQGVMLAAVYDFRDQTGQYKPIPSSNFSTAVPQSGTAFLAQALNDSSWFIPVEREGLQNLLTERKIVRAGLKGDANKLPQLNSAQILMEGGIVAYDTNVRTGGAGARYLGIGAATQFRVDTVTVNLRAVDIRTGRLLSSVTTTKSILSKEITAGVFKFIDAQELLESELGYTSNEPVSLCVASAIESAVVHMIADGIWKGAWNLADQASGLRSPVLQKY

SEQ ID NO: 85 - protein, 233 aa, Pseudomonas putida
QDSETPTLTPRASTYYDLINMPRPKGRLMAVVYGFRDQTGQYKPTPASSFSTSVTQGAASMLMDALSASGWFVVLEREGLQNLLTERKIIRASQKKPDVAENIMGELPPLQAANLMLEGGIIAYDTNVRSGGEGARYLGIDISREYRVDQVTVNLRAVDVRTGQVLANVMTSKTIYSVGRSAGVFKFIEFKKLLEAEVGYTTNEPAQLCVLSAIESAVGHLLAQGIEQRLWQV

SEQ ID NO: 86 - protein, 234 aa, unidentified
  source 1..234: note = Shewanella violacea DSS12
MPKSDTYYDLIGLPHPQGSMLAAVYDFRDQTGQYKAIPSSNFSTAVPQSGTAFLAQALNDSSWFVPVEREGLQNLLTERKIVRAGLKGEANQLPQLSSAQILMEGGIVAYDTNIKTGGAGARYLGIGVNSKFRVDTVTVNLRAVDIRTGRLLSSVTTTKSILSKEVSAGVFKFIDAQDLLESELGYTSNEPVSLCVAQAIESAVVHMIADGIWKRAWNLADTASGLNNPVLQKY

SEQ ID NO: 87 - protein, 245 aa, Marinobacterium jannaschii
LTRRMSTYQDLIDMPAPRGKIVTAVYSFRDQSGQYKPAPSSSFSTAVTQGAAAMLVNVLNDSGWFIPLEREGLQNILTERKIIRAALKKDNVPVNNSAGLPSLLAANIMLEGGIVGYDSNIHTGGAGARYFGIGASEKYRVDEVTVNLRAIDIRTGRILHSVLTSKKILSREIRSDVYRFIEFKHLLEMEAGITTNDPAQLCVLSAIESAVAHLIVDGVIKKSWSLADPNELNSPVIQAYQQQRI

SEQ ID NO: 88 - protein, 234 aa, unidentified
  source 1..234: note = Chryseobacterium oranimense G311
PSDPERSTMGELTPSTAELRNLPLPNEKIVIGVYKFRDQTGQYKPSENGNNWSTAVPQGTTTILIKALEDSRWFIPIERENIANLLNERQIIRSTRQEYMKDADKNSQSLPPLLYAGILLEGGVISYDSNTMTGGFGARYFGIGASTQYRQDRITIYLRAVSTLNGEILKTVYTSKTILSTSVNGSFFRYIDTERLLEAEVGLTQNEPVQLAVTEAIEKAVRSLIIEGTRDKIW

SEQ ID NO: 89 - protein, 45 aa, synthetic construct
CTMTFQFRNPNFGGNPNNGAFLLNSAQAQNSYKDPSYNDDFGIET

SEQ ID NO: 90 - protein, 29 aa, synthetic construct
CTMTFQFRNPNFGGNPNNGAFLLNSAQAQ

SEQ ID NO: 91 - protein, 35 aa, synthetic construct
CTMTFQFRNPNFGGNPNNGAFLLNSAQAQNSYKDP

SEQ ID NO: 92 - protein, 35 aa, synthetic construct
GTMTFQFRNPNFGGNPSNGAFLLNSAQAQNSYKDP

SEQ ID NO: 93 - protein, 35 aa, synthetic construct
CTMTFQFRNPNFGGNPNNGAFLLNSAQAQNSYKYP

SEQ ID NO: 94 - protein, 45 aa, synthetic construct
GTMTFQFRNPNFGGNPSNGAFLLNSAQAQNSYKDPSYNDDFGIET

SEQ ID NO: 95 - protein, 35 aa, synthetic construct
CTMTFQFRNPNFGGNPSNGAFLLNSAQAQNSYKYP

SEQ ID NO: 96 - protein, 39 aa, synthetic construct
CTMTFQFRNPNFGGNPNNGAFLLNSAQAQHHHHHHHHHH

SEQ ID NO: 97 - protein, 45 aa, synthetic construct
CTMTFQFRNPNFGGNPNNGAFLLNSAQAQNSYKDPHHHHHHHHHH

SEQ ID NO: 98 - protein, 35 aa, synthetic construct
GTMTFQFRNPNFGGNPSNGAFLLNSAQAQNSYKYP

SEQ ID NO: 99 - protein, 35 aa, synthetic construct
GTMTFQFRNPNFGGNPSNGAFLLNSAQAQNSYKNP

SEQ ID NO: 100 - protein, 35 aa, synthetic construct
GTMCFQFRNPNFGGNPSNGAFLLNSAQAQNSYKDP

SEQ ID NO: 101 - protein, 35 aa, synthetic construct
GTMCFQFRNPNFGGNPSNGAFLLNSAQAQNSYKYP

SEQ ID NO: 102 - protein, 35 aa, synthetic construct
GTMCFQFRNPNFGGNPSNGAFLLNSAQAQNSYKNP

SEQ ID NO: 103 - protein, 35 aa, synthetic construct
CTMCFQFRNPNFGGNPSNGAFLLNSAQAQNSYKDP

SEQ ID NO: 104 - protein, 35 aa, synthetic construct
CTMCFQFRNPNFGGNPSNGAFLLNSAQAQNSYKNP

SEQ ID NO: 105 - protein, 35 aa, synthetic construct
CTMCFQFRNPNFGGNPSNGAFLLNSAQAQNSYKYP

SEQ ID NO: 106 - protein, 35 aa, synthetic construct
GTMTFQFRNPNFGGNPVNGAFLLNSAQAQNSYKDP

SEQ ID NO: 107 - protein, 35 aa, synthetic construct
GTMTFQFRNPNFGGNPANGAFLLNSAQAQNSYKDP

SEQ ID NO: 108 - protein, 35 aa, synthetic construct
GTMTFQFRNPNFGGNPFNGAFLLNSAQAQNSYKDP

SEQ ID NO: 109 - protein, 30 aa, synthetic construct
GTMTFQFRNPNFGGNPSNGAFLLNSAQAQN

SEQ ID NO: 110 - protein, 30 aa, synthetic construct
GTMTFQFRNPNFGGNPVNGAFLLNSAQAQN

SEQ ID NO: 111 - protein, 30 aa, synthetic construct
GTMTFQFRNPNFGGNPANGAFLLNSAQAQN

SEQ ID NO: 112 - protein, 30 aa, synthetic construct
GTMTFQFRNPNFGGNPFNGAFLLNSAQAQN

SEQ ID NO: 113 - protein, 35 aa, synthetic construct
GTMTFQFRNPNFGGNPVNGAFLLNSAQAQNSYKYP

SEQ ID NO: 114 - protein, 35 aa, synthetic construct
GTMTFQFRNPNFGGNPANGAFLLNSAQAQNSYKYP

SEQ ID NO: 115 - protein, 35 aa, synthetic construct
GTMTFQFRNPNFGGNPFNGAFLLNSAQAQNSYKYP

SEQ ID NO: 116 - protein, 35 aa, synthetic construct
GTMTFQFRNPNFGGNPSNGQFLLNSAQAQNSYKDP

SEQ ID NO: 117 - protein, 262 aa, synthetic construct
CLTAPPKEAARPTLMPRAQSYKDLTHLPAPTGKIFVSVYNIQDETGQFKPYPASNFSTAVPQSATAMLVTALKDSRWFIPLERQGLQNLLNERKIIWAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEVGYTSNEPVMLCLMSAIETGVIFLINDGIDRGLWDLQNKAERQNDILVKYRHMSVPPES

SEQ ID NO: 118 - protein, 439 aa, unidentified
  source 1..439: note = Enterobacteria phage T4
MTFDDLTEGQKNAFNIVMKAIKEKKHHVTINGPAGTGKTTLTKFIIEALISTGGTGIILAAPTHAAKKILSKLSGKEASTIHSILKINPVTYEENVLFEQKEVPDLAKCRVLICDEVSMYDRKLFKILLSTIPPWCTIIGIGDNKQIRPVEPGENTAYISPFFTHKDFYQCELTEVKRSNAPIIDVATDVRNGKWNYDKVVDGHGVRGFTGDTALRDFMVNYFSIVKSLDDLFENRVMAFTNKSVDKLNSIIRKKIFETDKDFIVGEIIVMQEPLFKTYKIDGKPVSEIIFNNGQLVRIIEAEYTSTFVKARGVPGEYLIRHWDLTVETYGDDEYYREKIKIISSDEELYKFNLFLAKTAETYKNWNKGGKAPWSDFWDAKSQFSKVKALPASTFHKAQGMSVDRAFIYTPCIHYADVELAQQLLYVGVTRGRYDVFYV

SEQ ID NO: 119 - protein, 678 aa, Rhodothermus marinus
MEELSNEQQRVLDHVLAWLERNDAPPIFILTGSAGTGKTLLIRHLVRALQDRRIHYALAAPTGRAARILSERTGDHARTLHSLIYIFDRYQLVEEADRQTDEPLSLQLHFALRSAEHDARLIIVDEASMVSDTAGEEELYRFGSGRLLNDLLTFARLIPKRDRPPTTRLLFVGDPAQLPPVGQSVSPALSAQYLRDTFGLSAETAHLRSVYRQRKGHPILETATALRNALEKGHYHTFRLPEQPPDLRPVGLEEAIETTATDFRRQNPSVLLCRTNALARKLNAAVRARLWGREGLPPQPGDLLLVNRNAPLHGLFNGDLVLVETVGPLEHRRVGRRGRPPVDLYFRDVELLYPHEKPRNRIRCKLLENLLESPDGQLSPDIIQALLIDFYRRHPSLKHGSSEFRLMLANDAYFNALHVRYGYAMTVHKAQGGEWKRATVVFNDWRHFRHAEFFRWAYTAITRAREELLTIGAPSFEALSDMRWQPAPSVPAPEQAAENATRFPLKALETYHQRLSEALTAAGIETTGVELLQYAVRYHLARADRTTRIQYYYRGDGQISRIVTLGGADDPELTQQAYALFERILSEPPADSGELPENPLLREFLERAHLRLEGSGIRIVHWKEMPYALRLYFSADGENVTIDFYYNRRGVWTHAQEVGRSSSGALFARIQSLLQADS

SEQ ID NO: 120 - protein, 496 aa, unidentified
  source 1..496: note = Cyanothece ATCC51142
MSQSVVVPDELGEIITAVIEFYQDAVDKIEPKIVFLELRKNVVDWVSRTQLKIEEKEIQATGLTRQQQTAYKEMINFIENSSEQYFRLSGYAGTGKSFLMAKVIEWLKQEDYKYSVAAPTNKAAKNLTQIARSQGIKIEATTVAKLLKLQPTIDVDTGQQSFEFNSEKELELKDYDVIIIDEYSMLNKDNFRDLQQAVKGGESKFIFVGDSSQLPPVKEKEPIVANHPDIRKSANLTQIVRYDGEIVKVAESIRRNPRWNHQTYPFETVADGTIIKLNTEDWLQQALSHFEKEDWLSNPDYVRMITWRNKTADKYNQAIREALYGENVEQLVVGDRLIAKKPVFRSLPGGKKKEKKIILNNSEECKVIETPKINYNEKYKWEFYQVKVRTDEGGMIELRILTSESEEKRQKKLKELAKRAREEENYSEKKKQWAIYYELDELFDNMAYAYALTCHKAQGSSIDNVFLLVSDMHYCRDKTKMIYTGLTRAKKCCYVG

SEQ ID NO: 121 - protein, 421 aa, Salinibacter ruber
MSTFADAPFTEDQEEAYDHVYDRLAQGERFTGLRGYAGTGKTYLVSRLVEQLLDEDCTVTVCAPTHKAVQVLSDELGDAPVQMQTLHSFLGLRLQPKQDGEYELVAEEERNFAEGVVIVDEASMIGREEWSHIQDAPFWVQWLFVGDPAQLPPVNEDPSPALDVPGPTLETIHRQAADNPILELATKIRTGADGRFGSTFEDGKGVAVTRNREEFLDSILRAFDADAFAEDATHARVLAYRNKTVRRYNREIRAERYGADADRFVEGEWLVGTETWYYDGVQRLTNSEEVRVKKAQVETFEADDQSEWTVWELKIRTPGRGLTRTIHVLHEEERERYENALERRRGKAEDDPSKWDRFFELRERFARVDYAYATTVHRAQGSTYDTVFVDHRDLRVCRGEERGALLYVAVTRPSRRLALLV

SEQ ID NO: 122 - protein, 500 aa, unidentified
  source 1..500: note = Sullfurimonas gotlandica GD1
MKILNKETYKLSLHQEEVFTQIVSQLDTKVSSILKSTNIEDYLLSLTGPAGTGKTFLTTQIAKYLVEKRKESEYPMSSDFDFTITAPTHKAVGVLSKLLRENNIQSSCKTIHSFLGIKPFIDYTTGEEKFVVDKTNKRKDRTSILIVDESSMIGNTLYEYILEAIEDKRVNVVLFIGDPYQLLPIENSKNEIYDLPNRFFLSEVVRQAENSYIIRVATKLRERIKNQDFISLQQFFQENMEDEITFFHNKEAFLEDFYKEEEWYKENKILATYKNKDVDAFNKIIRNKFWEQKGNTTPSTLLAGDMIRFKDAYTVGDITIYHNGQELQLGSTEVKYHDSLHIEYWECKSIYALEQQVFRVVNPDSEAVFNQKLQSLATKAKQAKFPDNKKLWKLYYETRNMFANVQYIHASTIHKLQGSTYDVSYIDIFSLVHNHYMSDEEKYRLLYVAITRASKDIKIFMSAFDRTSDEKVIINNQNSETMNTLKQLHDIDIILKDLDL

SEQ ID NO: 123 - protein, 450 aa, unidentified
  source 1..450: note = Vibrio phage henriette 12B8
MADFELTLGQKTVLGEVISTILKPVNLNDTSRFHTMHGPAGSGKTTVLQRIISQIPAYKTIGFCSPTHKSVKVIRRMAREAGISHRVDIRTIHSALGLVMKPVRGDEVLVKEPFAEERIYDVLIIDEAGMLNDELIMYILESQSSKVIFVGDMCQIGPIQSNLPEEDGYTPTSTDDVSKVFTEVEMMSALTEVVRQAEGSPIIQLATEFRLAQDDIYADLPRIVTNTTPDGNGIITMPNGNWVDSAVARFQSDQFKEDPDHCRIVCYTNAMVDLCNDLVRKRLFGADVPEWLEDEILVAQEMGSTWNNADELRIVSIDDHFDQQYEVPCWRMQLESVEDHKLHNALVVKGDYIEDFKFRLNAIAERANTDKNMSGMHWKEFWGMRKKFNTFKNVYAGTAHKSQGSTFDYTYVFTPDFYKFGATMTIKRLLYTAITRSRYTTYFAMNTGAQ

SEQ ID NO: 124 - protein, 421 aa, unidentified
  source 1..421: note = Vibrio phage phi-pp2
MGLTNCQQGAMDAFLESDGHMTISGPAGSGKTFLMKSILEALESKGKNVTMVTPTHQAKNVLHKATGQEVSTIHSLLKIHPDTYEDQKHFTQSGEVEGLDEIDVLVVEEASMVDEELFQITGRTMPRKCRILAVGDKYQLQPVKHDPGVISPFFTKFTTFEMNEVVRQAKDNPLIQVATEVRNGQWLRTNWSKERRQGVLHVPNVNKMLDTYLSKVNSPEDLLDYRILAYTNDCVDTFNGIIREHVYNTSEPFIPGEYLVTQMPVMVSNGKYPVCVIENGEVVKILDVRQKTIDGMLPKVDNEAFDVAVLTVEKEDGNVYEFTVLWDDLQKERFARYLSVAAGTYKSMRGNTKRYWRAFWGLKEQMIETKSLGASTVHKSQGTTVKGVCLYTQDMGYAEPEILQQLVYVGLTRPTDWALYN

SEQ ID NO: 125 - protein, 434 aa, unidentified
  source 1..434: note = Aeromonas phage 65
MSESEITLTPSQNMAVNEVKNGTGHITISGPPGSGKTFLVKYLIKMLGDELGTVLAAPTHQAKIVLTEMSGIEACTIHSLMKIHPETLEDIQIFDQSKLPDLSNIRYLIVEEASMHSKTLFKITMKSIPPTCRIIAIGDKDQIQPEEHAQGELSPYFTDPRFSQIRLTDIMRQSLDNPIIQVATKIREGGWIEPNWNRDTKTGVYKVSGITDLVNSYLRAVKTPEDLTKYRFLAYTNKVVNKVNSIVREHVYKTKLPFIEGEKIVLQEPVMVEHEDDTIETIFTNGEVVTINEIEVFDRTIRIDGSPEFKVNAAKLSVSSDYSGIEHDFCVLYGSESRLEFEYQLSESAGNIKQMGKGGNQRSAWKSFWAAKKMFIETKSLGASTIHKSQGSTVKGVWLALHDIHYADEELKQQLVYVGVTRPTDFCLYFDGTK

SEQ ID NO: 126 - protein, 420 aa, unidentified
  source 1..420: note = Aeromonas phage CC2
MAVDAVQSGTGHITISGPPGSGKTFLVKYIIKMLGDELGTVLAAPTHQAKIVLTEMSGIEACTIHSLMKIHPETLEDIQIFDQSKMPDLSTVRYLIIEEASMHSKALFNITMKSIPPTCRIIAIGDKDQIQPVDHAPGELSPYFTDSRFTQIRMTDIMRQSLDNPIIQVATTIREGGWIYQNWNKEKKSGVYKVKSITDLINSYLRVVKTPEDLTKYRFLAFTNKVVDKVNSIVRKHVYKTDLPFIEGEKLVLQEPVMVEYDDDTIETIFTNGEVVTVDEIEVSDMNIRIDGSPAFSISVAKLKVTSDFSGVTHDIMSVYGEDSKAEFNYQLSEAAAVIKQMQRGQTKAAWASFWDAKKTFTETKSLGACTIHKSQGSTVKGVWLGLHDISYADTDLQQQLVYVGVTRPTDFCLYFDGSK

SEQ ID NO: 127 - protein, 443 aa, unidentified
  source 1..443: note = Cronobacter phage vB CsaM GAP161
MSELTFDDLSDDQKSAHDRVIHNIQNAIHTTITGGPGVGKTTLVKFVFNTLKGLGISGIWLTAPTHQAKNVLAAATGMDATTIHSALKISPVTNEELRVFEQQKGKKAPDLSTCRVFVVEEVSMVDMDLFRIIRRSIPSNAVILGLGDKDQIRPVNADGRVELSPFFDEEIFDVIRMDKIMRQAEGNPIIQVSRAVRDGKMLKPMSVGDLGVFQHANAVDFLRQYFRRVKTPDDLIENRMFAYTNDNVDKLNATIRKHLYKTTEPFILDEVIVMQEPLVQEMRLNGQIFTEIVYNNNEKIRVLEIIPRREVIKAEKCDEKIEIEFYLLKTVSLEEETEAQIQVVVDPVMKDRLGNYLAYVASTYKRIKQQTGYKAPWHSFWAIKNKFQDVKPLPVCTYHKSQGSTYDHAYMYTRDAYAFADYDLCKQLIYVGVTRARYTVDYV

SEQ ID NO: 128 - protein, 442 aa, unidentified
  source 1..442: note = Klebsiella phage KP15
MSELTFDDLSEDQKNAHDRVIKNIRNKIHTTITGGPGVGKTTLVKFVFETLKKLGISGIWLTAPTHQAKNVLSEAVGMDATTIHSALKISPVTNEELRVFEQQKGKKAADLSECRVFVVEEVSMVDKELFRIIKRTIPSCAVILGLGDKDQIRPVNTEGITELSPFFDEEIFDVIRMDKIMRQAEGNPIIQVSRAIRDGKPLMPLMNGELGVMKHENASDFLRRYFSRVKTPDDLNNNRMFAYTNANVDKLNAVIRKHLYKTDQPFIVGEVVVMQEPLVTEGRVNGVSFVEVIYNNNEQIKILEIIPRSDTIKADRCDPVQIDYFLMKTESMFEDTKADIQVIADPVMQERLGDYLNYVAFQYKKMKQETGYKAPWYSFWQIKNKFQTVKALPVCTYHKGQGSTYDHSYMYTRDAYAYADYELCKQLLYVGTTRARFTVDYV

SEQ ID NO: 129 - protein, 438 aa, unidentified
  source 1..438: note = Stenotrophomonas phage IME13
MVTYDDLTVGQKDAIEKALQAMRTKRHITIRGPAGSGKTTMTRFLLERLFQTGQQGIVLTAPTHQAKKELSKHALRKSYTIQSVLKINPSTLEENQIFEQKGTPDFSKTRVLICDEVSFYTRKLFDILMRNVPSHCVVIGIGDKAQIRGVSEDDTHELSPFFTDNRFEQVELTEVKRHQGPIIEVATDIRNGKWIYEKLDDSGNGVKQFHTVKDFLSKYFERTKTPNDLLENRIMAYTNNSVDKLNSVIRKQLYGANAAPFLPDEILVMQEPLMFDIDIGGQTLKEVIFNNGQNVRVINVKPSRKTLKAKGVGEIEVECTMLECESYEEDEDDYRRAWFTVVHDQNTQYAINEFLSIIAEKYRSREVFPNWKDFWAIRNTFVKVRPLGAMTFHKSQGSTFDNAYLFTPCLHQYCRDPDVAQELIYVGNTRARKNVCFV

SEQ ID NO: 130 - protein, 442 aa, unidentified
  source 1..442: note = Acinetobacter phage Ac42
MNFEDLTEGQKNAYTAAIKAIETVPSSSAEKRHLTINGPAGTGKTTLTKFLIAELIRRGERGVYLAAPTHQAKKVLSQHAGMEASTIHSLLKINPTTYEDSTTFEQKDVPDMSECRVLICDEASMYDLKLFQILMSSIPLCCTVIALGDIAQIRPVEPGAFEGQVSPFFTYEKFEQVSLTEVMRSNAPIIDVATSIRTGNWIYENVIDGAGVHNLTSERSVKSFMEKYFSIVKTPEDLFENRLLAFTNKSVDDLNKIVRKKIYNTLEPFIDGEVLVMQEPLIKSYTYEGKKVSEIVFNNGEMVKVLCCSQTSDEISVRGCSTKYMVRYWQLDLQSLDDPDLTGSINVIVDEAEINKLNLVLGKSAEQFKSGAVKAAWADWWKLKRNFHKVKALPCSTIHKSQGTSVDNVFLYTPCIHKADSQLAQQLLYVGATRARHNVYYI

SEQ ID NO: 131 - protein, 442 aa, unidentified
  source 1..442: note = Shigella phage SP18
MIKFEDLNTGQKEAFDYITEAIQRRSGECITLNGPAGTGKTTLTKFVIDHLVRNGVMGIVLAAPTHQAKKVLSKLSGQTANTIHSILKINPTTYEDQNIFEQREMPDMSKCNVLVCDEASMYDGSLFKIICNSVPEWCTILGIGDMHQLQPVDPGSTQQKISPFFTHPKFKQIHLTEVMRSNAPIIEVATEIRNGGWFRDCMYDGHGVQGFTSQTALKDFMVNYFGIVKDADMLMENRMYAYTNKSVEKLNNIIRRKLYETDKAFLPYEVLVMQEPHMKELEFEGKKFSETIFNNGQLVRIKDCKYTSTILRCKGESHQLVINYWDLEVESIDEDEEYQVDRIKVLPEDQQPKFQAYLAKVADTYKQMKAAGKRPEWKDFWKARRTFLKVRALPVSTIHKAQGVSVDKAFIYTPCIHMAEASLASQLAYVGITRARYDAYYV

SEQ ID NO: 132 - protein, 439 aa, unidentified
  source 1..439: note = Yersinia phage phiR1-RT
MITYDDLTDGQKSAFDNTMEAIKNKKGHITINGPAGTGKTTLTKFIIDHLIKTGEAGIILCAPTHQAKKVLSKLSGMDASTIHSVLKINPTTYEENQIFEQREVPDLAACRVLICDEASFYDRKLFGIILATVPSWCTVIALGDKDQLRPVTPGESEQQLSPFFSHAKFKQVHLTEIKRSNGPIIQVATDIRNGGWLSENIVDGEGVHAFNSNTALKDFMIRYFDVVKTADDLIESRMLAYTNKSVDKLNGIIRRKLYETDKPFINGEVLVMQEPLMKELEFDGKKFHEIVFNNGQLVKILYASETSTFISARNVPGEYMIRYWNLEVETADSDDDYATSQIQVICDPAEMTKFQMFLAKTADTYKNSGVKAYWKDFWSVKNKFKKVKALPVSTIHKSQGCTVNNTFLYTPCIHMADAQLAKQLLYVGATRARTNLYYI

SEQ ID NO: 133 - protein, 441 aa, unidentified
  source 1..441: note = Salmonella phage S16
MITFEQLTSGQKLAFDETIRAIKEKKNHVTINGPAGTGKTTLTKFIMEHLVSTGETGIILTAPTHAAKKVLTKLSGMEANTIHKILKINPTTYEESMLFEQKEVPDLASCRVLICDEASMWDRKLFKILMASIPKWCTIVAIGDVAQIRPVDPGETEAHISPFFIHKDFKQLNLTEVMRSNAPIIDVATDIRNGSWIYEKTVDGHGVHGFTSTTALKDFMMQYFSIVKSPEDLFENRMLAFTNKSVDKLNSIIRRRLYQTEEAFVVGEVIVMQEPLMRELVFEGKKFHETLFTNGQYVRILSADYTSSFLGAKGVSGEHLIRHWVLDVETYDDEEYAREKINVISDEQEMNKFQFFLAKTADTYKNWNKGGKAPWSEFWDAKRKFHKVKALPCSTFHKAQGISVDSSFIYTPCIHVSSDNKFKLELLYVGATRGRHDVFFV

(19) United States
(12) Patent Application Publication
(10) Pub. No.: US 2025/0382602 A1
(43) Pub. Date: Dec. 18, 2025

(54) NOVEL MODIFIED PROTEIN PORES AND ENZYMES

(71) Applicant: Oxford Nanopore Technologies PLC, Oxford (GB)

(72) Inventors: Rebecca Victoria Bowen, Oxford (GB); Mark John Bruce, Oxford (GB); Elizabeth Jayne Wallace, Oxford (GB); Paul Richard Moody, Oxford (GB); Francis Bursa, Oxford (GB); David Christopher Page, Oxford (GB); Majid Mosayebi, Oxford (GB); Andrew John Heron, Oxford (GB); Alberto Riera, Oxford (GB); Christopher Peter Youd, Oxford (GB); Richard Charles Foster, Oxford (GB)

(73) Assignee: Oxford Nanopore Technologies PLC, Oxford (GB)

(21) Appl. No.: 18/856,114

(22) PCT Filed: Apr. 14, 2023
(86) PCT No.: PCT/EP2023/059821

§ 371 Date: Oct. 11, 2024

(30) Foreign Application Priority Data
Apr. 14, 2022 (GB) 2205617.0

(51) Int. Cl. C12N 9/90; C12Q 1/6869

(52) U.S. Cl. CPC C12N 9/90; C12Q 1/6869

(57) ABSTRACT

The present invention relates to modified Dda helicases which can be used to control the movement of analytes such as polynucleotides. The modified Dda helicases are used in analyte detection and characterisation. The present invention also relates to novel protein pores and their uses in analyte detection and characterisation. The invention particularly relates to an isolated pore complex formed by a CsgG-like pore and a modified CsgF peptide, or a homologue or mutant thereof, thereby incorporating an additional channel constriction or reader head in the nanopore.

### RELATED APPLICATIONS

This application is a national stage filing under 35 U.S.C. § 371 of international application number PCT/EP2023/059821, filed Apr. 14, 2023, which claims the benefit of United Kingdom application number GB 2205617.0, filed Apr. 14, 2022, each of which is herein incorporated by reference in its entirety.

### REFERENCE TO AN ELECTRONIC SEQUENCE LISTING

The contents of the electronic sequence listing (0036670156US00-SUBSEQ-KZM.xml; Size: 140,058 bytes; and Date of Creation: Oct. 15, 2024) is herein incorporated by reference in its entirety.

### TECHNICAL FIELD

The present invention relates to modified Dda helicases which can be used to control the movement of analytes such as polynucleotides. The modified Dda helicases are used in analyte detection and characterisation. The present invention also relates to novel protein pores or pore complexes and their uses in analyte detection and characterisation.

### BACKGROUND

Two of the essential components of analyte, especially polymer, characterization using nanopore sensing are (1) the control of polymer movement through the pore and (2) the discrimination of the composing building blocks as the polymer is moved through the pore. During nanopore sensing, the narrowest part of the pore typically corresponds to the most discriminating part of the nanopore with respect to the change in measurement signal as a function of the analyte moving with respect to the nanopore. CsgG was identified as an ungated, non-selective protein secretion channel from Escherichia coli (Goyal et al., 2014) and has been used as a nanopore for detecting and characterising analytes. Mutations to the wild-type CsgG pore that improve the properties of the pore in this context have also been disclosed (WO2016/034591, WO2017/149316, WO2017/149317, WO2017/149318, WO2018/211241, and WO2019/002893, incorporated by reference herein in their entirety). WO2015/055981, WO2015/166276 and WO2016/055777, also incorporated by reference herein in their entirety, describe polynucleotide binding proteins, specifically Dda helicases, which can be used to control the movement of analytes with respect to a transmembrane protein pore such as the CsgG pores described herein.

### SUMMARY OF THE INVENTION

The inventors have surprisingly identified specific Dda mutants which have an improved ability to control the movement of an analyte through a pore. When sequencing a polynucleotide using a pore, the system jointly estimates the number and identity of bases/nucleotides passing through the pore. Better control over variability in the speed of movement can reduce one of the sources of statistical noise and simplify the estimation task. Runs of consecutive short dwells of a polynucleotide in the pore may trigger a failure to call the underlying nucleotides/bases resulting in a deletion error. Unusually long dwells may lead to insertion errors. Ensuring that each nucleotide/base spends a sufficient time interval in the pore is helpful for resolving statistical uncertainty in the nucleotide/base identity from noisy signal levels. Further information can be extracted from dependence of dwell times on nucleotide/base identities, for example via interactions with the motor enzyme. Reducing the overall variability in dwell times can help to extract more precise information through this channel. During regions in which signal levels provide limited information about movement (e.g., long homopolymer regions) multi-nucleotide/base dwell times can be used to infer the number of bases traversing the pore. Reducing variability in dwell times can make these inferences more precise.

In some embodiments the mutants of the invention display improved accuracy when used in methods of controlling the movement of an analyte through a transmembrane pore and in methods of characterising an analyte using a transmembrane pore. In the context of analyte characterisation (particularly polynucleotides), accuracy is interpreted to mean raw read simplex accuracy; that is a single pass of a single molecule through a transmembrane pore. Accuracy is a useful measure to track platform improvements of sequencing devices. Accuracy can also refer to consensus accuracy or to the accuracy in detecting something specific such as a mutation in a polynucleotide analyte for example. Additionally or alternatively, accuracy is interpreted to mean the percentage of bases above a certain confidence level, where the confidence level has been pre-calibrated. In some embodiments the mutants of the invention display improved accuracy with minimal to no changes in speed. In some embodiments accuracy is improved to give less than 10% error, less than 5% error, less than 4% error, less than 3% error, less than 2% error, less than 1% error, less than 0.1% error. The mutants identified by the inventors typically comprise a combination of mutations, namely one or more modifications in the part of the mutant which interacts with a transmembrane pore. Accuracy may also by influenced by the speed which the polymer translocates the pore under enzyme control and the speed may be altered by altering the concentration of ATP provided to the enzyme. The inventors have surprisingly realised that the enzyme can exhibit changes in speed during successive polymer translocations within the same sequencing run under the same conditions which can give rise to a decrease in accuracy.

Accuracy may be influenced by a number of factors such as the nanopore shape and composition, the enzyme as well as the interaction between the enzyme and nanopore. It is also influenced by the speed at which the polymer translocates the pore under enzyme control and the translocation speed may be increased or lowered by altering the concentration of ATP provided to the enzyme. The inventors have surprisingly realised that changes in speed occur during successive polymer translocations within the same sequencing run under the same sequencing conditions, which can give rise to a decrease in sequencing accuracy. The variation in sequencing speed for a number of polymers may be measured to obtain a normalised speed distribution and the inventors have surprisingly realised that some modified enzymes can give rise to a lower normalised speed distribution and therefore an increased sequencing accuracy.

The invention provides:
a modified DNA dependent ATPase (Dda) helicase, wherein the helicase comprises a modification or substitution at one or more of the positions corresponding to amino acid positions 55, 114, 156, 177, 210, 221, 350 and 358 in Dda 1993;
a construct comprising a helicase of the invention and an additional polynucleotide binding moiety, wherein the helicase is attached to the polynucleotide binding moiety and the construct has the ability to control the movement of an analyte;
a polynucleotide which comprises a sequence which encodes a helicase of the invention or a construct of the invention;
a vector which comprises a polynucleotide of the invention operably linked to a promoter;
a host cell comprising a vector of the invention;
a method of making a helicase of the invention or a construct of the invention, which comprises expressing a polynucleotide of the invention, transfecting a cell with a vector of the invention or culturing a host cell of the invention;
a method of controlling the movement of an analyte, comprising contacting the analyte with a helicase of the invention or a construct of the invention and thereby controlling the movement of the analyte;
a method of characterising a target analyte, comprising:
   (a) contacting the target analyte with a transmembrane pore and a helicase of the invention or a construct of the invention such that the helicase or construct controls the movement of the target analyte through the pore; and
   (b) taking one or more measurements as the polynucleotide moves with respect to the pore wherein the measurements are indicative of one or more characteristics of the target analyte and thereby characterising the target analyte;
a method of forming a sensor for characterising a target analyte, comprising forming a complex between (a) a pore and (b) a helicase of the invention or a construct of the invention and thereby forming a sensor for characterising the target analyte;
a sensor for characterising a target analyte, comprising a complex between (a) a pore and (b) a helicase of the invention or a construct of the invention;
use of a helicase of the invention or a construct of the invention to control the movement of a target analyte through a pore;
a kit for characterising a target analyte comprising
   (a) a pore and a helicase of the invention or a construct of the invention; or
   (b) a helicase of the invention or a construct of the invention and one or more loading moieties;
an apparatus for characterising target analytes in a sample, comprising (a) a plurality of pores and (b) a plurality of helicases of the invention or a plurality of constructs of the invention;
a method of producing a helicase of the invention, comprising:
   (a) providing a helicase; and
   (b) modifying the helicase to produce a helicase of the invention;
a method of producing a construct of the invention, comprising attaching a helicase of the invention to an additional polynucleotide binding moiety and thereby producing the construct; a series of two or more helicases attached to a polynucleotide, wherein at least one of the two or more helicases is a helicase of the invention; and a method of improving the movement of a target analyte with respect to a transmembrane pore when the movement is controlled by a DNA dependent ATPase (Dda) helicase, wherein the DNA dependent ATPase (Dda) helicase is modified to comprise a substitution at one or more of the positions corresponding to amino acid positions 55, 114, 156, 177, 210, 221, 350 and 358 in Dda 1993 and/or the corresponding to amino acid position 40 in Dda 1993 which improves the movement of the target analyte with respect to the transmembrane pore.

The inventors have also surprisingly identified new transmembrane pore mutations which improve or alter the speed at which an analyte passes through/relative to it, preferably wherein the movement of the analyte is under the control of a polynucleotide binding protein. In one embodiment of the invention the transmembrane pore mutation increases the speed at which an analyte passes through/relative to it. In another embodiment the transmembrane pore mutation decreases the speed at which an analyte passes through/relative it. The speed at which an analyte passes through/relative to the pore may be increased by 5%, 10%, 15%, 20%, 25%, 30%, 35%, 40%, 45%, 50%, 55%, 60%, 65%, 70%, 75%, 80%, 85%, 90%, 100%, 150%, 200% or 300% or greater relative to the speed at which the analyte moves with respect to a pore which does not comprise the mutation of the invention. The speed at which an analyte passes through/relative to the pore may be decreased by 5%, 10%, 15%, 20%, 25%, 30%, 35%, 40%, 45%, 50%, 55%, 60%, 65%, 70%, 75%, 80%, 85%, 90% relative to the speed at which the analyte moves with respect to a pore which does not comprise the mutation of the invention. The inventors have surprisingly found that these alterations in speed, such as increases or decreases in speed, caused by modifications to the pore, have minimal or no effect on accuracy readings. This is particularly advantageous in a method of characterising an analyte wherein an analyte is contacted with the pore and a polynucleotide binding protein, such as a helicase of the invention, such that the polynucleotide binding protein controls the movement of the target analyte through/relative to the pore. In one embodiment, the mutant pore interacts with the polynucleotide binding protein in a different way to other transmembrane pores that do not comprise the mutation. The pore mutants may alter the distribution of speeds by which the DNA translocates through the pore such that the distribution of speeds is tighter leading to reduced sequencing error when compared to other transmembrane pores that do not comprise the mutation. In a preferred embodiment of the invention the modified DNA-dependent ATPase (Dda) helicase of the invention is used to control the movement of an analyte such as a polynucleotide through the transmembrane pore of the invention.

The invention provides an isolated CsgG pore or a homologue or mutant thereof, or an isolated pore complex comprising a CsgG pore, or a homologue or mutant thereof, and a modified CsgF peptide, or a homologue or mutant thereof, wherein the CsgG pore comprises at least one monomer comprising a modification at one or more of positions W97, Q100, E101, N102, and T104 in SEQ ID NO: 117;

In one aspect, the CsgF peptide comprises a CsgG-binding region, and a region that forms a constriction in the pore. In one aspect the CsgF peptide is a truncated CsgF peptide lacking the C-terminal head domain of CsgF. In another aspect the CsgF peptide is a truncated CsgF peptide lacking the C-terminal head and a portion of the neck domain of CsgF. In another aspect the CsgF peptide is a truncated CsgF peptide lacking the C-terminal head and neck domains of CsgF. The CsgG/CsgF pore is also referred to herein as a pore complex and as an isolated pore complex. The isolated pore complex comprises a CsgG pore, or a homologue or mutant thereof, and a modified CsgF peptide, or a homologue or mutant thereof, in particular truncated CsgF fragments, or homologues or mutants thereof. In one embodiment, said modified CsgF peptide, or homologues or mutants, is located in the lumen of the CsgG pore, or homologues or mutants thereof. In another embodiment, said isolated pore complex has two or more channel constrictions, one located or provided by the CsgG pore, formed by its constriction loop, and another additional channel constriction or reader head, introduced by the modified CsgF peptide or its homologues or mutants. In one embodiment, said CsgG-pore or CsgG-like pore, is not a wild-type pore, it is a mutant CsgG pore, with in particular embodiments mutations being present, for example, in said channel constriction loop. In other embodiments the mutations are alternatively or additionally present at the top of the pore, at a region where the pore interacts with a polynucleotide binding protein. The mutations may affect how the polynucleotide binding protein interacts with the pore and/or how the pore interacts with the polynucleotide binding protein. In another embodiment, the isolated pore complex, comprising the modified CsgF peptide, or a homologue or mutant thereof, has a CsgF channel constriction with a diameter in the range from 0.5 nm to 2.0 nm. In one embodiment, the pore complex comprises: (i) a CsgG pore comprising a first opening, a mid-section comprising a beta barrel, a second opening, and a lumen extending from the first opening through the mid-section to the second opening, wherein a luminal surface of the mid-section defines a CsgG constriction; and (ii) a plurality of modified CsgF peptides, each having a CsgF constriction region and a CsgF binding region (also referred to herein as a CsgG-binding domain or region of CsgF), wherein the modified CsgF peptides form a CsgF constriction within the beta barrel of the CsgG pore and wherein the CsgG constriction and the CsgF constriction are co-axially spaced apart within the beta barrel of the CsgG pore. The luminal surface of the CsgG pore may comprise one or more loop regions of CsgG monomers that define the CsgG constriction. The CsgF constriction region and the CsgF binding region typically correspond to a N-terminal portion of a CsgF mature peptide. In one embodiment, the pore complex excludes CsgA, CsgB and CsgE.

One embodiment relates to a pore comprising a CsgG pore and a modified CsgF peptide, wherein the modified CsgF peptide is bound to CsgG and forms a constriction in the pore and wherein the pore is mutated to alter the interaction of the pore and a polynucleotide binding enzyme and/or said pore is mutated to improve the speed at which an analyte passes through the pore. In one embodiment of the invention, the speed at which an analyte passes through the pore is increased. In another embodiment of the invention, the speed at which an analyte passes through the pore is decreased.

Another embodiment relates to the isolated pore complex wherein the modified CsgF peptide and the CsgG pore or a monomer of said pore, or homologues or mutants thereof, are covalently coupled. And even more particularly, said coupling is made via a cysteine residue or via a non-native reactive or photo-reactive amino acid in a CsgG monomer at a position corresponding to 132, 133, 136, 138, 140, 142, 144, 145, 147, 149, 151, 153, 155, 183, 185, 187, 189, 191, 201, 203, 205, 207 or 209 of SEQ ID NO: 117 or SEQ ID NO: 3, or of a homologue thereof.

The invention also provides an isolated transmembrane pore or pore complex, or a membranous composition, which comprises the isolated pore or pore complex of the invention, and the components of a membrane. Particularly, said transmembrane pore or pore complex or membranous composition consists of the isolated pore or pore complex of the invention, and the components of a membrane or an insulating layer.

The invention also provides:
a membrane comprising a pore or pore complex of the invention;
an array comprising a plurality of membranes of the invention;
a system comprising (a) a membrane of the invention or an array of the invention, (b) means for applying a potential across the membrane(s) and (c) means for detecting electrical or optical signals across the membrane(s).

The invention also provides a method for producing a transmembrane pore complex of the invention, comprising co-expressing the CsgG pore, or the homologue or mutant thereof, and the modified CsgF peptide, or a homologue or mutant thereof, in a suitable host cell, thereby allowing in vivo transmembrane pore complex formation.

The invention also provides a method for producing an isolated pore complex of the invention, comprising contacting the CsgG monomers, or the homologue or mutant thereof, with the modified CsgF peptide, or the homologue or mutant thereof, thereby allowing in vitro reconstitution of the isolated pore complex. The modified CsgF peptide may be a peptide comprising an enzyme cleavage site at a suitable position in the amino acid sequence, that is cleaved before or after formation of the pore.

In specific embodiments, said modified CsgF peptide, or homologue or mutant thereof, comprises SEQ ID NO: 12 or SEQ ID NO:14, or a homologue or mutant thereof. In particular embodiments, modified CsgF peptides of said method comprise SEQ ID NO:15 or SEQ ID NO:16, or homologues or mutants thereof.

The invention also provides a method for determining the presence, absence or one or more characteristics of a target analyte, comprising the steps of:
(i) contacting the target analyte with an isolated pore or an isolated pore complex of the invention or a transmembrane pore complex of the invention, such that the target analyte moves into the pore channel; and
(ii) taking one or more measurements as the analyte moves through the pore channel and thereby determining the presence, absence or one or more characteristics of the analyte. In one embodiment, said analyte is a polynucleotide. In particular, said method using a polynucleotide as an analyte alternatively comprises determining one or more characteristics selected from (i) the length of the analyte or polynucleotide, (ii) the identity of the analyte or polynucleotide, (iii) the sequence of the analyte or polynucleotide, (iv) the secondary structure of the analyte or polynucleotide and (v) whether or not the analyte or polynucleotide is modified.

In another embodiment, the analyte is a protein, (poly)peptide or peptide. In further embodiments, said analyte is a polymer, oligosaccharide, polysaccharide, or a small organic or inorganic compound, such as for instance but not limited to pharmacologically active compounds, toxic compounds and pollutants.

The invention also provides a method for characterising a polynucleotide or a (poly)peptide using an isolated pore or an isolated pore complex of the invention or a transmembrane pore complex of the invention. In particular, said CsgG pore, or homologue or mutant thereof, comprises six to ten CsgG monomers forming the CsgG pore channel.

The invention also provides use of an isolated pore or isolated pore complex of the invention or a transmembrane pore complex of the invention to determine the presence, absence or one or more characteristics of a target analyte. Furthermore, the invention also relates to a kit for characterising a target analyte comprising (a) said isolated pore or pore complex and (b) the components of a membrane.

The invention also provides:
a method of altering the speed at which a target analyte passes through a pore comprising contacting the target analyte with an isolated pore or an isolated pore complex of the invention or with a transmembrane pore complex of the invention, such that the target analyte moves relative to, or into the pore complex;
a kit for characterising a target analyte comprising (a) an isolated pore or an isolated pore complex of the invention and one or both of (b) the components of a membrane and (c) a polynucleotide binding protein;
a method of characterising a target analyte, comprising:
(a) contacting the target analyte with an isolated pore or isolated pore complex of the invention and a DNA dependent ATPase (Dda) helicase of the invention or a helicase construct of the invention such that the helicase or construct controls the movement of the target analyte through the pore or pore complex; and
(b) taking one or more measurements as the polynucleotide moves with respect to the pore or pore complex wherein the measurements are indicative of one or more characteristics of the target analyte and thereby characterising the target analyte;
a kit for characterising a target analyte comprising (a) a DNA dependent ATPase (Dda) helicase of the invention or a helicase construct of the invention (b) an isolated CsgG pore or isolated pore complex of the invention;
an apparatus comprising a pore or pore complex of the invention inserted into an in vitro membrane;
an apparatus produced by a method comprising: (i) obtaining an isolated pore or an isolated pore complex of the invention and (ii) contacting the isolated pore or isolated pore complex with an in vitro membrane such that the pore is inserted in the in vitro membrane.

### DETAILED DESCRIPTION

All publications, patents and patent applications cited herein, whether supra or infra, are hereby incorporated by reference in their entirety. All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may do so. Similarly, it should be appreciated that in the description of exemplary embodiments of the invention, various features of the invention are sometimes grouped together in a single embodiment, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

In addition as used in this specification and the appended claims, the singular forms “a”, “an”, and “the” include plural referents unless the content clearly dictates otherwise. Thus, for example, reference to “a polynucleotide” includes two or more polynucleotides, reference to “a polynucleotide binding protein” includes two or more such proteins, reference to “a helicase” includes two or more helicases, reference to “a monomer” refers to two or more monomers, reference to “a pore” includes two or more pores and the like.

In all of the discussion herein, the standard one letter codes for amino acids are used. These are as follows: alanine (A), arginine (R), asparagine (N), aspartic acid (D), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y) and valine (V). Standard substitution notation is also used, i.e. Q42R means that Q at position 42 is replaced with R.

In the paragraphs herein where different amino acids at a specific position are separated by the / symbol, the / symbol means “or”. For instance, Q87R/K means Q87R or Q87K. In the paragraphs herein where different positions are separated by the / symbol, the / symbol means “and” such that Y51/N55 is Y51 and N55.

### Definitions

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Nucleotide sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. The term “nucleic acid” as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may be manufactured synthetically in vitro or isolated from natural sources. Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Sizes of nucleic acids, also referred to herein as “polynucleotides” are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides” and may comprise primers for use in manipulation of DNA such as via polymerase chain reaction (PCR).

“Gene” as used here includes both the promoter region of the gene as well as the coding sequence. It refers both to the genomic sequence (including possible introns) as well as to the cDNA derived from the spliced messenger, operably linked to a promoter sequence.

“Coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances.

The term “amino acid” in the context of the present disclosure is used in its broadest sense and is meant to include organic compounds containing amine (NH₂) and carboxyl (COOH) functional groups, along with a side chain (e.g., a R group) specific to each amino acid. In some embodiments, the amino acids refer to naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as 3-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference. The terms “protein”, “polypeptide”, and “peptide” are interchangeably used further herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

Polypeptides can also undergo maturation or post-translational modification processes that may include, but are not limited to: glycosylation, proteolytic cleavage, lipidization, signal peptide cleavage, propeptide cleavage, phosphorylation, and such like. By “recombinant polypeptide” is meant a polypeptide made using recombinant techniques, e.g., through the expression of a recombinant or synthetic polynucleotide. When the chimeric polypeptide or biologically active portion thereof is recombinantly produced, it is also preferably substantially free of culture medium, e.g., culture medium represents less than about 20%, more preferably less than about 10%, and most preferably less than about 5% of the volume of the protein preparation. By “isolated” is meant material that is substantially or essentially free from components that normally accompany it in its native state. For example, an “isolated polypeptide”, as used herein, refers to a polypeptide, which has been purified from the molecules which flank it in a naturally-occurring state, e.g., a protein complex or CsgF peptide which has been removed from the molecules present in the production host that are adjacent to said polypeptide. An isolated CsgF peptide (optionally a truncated CsgF peptide) can be generated by amino acid chemical synthesis or can be generated by recombinant production. An isolated complex can be generated by in vitro reconstitution after purification of the components of the complex, e.g. a CsgG pore and the CsgF peptide(s), or can be generated by recombinant co-expression.

“Orthologues” and “paralogues” encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

“Homologue”, “Homologues” of a protein encompass peptides, oligopeptides, polypeptides, proteins and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

The term “CsgG pore” defines a pore comprising multiple CsgG monomers. Each CsgG monomer may be a wild-type monomer from E. coli (SEQ ID NO: 3), wild-type homologues of E. coli CsgG, such as for example, monomers having any one of the amino acid sequences shown in SEQ ID NOS: 68 to 88. or a variant of any thereof (e.g. a variant of any one of SEQ ID NOs: 3, 117 and 68 to 88). The variant CsgG monomer may also be referred to as a modified CsgG monomer or a mutant CsgG monomer. The modifications, or mutations, in the variant include but are not limited to any one or more of the modifications disclosed herein, or combinations of said modifications.

For all aspects and embodiments of the present invention, a CsgG homologue is referred to as a polypeptide that has at least 40%, 50%, 60%, 70%, 80%, 85%, 90%, 95% or 99% complete sequence identity to wild-type E. coli CsgG as shown in SEQ ID NO: 117 or SEQ ID NO: 3. A CsgG homologue is also referred to as a polypeptide that contains the PFAM domain PF03783, which is characteristic for CsgG-like proteins. A list of presently known CsgG homologues and CsgG architectures can be found at http://pfam.xfam.org//family/PF03783. Likewise, a CsgG homologous polynucleotide can comprise a polynucleotide that has at least 40%, 50%, 60%, 70%, 80%, 90%, 95% or 99% complete sequence identity to wild-type E. coli CsgG as shown in SEQ ID NO: 1. Examples of homologues of CsgG shown in SEQ ID NO:3 have the sequences shown in SEQ ID NOS: 68 to 88.

The term “modified CsgF peptide” or “CsgF peptide” defines CsgF peptide that has been truncated from its C-terminal end (e.g. is an N-terminal fragment) and/or is modified to include a cleavage site. The CsgF peptide may be a fragment of wild-type E. coli CsgF (SEQ ID NO: 5 or SEQ ID NO: 6), or of a wild-type homologue of E. coli CsgF, such as for example, a peptide comprising any one of the amino acid sequences shown in SEQ ID NOS: 17 to 36. or a variant (e.g. one modified to include a cleavage site) of any thereof.

For all aspects and embodiments of the present invention, a CsgF homologue is referred to as a polypeptide that has at least 40%, 50%, 60%, 70%, 80%, 90%, 95% or 99% complete sequence identity to wild-type E. coli CsgF as shown in SEQ ID NO: 6. In some embodiments, a CsgF homologue is also referred to as a polypeptide that contains the PFAM domain PF10614, which is characteristic for CsgF-like proteins. A list of presently known CsgF homologues and CsgF architectures can be found at 0.1. Likewise, a CsgF homologous polynucleotide can comprise a polynucleotide that has at least 40%, 50%, 60%, 70%, 80%, 90%, 95% or 99% complete sequence identity to wild-type E. coli CsgF as shown in SEQ ID NO: 4. Examples of truncated regions of homologues of CsgF shown in SEQ ID NO:6 have the sequences shown in SEQ ID NOs:17 to 36.

The term “N-terminal portion of a CsgF mature peptide” refers to a peptide having an amino acid sequence that corresponds to the first 60, 50, or 40 amino acid residues starting from the N-terminus of a CsgF mature peptide (without a signal sequence). The CsgF mature peptide can be a wild-type or mutant (e.g., with one or more mutations).

Sequence identity can also be to a fragment or portion of the full length polynucleotide or polypeptide. Hence, a sequence may have only 50% overall sequence identity with a full length reference sequence, but a sequence of a particular region, domain or subunit could share 80%, 90%, or as much as 99% sequence identity with the reference sequence. Homology to the nucleic acid sequence of SEQ ID NO: 1 for CsgG homologues or SEQ ID NO:4 for CsgF homologues, respectively, is not limited simply to sequence identity. Many nucleic acid sequences can demonstrate biologically significant homology to each other despite having apparently low sequence identity. Homologous nucleic acid sequences are considered to be those that will hybridise to each other under conditions of low stringency (M. R. Green, J. Sambrook, 2012, Molecular Cloning: A Laboratory Manual, Fourth Edition, Books 1-3, Cold Spring Harbor Laboratory Press, Cold Spring Harbor, NY).

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence (e.g., substitutions, truncations, or insertions), post-translational modifications and/or functional properties (e.g., altered characteristics) when compared to the wild-type gene or gene product. It is noted that naturally occurring mutants can be isolated; these are identified by the fact that they have altered characteristics when compared to the wild-type gene or gene product. Methods for introducing or substituting naturally-occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. Methods for introducing or substituting non-naturally-occurring amino acids are also well known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. They may also be produced by naked ligation if the mutant monomer is produced using partial peptide synthesis. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid.

Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

A mutant or modified protein, monomer or peptide can also be chemically modified in any way and at any site. A mutant or modified monomer or peptide is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant of modified protein, monomer or peptide may be chemically modified by the attachment of any molecule. For instance, the mutant of modified protein, monomer or peptide may be chemically modified by attachment of a dye or a fluorophore. In some embodiments, the mutant or modified monomer or peptide is chemically modified with a molecular adaptor that facilitates the interaction between a pore comprising the monomer or peptide and a target nucleotide or target polynucleotide sequence. The molecular adaptor is preferably a cyclic molecule, a cyclodextrin, a species that is capable of hybridization, a DNA binder or interchelator, a peptide or peptide analogue, a synthetic polymer, an aromatic planar molecule, a small positively-charged molecule or a small molecule capable of hydrogen-bonding.

The presence of the adaptor improves the host-guest chemistry of the pore and the nucleotide or polynucleotide sequence and thereby improves the sequencing ability of pores formed from the mutant monomer. The principles of host-guest chemistry are well-known in the art. The adaptor has an effect on the physical or chemical properties of the pore that improves its interaction with the nucleotide or polynucleotide sequence. The adaptor may alter the charge of the barrel or channel of the pore or specifically interact with or bind to the nucleotide or polynucleotide sequence thereby facilitating its interaction with the pore. Hence a modified CsgF peptide, as provided in the disclosure, may be coupled to enzymes or proteins providing better proximity of said proteins or enzymes to the pore, which may facilitate certain applications of the pore complex comprising the modified CsgF peptide.

In this context, proteins can also be fusion proteins, referring in particular to genetic fusion, made e.g., by recombinant DNA technology. Proteins can also be conjugated, or “conjugated to”, as used herein, which refers, in particular, to chemical and/or enzymatic conjugation resulting in a stable covalent link.

Proteins may form a protein complex when several polypeptides or protein monomers bind to or interact with each other. “Binding” means any interaction, be it direct or indirect. A direct interaction implies a contact between the binding partners, for instance through a covalent link or coupling. An indirect interaction means any interaction whereby the interaction partners interact in a complex of more than two compounds. The interaction can be completely indirect, with the help of one or more bridging molecules, or partly indirect, where there is still a direct contact between the partners, which is stabilized by the additional interaction of one or more compounds. The “complex” as referred to in this disclosure is defined as a group of two or more associated proteins, which might have different functions. The association between the different polyptides of the protein complex might be via non-covalent interactions, such as hydrophobic or ionic forces, or may as well be a covalent binding or coupling, such as disulphide bridges, or peptidic bounds. Covalent “binding” or “coupling” are used interchangeably herein, and may also involve “cysteine coupling” or “reactive or photoreactive amino acid coupling”, referring to a bioconjugation between cysteines or between (photo)reactive amino acids, respectively, which is a chemical covalent link to form a stable complex. Examples of photoreactive amino acids include azidohomoalanine, homopropargylglycyine, homoallelglycine, p-acetyl-Phe, p-azido-Phe, p-propargyloxy-Phe and p-benzoyl-Phe (Wang et al. 2012, in Protein Engineering, DOI: 10.5772/28719; Chin et al. 2002, Proc. Nat. Acad. Sci. USA 99(17); 11020-24).

A “biological pore” is a transmembrane protein structure defining a channel or hole that allows the translocation of molecules and ions from one side of the membrane to the other. The translocation of ionic species through the pore may be driven by an electrical potential difference applied to either side of the pore. A “nanopore” is a biological pore in which the minimum diameter of the channel through which molecules or ions pass is in the order of nanometres (10-9 nanometres). In some embodiments, the biological pore can be a transmembrane protein pore.

The transmembrane protein structure of a biological pore may be monomeric or oligomeric in nature. Typically, the pore comprises a plurality of polypeptide subunits arranged around a central axis thereby forming a protein-lined channel that extends substantially perpendicular to the membrane in which the nanopore resides. The number of polypeptide subunits is not limited. Typically, the number of subunits is from 5 to up to 30, suitably the number of subunits is from 6 to 10. Alternatively, the number of subunits is not defined as in the case of perfringolysin or related large membrane pores. The portions of the protein subunits within the nanopore that form protein-lined channel typically comprise secondary structural motifs that may include one or more trans-membrane β-barrel, and/or α-helix sections.

The term “pore”, “pore complex”, or “complex pore”, as used interchangeably herein, refer to an oligomeric pore, wherein for instance at least a CsgG monomer (including, e.g., one or more CsgG monomers such as two or more CsgG monomers, three or more CsgG monomers) or a CsgG pore (comprised of CsgG monomers), and a CsgF peptide (e.g., a modified or truncated CsgF peptide) are associated in the complex and together form a pore or a nanopore. The pore complex of the disclosure has the features of a biological pore, i.e. it has a typical transmembrane protein structure. When the pore complex is provided in an environment having membrane components, membranes, cells, or an insulating layer, the pore complex will insert in the membrane or the insulating layer, and form a “transmembrane pore complex”.

The pore, pore complex, transmembrane pore or transmembrane pore complex of the disclosure is suited for analyte characterization. In some embodiments, the pore, pore complex, transmembrane pore or transmembrane pore complex described herein can be used for sequencing polynucleotide sequences e.g., because it can discriminate between different nucleotides with a high degree of sensitivity. The pore or pore complex may be isolated, substantially isolated, purified or substantially purified. A pore or pore complex is “isolated” or purified if it is completely free of any other components, such as lipids or other pores, or other proteins with which it is normally associated in its native state e.g., CsgE, CsgA CsgB, or if it is sufficiently enriched from a membranous compartment. A pore or pore complex is substantially isolated if it is mixed with carriers or diluents which will not interfere with its intended use. For instance, a pore or pore complex is substantially isolated or substantially purified if it is present in a form that comprises less than 10%, less than 5%, less than 2% or less than 1% of other components, such as triblock copolymers, lipids or other pores. Alternatively, a pore complex of the disclosure may be a transmembrane pore or transmembrane pore complex, when present in a membrane. The disclosure provides isolated pores and isolated pore complexes comprising a homo-oligomeric pore derived from CsgG comprising identical mutant monomers, which may also contain a mutant form of the CsgG monomer, as a homologue thereof. Alternatively, an isolated pore or isolated pore complex comprising a hetero-oligomeric CsgG pore is provided, which can be CsgG pore consisting of mutant and wild-type CsgG monomers, or of different forms of CsgG variants, mutants or homologues. The isolated pore complex typically comprises at least 7, at least 8, at least 9 or at least 10 CsgG monomers, and 1 or more (modified) CsgF peptides, such as 2, 3, 4, 5, 6, 7, 8, 9, 10 CsgF peptides. The pore complex may comprise any ratio of CsG monomer:CsgF peptide. In one embodiment, the ratio of CsG monomer:CsgF peptide is 1:1.

The “constriction”, “orifice”, “constriction region”, “channel constriction”, or “constriction site”, as used interchangeably herein, refers to an aperture defined by a luminal surface of a pore or pore complex, which acts to allow the passage of ions and target molecules (e.g., but not limited to polynucleotides or individual nucleotides) but not other non-target molecules through the pore or pore complex channel. In some embodiments, the constriction(s) are the narrowest aperture(s) within a pore or pore complex. In this embodiment, the constriction(s) may serve to limit the passage of molecules through the pore. The size of the constriction is typically a key factor in determining suitability of a nanopore for nucleic acid sequencing applications. If the constriction is too small, the molecule to be sequenced will not be able to pass through. However, to achieve a maximal effect on ion flow through the channel, the constriction should not be too large. For example, the constriction should not be wider than the solvent-accessible transverse diameter of a target analyte. Ideally, any constriction should be as close as possible in diameter to the transverse diameter of the analyte passing through. For sequencing of nucleic acids and nucleic acid bases, suitable constriction diameters are in the nanometre range (10-9 meter range). Suitably, the diameter should be in the region of 0.5 to 2.0 nm, typically, the diameter is in the region of 0.7 to 1.2 nm. The constriction in wild type E. coli CsgG has a diameter of approximately 9 Å (0.9 nm). The CsgF constriction formed in the pore complex comprising the CsgG-like pore and the modified CsgF peptide, or homologues or mutants thereof, has a diameter in the range of 0.5 to 2 nm or in the range of 0.7 to 1.2 nm and is hence suitable for nucleic acid sequencing.

When two or more constrictions are present and spaced apart each constriction may interact or “read” separate nucleotides within the nucleic acid strand at the same time. In this situation, the reduction in ion flow through the channel will be the result of the combined restriction in flow of all the constrictions containing nucleotides. Hence, in some instances a double constriction may lead to a composite current signal. In certain circumstances, the current read-out for one constriction, or “reading head”, may not be able to be determined individually when two such reading heads are present. The constriction of wildtype E. coli CsgG (SEQ ID NO:3) is composed of two annular rings formed by juxtaposition of tyrosine residues at position 51 (Tyr 51) in the adjacent protein monomers, and also the phenylalanine and asparagine residues at positions 56 and 55 respectively (Phe 56 and Asn 55). The wild-type pore structure of CsgG is in most cases being re-engineered via recombinant genetic techniques to widen, alter, or remove one of the two annular rings that make up the CsgG constriction (mentioned as “CsgG channel constriction” herein), to leave a single well-defined reading head. The constriction motif in the CsgG oligomeric pore is located at amino acid residues at position 38 to 63 in the wild type monomeric E. coli CsgG polypeptide, depicted in SEQ ID NO: 3. In considering this region, mutations at any of the amino acid residue positions 50 to 53, 54 to 56 and 58 to 59, as well as key of positioning of the sidechains of Tyr51, Asn55, and Phe56 within the channel of the wild-type CsgG structure, was shown to be advantageous in order to modify or alter the characteristics of the reading head. The present disclosure relating to a pore complex comprising a CsgG-pore and a modified CsgF peptide, or homologues or mutants thereof, surprisingly added another constriction (mentioned as “CsgF channel constriction” herein) to the CsgG-containing pore complex, forming a suitable additional, second reader head in the pore, via complex formation with the modified CsgF peptide. Said additional CsgF channel constriction or reader head is positioned adjacent to the constriction loop of the CsgG pore, or of the mutated GcsG pore. Said additional CsgF channel constriction or reader head is positioned approximately 10 nm or less, such as 5 nm or less, such as 1, 2, 3, 4, 5, 6, 7, 8, 9 nm from the constriction loop of the CsgG pore, or of the mutated GcsG pore. The pore complex or transmembrane pore complex of the disclosure includes pore complexes with two reader heads, meaning, channel constrictions positioned in such a way to provide a suitable separate reader head without interfering the accuracy of other constriction channel reader heads. Said pore complexes therefore may include CsgG mutant pores WO2016/034591, WO2017/149316, WO2017/149317, WO2019/002893, WO2017/149318, WO 2018/211241, WO2019/002893 (herein all incorporated by reference in their entirety) each of which lists mutations to the wild-type CsgG pore that improve the properties of the pore) as well as wild-type CsgG pores, or homologues thereof, together with a modified CsgF peptide, or homologue or mutant thereof, wherein said CsgF peptide has another constriction channel forming a reader head.

### Pores and Pore Complexes

The invention provides an isolated CsgG pore or a homologue or mutant thereof, or an isolated pore complex comprising a CsgG pore, or a homologue or mutant thereof, and a modified CsgF peptide, or a homologue or mutant thereof, wherein the CsgG pore comprises at least one mutant CsgG monomer comprising a modification at one or more of positions W97, Q100, E101, N102, and T104 in SEQ ID NO: 117. The CsgG pore may be a pore of SEQ ID NO: 3 or 117 or a homologue or mutant thereof. The at least one mutant monomer preferably comprises a variant of SEQ ID NO: 117 comprising a modification at one or more of positions W97, Q100, E101, N102, and T104.

The invention provides an isolated CsgG pore or a homologue or mutant thereof, wherein the CsgG pore comprises at least one mutant CsgG monomer comprising a modification at one or more of positions W97, Q100, E101, N102, and T104 in SEQ ID NO: 117. The CsgG pore may be a pore of SEQ ID NO: 3 or 117 or a homologue or mutant thereof. The at least one mutant monomer preferably comprises a variant of SEQ ID NO: 117 comprising a modification at one or more of positions W97, Q100, E101, N102, and T104.

The invention provides an isolated pore complex comprising a CsgG pore, or a homologue or mutant thereof, and a modified CsgF peptide, or a homologue or mutant thereof, wherein the CsgG pore comprises at least one mutant CsgG monomer comprising a modification at one or more of positions W97, Q100, E101, N102 and T104 in SEQ ID NO: 117. The CsgG pore may be a pore of SEQ ID NO: 3 or 117 or a homologue or mutant thereof. The at least one mutant monomer preferably comprises a variant of SEQ ID NO: 117 comprising a modification at one or more of positions W97, Q100, E101, N102, and T104.

The at least one mutant monomer or variant may comprise any number and combination of modifications at one or more of positions (a) W97, (b) Q100, (c) E101, (d) N102 and (e) T104 in SEQ ID NO: 117. For instance, the at least one mutant monomer may comprise modifications at (a); (b); (c); (d); (e); (a) and (b); (a) and (c); (a) and (d); (a) and (e); (b) and (c); (b) and (d); (b) and (e); (c) and (d); (c) and (e); (d) and (e); (a), (b) and (c); (a), (b) and (d); (a), (b) and (e); (a), (c) and (d); (a), (c) and (e); (a), (d) and (e); (b), (c) and (d); (b), (c) and (e); (b), (d) and (e); (c), (d) and (e); (a), (b), (c) and (d); (a), (b), (c) and (e); (a), (b), (d) and (e); (a), (c), (d) and (e); (b), (c), (d) and (e); and (a), (b), (c), (d) and (e). The at least one mutant monomer or variant preferably comprises modifications at one or more positions (a) W97, (b) Q100, (c) E101 and (d) N102 in SEQ ID NO: 117, including all combinations of (a) to (d) set out above.

The modification at one or more of positions W97, Q100, E101, N102 and T104 in SEQ ID NO: 117 may be any of the modifications discussed in more detail below. The modification may be a deletion, such as a deletion of E101. Deletion of E101 increases the speed of movement through the pore (Example 1). The modification is preferably a substitution.

The W at position 97 is preferably substituted with R, H, K, A, V, I, L, M, F, Y, S, T, Q, D, E, N, C, P or G. The W at position 97 is more preferably substituted with D, G, N, R or S. These substitutions increase the speed at which an analyte passes through/relative to the pore (Example 1). The W at position 97 is more preferably substituted with D or R. These substitutions increase the speed at which an analyte passes through/relative to the pore and increase the normalised speed distribution (Example 1). The W at position 97 is more preferably substituted with G, N or S. These substitutions increase the speed at which an analyte passes through/relative to the pore and decrease the normalised speed distribution (Example 1).

The Q at position 100 is preferably substituted with R, H, K, W, A, V, I, L, M, F, Y, T, N or S. The Q at position 100 is more preferably substituted with A, K or S. These substitutions increase the speed at which an analyte passes through/relative to the pore (Example 1). The Q at position 100 is more preferably substituted with K (Q100K). This substitution increases the speed at which an analyte passes through/relative to the pore and increases the normalised speed distribution (Example 1). The Q at position 100 is more preferably substituted with A or S (Q100A or Q100S). These substitutions increase the speed at which an analyte passes through/relative to the pore and decrease the normalised speed distribution (Example 1). The Q at position 100 is most preferably substituted with A (Q100A). This substitution also has the effects shown in Example 4 when used with a modified Dda helicase of the invention.

The E at position 101 is preferably substituted with A, V, I, L, M, F, Y or W. The E at position 101 is more preferably substituted with A (E101A). This substitution decreases the speed at which an analyte passes through/relative to the pore and decrease the normalised speed distribution (Example 1).

The E at position 101 is preferably substituted with S, T, N, Q, C, G or P. The E at position 101 is more preferably substituted with G or S. These substitutions increase the speed at which an analyte passes through/relative to the pore (Example 1). The E at position 101 is more preferably substituted with G (E101G). This substitution increases the speed at which an analyte passes through/relative to the pore and decreases the normalised speed distribution (Example 1). The E at position 101 is more preferably substituted with S (E101S). This substitution increases the speed at which an analyte passes through/relative to the pore and increases the normalised speed distribution (Example 1).

The N at position 102 is preferably substituted with D, E, R, H, K, S, T, Q, V, I, L, M, F, Y, W or A. The N at position 102 is more preferably substituted with A, D, R, S or W. These substitutions increase the speed at which an analyte passes through/relative to the pore (Example 1). The N at position 102 is preferably substituted with A, R or S. These substitutions increase the speed at which an analyte passes through/relative to the pore and decrease the normalised speed distribution (Example 1). The N at position 102 is preferably substituted with D or W (N102D or N102W). These substitutions increase the speed at which an analyte passes through/relative to the pore and increase the normalised speed distribution (Example 1). The N at position 102 is most preferably substituted with A or S (N102A or N102S). These substitutions also have the effects shown in Example 4 when used with a modified Dda helicase of the invention.

The T at position 104 is preferably substituted with R, H or K.

As explained in more detail below, the pore preferably comprises six to ten monomers. Any number of these, such as 6, 7, 8, 9 or 10, may be a mutant monomer comprising a modification at one or more of positions W97, Q100, E101, N102, and T104 in SEQ ID NO: 117 or may comprise a variant of SEQ ID NO: 117 comprising a modification at one or more of positions W97, Q100, E101, N102, and T104. All six to ten monomers may comprise a modification at one or more of positions W97, Q100, E101, N102, and T104 in SEQ ID NO: 117 or may comprise a variant of SEQ ID NO: 117 comprising a modification at one or more of positions W97, Q100, E101, N102, and T104.

A mutant CsgG monomer is a monomer whose sequence varies from that of a wild-type CsgG monomer and which retains the ability to form a pore. A mutant monomer may also be referred to herein as a variant. Methods for confirming the ability of mutant monomers to form pores are well-known in the art and are discussed in more detail below. The at least one mutant monomer or variant may have any of the % s of homology/sequence identity to SEQ ID NO: 117 or SEQ ID NO: 3 set out below. The at least one mutant monomer may contain any of the additional modifications, mutations or substitutions described below, including the types of modifications and substitutions described with reference to the Dda helicases of the invention. The at least one mutant monomer may contain any of the additional modifications, mutations or substitutions described in WO2016/034591, WO2017/149316, WO2017/149317 and, WO2017/149318, WO2018/211241, and WO2019/002893 (all incorporated by reference herein in their entirety). The invention relates to CsgG pores, optionally complexed with an extracellularly located CsgF peptide that surprisingly introduces an additional channel constriction or reader head in the pore complex. Moreover, the disclosure provides positional information for the constriction made by the CsgF peptide within the pore complex, the peptide being inserted in the lumen of the CsgG pore, and the constriction site being in the N-terminal part of the CsgF protein. Furthermore, modified or truncated CsgF peptides of the disclosure were shown to be sufficient for pore complex formation, and provide means and methods for biosensing applications. The disclosure comprises wildtype and mutant CsgG pores (as disclosed in e.g., WO2016/034591, WO2017/149316, WO2017/149317, WO2017/149318 and International patent application no. PCT/GB2018/051191), or homologues or mutants thereof, optionally combined with modified or truncated CsgF peptides and its mutants or homologues, all together improving the ability of the CsgG pore or CsgG-like pore complex to interact with an analyte, such as a polynucleotide. The additional constriction introduced in the CsgG-like nanopore channel by complex formation with (modified or truncated) CsgF peptides expands the contact surface with passing analytes and can act as a second reader head for analyte detection and characterization. Pores comprising mutant CsgG monomers combined with novel mutant or modified forms of CsgF can improve the characterisation of analytes, such as polynucleotides, providing a more discriminating direct relationship between the observed current as the polynucleotide moves through the pore. In particular, by having two stacked reader heads spaced at a defined distance, the CsgG:CsgF pore complex may facilitate characterization of polynucleotides that contain at least one homopolymeric stretch, e.g., several consecutive copies of the same nucleotide that otherwise exceed the interaction length of the single CsgG reader head. Additionally, by having two stacked constrictions at a defined distance, small molecule analytes including organic or inorganic drugs and pollutants passing through the CsgG:CsgF complex pore will consecutively pass two independent reader heads. The chemical nature of either reader head can be independently modified, each giving unique interaction properties with the analyte, thus providing additional discriminating power during analyte detection.

The invention relates to an isolated pore complex, comprising a CsgG pore, or a homologue or mutant thereof, or a CsgG-like pore, and a modified CsgF peptide, or a homologue or mutant thereof. In fact, the disclosure relates to a modified CsgG biological pore, comprising a modified CsgF peptide, which can be a truncated, mutant and/or variant thereof. In one embodiment, the interaction region between said modified CsgF peptide or homologue or mutant thereof, is located at the lumen of the CsgG pore, or its homologues or mutants. In another embodiment, the pore complex has two or more constriction sites or reader heads, provided by at least one constriction of the CsgG pore, and by at least one being introduced by the CsgF peptide, forming a complex with the CsgG pore. N-terminal CsgF positions with the inclusion of positions in the range of amino acid residues 39-64 of SEQ ID NO:5, or more particularly of amino acid residues 49-64 of SEQ ID NO:5, were shown to allow detectable amounts of a stable CsgG:CsgF complex. In one embodiment, the CsgF constriction produced by a modified CsgF peptide (e.g., the ones described herein) is adjacent or head-to-head of the first constriction in the CsgG pore of the pore complex. For CsgG or CsgG-like protein pores, the constriction site has been determined to be formed by a loop region of a beta strand.

In one embodiment, the modified CsgF peptide is a peptide wherein said modification in particular refers to a truncated CsgF protein or fragment, comprising an N-terminal CsgF peptide fragment defined by the limitation to contain the constriction region and to bind CsgG monomers, or homologues or mutants thereof. Said modified CsgF peptide may additionally comprise mutations or homologous sequences, which may facilitate certain properties of the pore complex. In a particular embodiment, modified CsgF peptides comprise CsgF protein truncations as compared to the wild-type preprotein (SEQ ID NO:5) or mature protein (SEQ ID NO:6) sequence, or homologues thereof. These modified peptides are intended to function as a pore complex component introducing an additional constriction site or reader head, within the CsgG-like pore formed by CsgG and the modified or truncated CsgF peptide. Examples of truncated modified peptides are described below.

Examples of homologues of the modified CsgF peptides are disclosed in WO2019/002893 (incorporated by reference herein in its entirety) and reveal CsgF-like proteins or CsgF peptides comprising a homologous or similar constriction region in different bacterial strains, which may be useful in the use of similar pore complexes. The structural properties and CsgG-binding elements in the CsgF peptides derived from various CsgF homologues are conserved, such that CsgF peptides can be used in combination with different wildtype or mutant CsgG pores. This includes complexes of CsgG pores with non-cognate CsgF, meaning that the CsgG pore and the parental CsgF homologue from which the CsgF is derived do not need to originate from the same operon, bacterial species or strain.

In alternative embodiments, the CsgG pore within the pore complex is not a wild-type pore, but comprises mutations or modifications to increase pore properties as well. The isolated pore complex of the disclosure, formed by the CsgG pore, or a homologue thereof, and the modified CsgF peptide, or a homologue thereof, may be formed by the wild-type form of the CsgG pore or may be further modified in the CsgG pore, such as by directed mutagenesis of particular amino acid residues, to further enhance the desired properties of the CsgG pore for use within the pore complex. For example, in embodiments of the present invention mutations are contemplated to alter the number, size, shape, placement or orientation of the constriction within the channel. The pore complex comprising a modified mutant CsgG pore may be prepared by known genetic engineering techniques that result in the insertion, substitution and/or deletion of specific targeted amino acid residues in the polypeptide sequence. In the case of the oligomeric CsgG pore, the mutations may be made in each monomeric polypeptide subunit, or any one of the monomers, or all of the monomers. Suitably, in one embodiment of the invention the mutations described are made to all monomeric polypeptides within the oligomeric protein structure. A mutant CsgG monomer is a monomer whose sequence varies from that of a wild-type CsgG monomer and which retains the ability to form a pore. Methods for confirming the ability of mutant monomers to form pores are well-known in the art. The disclosure comprises wild type and mutant CsgG pores (e.g., as disclosed in WO2016/034591, WO2017/149316, WO2017/149317, WO2017/149318 and International patent application no. PCT/GB2018/051191), or homologues thereof, combined with modified or truncated CsgF peptides and their mutants or homologues, all together improving the ability of the CsgG-like pore complex to interact with an analyte, such as a polynucleotide. Mutant CsgG pores may comprise one or more mutant monomers. The CsgG pore may be a homopolymer comprising identical monomers, or a heteropolymer comprising two or more different monomers. The monomers may have one or more of the mutations described below in any combination.

The nanopore complex comprising a modified CsgF peptide differs as compared to the wild-type CsgF protein depicted in SEQ ID NO:6 since the modified CsgF peptide only comprises N-terminal fragments or truncates of the wild-type CsgF protein in certain embodiments. The modified CsgF peptide however may be additionally or alternatively mutated CsgF peptide in the sense that mutations as amino acid substitutions are made to allow for a better second constriction site in the pore formed by the complex comprising the CsgG pore and the modified CsgF peptide. The mutant monomers might as such have improved polynucleotide reading properties when said complex is used in nucleotide sequencing i.e. display improved polynucleotide capture and nucleotide discrimination, in addition to the improved feature of the complex to comprise two reader heads. In particular, pores constructed from the mutant peptides capture nucleotides and polynucleotides more easily than the wild type. In addition, pores constructed from the mutant peptides may display an increased current range, which makes it easier to discriminate between different nucleotides, and a reduced variance of states, which increases the signal-to-noise ratio. In addition, the number of nucleotides contributing to the current as the polynucleotide moves through pores constructed from the mutants may be decreased. This makes it easier to identify a direct relationship between the observed current as the polynucleotide moves through the pore and the polynucleotide sequence. In addition, pores constructed from the mutant peptides may display an increased throughput, e.g., are more likely to interact with an analyte, such as a polynucleotide. This makes it easier to characterise analytes using the pores. Pores constructed from the mutant peptides may insert into a membrane more easily, or may provide easier way to retain additional proteins in close vicinity of the pore complex.

In an alternative embodiment, the CsgF constriction site provided in the pore complex of the invention has a diameter in the range of 0.5 nm to 2.0 nm, thereby providing a pore complex suitable for nucleic acid sequencing, as described above.

The pore may be stabilised by covalent attachment of the CsgF peptide to the CsgG pore. The covalent linkage may for example be a disulphide bond, or click chemistry. The CsgF peptide and CsgG pore may, for example, be covalently linked via residues at a position corresponding to one or more of the following pairs of positions of SEQ ID NO: 6 and SEQ ID NO: 3 or SEQ ID NO: 117, respectively: 1 and 153, 4 and 133, 5 and 136, 8 and 187, 8 and 203, 9 and 203, 11 and 142, 11 and 201, 12 and 149, 12 and 203, 26 and 191, and 29 and 144.

In the pore, the interaction between the CsgF peptide and the CsgG pore may, for example, be stabilised by hydrophobic interactions or electrostatic interactions at a position corresponding to one or more of the following pairs of positions of SEQ ID NO: 6 and SEQ ID NO: 3 or SEQ ID NO: 117, respectively: 1 and 153, 4 and 133, 5 and 136, 8 and 187, 8 and 203, 9 and 203, 11 and 142, 11 and 201, 12 and 149, 12 and 203, 26 and 191, and 29 and 144.

The residues in CsgF and/or CsgG at one or more of the positions listed above may be modified in order to enhance the interaction between CsgG and CsgF in the pore.

In one embodiment, the pore of the invention may be isolated, substantially isolated, purified or substantially purified. A pore of the invention is isolated or purified if it is completely free of any other components, such as lipids or other pores. A pore is substantially isolated if it is mixed with carriers or diluents which will not interfere with its intended use. For instance, a pore is substantially isolated or substantially purified if it is present in a form that comprises less than 10%, less than 5%, less than 2% or less than 1% of other components, such as triblock copolymers, lipids or other pores. Alternatively, a pore of the invention may be present in a membrane. Suitable membranes are discussed below.

A pore of the invention may be present as an individual or single pore. Alternatively, a pore of the invention may be present in a homologous or heterologous population of two or more pores.

### CsgF Peptide

The isolated pore complex of the invention includes modified CsgF monomers (peptides), or truncated CsgF proteins, or a modified or truncated peptide of a CsgF homologue or mutant. Those novel modified CsgF peptides may be used in a pore complex to integrate a second or additional reader head. Said modification or truncation is preferably resulting in a fragment of the wild-type CsgF, or of mutant or homologue CsgF protein, more preferably an N-terminal fragment. The modified CsgF peptide of SEQ ID NO 5, or a homologue or mutant thereof, may be any of those disclosed in WO2016/034591, WO2017/149316, WO2017/149317, WO2017/149318, WO2018/211241, and WO2019/002893 (all incorporated by reference herein in their entirety).

The CsgF peptide which forms part of the invention is a truncated CsgF peptide lacking the C-terminal head; lacking the C-terminal head and a part of the neck domain of CsgF (e.g., the truncated CsgF peptide may comprise only a portion of the neck domain of CsgF); or lacking the C-terminal head and neck domains of CsgF. The CsgF peptide may lack part of the CsgF neck domain, e.g. the CsgF peptide may comprise a portion of the neck domain, such as for example, from amino acid residue 36 at the N-terminal end of the neck domain (see SEQ ID:NO:6) (e.g. residues 36-40, 36-41, 36-42, 36-43, 36-45, 36-46 up to residues 36-50 or 36-60 of SEQ ID NO: 6). The CsgF peptide preferably comprises a CsgG-binding region and a region that forms a constriction in the pore. The CsgG-binding region typically comprises residues 1 to 8 and/or 29 to 32 of the CsgF protein (SEQ ID NO: 6 or a homologue from another species) and may include one or more modifications. The region that forms a constriction in the pore typically comprises residues 9 to 28 of the CsgF protein (SEQ ID NO: 6 or a homologue from another species) and may include one or more modifications. Residues 9 to 17 comprise the conserved motif N9PXFGGXXX17 and form a turn region. Residues 9 to 28 form an alpha-helix. X17 (N17 in SEQ ID NO: 6) forms the apex of the constriction region, corresponding to the narrowest part of the CsgF constriction in the pore. The CsgF constriction region also makes stabilising contacts with the CsgG beta-barrel, primarily at residues 9, 11, 12, 18, 21 and 22 of SEQ ID NO: 6.

The CsgF peptide typically has a length of from 28 to 50 amino acids, such as 29 to 49, 30 to 45 or 32 to 40 amino acids. Preferably the CsgF peptide comprises from 29 to 35 amino acids, or 29 to 45 amino acids. The CsgF peptide comprises all or part of the FCP, which corresponds to residues 1 to 35 of SEQ ID NO: 6. Where the CsgF peptide is shorter that the FCP, the truncation is preferably made at the C-terminal end.

The CsgF fragment of SEQ ID NO:6 or of a homologue or mutant thereof may have a length of 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55 amino acids.

The CsgF peptide may comprise the amino acid sequence of SEQ ID NO: 6 from residue 1 up to any one of residues 25 to 60, such as 27 to 50, for example, 28 to 45 of SEQ ID NO: 6, or the corresponding residues from a homologue of SEQ ID NO: 6, or variant of either thereof. More specifically, the CsgF peptide may comprise SEQ ID NO: 39 (residues 1 to 29 of SEQ ID NO: 6), or a homologue or variant thereof.

Examples of such CsgF peptides comprises, consist essentially of or consist of SEQ ID NO: 15 (residues 1 to 34 of SEQ ID NO: 6), SEQ ID NO: 54 (residues 1 to 30 of SEQ ID NO: 6), SEQ ID NO: 40 (residues 1 to 45 of SEQ ID NO: 6), or SEQ ID NO: 55 (residues 1 to 35 of SEQ ID NO: 6) and homologues or variants of any thereof. Other Examples of CsgF peptides comprise, consist essentially of or consist of SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 16.

In the CsgF peptide, one or more residues e.g., in SEQ ID NO: 15, SEQ ID NO: 39, SEQ ID NO: 40, SEQ ID NO: 54, or SEQ ID NO: 55 may be modified.

For example, the CsgF peptide may comprise a modification at a position corresponding to one or more of the following positions in SEQ ID NO: 6: G1, T4, F5, R8, N9, N11, F12, A26 and Q29.

The CsgF peptide may be modified to introduce a cysteine, a hydrophobic amino acid, a charged amino acid, a non-native reactive amino acid, or photoreactive amino acid, for example at a position corresponding to one or more of the following positions in SEQ ID NO: 6: G1, T4, F5, R8, N9, N11, F12, A26 and Q29.

For example, the CsgF peptide may comprise a modification at a position corresponding to one or more of the following positions in SEQ ID NO: 6: N15, N17, A20, N24 and A28. The CsgF peptide may comprise a modification at a position corresponding to D34 to stabilise the CsgG-CsgF complex. In particular embodiments, the CsgF peptide comprises one or more of the substitutions: N15S/A/T/Q/G/L/V/I/F/Y/W/R/K/D/C, N17S/A/T/Q/G/L/V/I/F/Y/W/R/K/D/C, A20S/T/Q/N/G/L/V/I/F/Y/W/R/K/D/C, N24S/T/Q/A/G/L/V/I/F/Y/W/R/K/D/C, A28S/T/Q/N/G/L/V/I/F/Y/W/R/K/D/C and D34F/Y/W/R/K/N/Q/C. The CsgF peptide may, for example, comprise one or more of the following substitutions: G1C, T4C, N17S, and D34Y or D34N.

The CsgF peptide may be produced by cleavage of a longer protein, such as full-length CsgF using an enzyme. Cleavage at a particular site may be directed by modifying the longer protein, such as full-length CsgF, to include an enzyme cleavage site at an appropriate position.

Examples of CsgF amino acid sequences that have been modified to include such enzyme cleavage sites are shown in SEQ ID NOs: 56 to 67. Following cleavage all or part of the added enzyme cleavage site may be present in the CsgF peptide that associates with CsgG to form a pore. Thus the CsgF peptide may further comprise all or part of an enzyme cleavage site at its C-terminal end.

Some examples of suitable CsgF peptides are shown in Table 3 below:

TABLE 3
CsgF peptides

| | | SEQ |
|---|---|---|
| Technical | | ID |
| description | Protein sequence | NO: |
| CsgF-(1-45) | GTMTFQFRNPNFGGNPNNGAFLLNSAQ | 40 |
| | AQNSYKDPSYNDDFGIET | |
| CsgF-(1-29) | GTMTFQFRNPNFGGNPNNGAFLLNSAQ | 39 |
| | AQ | |
| CsgF-(1-35) | GTMTFQFRNPNFGGNPNNGAFLLNSAQ | 55 |
| | AQNSYKDP | |
| CsgF-G1C-(1-45) | CTMTFQFRNPNFGGNPNNGAFLLNSAQ | 89 |
| | AQNSYKDPSYND DFGIET | |
| CsgF-G1C-(1-30) | CTMTFQFRNPNFGGNPNNGAFLLNSAQ | 90 |
| | AQ | |
| CsgF-G1C-(1-35) | CTMTFQFRNPNFGGNPNNGAFLLNSAQ | 91 |
| | AQNSYKDP | |
| CsgF-N17S-(1-35) | GTMTFQFRNPNFGGNPSNGAFLLNSAQ | 92 |
| | AQNSYKDP | |
| CsgF-G1C/D34Y- | CTMTFQFRNPNFGGNPNNGAFLLNSAQ | 93 |
| (1-35) | AQNSYKYP | |
| CsgF-N17S-(1-45) | GTMTFQFRNPNFGGNPSNGAFLLNSAQ | 94 |
| | AQNSYKDPSYND DFGIET | |
| CsgF-G1C/N17S/ | CTMTFQFRNPNFGGNPSNGAFLLNSAQ | 95 |
| D34Y-(1-35) | AQNSYKYP | |
| CsgF-G1C-(1-30)- | CTMTFQFRNPNFGGNPNNGAFLLNSAQ | 96 |
| H10 | AQHHHHHHHHHH | |
| CsgF-G1C-(1-35)- | CTMTFQFRNPNFGGNPNNGAFLLNSAQ | 97 |
| H10 | AQNSYKDPHHHHHHHHHH | |
| CsgF-N17S/D34Y- | GTMTFQFRNPNFGGNPSNGAFLLNSAQ | 98 |
| (1-35) | AQNSYKYP | |
| CsgF-N17S/D34N- | GTMTFQFRNPNFGGNPSNGAFLLNSAQ | 99 |
| (1-35) | AQNSYKNP | |
| CsgF-T4C/N17S- | GTMCFQFRNPNFGGNPSNGAFLLNSAQ | 100 |
| (1-35) | AQNSYKDP | |
| CsgF-T4C/N17S/ | GTMCFQFRNPNFGGNPSNGAFLLNSAQ | 101 |
| D34Y-(1-35) | AQNSYKYP | |
| CsgF-T4C/N17S/ | GTMCFQFRNPNFGGNPSNGAFLLNSAQ | 102 |
| D34N-(1-35) | AQNSYKNP | |
| CsgF-G1C/T4C/ | CTMCFQFRNPNFGGNPSNGAFLLNSAQ | 103 |
| N17S-(1-35) | AQNSYKDP | |
| CsgF-G1C/T4C/ | CTMCFQFRNPNFGGNPSNGAFLLNSAQ | 104 |
| N17S/D34N-(1-35) | AQNSYKNP | |
| CsgF-G1C/T4C/ | CTMCFQFRNPNFGGNPSNGAFLLNSAQ | 105 |
| N17S/D34Y-(1-35) | AQNSYKYP | |
| CsgF-N17V-(1-35) | CTMTFQFRNPNFGGNPVNGAFLLNSAQ | 106 |
| | AQNSYKDP | |
| CsgF-N17A-(1-35) | GTMTFQFRNPNFGGNPANGAFLLNSAQ | 107 |
| | AQNSYKDP | |
| CsgF-N17F-(1-35) | GTMTFQFRNPNFGGNPFNGAFLLNSAQ | 108 |
| | AQNSYKDP | |
| CsgF-N17S-(1-30) | GTMTFQFRNPNFGGNPSNGAFLLNSAQ | 109 |
| | AQN | |
| CsgF-N17V-(1-30) | GTMTFQFRNPNFGGNPVNGAFLLNSAQ | 110 |
| | AQN | |
| CsgF-N17A-(1-30) | GTMTFQFRNPNFGGNPANGAFLLNSAQ | 111 |
| | AQN | |
| CsgF-N17F-(1-30) | GTMTFQFRNPNFGGNPFNGAFLLNSAQ | 112 |
| | AQN | |
| CsgF-N17V/D34Y- | GTMTFQFRNPNFGGNPVNGAFLLNSAQ | 113 |
| (1-35) | AQNSYKYP | |
| CsgF-N17A/D34Y- | GTMTFQFRNPNFGGNPANGAFLLNSAQ | 114 |
| (1-35) | AQNSYKYP | |
| CsgF-N17F/D34Y- | GTMTFQFRNPNFGGNPFNGAFLLNSAQ | 115 |
| (1-35) | AQNSYKYP | |
| CsgF-N17S/A20Q- | GTMTFQFRNPNFGGNPSNGQFLLNSAQ | 116 |
| (1-35) | AQNSYKDP | |

In particular embodiments, said CsgF fragment comprises the amino acid sequence SEQ ID NO:39, or mutant or homologue thereof. In particular, SEQ ID NO:39 comprises the first 29 amino acids of the mature CsgF peptide (SEQ ID NO:6). In another embodiment, the modified CsgF peptide of the invention is a truncated peptide comprising SEQ ID NO:40. In particular, SEQ ID NO:40 comprises the first 45 amino acids of the mature CsgF peptide (SEQ ID NO:6). In particular, the CsgF constriction site and binding site to the CsgG are located within the N-terminal CsgF peptide region, further characterised in that amino acid 39 to 64 of SEQ ID NO:5 (present in SEQ ID NO:39 and SEQ ID NO:40), or in particular amino acid 49 to 64 of SEQ ID NO:5 (present in SEQ ID NO:40, but not in SEQ ID NO:39, the latter fragment encoded by SEQ ID NO:39 showing a weaker interaction with CsgG (see Examples)), confer a higher stability to the complex. Hence, the disclosure provides a modification of the CsgF protein by truncating the protein to said peptides or peptides comprising said N-terminal fragments or constriction site region to allow complex formation with the CsgG pore, or homologues or mutants thereof, in vivo. Further limitation is provided in one embodiment relating to a modified CsgF peptide comprising SEQ ID NO:37 or SEQ ID NO:38. Finally, identification of CsgF homologous peptides, especially aligned within the constriction region (FCP peptides), also provide modified CsgF peptide homologues that may form a part of said isolated complex.

A further embodiment relates to the modified or truncated CsgF peptides comprising SEQ ID NO:15, wherein said SEQ ID NO:15 contains the region of the CsgF protein including several residues from the region of the CsgG binding and/or constriction site, sufficient for in vitro reconstitution of the complex pore comprising CsgG or a homologue thereof, and a modified CsgF peptide, to result in an isolated pore complex comprising a CsgF channel constriction. Another embodiment describes said modified CsgF peptide comprising SEQ ID NO:16, which contains an N-terminal fragment of the CsgF protein, and two additional amino acids (KD), which will increase solubility and stability of the (synthetic) peptide, as well to allow in vitro reconstitution of said complex pore. Further embodiments are provided wherein said modified CsgF peptide comprises SEQ ID NO:15, SEQ ID NO:16 or a homologue or mutant thereof, wherein said modified CsgF peptide is further mutated, but still retains a minimal of 35% amino acid identity to SEQ ID NO:15, or SEQ ID NO:16, respectively, within the region of the modified CsgF peptide corresponding to said SEQ ID NO:15 or 16 e.g., 40%, 50%, 60%, 70%, 80% 85%, 90% amino acid identity. Further embodiments are provided wherein said modified CsgF peptide comprises SEQ ID NO:15, SEQ ID NO:16 or a homologue or mutant thereof, wherein said modified CsgF peptide is further mutated, but still retains a minimal of 40%, 45%, 50%, 60%, 70%, 80% 85% or 90% amino acid identity to SEQ ID NO:15, or SEQ ID NO:16, respectively, within the region of the modified CsgF peptide corresponding to said SEQ ID NO:15 or 16. Those mutated regions are intended to alter and/or improve the characteristics of the CsgF constriction site, as discussed above, so for instance a more accurate target analysis can be obtained. Another embodiment discloses modified CsgF peptides wherein one or more positions in the regions comprising SEQ ID NO:39, SEQ ID NO:40, SEQ ID NO:54 or SEQ ID NO:55 are modified, and wherein said mutation(s) retain a minimal of 35% amino acid identity, or 40%, 50%, 60%, 70%, 80%, 85%, 90%, 95% amino acid identity to SEQ ID NO:39, SEQ ID NO:40, SEQ ID NO:54 or SEQ ID NO:55 in the peptide fragment corresponding to the region comprising SEQ ID NO:39, SEQ ID NO:40, SEQ ID NO:54 or SEQ ID NO:55.

Additional embodiments relate to an isolated pore complex, wherein said CsgG pore, at least via one monomer, and the modified CsgF peptide, are coupled via covalent binding. Said covalent link or binding is in one instance possible via cysteine linkage, wherein the sulfhydryl side group of cysteine covalently links with another amino acid residue or moiety. In a second possibility, the covalent linkage is obtained via an interaction between non-native (photo)reactive amino acids. (Photo-)reactive amino acids are referring to artificial analogs of natural amino acids that can be used for crosslinking of protein complexes, and may be incorporated into proteins and peptides in vivo or in vitro. Photo-reactive amino acid analogs in common use are photoreactive diazirine analogs to leucine and methionine, and para-benzoyl-phenyl-alanine, as well as azidohomoalanine, homopropargylglycyine, homoallelglycine, p-acetyl-Phe, p-azido-Phe, p-propargyloxy-Phe and p-benzoyl-Phe (Wang et al. 2012; Chin et al. 2002). Upon exposure to ultraviolet light, they are activated and covalently bind to interacting proteins that are within a few angstroms of the photo-reactive amino acid analog. However, the positions in the CsgG monomer where said covalent linkages may take place is dependent on the exposure to the modified CsgF peptide. Several amino acids are in the position to provide the covalent linkage, namely positions 132, 133, 136, 138, 140, 142, 144, 145, 147, 149, 151, 153, 155, 183, 185, 187, 189, 191, 201, 203, 205, 207 or 209 of SEQ ID NO: 3 or SEQ ID NO: 117, or of homologues thereof.

Another aspect of the invention relates to constructs comprising said modified CsgF peptide, wherein said peptide is covalently attached. A “construct” comprises two or more covalently attached monomers derived from modified CsgF and/or CsgG, or a homologue thereof. In other words, a construct may contain more than one monomer. In another aspect, the invention also provides a pore complex comprising at least one construct of the invention. The pore complex contains sufficient constructs and, if necessary, monomers to form the pore. For instance, an octameric pore may comprise (a) four constructs each comprising two monomers, (b) two constructs each comprising four monomers, (c) one construct comprising two monomers and six monomers that do not form part of a construct, or (d) one or two CsgF monomers in one construct, and one construct with six to seven CsgG monomers or even (e) a construct with CsgF and CsgG monomer in addition to another construct solely comprising CsgG monomers. Same and additional possibilities are provided for a nonameric pore for instance. Other combinations of constructs and monomers can be envisaged by the skilled person. One or more constructs of the invention may be used to form a pore complex for characterising, such as sequencing, polynucleotides. The construct may comprise at least 2, at least 3, at least 4, at least 5, at least 6, at least 7, at least 8, at least 9 or at least 10 monomers. The construct preferably comprises two monomers. The two or more monomers may be the same or different, may be CsgF, CsgG, CsgG/CsgF fusion monomers or homologues thereof, or any combination thereof.

Another embodiment relates to the polynucleotide or nucleic acid molecule encoding said pore or pore complex the invention, or homologues or mutants thereof, or polynucleotides encoding a construct as described above.

Certain embodiments relate to an isolated transmembrane pore complex comprising the isolated pore complex or isolated pore complex of the invention, and the components of a membrane. Said isolated transmembrane pore complex is directly applicable for use in molecular sensing, such as nucleic acid sequencing. Alternatively, a membranous composition is provided, comprising a modified CsgG/CsgF biological pore as described herein, according to the isolated pore complex of the invention, and a membrane, membrane components, or an insulating layer. One embodiment relates to an isolated transmembrane pore complex consisting of the isolated pore complex according to the invention, and the components of a membrane.

Although the CsgG:CsgF complex is very stable, when CsgF is truncated, the stability of CsgG:CsgF complexes decrease compared to a complex comprising full length CsgF. Therefore, disulphide bonds can be made between CsgG and CsgF to make the complex more stable, for example following introduction of cysteine residues at the positions identified herein. The pore complex can be made in any of the previously mentioned methods and disulphide bond formation can be induced by using oxidising agents (eg: Copper-orthophenanthroline). Other interactions (eg: hydrophobic interactions, charge-charge interactions/electrostatic interactions) can also be used in those positions instead of cysteine interactions.

In another embodiment, unnatural amino acids can also be incorporated in those positions. In this embodiment, covalent bonds made be made by via click chemistry. For example, unnatural amino acids with azide or alkyne or with a dibenzocyclooctyne (DBCO) group and/or a bicyclo[6.1.0]nonyne (BCN) group may be introduced at one or more of these positions.

Such stabilising mutations can be combined with any other modifications to CsgG and/or CsgF, for example the modifications disclosed herein.

The CsgG pore may comprise at least one, such as 2, 3, 4, 5, 6, 7, 8, 9 or 10, CsgG monomers that is/are modified to facilitate attachment to the CsgF peptide. For example a cysteine residue may be introduced at one or more of the positions corresponding to positions 132, 133, 136, 138, 140, 142, 144, 145, 147, 149, 151, 153, 155, 183, 185, 187, 189, 191, 201, 203, 205, 207 and 209 of SEQ ID NO: 3 or SEQ ID NO: 117 to facilitate covalent attachment to CsgG. As an alternative or addition to covalent attachment via cysteine residues, the pore may be stabilised by hydrophobic interactions or electrostatic interactions. To facilitate such interactions, a non-native reactive or photoreactive amino acid at a position corresponding to one or more of positions 132, 133, 136, 138, 140, 142, 144, 145, 147, 149, 151, 153, 155, 183, 185, 187, 189, 191, 201, 203, 205, 207 and 209 of SEQ ID NO: 3 or SEQ ID NO: 117.

The CsgF peptide may be modified to facilitate attachment to the CsgG pore. For example a cysteine residue may be introduced at one or more of the positions corresponding to positions 1, 4, 5, 8, 9, 11, 12, 26 or 29 of SEQ ID NO: 6 to facilitate covalent attachment to CsgG. As an alternative or addition to covalent attachment via cysteine residues, the pore may be stabilised by hydrophobic interactions or electrostatic interactions. To facilitate such interactions, a non-native reactive or photoreactive amino acid at a position corresponding to one or more of positions 1, 4, 5, 8, 9, 11, 12, 26 or 29 of SEQ ID NO: 6.

Preferred exemplary CsgF peptides include comprise the following mutations relative to SEQ ID NO: 6: N15X1/N17X2/A20X3/N24X4/A28X5/D34X6, wherein X1 is N/S/A/T/Q/G/L/V/I/F/Y/W/R/K/D/C, X2 is N/S/A/T/Q/G/L/V/I/F/Y/W/R/K/D/C, X3 is A/S/T/Q/N/G/L/V/I/F/Y/W/R/K/D/C, X4 is N/S/T/Q/A/G/L/V/I/F/Y/W/R/K/D/C, X5 is A/S/T/Q/N/G/L/V/I/F/Y/W/R/K/D/C and X5 is D/F/Y/W/R/K/N/Q/C. The mutations at positions N15, N17, A20, N24 and A28 are constriction mutations and the mutation at position 34 affects the interaction pf CsgF with the bottom of the CsgG pore to stabilise the interaction.

### CsgG Pore

The CsgG pore may be a homo-oligomeric pore comprising identical mutant monomers of the invention. The CsgG pore may be a hetero-oligomeric pore derived from CsgG, for example comprising at least one mutant monomer as disclosed herein.

The CsgG pore may contain any number of mutant monomers. The pore typically comprises at least 7, at least 8, at least 9 or at least 10 identical mutant monomers, such as 7, 8, 9 or 10 mutant monomers. The CsgG pore preferably comprises eight or nine identical mutant monomers.

In a preferred embodiment, all of the monomers in the hetero-oligomeric CsgG pore (such as 10, 9, 8 or 7 of the monomers) are mutant monomers as disclosed herein, wherein at least one of them differs from the others. They may all differ from one another.

The mutant monomers in the CsgG pore are preferably all approximately the same length or are the same length. The barrels of the mutant monomers of the invention in the pore are preferably approximately the same length or are the same length. Length may be measured in number of amino acids and/or units of length.

A mutant monomer may be a variant of SEQ ID NO: 3 or SEQ ID NO: 117 comprising a modification at one or more of positions W97, Q100, E101, N102, and T104. Over the entire length of the amino acid sequence of SEQ ID NO: 3 or SEQ ID NO: 117, a variant will preferably be at least 40% homologous to that sequence based on amino acid identity. More preferably, the variant may be at least 45%, at least 50%, at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 3 or SEQ ID NO: 117 over the entire sequence. Over the entire length of the amino acid sequence of SEQ ID NO: 3 or SEQ ID NO: 117, a variant will preferably be at least 40% identical to that sequence. More preferably, the variant may be at least 45%, at least 50%, at least 55%, at least 60%, at least 65%, at least 70%, at least 75%, at least 80%, at least 85%, at least 90% and more preferably at least 95%, 97% or 99% identical to SEQ ID NO: 3 or SEQ ID NO: 117 over the entire sequence. There may be at least 80%, for example at least 85%, 90% or 95%, amino acid identity over a stretch of 100 or more, for example 125, 150, 175 or 200 or more, contiguous amino acids (“hard homology”).

CsgG monomers are highly conserved (as can be readily appreciated from FIGS. 45 to 47 of WO2017/149317). Furthermore, from knowledge of the mutations in relation to SEQ ID NO: 3 or SEQ ID NO: 117 it is possible to determine the equivalent positions for mutations of CsgG monomers other than that of SEQ ID NO: 3 or SEQ ID NO: 117.

Thus reference to a mutant CsgG monomer comprising a variant of the sequence as shown in SEQ ID NO: 3 or SEQ ID NO: 117 and specific amino-acid mutations thereof as set out in the claims and elsewhere in the specification also encompasses a mutant CsgG monomer comprising a variant of the sequence as shown in SEQ ID NOs: 68 to 88 and corresponding amino-acid mutations thereof. Likewise reference to a construct, pore or method involving the use of a pore relating to a mutant CsgG monomer comprising a variant of the sequence as shown in SEQ ID NO: 3 or SEQ ID NO: 117 and specific amino-acid mutations thereof as set out in the claims and elsewhere in the specification also encompasses a construct, pore or method relating to a mutant CsgG monomer comprising a variant of the sequence according the above disclosed SEQ ID NOs and corresponding amino-acid mutations thereof. If will further be appreciated that the invention extends to other variant CsgG monomers not expressly identified in the specification that show highly conserved regions.

Standard methods in the art may be used to determine homology. For example the UWGCG Package provides the BESTFIT program which can be used to calculate homology, for example used on its default settings (Devereux et al (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (http://www.ncbi.nlm.nih.gov/). SEQ ID NO: 3 is the wild-type CsgG monomer from Escherichia coli Str. K-12 substr. MC4100. A variant of SEQ ID NO: 3 or SEQ ID NO: 117 may comprise any of the substitutions present in another CsgG homologue. Preferred CsgG homologues are shown in SEQ ID NOs: 68 to 88. The variant may comprise combinations of one or more of the substitutions present in SEQ ID NOs: 68 to 88 compared with SEQ ID NO: 3 or SEQ ID NO: 117. For example, mutations may be made at any one or more of the positions in SEQ ID NO: 3 or SEQ ID NO: 117 that differ between SEQ ID NO: 3 or SEQ ID NO: 117 and any one of SEQ ID NOs: 68 to 88. Such a mutation may be a substitution of an amino acid in SEQ ID NO: 3 or SEQ ID NO: 117 with an amino acid from the corresponding position in any one of SEQ ID NOs: 68 to 88. Alternatively, the mutation at any one of these positions may be a substitution with any amino acid, or may be a deletion or insertion mutation, such as deletion or insertion of 1 to 10 amino acids, such as of 2 to 8 or 3 to 6 amino acids. Other than the mutations disclosed herein, the amino acids that are conserved between SEQ ID NO: 3 or SEQ ID NO: 117 and all of SEQ ID NOs: 66 to 88 are preferably present in a variant of the invention. However, conservative mutations may be made at any one or more of these positions that are conserved between SEQ ID NO: 3 or SEQ ID NO: 117 and all of SEQ ID NOs: 66 to 88.

The invention provides a pore-forming CsgG mutant monomer that comprises any one or more of the amino acids described herein as being substituted into a specific position of SEQ ID NO: 3 or SEQ ID NO: 117 at a position in the structure of the CsgG monomer that corresponds to the specific position in SEQ ID NO: 3 or SEQ ID NO: 117. Corresponding positions may be determined by standard techniques in the art. For example, the PILEUP and BLAST algorithms mentioned above can be used to align the sequence of a CsgG monomer with SEQ ID NO: 3 or SEQ ID NO: 117 and hence to identify corresponding residues.

The pore-forming mutant monomer typically retains the ability to form the same 3D structure as the wild-type CsgG monomer, such as the same 3D structure as a CsgG monomer having the sequence of SEQ ID NO: 3 or SEQ ID NO: 117. The 3D structure of CsgG is known in the art and is disclosed, for example, in Goyal et al (2014) Nature 516(7530):250-3. Any number of mutations may be made in the wild-type CsgG sequence in addition to the mutations described herein provided that the CsgG mutant monomer retains the improved properties imparted on it by the mutations of the present invention.

Typically the CsgG monomer will retain the ability to form a structure comprising three alpha-helices and five beta-sheets. Mutations may be made at least in the region of CsgG which is N-terminal to the first alpha helix (which starts at S63 in SEQ ID NO:3), in the second alpha helix (from G85 to A99 of SEQ ID NO: 3 or SEQ ID NO: 117), in the loop between the second alpha helix and the first beta sheet (from Q100 to N120 of SEQ ID NO: 3 or SEQ ID NO: 117), in the fourth and fifth beta sheets (S173 to R192 and R198 to T107 of SEQ ID NO: 3 or SEQ ID NO: 117, respectively) and in the loop between the fourth and fifth beta sheets (F193 to Q197 of SEQ ID NO: 3 or SEQ ID NO: 117) without affecting the ability of the CsgG monomer to form a transmembrane pore, which transmembrane pore is capable of translocating polypeptides. Therefore, it is envisaged that further mutations may be made in any of these regions in any CsgG monomer without affecting the ability of the monomer to form a pore that can translocate polynucleotides. It is also expected that mutations may be made in other regions, such as in any of the alpha helices (S63 to R76, G85 to A99 or V211 to L236 of SEQ ID NO: 3 or SEQ ID NO: 117) or in any of the beta sheets (I121 to N133, K135 to R142, I146 to R162, S173 to R192 or R198 to T107 of SEQ ID NO: 3 or SEQ ID NO: 117) without affecting the ability of the monomer to form a pore that can translocate polynucleotides. It is also expected that deletions of one or more amino acids can be made in any of the loop regions linking the alpha helices and beta sheets and/or in the N-terminal and/or C-terminal regions of the CsgG monomer without affecting the ability of the monomer to form a pore that can translocate polynucleotides.

Amino acid substitutions may be made to the amino acid sequence of SEQ ID NO: 3 or SEQ ID NO: 117 in addition to those discussed above, for example up to 1, 2, 3, 4, 5, 10, 20 or 30 substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 above. Where amino acids have similar polarity, this can also be determined by reference to the hydropathy scale for amino acid side chains in Table 2.

One or more amino acid residues of the amino acid sequence of SEQ ID NO: 3 or SEQ ID NO: 117 may additionally be deleted from the polypeptides described above. Up to 1, 2, 3, 4, 5, 10, 20 or 30 or more residues may be deleted.

Variants may include fragments of SEQ ID NO: 3 or SEQ ID NO: 117. Such fragments retain pore forming activity. Fragments may be at least 50, at least 100, at least 150, at least 200 or at least 250 amino acids in length. Such fragments may be used to produce the pores. A fragment preferably comprises the membrane spanning domain of SEQ ID NO: 3 or SEQ ID NO: 117, namely K135-Q153 and S183-S208.

One or more amino acids may be alternatively or additionally added to the polypeptides described above. An extension may be provided at the amino terminal or carboxy terminal of the amino acid sequence of SEQ ID NO: 3 or SEQ ID NO: 117 or polypeptide variant or fragment thereof. The extension may be quite short, for example from 1 to 10 amino acids in length. Alternatively, the extension may be longer, for example up to 50 or 100 amino acids. A carrier protein may be fused to an amino acid sequence according to the invention. Other fusion proteins are discussed in more detail below.

A CsgG pore as described herein includes a wild type CsgG pore, or a homologue or a mutant/variant thereof. A variant is a polypeptide that has an amino acid sequence which varies from that of SEQ ID NO: 3 or SEQ ID NO: 117 and which retains its ability to form a pore. A variant typically contains the regions of SEQ ID NO: 3 or SEQ ID NO: 117 that are responsible for pore formation. The pore forming ability of CsgG, which contains a □-barrel, is provided by □-sheets in each subunit. A variant of SEQ ID NO: 3 or SEQ ID NO: 117 typically comprises the regions in SEQ ID NO: 3 or SEQ ID NO: 117 that form □-sheets, namely K134-Q154 and S183-S208. One or more modifications can be made to the regions of SEQ ID NO: 3 or SEQ ID NO: 117 that form □-sheets as long as the resulting variant retains its ability to form a pore. A variant of SEQ ID NO: 3 or SEQ ID NO: 117 preferably includes one or more modifications, such as substitutions, additions or deletions, within its α-helices and/or loop regions.

The mutant CsgG monomers may be a mutant CsgG monomer, which is a monomer whose sequence varies from that of a wild-type CsgG monomer and which retains the ability to form a pore. A mutant monomer may also be referred to herein as a variant. Methods for confirming the ability of mutant monomers to form pores are well-known in the art and are discussed in more detail below.

The at least one monomer, or any or all of the six to ten monomers, in the CsgG pore or pore complex of the invention may have any of the particular modifications or substitutions disclosed in WO2016/034591, WO2017/149316, WO2017/149317, WO2017/149318, WO2018/211241, and WO2019/002893 (all incorporated by reference herein in their entirety).

Preferred additional modifications in the at least one monomer/variant of SEQ ID NO: 117 in the pore or pore complex of the invention include, but are not limited to, one or more of, such as 2 or more, 3 or more, 4 or more, 5 or more, 6 or more, 7 or more, 8 or more or all of:
(a) a substitution at position Y51, such as Y51A, Y51I, Y51L or Y51T;
(b) a substitution at position N55, such as N55V, N55Q, N55S or N55A;
(c) a substitution at position F56, such as F56Q, F56A or F56N;
(d) a substitution at position L90, such as L90N, L90R or L90K;
(e) a substitution at position N91, such as N91N, N91R or N91K;
(f) a substitution at position K94, such as K94Q, K94R, K94F, K94Y, K94W, K94L, K94S or K94N;
(g) a substitution at position R192, such as R192D, R192Q, R192F, R192S or R192T;
(h) a substitution at position Q153, such as Q153C; and
(i) a substitution at position C215, such as C215T.

The at least one monomer/variant of SEQ ID NO: 117 may further comprise a deletion of one or more positions, such as a deletion of V105-I107, a deletion of F193-L199 or a deletion of F195-L199.

Any number of the monomers in the pore or pore complex, such as 6, 7, 8, 9 or 10, may be a mutant monomer/variant of SEQ ID NO: 117 further comprising one or more of these additional modifications in addition to a modification at one or more of positions W97, Q100, E101, N102, and T104 in SEQ ID NO: 117. All six to ten monomers in the pore or pore complex may be mutant monomers/variants of SEQ ID NO: 117 further comprising one or more of these additional substitutions in addition to a modification at one or more of positions W97, Q100, E101, N102, and T104 in SEQ ID NO: 117.

### Double Pore Complexes

The pore or pore complex of the invention may be a double pore complex comprising a first pore or complex and a second pore or complex. The double pore complex may comprise pore-pore, pore-pore complex or pore complex-pore complex. Any of the pore or complexes may be a pore or complex of the invention. In one embodiment both the first pore complex and the second pore complex are CsgG/CsgF pore complexes of the invention. In another embodiment both the first pore and the second pore are CsgG pores of the invention. The first and second pores or pore complexes may be the same or different. In addition to any of the mutations disclosed herein, in a double pore, the at least one CsgG monomer may comprise one or more of the additional mutations described in WO2016/034591, WO2017/149316, WO2017/149317 and, WO2017/149318, WO2018/211241, and WO2019/002893 (all incorporated by reference herein in their entirety). The at least one CsgG monomer preferably comprises any of the additional substitutions disclosed above.

### Method for Making Modified Proteins

Methods for introducing or substituting non-naturally-occurring amino acids are also well known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. They may also be produced by naked ligation if the mutant monomer is produced using partial peptide synthesis.

The monomers derived from CsgG may be modified to assist their identification or purification, for example by the addition of a streptavidin tag or by the addition of a signal sequence to promote their secretion from a cell where the monomer does not naturally contain such a sequence. Other suitable tags are discussed in more detail below. The monomer may be labelled with a revealing label. The revealing label may be any suitable label which allows the monomer to be detected. Suitable labels are described below.

The monomer derived from CsgG may also be produced using D-amino acids. For instance, the monomer derived from CsgG may comprise a mixture of L-amino acids and D-amino acids. This is conventional in the art for producing such proteins or peptides.

The monomer derived from CsgG contains one or more specific modifications to facilitate nucleotide discrimination. The monomer derived from CsgG may also contain other non-specific modifications as long as they do not interfere with pore formation. A number of non-specific side chain modifications are known in the art and may be made to the side chains of the monomer derived from CsgG. Such modifications include, for example, reductive alkylation of amino acids by reaction with an aldehyde followed by reduction with NaBH4, amidination with methylacetimidate or acylation with acetic anhydride.

The monomer derived from CsgG can be produced using standard methods known in the art. The monomer derived from CsgG may be made synthetically or by recombinant means. For example, the monomer may be synthesised by in vitro translation and transcription (IVTT). Suitable methods for producing pores and monomers are discussed in the International applications WO 2010/004273, WO 2010/004265, or WO 2010/086603 (incorporated herein by reference in their entirety). Methods for inserting pores into membranes are known.

Two or more CsgG monomers in the pore may be covalently attached to one another. For example, at least 2, at least 3, at least 4, at least 5, at least 6, at least 7, at least 8, at least 9 or at least 10 monomers may be covalently attached. The covalently attached monomers may be the same or different.

The monomers may be genetically fused, optionally via a linker, or chemically fused, for instance via a chemical crosslinker. Methods for covalently attaching monomers are disclosed in WO2017/149316, WO2017/149317, and WO2017/149318 (incorporated herein by reference in their entirety).

In some embodiments, the mutant monomer is chemically modified. The mutant monomer can be chemically modified in any way and at any site. The mutant monomer is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant monomer may be chemically modified by the attachment of any molecule. For instance, the mutant monomer may be chemically modified by attachment of a dye or a fluorophore.

In some embodiments, the mutant monomer is chemically modified with a molecular adaptor that facilitates the interaction between a pore comprising the monomer and a target nucleotide or target polynucleotide sequence. The presence of the adaptor improves the host-guest chemistry of the pore and the nucleotide or polynucleotide sequence and thereby improves the sequencing ability of pores formed from the mutant monomer. The principles of host-guest chemistry are well-known in the art. The adaptor has an effect on the physical or chemical properties of the pore that improves its interaction with the nucleotide or polynucleotide sequence. The adaptor may alter the charge of the barrel or channel of the pore or specifically interact with or bind to the nucleotide or polynucleotide sequence thereby facilitating its interaction with the pore.

The molecular adaptor is preferably a cyclic molecule, a cyclodextrin, a species that is capable of hybridization, a DNA binder or interchelator, a peptide or peptide analogue, a synthetic polymer, an aromatic planar molecule, a small positively-charged molecule or a small molecule capable of hydrogen-bonding.

The adaptor may be cyclic. A cyclic adaptor preferably has the same symmetry as the pore. The adaptor preferably has eight-fold or nine-fold symmetry since CsgG typically has eight or nine subunits around a central axis. This is discussed in more detail below.

The adaptor typically interacts with the nucleotide or polynucleotide sequence via host-guest chemistry. The adaptor is typically capable of interacting with the nucleotide or polynucleotide sequence. The adaptor comprises one or more chemical groups that are capable of interacting with the nucleotide or polynucleotide sequence. The one or more chemical groups preferably interact with the nucleotide or polynucleotide sequence by non-covalent interactions, such as hydrophobic interactions, hydrogen bonding, Van der Waal's forces, □-cation interactions and/or electrostatic forces. The one or more chemical groups that are capable of interacting with the nucleotide or polynucleotide sequence are preferably positively charged. The one or more chemical groups that are capable of interacting with the nucleotide or polynucleotide sequence more preferably comprise amino groups. The amino groups can be attached to primary, secondary or tertiary carbon atoms. The adaptor even more preferably comprises a ring of amino groups, such as a ring of 6, 7 or 8 amino groups. The adaptor most preferably comprises a ring of eight amino groups. A ring of protonated amino groups may interact with negatively charged phosphate groups in the nucleotide or polynucleotide sequence.

The correct positioning of the adaptor within the pore can be facilitated by host-guest chemistry between the adaptor and the pore comprising the mutant monomer. The adaptor preferably comprises one or more chemical groups that are capable of interacting with one or more amino acids in the pore. The adaptor more preferably comprises one or more chemical groups that are capable of interacting with one or more amino acids in the pore via non-covalent interactions, such as hydrophobic interactions, hydrogen bonding, Van der Waal's forces, □-cation interactions and/or electrostatic forces. The chemical groups that are capable of interacting with one or more amino acids in the pore are typically hydroxyls or amines. The hydroxyl groups can be attached to primary, secondary or tertiary carbon atoms. The hydroxyl groups may form hydrogen bonds with uncharged amino acids in the pore. Any adaptor that facilitates the interaction between the pore and the nucleotide or polynucleotide sequence can be used.

Suitable adaptors include, but are not limited to, cyclodextrins, cyclic peptides and cucurbiturils. The adaptor is preferably a cyclodextrin or a derivative thereof. The cyclodextrin or derivative thereof may be any of those disclosed in Eliseev, A. V., and Schneider, H-J. (1994) J. Am. Chem. Soc. 116, 6081-6088. The adaptor is more preferably heptakis-6-amino-3-cyclodextrin (am7-βCD), 6-monodeoxy-6-monoamino-β-cyclodextrin (am1-□CD) or heptakis-(6-deoxy-6-guanidino)-cyclodextrin (gu7-βCD). The guanidino group in gu7-βCD has a much higher pKa than the primary amines in am7-βCD and so it is more positively charged. This gu7-βCD adaptor may be used to increase the dwell time of the nucleotide in the pore, to increase the accuracy of the residual current measured, as well as to increase the base detection rate at high temperatures or low data acquisition rates.

If a succinimidyl 3-(2-pyridyldithio)propionate (SPDP) crosslinker is used as discussed in more detail below, the adaptor is preferably heptakis(6-deoxy-6-amino)-6-N-mono(2-pyridyl)dithiopropanoyl-β-cyclodextrin (am6amPDP1-βCD).

More suitable adaptors include 7-cyclodextrins, which comprise 9 sugar units (and therefore have nine-fold symmetry). The 7-cyclodextrin may contain a linker molecule or may be modified to comprise all or more of the modified sugar units used in the β-cyclodextrin examples discussed above.

The molecular adaptor may be covalently attached to the mutant monomer. The adaptor can be covalently attached to the pore using any method known in the art. The adaptor is typically attached via chemical linkage. If the molecular adaptor is attached via cysteine linkage, the one or more cysteines have preferably been introduced to the mutant, for instance in the barrel, by substitution. The mutant monomer may be chemically modified by attachment of a molecular adaptor to one or more cysteines in the mutant monomer. The one or more cysteines may be naturally-occurring, i.e. at positions 1 and/or 215 in SEQ ID NO: 3 or SEQ ID NO: 117. Alternatively, the mutant monomer may be chemically modified by attachment of a molecule to one or more cysteines introduced at other positions. The cysteine at position 215 may be removed, for instance by substitution, to ensure that the molecular adaptor does not attach to that position rather than the cysteine at position 1 or a cysteine introduced at another position.

The reactivity of cysteine residues may be enhanced by modification of the adjacent residues. For instance, the basic groups of flanking arginine, histidine or lysine residues will change the pKa of the cysteines thiol group to that of the more reactive S-group. The reactivity of cysteine residues may be protected by thiol protective groups such as dTNB. These may be reacted with one or more cysteine residues of the mutant monomer before a linker is attached.

The molecule may be attached directly to the mutant monomer. The molecule is preferably attached to the mutant monomer using a linker, such as a chemical crosslinker or a peptide linker.

Suitable chemical crosslinkers are well-known in the art. Preferred crosslinkers include 2,5-dioxopyrrolidin-1-yl 3-(pyridin-2-yldisulfanyl)propanoate, 2,5-dioxopyrrolidin-1-yl 4-(pyridin-2-yldisulfanyl)butanoate and 2,5-dioxopyrrolidin-1-yl 8-(pyridin-2-yldisulfanyl)octananoate. The most preferred crosslinker is succinimidyl 3-(2-pyridyldithio)propionate (SPDP). Typically, the molecule is covalently attached to the bifunctional crosslinker before the molecule/crosslinker complex is covalently attached to the mutant monomer but it is also possible to covalently attach the bifunctional crosslinker to the monomer before the bifunctional crosslinker/monomer complex is attached to the molecule.

The linker is preferably resistant to dithiothreitol (DTT). Suitable linkers include, but are not limited to, iodoacetamide-based and Maleimide-based linkers.

In other embodiment, the monomer may be attached to a polynucleotide binding protein. This forms a modular sequencing system that may be used in the methods of sequencing of the invention. Polynucleotide binding proteins are discussed below.

The polynucleotide binding protein is preferably covalently attached to the mutant monomer. The protein can be covalently attached to the monomer using any method known in the art. The monomer and protein may be chemically fused or genetically fused. The monomer and protein are genetically fused if the whole construct is expressed from a single polynucleotide sequence. Genetic fusion of a monomer to a polynucleotide binding protein is discussed in WO 2010/004265 (incorporated herein by reference in its entirety).

If the polynucleotide binding protein is attached via cysteine linkage, the one or more cysteines have preferably been introduced to the mutant by substitution. The one or more cysteines are preferably introduced into loop regions which have low conservation amongst homologues indicating that mutations or insertions may be tolerated. They are therefore suitable for attaching a polynucleotide binding protein. In such embodiments, the naturally-occurring cysteine at position 251 may be removed. The reactivity of cysteine residues may be enhanced by modification as described above.

The polynucleotide binding protein may be attached directly to the mutant monomer or via one or more linkers. The molecule may be attached to the mutant monomer using the hybridization linkers described in as WO 2010/086602 (incorporated herein by reference in its entirety). Alternatively, peptide linkers may be used. Peptide linkers are amino acid sequences. The length, flexibility and hydrophilicity of the peptide linker are typically designed such that it does not to disturb the functions of the monomer and molecule. Preferred flexible peptide linkers are stretches of 2 to 20, such as 4, 6, 8, 10 or 16, serine and/or glycine amino acids. More preferred flexible linkers include (SG)1, (SG)2, (SG)3, (SG)4, (SG)5 and (SG)8 wherein S is serine and G is glycine. Preferred rigid linkers are stretches of 2 to 30, such as 4, 6, 8, 16 or 24, proline amino acids. More preferred rigid linkers include (P)12 wherein P is proline.

### Chemical Modification

The mutant CsgG monomer or CsgF peptide may be chemically modified with a molecular adaptor and a polynucleotide binding protein.

The molecule (with which the monomer or peptide is chemically modified) may be attached directly to the monomer or peptide or attached via a linker as disclosed in WO 2010/004273, WO 2010/004265 or WO 2010/086603 (incorporated herein by reference in their entirety).

Any of the proteins described herein, such as the CsgG monomers and/or CsgF peptides, may be modified to assist their identification or purification, for example by the addition of histidine residues (a his tag), aspartic acid residues (an asp tag), a streptavidin tag, a flag tag, a SUMO tag, a GST tag or a MBP tag, or by the addition of a signal sequence to promote their secretion from a cell where the polypeptide does not naturally contain such a sequence. An alternative to introducing a genetic tag is to chemically react a tag onto a native or engineered position on the protein. An example of this would be to react a gel-shift reagent to a cysteine engineered on the outside of the protein. This has been demonstrated as a method for separating hemolysin hetero-oligomers (Chem Biol. 1997 July; 4(7):497-505).

Any of the proteins described herein, such as the CsgG monomers and/or CsgF peptides, may be labelled with a revealing label. The revealing label may be any suitable label which allows the protein to be detected. Suitable labels include, but are not limited to, fluorescent molecules, radioisotopes, e.g. 125I, 35S, enzymes, antibodies, antigens, polynucleotides and ligands such as biotin.

Any of the proteins described herein, such as the CsgG monomers and/or CsgF peptides, may be made synthetically or by recombinant means. For example, the protein may be synthesised by in vitro translation and transcription (IVTT). The amino acid sequence of the protein may be modified to include non-naturally occurring amino acids or to increase the stability of the protein. When a protein is produced by synthetic means, such amino acids may be introduced during production. The protein may also be altered following either synthetic or recombinant production.

Proteins may also be produced using D-amino acids. For instance, the protein may comprise a mixture of L-amino acids and D-amino acids. This is conventional in the art for producing such proteins or peptides.

The protein may also contain other non-specific modifications as long as they do not interfere with the function of the protein. A number of non-specific side chain modifications are known in the art and may be made to the side chains of the protein(s). Such modifications include, for example, reductive alkylation of amino acids by reaction with an aldehyde followed by reduction with NaBH4, amidination with methylacetimidate or acylation with acetic anhydride.

Any of the proteins described herein, such as the CsgG monomers and/or CsgF peptides, can be produced using standard methods known in the art. Polynucleotide sequences encoding a protein may be derived and replicated using standard methods in the art. Polynucleotide sequences encoding a protein may be expressed in a bacterial host cell using standard techniques in the art.

The protein may be produced in a cell by in situ expression of the polypeptide from a recombinant expression vector. The expression vector optionally carries an inducible promoter to control the expression of the polypeptide. These methods are described in Sambrook, J. and Russell, D. (2001). Molecular Cloning: A Laboratory Manual, 3rd Edition. Cold Spring Harbor Laboratory Press, Cold Spring Harbor, NY.

Proteins may be produced in large scale following purification by any protein liquid chromatography system from protein producing organisms or after recombinant expression. Typical protein liquid chromatography systems include FPLC, AKTA systems, the Bio-Cad system, the Bio-Rad BioLogic system and the Gilson HPLC system.

### Method of Producing Pores

The invention provides methods to in vivo and in vitro produce CsgG: modified CsgF pore complex holding two or more constriction sites. One embodiment provides a method for producing a transmembrane pore complex, comprising a CsgG pore, or homologue or mutant form thereof, and the modified CsgF peptide, or its homologue or mutant, via co-expression. Said method comprising the steps of expressing CsgG monomers (expressed as preprotein provided in SEQ ID NO: 2, or a homologue or mutant thereof), and expressing modified or truncated CsgF monomers, both in a suitable host cell, allowing in vivo complex pore formation. Said complex comprises modified CsgF peptides, in complex with the CsgG pore, to provide the pore with an additional reader head. The resulting pore complex produced by said method using modified CsgF peptides provides a structure that is sufficient for a use of the pore complex in characterization of target analytes such as nucleic acid sequencing, as it allows passage of the analytes, in particular polynucleotide strands, and comprises two or more reader heads for improved reading of said polynucleotide sequence, when used in the appropriate settings for said application. Methods for making the pores and complexes of the invention and ways of tagging them are disclosed in WO2016/034591, WO2017/149316, WO2017/149317 and, WO2017/149318, WO2018/211241, and WO2019/002893 (all incorporated by reference herein in their entirety).

### Methods of Characterising an Analyte

The invention provides a method of determining the presence, absence or one or more characteristics of a target analyte. The method involves contacting the target analyte with an isolated pore complex, or transmembrane pore, such as a pore of the invention, such that the target analyte moves with respect to, such as into or through, the pore channel and taking one or more measurements as the analyte moves with respect to the pore and thereby determining the presence, absence or one or more characteristics of the analyte. The target analyte may also be called the template analyte or the analyte of interest. The isolated pore complex typically comprises at least 7, at least 8, at least 9 or at least 10 monomers, such as 7, 8, 9 or 10 CsgG monomers. The isolated pore complex preferably comprises eight or nine identical CsgG monomers. One or more, such as 2, 3, 4, 5, 6, 7, 8, 9 or 10, of the CsgG monomers is preferably chemically modified, or the CsgF peptide is chemically modified. The isolated pore complex monomers, such as the CsgG monomers, or homologues or mutants thereof, and the modified CsgF monomers, or homologues or mutants thereof, may be derived from any organism. The analyte may pass through the CsgG constriction, followed by the CsgF constriction. In an alternative embodiment the analyte may pass through the CsgF constriction, followed by the CsgG constriction, depending on the orientation of the CsgG/CsgF complex in the membrane. The method is for determining the presence, absence or one or more characteristics of a target analyte. The method may be for determining the presence, absence or one or more characteristics of at least one analyte. The method may concern determining the presence, absence or one or more characteristics of two or more analytes. The method may comprise determining the presence, absence or one or more characteristics of any number of analytes, such as 2, 5, 10, 15, 20, 30, 40, 50, 100 or more analytes. Any number of characteristics of the one or more analytes may be determined, such as 1, 2, 3, 4, 5, 10 or more characteristics.

The binding of a molecule in the channel of the pore complex, or in the vicinity of either opening of the channel will have an effect on the open-channel ion flow through the pore, which is the essence of “molecular sensing” of pore channels. In a similar manner to the nucleic acid sequencing application, variation in the open-channel ion flow can be measured using suitable measurement techniques by the change in electrical current (for example, WO 2000/28312 and D. Stoddart et al., Proc. Natl. Acad. Sci., 2010, 106, 7702-7 or WO 2009/077734; all incorporated herein by reference in their entirety). The degree of reduction in ion flow, as measured by the reduction in electrical current, is related to the size of the obstruction within, or in the vicinity of, the pore. Binding of a molecule of interest, also referred to as an “analyte”, in or near the pore therefore provides a detectable and measurable event, thereby forming the basis of a “biological sensor”. Suitable molecules for nanopore sensing include nucleic acids; proteins; peptides; polysaccharides and small molecules (refers here to a low molecular weight (e.g., <900 Da or <500 Da) organic or inorganic compound) such as pharmaceuticals, toxins, cytokines, and pollutants. Detecting the presence of biological molecules finds application in personalised drug development, medicine, diagnostics, life science research, environmental monitoring and in the security and/or the defence industry.

In another aspect, the isolated pore complex, or the transmembrane pore complex containing a wild type or modified E. coli CsgG nanopore, or homologue or mutant thereof, and a modified CsgF peptide providing a channel constriction to the pore within the complex, may serve as a molecular or biological sensor. In some embodiments, the CsgG nanopore can be derived or isolated from bacterial proteins (e.g., E. coli, Salmonella typhi). In some embodiments, the CsgG nanopore can be recombinantly produced. Procedures for analyte detection are described in Howorka et al. Nature Biotechnology (2012) Jun. 7; 30(6):506-7. The analyte molecule that is to be detected may bind to either face of the channel, or within the lumen of the channel itself. The position of binding may be determined by the size of the molecule to be sensed.

The target analyte is preferably a metal ion, an inorganic salt, a polymer, an amino acid, a peptide, a polypeptide, a protein, a nucleotide, an oligonucleotide, a polynucleotide, a polysaccharide, a dye, a bleach, a pharmaceutical, a diagnostic agent, a recreational drug, an explosive, a toxic compound, or an environmental pollutant. The method may concern determining the presence, absence or one or more characteristics of two or more analytes of the same type, such as two or more proteins, two or more nucleotides or two or more pharmaceuticals. Alternatively, the method may concern determining the presence, absence or one or more characteristics of two or more analytes of different types, such as one or more proteins, one or more nucleotides and one or more pharmaceuticals.

The target analyte can be secreted from cells. Alternatively, the target analyte can be an analyte that is present inside cells such that the analyte must be extracted from the cells before the method can be carried out.

A wild-type pore may act as sensor, but is often modified via recombinant or chemical methods to increase the strength of binding, the position of binding, or the specificity of binding of the molecule to be sensed. Typical modifications include addition of a specific binding moiety complimentary to the structure of the molecule to be sensed. Where the analyte molecule comprises a nucleic acid, this binding moiety may comprise a cyclodextrin or an oligonucleotide; for small molecules this may be a known complimentary binding region, for example the antigen binding portion of an antibody or of a non-antibody molecule, including a single chain variable fragment (scFv) region or an antigen recognition domain from a T-cell receptor (TCR); or for proteins, it may be a known ligand of the target protein. In this way the wild type or modified E. coli CsgG nanopore, or homologue thereof, may be rendered capable of acting as a molecular sensor for detecting presence in a sample of suitable antigens (including epitopes) that may include cell surface antigens, including receptors, markers of solid tumours or haematologic cancer cells (e.g. lymphoma or leukaemia), viral antigens, bacterial antigens, protozoal antigens, allergens, allergy related molecules, albumin (e.g. human, rodent, or bovine), fluorescent molecules (including fluorescein), blood group antigens, small molecules, drugs, enzymes, catalytic sites of enzymes or enzyme substrates, and transition state analogues of enzyme substrates. As described above, modifications may be achieved using known genetic engineering and recombinant DNA techniques. The positioning of any adaptation would be dependent on the nature of the molecule to be sensed, for example, the size, three-dimensional structure, and its biochemical nature. The choice of adapted structure may make use of computational structural design. Determination and optimization of protein-protein interactions or protein-small molecule interactions can be investigated using technologies such as a BIAcore® which detects molecular interactions using surface plasmon resonance (BIAcore, Inc., Piscataway, NJ; see also www.biacore.com).

In one embodiment, the analyte is an amino acid, a peptide, a polypeptides or protein. The amino acid, peptide, polypeptide or protein can be naturally-occurring or non-naturally-occurring. The polypeptide or protein can include within them synthetic or modified amino acids. Several different types of modification to amino acids are known in the art. Suitable amino acids and modifications thereof are above. It is to be understood that the target analyte can be modified by any method available in the art.

In another embodiment, the analyte is a polynucleotide, such as a nucleic acid, which is defined as a macromolecule comprising two or more nucleotides. Nucleic acids are particularly suitable for nanopore sequencing. The naturally-occurring nucleic acid bases in DNA and RNA may be distinguished by their physical size. As a nucleic acid molecule, or individual base, passes through the channel of a nanopore, the size differential between the bases causes a directly correlated reduction in the ion flow through the channel. The variation in ion flow may be recorded. Suitable electrical measurement techniques for recording ion flow variations are discussed above. Through suitable calibration, the characteristic reduction in ion flow can be used to identify the particular nucleotide and associated base traversing the channel in real-time. In typical nanopore nucleic acid sequencing, the open-channel ion flow is reduced as the individual nucleotides of the nucleic sequence of interest sequentially pass through the channel of the nanopore due to the partial blockage of the channel by the nucleotide. It is this reduction in ion flow that is measured using the suitable recording techniques described above. The reduction in ion flow may be calibrated to the reduction in measured ion flow for known nucleotides through the channel resulting in a means for determining which nucleotide is passing through the channel, and therefore, when done sequentially, a way of determining the nucleotide sequence of the nucleic acid passing through the nanopore. For the accurate determination of individual nucleotides, it has typically required for the reduction in ion flow through the channel to be directly correlated to the size of the individual nucleotide passing through the constriction (or “reading head”). It will be appreciated that sequencing may be performed upon an intact nucleic acid polymer that is ‘threaded’ through the pore via the action of an associated polymerase, for example. Alternatively, sequences may be determined by passage of nucleotide triphosphate bases that have been sequentially removed from a target nucleic acid in proximity to the pore (see for example WO 2014/187924 incorporated herein by reference in its entirety).

The polynucleotide or nucleic acid may comprise any combination of any nucleotides. The nucleotides can be naturally occurring or artificial. One or more nucleotides in the polynucleotide can be oxidized or methylated. One or more nucleotides in the polynucleotide may be damaged. For instance, the polynucleotide may comprise a pyrimidine dimer. Such dimers are typically associated with damage by ultraviolet light and are the primary cause of skin melanomas. One or more nucleotides in the polynucleotide may be modified, for instance with a label or a tag, for which suitable examples are known by a skilled person. The polynucleotide may comprise one or more spacers. A nucleotide typically contains a nucleobase, a sugar and at least one phosphate group. The nucleobase and sugar form a nucleoside. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The sugar is preferably a deoxyribose. The polynucleotide preferably comprises the following nucleosides: deoxyadenosine (dA), deoxyuridine (dU) and/or thymidine (dT), deoxyguanosine (dG) and deoxycytidine (dC). The nucleotide is typically a ribonucleotide or deoxyribonucleotide. The nucleotide typically contains a monophosphate, diphosphate or triphosphate. The nucleotide may comprise more than three phosphates, such as 4 or 5 phosphates. Phosphates may be attached on the 5′ or 3′ side of a nucleotide. The nucleotides in the polynucleotide may be attached to each other in any manner. The nucleotides are typically attached by their sugar and phosphate groups as in nucleic acids. The nucleotides may be connected via their nucleobases as in pyrimidine dimers. The polynucleotide may be single stranded or double stranded. At least a portion of the polynucleotide is preferably double stranded. The polynucleotide is most preferably ribonucleic nucleic acid (RNA) or deoxyribonucleic acid (DNA). In particular, said method using a polynucleotide as an analyte alternatively comprises determining one or more characteristics selected from (i) the length of the polynucleotide, (ii) the identity of the polynucleotide, (iii) the sequence of the polynucleotide, (iv) the secondary structure of the polynucleotide and (v) whether or not the polynucleotide is modified.

The polynucleotide can be any length (i). For example, the polynucleotide can be at least 10, at least 50, at least 100, at least 150, at least 200, at least 250, at least 300, at least 400 or at least 500 nucleotides or nucleotide pairs in length. The polynucleotide can be 1000 or more nucleotides or nucleotide pairs, 5000 or more nucleotides or nucleotide pairs in length or 100000 or more nucleotides or nucleotide pairs in length. Any number of polynucleotides can be investigated. For instance, the method may concern characterising 2, 3, 4, 5, 6, 7, 8, 9, 10, 20, 30, 50, 100 or more polynucleotides. If two or more polynucleotides are characterised, they may be different polynucleotides or two instances of the same polynucleotide. The polynucleotide can be naturally occurring or artificial. For instance, the method may be used to verify the sequence of a manufactured oligonucleotide. The method is typically carried out in vitro.

Nucleotides can have any identity (ii), and include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP, TMP, GMP, CMP, UMP, dAMP, dTMP, dGMP, dCMP and dUMP. A nucleotide may be abasic (i.e. lack a nucleobase). A nucleotide may also lack a nucleobase and a sugar (i.e. is a C3 spacer). The sequence of the nucleotides (iii) is determined by the consecutive identity of following nucleotides attached to each other throughout the polynucleotide strain, in the 5′ to 3′ direction of the strand.

The pores comprising a CsgG pore and CsgF peptides are particularly useful in analysing homopolymers. For example, the pores may be used to determine the sequence of a polynucleotide comprising two or more, such as at least 3, 4, 5, 6, 7, 8, 9 or 10, consecutive nucleotides that are identical. For example, the pores may be used to sequence a polynucleotide comprising a polyA, polyT, polyG and/or polyC region.

The CsgG pore constriction is made of the residues at the 51, 55 and 56 positions of SEQ ID NO: 3 or SEQ ID NO: 117. The reader head of CsgG and its constriction mutants are generally sharp. When DNA is passing through the constriction, interactions of approximately 5 bases of DNA with the reader head of the pore at any given time dominate the current signal. Although these sharper reader heads are very good in reading mixed sequence regions of DNA (when A, T, G and C are mixed), the signal becomes flat and lack information when there is a homopolymeric region within the DNA (eg: polyT, polyG, polyA, polyC). Because 5 bases dominate the signal of the CsgG and its constriction mutants, its difficult to discriminate photopolymers longer than 5 without using additional dwell time information. However, if DNA is passing through a second reader head, more DNA bases will interact with the combined reader heads, increasing the length of the homopolymers that can be discriminated.

### Modified Dda Helicases

The present invention provides a modified Dda helicase. The one or more specific modifications are discussed in more detail below. Modifications according to the invention include one or more substitutions as discussed below.

### One or More of Positions 55, 114, 156, 177, 210, 221, 350 and 358 of Dda 1993

The invention provides a modified DNA dependent ATPase (Dda) helicase in which one or more of the positions corresponding to amino acid positions 55, 114, 156, 177, 210, 221, 350 and 358 in Dda 1993 are modified or substituted. Dda helicases and the positions corresponding to positions 55, 114, 156, 177, 210, 221, 350 and 358 in Dda 1993 are discussed in more detail below. Positions 55, 114, 156 and 177 are in the 1A domain of Dda 1993. Positions 210 and 221 are in the 2A domain of Dda 1993. Positions 350 and 358 are in the tower domain of Dda 1993.

The modified Dda helicase of the invention may comprise a modification or substitution at any number and combination of the positions corresponding to amino acid positions (a) 55, (b) 114, (c) 156, (d) 177, (e) 210, (f) 221, (g) 350 and (h) 358, including at (a); (b); (c); (d); (e); (f); (g); (h); (a) and (b); (a) and (c); (a) and (d); (a) and (e); (a) and (f); (a) and (g); (a) and (h); (b) and (c); (b) and (d); (b) and (e); (b) and (f); (b) and (g); (b) and (h); (c) and (d); (c) and (e); (c) and (f); (c) and (g); (c) and (h); (d) and (e); (d) and (f); (d) and (g); (d) and (h); (e) and (f); (e) and (g); (e) and (h); (f) and (g); (f) and (h); (g) and (h); (a), (b) and (c); (a), (b) and (d); (a), (b) and (e); (a), (b) and (f); (a), (b) and (g); (a), (b) and (h); (a), (c) and (d); (a), (c) and (e); (a), (c) and (f); (a), (c) and (g); (a), (c) and (h); (a), (d) and (e); (a), (d) and (f); (a), (d) and (g); (a), (d) and (h); (a), (e) and (f); (a), (e) and (g); (a), (e) and (h); (a), (f) and (g); (a), (f) and (h); (a), (g) and (h); (b), (c) and (d); (b), (c) and (e); (b), (c) and (f); (b), (c) and (g); (b), (c) and (h); (b), (d) and (e); (b), (d) and (f); (b), (d) and (g); (b), (d) and (h); (b), (e) and (f); (b), (e) and (g); (b), (e) and (h); (b), (f) and (g); (b), (f) and (h); (b), (g) and (h); (c), (d) and (e); (c), (d) and (f); (c), (d) and (g); (c), (d) and (h); (c), (e) and (f); (c), (e) and (g); (c), (e) and (h); (c), (f) and (g); (c), (f) and (h); (c), (g) and (h); (d), (e) and (f); (d), (e) and (g); (d), (e) and (h); (d), (f) and (g); (d), (f) and (h); (d), (g) and (h); (e), (f) and (g); (e), (f) and (h); (e), (g) and (h); (f), (g) and (h); (a), (b), (c) and (d); (a), (b), (c) and (e); (a), (b), (c and (f); (a), (b), (c) and (g); (a), (b), (c) and (h); (a), (b), (d) and (e); (a), (b), (d) and (f); (a), (b), (d) and (g); (a), (b), (d) and (h); (a), (b), (e) and (f); (a), (b), (e) and (g); (a), (b), (e) and (h); (a), (b), (f) and (g); (a), (b), (f) and (h); (a), (b), (g) and (h); (a), (c), (d) and (e); (a), (c), (d) and (f); (a), (c), (d) and (g); (a), (c), (d) and (h); (a), (c), (e) and (f); (a), (c), (e) and (g); (a), (c), (e) and (h); (a), (c), (f) and (g); (a), (c), (f) and (h); (a), (c), (g) and (h); (a), (d), (e) and (f); (a), (d), (e) and (g); (a), (d), (e) and (h); (a), (d), (f) and (g); (a), (d), (f) and (h); (a), (d), (g) and (h); (a), (e), (f) and (g); (a), (e), (f) and (h); (a), (e), (g) and (h); (a), (f), (g) and (h); (b), (c), (d) and (e); (b), (c), (d) and (f); (b), (c), (d) and (g); (b), (c), (d) and (h); (b), (c), (e) and (f); (b), (c), (e) and (g); (b), (c), (e) and (h); (b), (c), (f) and (g); (b), (c), (f) and (h); (b), (c), (g) and (h); (b), (d), (e) and (f); (b), (d), (e) and (g); (b), (d), (e) and (h); (b), (d), (f) and (g); (b), (d), (f) and (h); (b), (d), (g) and (h); (b), (e), (f) and (g); (b), (e), (f) and (h); (b), (e), (g) and (h); (b), (f), (g) and (h); (c), (d), (e) and (f); (c), (d), (e) and (g); (c), (d), (e) and (h); (c), (d), (f) and (g); (c), (d), (f) and (h); (c), (d), (g) and (h); (c), (e), (f) and (g); (c), (e), (f) and (h); (c), (e), (g) and (h); (c), (f), (g) and (h); (d), (e), (f) and (g); (d), (e), (f) and (h); (d), (e), (g) and (h); (d), (f), (g) and (h); (e), (f), (g) and (h); (a), (b), (c), (d) and (e); (a), (b), (c), (d) and (f); (a), (b), (c), (d) and (g); (a), (b), (c), (d) and (h); (a), (b), (c), (e) and (f); (a), (b), (c), (e) and (g); (a), (b), (c), (e) and (h); (a), (b), (c), (f) and (g); (a), (b), (c), (f) and (h); (a), (b), (c), (g) and (h); (a), (b), (d), (e) and (f); (a), (b), (d), (e) and (g); (a), (b), (d), (e) and (h); (a), (b), (d), (f) and (g); (a), (b), (d), (f) and (h); (a), (b), (d), (g) and (h); (a), (b), (e), (f) and (g); (a), (b), (e), (f) and (h); (a), (b), (e), (g) and (h); (a), (b), (f), (g) and (h); (a), (c), (d), (e) and (f); (a), (c), (d), (e) and (g); (a), (c), (d), (e) and (h); (a), (c), (d), (f) and (g); (a), (c), (d), (f) and (h); (a), (c), (d), (g) and (h); (a), (c), (e), (f) and (g); (a), (c), (e), (f) and (h); (a), (c), (e), (g) and (h); (a), (c), (f), (g) and (h); (a), (d), (e), (f) and (g); (a), (d), (e), (f) and (h); (a), (d), (e), (g) and (h); (a), (d), (f), (g) and (h); (a), (e), (f), (g) and (h); (b), (c), (d), (e) and (f); (b), (c), (d), (e) and (g); (b), (c), (d), (e) and (h); (b), (c), (d), (f) and (g); (b), (c), (d), (f) and (h); (b), (c), (d), (g) and (h); (b), (c), (e), (f) and (g); (b), (c), (e), (f) and (h); (b), (c), (e), (g) and (h); (b), (c), (f), (g) and (h); (b), (d), (e), (f) and (g); (b), (d), (e), (f) and (h); (b), (d), (e), (g) and (h); (b), (d), (f), (g) and (h); (b), (e), (f), (g) and (h); (c), (d), (e), (f) and (g); (c), (d), (e), (f) and (h); (c), (d), (e), (g) and (h); (c), (d), (f), (g) and (h); (c), (e), (f), (g) and (h); (d), (e), (f), (g) and (h); (a), (b), (c), (d), (e) and (f); (a), (b), (c), (d), (e) and (g); (a), (b), (c), (d), (e) and (h); (a), (b), (c), (d), (f) and (g); (a), (b), (c), (d), (f) and (h); (a), (b), (c), (d), (g) and (h); (a), (b), (c), (e), (f) and (g); (a), (b), (c), (e), (f) and (h); (a), (b), (c), (e), (g) and (h); (a), (b), (c), (f), (g) and (h); (a), (b), (d), (e), (f) and (g); (a), (b), (d), (e), (f) and (h); (a), (b), (d), (e), (g) and (h); (a), (b), (d), (f), (g) and (h); (a), (b), (e), (f), (g) and (h); (a), (c), (d), (e), (f) and (g); (a), (c), (d), (e), (f) and (h); (a), (c), (d), (e), (g) and (h); (a), (c), (d), (f), (g) and (h); (a), (c), (e), (f), (g) and (h); (a), (d), (e), (f), (g) and (h); (b), (c), (d), (e), (f) and (g); (b), (c), (d), (e), (f) and (h); (b), (c), (d), (e), (g) and (h); (b), (c), (d), (f), (g) and (h); (b), (c), (e), (f), (g) and (h); (b), (d), (e), (f), (g) and (h); (c), (d), (e), (f), (g) and (h); (a), (b), (c), (d), (e), (f) and (g); (a), (b), (c), (d), (e), (f) and (h); (a), (b), (c), (d), (e), (g) and (h); (a), (b), (c), (d), (f), (g) and (h); (a), (b), (c), (e), (f), (g) and (h); (a), (b), (d), (e), (f), (g) and (h); (a), (c), (d), (e), (f), (g) and (h); (b), (c), (d), (e), (f), (g) and (h); or (a), (b), (c), (d), (e), (f), (g) and (h).

The invention provides a modified DNA dependent ATPase (Dda) helicase in which one or more of the positions corresponding to amino acid positions 114, 177, 350 and 358 in Dda 1993 are modified or substituted. Dda helicases and the positions corresponding to positions 114, 177, 350 and 358 in Dda 1993 are discussed in more detail below. Positions 114 and 177 are in the 1A domain of Dda 1993. Positions Y350 and K358 are in the tower domain of Dda 1993. The modified Dda helicase of the invention may comprise a modification or substitution at any number and combination of the positions corresponding to amino acid positions (a) 114, (b) 177, (c) 350 and (d) 358 in Dda 1993, including at (a); (b); (c); (d); (a) and (b); (a) and (c); (a) and (d); (b) and (c); (b) and (d); (c) and (d); (a), (b) and (c); (a), (b) and (d); (a), (c) and (d); (b), (c) and (d); or (a), (b), (c) and (d).

The position corresponding to amino acid position 55 in Dda 1993 is preferably substituted with D, E, K, N or S. The position corresponding to amino acid position 114 in Dda 1993 is preferably substituted with K (T55K). These substitutions increase the speed and increase the accuracy when used to characterise a polynucleotide analyte (Example 5). These substitutions also decrease the normalised speed distribution when used to characterise a polynucleotide analyte (Example 5).

The position corresponding to amino acid position 114 in Dda 1993 is preferably substituted with A, V, I, L, M, F, Y, W, G, P, S, T, N or Q. The position corresponding to amino acid position 114 in Dda 1993 is preferably substituted with A, G, I, L, M, P, S, T or V. The position corresponding to amino acid position 114 in Dda 1993 is preferably substituted with G, L, S or T. These substitutions decrease the speed when used to characterise a polynucleotide analyte (Example 2). The position corresponding to amino acid position 114 in Dda 1993 is preferably substituted with A, I, M, P, or V. These substitutions increase the speed when used to characterise a polynucleotide analyte (Example 2). The position corresponding to amino acid position 114 in Dda 1993 is preferably substituted with G (C11G). This substitution decreases the speed and increases the accuracy when used to characterise a polynucleotide analyte (Example 2). The position corresponding to amino acid position 114 in Dda 1993 is preferably substituted with I or P. These substitutions increase the speed and decrease the accuracy when used to characterise a polynucleotide analyte (Example 2). The position corresponding to amino acid position 114 in Dda 1993 is preferably substituted with G, I or P. These substitutions decrease the normalised speed distribution when used to characterise a polynucleotide analyte (Example 2). The position corresponding to amino acid position 114 in Dda 1993 is most preferably substituted with I (C114I).

The position corresponding to amino acid position 156 in Dda 1993 is preferably substituted with A, E, F, G, I, L, M, P, S, V, Y, D, K or N. The position corresponding to amino acid position 156 in Dda 1993 is preferably substituted with F (T156F). This substitution increases the speed and increases the accuracy when used to characterise a polynucleotide analyte (Example 5). This substitution also decreases the normalised speed distribution when used to characterise a polynucleotide analyte (Example 5).

The position corresponding to amino acid position 177 in Dda 1993 is preferably substituted with D, E, F, G, H, I, L, M, N, Q, R, S, T, V, W or Y. The position corresponding to amino acid position 177 in Dda 1993 is preferably substituted with F, G, S, V, W or Y. These substitutions decrease the speed when used to characterise a polynucleotide analyte (Example 2). The position corresponding to amino acid position 177 in Dda 1993 is preferably substituted with D, E, G, H, I, L, M, N, Q, R, or T. These substitutions increase the speed when used to characterise a polynucleotide analyte (Example 2). The position corresponding to amino acid position 177 in Dda 1993 is preferably substituted with F, H, I, L, M, N or W. These substitutions decrease the accuracy and the normalised speed distribution when used to characterise a polynucleotide analyte (Example 2). They have different effects on the speed (Example 2). The position corresponding to amino acid position 177 in Dda 1993 is preferably substituted with N (K177N). This substitution decreases the accuracy and increases the normalised speed distribution when used to characterise a polynucleotide analyte (Example 2). The position corresponding to amino acid position 177 in Dda 1993 is most preferably substituted with M (K177M).

The position corresponding to amino acid position 210 in Dda 1993 is preferably substituted with D, E, K, S, N, R, H or Y. The position corresponding to amino acid position 210 in Dda 1993 is preferably substituted with R (T210R), H (T210H) or K (T210K). The position corresponding to amino acid position 210 in Dda 1993 is preferably substituted with K (T210K). This substitution increases the speed and increases the accuracy when used to characterise a polynucleotide analyte (Example 5). This substitution also decreases the normalised speed distribution when used to characterise a polynucleotide analyte (Example 5).

The position corresponding to amino acid position 221 in Dda 1993 is preferably substituted with D, K, E, Q, R, A, H, L, T or Y. The position corresponding to amino acid position 221 in Dda 1993 is preferably substituted with D (N221D) or E (N221E). The position corresponding to amino acid position 221 in Dda 1993 is preferably substituted with E (N221E). This substitution increases the speed and increases the accuracy when used to characterise a polynucleotide analyte (Example 5). This substitution also decreases the normalised speed distribution when used to characterise a polynucleotide analyte (Example 5).

The position corresponding to amino acid position 350 in Dda 1993 is preferably substituted with D, E, A, V, I, L, M, F, W, R, H, K, L, S, T, N or Q. The position corresponding to amino acid position 350 in Dda 1993 is preferably substituted with I, F, W or S. The position corresponding to amino acid position 350 in Dda 1993 is preferably substituted with I or S (Y350I or Y350S). The position corresponding to amino acid position 350 in Dda 1993 is preferably substituted with I (Y350I). This substitution increases the speed and decreases the accuracy and normalised speed distribution when used to characterise a polynucleotide analyte (Example 3). The position corresponding to amino acid position 350 in Dda 1993 is preferably substituted with I or S (Y350I or Y350S). These substitutions have the effects shown in Example 4 when used with a pore complex of the invention.

The position corresponding to amino acid position 350 in Dda 1993 is preferably substituted with A, D, E, G, K, L, N, Q, R, T, V, H or M. The position corresponding to amino acid position 350 in Dda 1993 is preferably substituted with D (Y350D) or E (Y350E). The position corresponding to amino acid position 350 in Dda 1993 is preferably substituted with E (Y350E). This substitution increases the speed and increases the accuracy when used to characterise a polynucleotide analyte (Example 5). This substitution also decreases the normalised speed distribution when used to characterise a polynucleotide analyte (Example 5).

The position corresponding to amino acid position 358 in Dda 1993 is preferably substituted with D, E, A, V, I, L, M, F, Y, W, R, H, L, S, T, N or Q.

The position corresponding to amino acid position 358 in Dda 1993 is preferably substituted with E, I, L or M. These substitutions decrease the speed when used to characterise a polynucleotide analyte (Example 2). The position corresponding to amino acid position 358 in Dda 1993 is preferably substituted with I or M. These substitutions decrease the speed and increase the accuracy when used to characterise a polynucleotide analyte (Example 2). The position corresponding to amino acid position 358 in Dda 1993 is preferably substituted M (K358M). This substitution decreases the speed and increase the accuracy and the normalised speed distribution when used to characterise a polynucleotide analyte (Example 2). The position corresponding to amino acid position 358 in Dda 1993 is preferably substituted with I (K358I). This substitution decreases the speed and normalised speed distribution and increases the accuracy when used to characterise a polynucleotide analyte (Example 2). Example 2 uses a CsgG pore without a CsgF peptide.

The position corresponding to amino acid position 358 in Dda 1993 is preferably substituted with A, E, F, I, M or S. These substitutions increase the accuracy when used to characterise a polynucleotide analyte (Example 3). The position corresponding to amino acid position 358 in Dda 1993 is preferably substituted with A, E, F, I or M. These substitutions decrease the speed when used to characterise a polynucleotide analyte (Example 3). The position corresponding to amino acid position 358 in Dda 1993 is preferably substituted S (K358S). This substitution increases the speed when used to characterise a polynucleotide analyte (Example 3). The position corresponding to amino acid position 358 in Dda 1993 is preferably substituted with A, E, I, M or S. These substitutions decrease the normalised speed distribution when used to characterise a polynucleotide analyte (Example 3). The position corresponding to amino acid position 358 in Dda 1993 is preferably substituted with (K358F). These substitutions increase the normalised speed distribution when used to characterise a polynucleotide analyte (Example 3). Example 3 uses a CsgG pore complex containing a CsgF peptide.

The position corresponding to amino acid position 358 in Dda 1993 is preferably substituted with I, L or Q. These substitutions decrease the speed and increase the accuracy and normalised speed distribution when used to characterise a polynucleotide analyte (Example 4). Example 4 uses a pore complex of the invention.

The position corresponding to amino acid position 358 in Dda 1993 is most preferably substituted with I (K358I).

The modified helicase of the invention may further comprise any of the modifications, mutations or substitutions discussed below.

The Dda helicase that is modified in accordance with the invention may be any of SEQ ID NOs: 118 to 133. SEQ ID NO: 118 is Dda 1993. The modified helicase preferably comprises a variant of any of SEQ ID NOs: 118 to 133. The variant may have any % of the sequence homologies/identities to any of SEQ ID NOs: 118 to 113 set out below.

Table 4 below summarises the preferred Dda helicases which may be modified in accordance with the invention.

| | | | | | Sequence | Number of | |
|---|---|---|---|---|---|---|---|
| Dda Homologue | | | | | Identity | D/E vs. K/R | # |
| (SEQ ID NO:) | | Habitat | Uniprot | Length | to 1993/% | amino acids | C |
| Rma-DSM | Rhodothermus | Mild halophile, | D0MKQ2 | 678 | 21 | −84/+85 | 2 |
| (SEQ ID | marinus | moderate | | | | | |
| NO: 119) | | thermophile >65° C. | | | | | |
| Csp | Cyanothece sp. | Marine | B1X365 | 496 | 24 | −76/+76 | 5 |
| (SEQ ID | (strain ATCC | bacterium | | | | | |
| NO: 120) | 51142) | | | | | | |
| Sru | Salinibacter | Extremely | Q2S429 | 42 | 26 | −78/+54 | 3 |
| (SEQ ID | ruber | halophilic, | | | | | |
| NO: 121) | | 35-45° C. | | | | | |
| Sgo | Sulfurimonas | Habitat: | B6BJ43 | 500 | 27 | −72/+64 | 2 |
| (SEQ ID | gotlandica | hydrothermal | | | | | |
| NO: 122) | GD1 | vents, coastal | | | | | |
| | | sediments | | | | | |
| Vph12B8 | Vibrio phage | Host found in | M4MBC3 | 450 | 27 | −62/+47 | 6 |
| (SEQ ID | henriette | saltwater, | | | | | |
| NO: 123) | 12B8 | stomach bug | | | | | |
| Vph | Vibrio | Host found in | 16XGX8 | 421 | 39 | −55/+45 | 5 |
| (SEQ ID | phage phi-pp2 | saltwater, | | | | | |
| NO: 124) | | stomach bug | | | | | |
| Aph65 | Aeromonas | Host found in | E5DRP6 | 434 | 40 | −57/+48 | 4 |
| (SEQ ID | phage 65 | fresh/brackish | | | | | |
| NO: 125) | | water, stomach | | | | | |
| | | bug | | | | | |
| AphCC2 | Aeromonas | Host found in | I6XH64 | 420 | 41 | −53/+44 | 4 |
| (SEQ ID | phage CC2 | fresh/brackish | | | | | |
| NO: 126) | | water, stomach | | | | | |
| | | bug | | | | | |
| Cph | Cronobacter | Host member of | K4FBD0 | 443 | 42 | −59/+57 | 4 |
| (SEQ ID | phage vB CsaM | enterobacteriaceae | | | | | |
| NO: 127) | GAP161 | | | | | | |
| Kph | Klebsiella | Host member of | D5JF67 | 442 | 44 | −59/+58 | 5 |
| (SEQ ID | phage KP15 | enterobacteriaceae | | | | | |
| NO: 128) | | | | | | | |
| SphIME13 | Stenotrophomonas | Host found in | J7HXT5 | 438 | 51 | −58/+59 | 7 |
| (SEQ ID | phage IME13 | soil | | | | | |
| NO: 129) | | | | | | | |
| AphAc42 | Acinetobacter | Host found in | E5EYE6 | 442 | 59 | −53/+49 | 9 |
| (SEQ ID | phage Ac42 | soil | | | | | |
| NO: 130) | | | | | | | |
| SphSP18 | Shigella | Host member of | E3SFA5 | 442 | 59 | −55/+55 | 9 |
| (SEQ ID | phage SP18 | enterobacteriaceae | | | | | |
| NO: 131) | | | | | | | |
| Yph | Yersinia | Host member of | I7J3V8 | 439 | 64 | −52/+52 | 7 |
| (SEQ ID | phage phiR1-RT | enterobacteriaceae | | | | | |
| NO: 132) | | | | | | | |
| SphS16 | Salmonella | Host member of | M1EA88 | 441 | 72 | −56/+55 | 5 |
| (SEQ ID | phage S16 | enterobacteriaceae | | | | | |
| NO: 133) | | | | | | | |
| 1993 | Enterobateria | Host member of | P32270 | 439 | 100 | −57/+58 | 5 |
| (SEQ ID | phage T4 | enterobacteriaceae | | | | | |
| NO: 118) | | | | | | | |

Table 5 shows the amino acids in SEQ ID NOs: 119 to 133 which correspond to positions 40, 55, 114, 156, 177, 210, 221, 350 and 358 in SEQ ID NO: 118.

| SEQ ID NO: | | | | | | | | | |
|---|---|---|---|---|---|---|---|---|---|
| 118 | T40 | T55 | C114 | T156 | K177 | T210 | N221 | Y350 | K358 |
| 119 | L40 | I54 | V124 or | P187 | Y211 | A255 | Q266 | L378 | L386 |
| | | | D125 | | | | | | |
| 120 | F98 | Y112 | I180 or | I223 | Q238 | I274 | Q285 | E407 | E415 |
| | | | D171 | | | | | | |
| 121 | Y43 | C57 | V119 or | P160 | H173 | A207 | S218 | R334 or | A340 |
| | | | D120 | | | | | R343 | |
| 122 | F56 | F80 | V147 or | E191 | V205 | T245 | D256 | A367 | S375 |
| | | | D148 | | | | | | |
| 123 | T46 | K59 | I125 or | E165 | V194 | I234 | S245 | E354 or | A362 |
| | | | D126 | | | | | E256 | |
| 124 | F33 | K47 | V107 or | P147 | V166 | L200 | T211 | E332 | V340 |
| | | | E108 | | | | | | |
| 125 | F38 | L51 | V110 or | Q150 | M171 | Y205 | S216 | L339 | E347 |
| | | | E111 | | | | | | |
| 126 | F25 | L38 | 197 or | P137 | M158 | Y192 | S203 | A326 or | E334 or |
| | | | E98 | | | | | A337 | E345 |
| 127 | T42 | S57 | V119 or | R160 | M181 | F213 | Q224 | D351 or | Y359 or |
| | | | E120 | | | | | D326 | Y334 |
| 128 | T42 | S57 | V119 or | I160 | M181 or | M213 | R224 | E350 | Y358 |
| | | | E120 | | M182 | | | | |
| 129 | T40 | Q55 | C114 or | T155 | K176 | — | K218 | Y349 or | I357 or |
| | | | D115 | | | | | Y347 | I355 |
| 130 | T46 | R61 | C120 or | E162 | M183 | T216 | K227 | N355 or | K363 or |
| | | | D121 | | | | | N353 | K361 |
| 131 | T42 | M57 | C116 or | Q158 | M179 | T212 | N223 | P352 or | K360 or |
| | | | D117 | | | | | P355 | K363 |
| 132 | T41 | T56 | C115 or | E157 | K178 | N211 | R222 | T352 or | K360 or |
| | | | D116 | | | | | T350 | K358 |
| 133 | T41 | T56 | C115 or | E157 | M178 | T211 | Q222 | N351 | K359 |
| | | | D116 | | | | | | |

The helicase of the invention preferably comprises a variant of SEQ ID NO: 118 comprising one or more of (a)-(h) as follows:
(a)
T551D, T55E, T55K, T55N or T55S, or
T55K.
(b)
C114A, C114V, C114I, C114L, C114M, C114F, C114Y, C114W, C114G, C114P, C114S,
C114T, C114N or C114Q,
C114A, C114G, C114I, C114L, C114M, C114P, C114S, C114T or C114V,
C114G, C114L, C114S or C114T,
C114A, C114I, C114M, C114P, or C114V
C11G,
C114J or C114P
C114G, C114J or C114P, or
C114I;
(c)
T156A, T156E, T156F, T156G, T156I, T156L, T156M, T156P, T156S, T156V, T156Y, T156D,
T156K or T156N, or
T156F;
(d)
K177D, K177E, K177F, K177G, K177H, K177I, K177L, K177M, K177N, K177Q, K177R,
K177S, K177T, K177V, K177W or K177Y,
K177F, K177G, K177S, K177V, K177W or K177Y,
K177D, K177E, K177G, K177H, K177I, K177L, K177M, K177N, K177Q, K177R, or K177T,
K177F, K177H, K177I, K177L, K177M, K177N or K177W,
K177N, or
K177M;
(e)
T210D, T210E, T210K, T210S, T210N, T210R, T210H or T210Y,
T210R, T210H or T210Y, or
T210K;
(f)
N221D, N221K, N221E, N221Q, N221R, N221A, N221H, N221L, N221T or N221Y,
N221D or N221E, or
N221E;
(g)
Y350D, Y350E, Y350A, Y350V, Y350I, Y350L, Y350M, Y350F, Y350W, Y350R, Y350H,
Y350K, Y350L, Y350S, Y350T, Y350N or Y350Q,
Y350I or Y350S,
Y350I,
Y350S,
Y350I, Y350F, Y350W or Y350S,
Y350A, Y350D, Y350E, Y350G, Y350K, Y350L, Y350N, Y350Q, Y350R, Y350T, Y350V,
Y350H or Y350M,
Y350D or Y350E, or
Y350E; and
(h)
K358D, K358E, K358A, K358V, K358I, K358L, K358M, K358F, K358Y, K358W, K358R,
K358H, K358L, K358S, K358T, K358N or K358Q,
K358E, K358I, K358L or K358M,
K358I or K358M,
K358M,
K358I,
K358A, K358E, K358F, K358I, K358M or K358S,
K358A, K358E, K358F, K358I or K358M,
K358S,
K358A, K358E, K358I, K358M or K358S,
K358F,
K358I, K358L or K358Q, or
K358I.

The variant may include any combination and permutation of (a)-(h) as set out above.

The helicase of the invention preferably comprises a variant of SEQ ID NO: 118 comprising one or more of (a), (b), (c) and (d) as follows:
(a)
C114A, C114V, C114I, C114L, C114M, C114F, C114Y, C114W, C114G, C114P, C114S,
C114T, C114N or C114Q,
C114A, C114G, C114I, C114L, C114M, C114P, C114S, C114T or C114V,
C114G, C114L, C114S or C114T,
C114A, C114I, C114M, C114P, or C114V
C11G,
C114I or C114P
C114G, C114I or C114P, or
C114I;
(b)
K177D, K177E, K177F, K177G, K177H, K177I, K177L, K177M, K177N, K177Q, K177R,
K177S, K177T, K177V, K177W or K177Y,
K177F, K177G, K177S, K177V, K177W or K177Y,
K177D, K177E, K177G, K177H, K177I, K177L, K177M, K177N, K177Q, K177R, or K177T,
K177F, K177H, K177I, K177L, K177M, K177N or K177W,
K177N, or
K177M;
(c)
Y350D, Y350E, Y350A, Y350V, Y350I, Y350L, Y350M, Y350F, Y350W, Y350R, Y350H,
Y350K, Y350L, Y350S, Y350T, Y350N or Y350Q,
Y350I or Y350S,
Y350I, or
Y350S; and
(d)
K358D, K358E, K358A, K358V, K358I, K358L, K358M, K358F, K358Y, K358W, K358R,
K358H, K358L, K358S, K358T, K358N or K358Q,
K358E, K358I, K358L or K358M,
K358I or K358M,
K358M,
K358I,
K358A, K358E, K358F, K358I, K358M or K358S,
K358A, K358E, K358F, K358I or K358M,
K358S,
K358A, K358E, K358I, K358M or K358S,
K358F,
K358I, K358L or K358Q, or
K358I.

The variant may include (a); (b); (c); (d); (a) and (b); (a) and (c); (a) and (d); (b) and (c); (b) and (d); (c) and (d); (a), (b) and (c); (a), (b) and (d); (a), (c) and (d); (b), (c) and (d); or (a), (b), (c) and (d).

A preferred variant of SEQ ID NO: 118 comprises: C114I; K177M; Y350I; K358I; C114I and K177M; C114I and Y350I; C114I and K358I; K177M and Y350I; K177M and K358I; Y350I and K358I; C114I, K177M and Y350I; C114I, K177M and K358I; C114I, Y350I and K358I; K177M, Y350I and K358I; or C114I, K177M, Y350I and K358I.

The helicase preferably comprises a variant of SEQ ID NO: 119, 120, 121, 122, 123, 124, 125, 126, 127, 128, 129, 130, 131, 132 or 133 wherein one or more of the positions corresponding to amino acid positions 55, 114, 156, 177, 210, 221, 350 and 358 in Dda 1993 are modified or substituted as defined above (including specific substitutions). Various combinations and permutations of one or more of positions 55, 114, 156, 177, 210, 221, 350 and 358 in Dda 1993 are defined above with reference to (a)-(h).

The helicase preferably comprises a variant of SEQ ID NO: 119, 120, 121, 122, 123, 124, 125, 126, 127, 128, 129, 130, 131, 132 or 133 wherein one or more of the positions corresponding to amino acid positions 114, 177, 350 and 358 in Dda 1993 are modified or substituted as defined above (including specific substitutions). Various combinations and permutations of one or more of positions 114, 177, 350 and 358 in Dda 1993 are defined above with reference to (a)-(d).

The helicase preferably comprises a variant of SEQ ID NO: 119, 120, 121, 122, 123, 124, 125, 126, 127, 128, 129, 130, 131, 132 or 133 wherein one or more of the positions corresponding to amino acid positions 114, 177 and 358 in Dda 1993 are modified or substituted as defined above (including specific substitutions).

### Position 40 in Dda 1993

Any of the modified helicases of the invention may further comprise a modification or substitution at the position corresponding to amino acid position 40 in Dda 1993. Position 40 or the corresponding position may be substituted with as A, V, I, L, M, F, Y or W. Positions which correspond to position T40 in Dda 1993 are shown in Table 5 above. The helicase of the invention preferably comprises a variant of SEQ ID NO: 118 which, in addition to the modifications/substitution set out above, further comprises a substitution at T40, such as T40A, T40V, T40I, T40L, T40M, T40F, T40Y or T40W. The substitution is preferably T40Y.

The invention provides a modified DNA dependent ATPase (Dda) helicase in which the position corresponding to amino acid position 40 in Dda 1993 is modified or substituted. Position T40 is in the tower domain of Dda 1993. The helicase of the invention preferably comprises a variant of SEQ ID NO: 118 which comprises a substitution at T40, such as T40A, T40V, T40I, T40L, T40M, T40F, T40Y or T40W. The substitution is preferably T40Y. The modified Dda helicase of the invention may further comprise a modification or substitution at one or more of the positions corresponding to amino acid positions (a) 55, (b) 114, (c) 156, (d) 177, (e) 210, (f) 221, (g) 350 and (h) 358, including any of the combinations and permutations of (a)-(h) set out above. The modified Dda helicase of the invention may further comprise a modification or substitution at one or more of the positions corresponding to amino acid positions (a) 114, (b) 177, (c) 350 and (d) 358 in Dda 1993, including at (a); (b); (c); (d); (a) and (b); (a) and (c); (a) and (d); (b) and (c); (b) and (d); (c) and (d); (a), (b) and (c); (a), (b) and (d); (a), (c) and (d); (b), (c) and (d); or (a), (b), (c) and (d). The helicase preferably comprises a variant of SEQ ID NO: 119, 120, 121, 122, 123, 124, 125, 126, 127, 128, 129, 130, 131, 132 or 133 wherein the position corresponding to amino acid position 40 in Dda 1993 is modified or substituted as defined above (including specific substitutions).

The modified helicases of the invention may further comprise any of the modifications, substitutions, combinations of modifications or combination of substitutions discussed below.

### Other Helicases of the Invention

The invention also provides a modified DNA dependent ATPase (Dda) helicase having any of the modifications, substitutions, combinations of modifications or combination of substitutions discussed below in isolation. In other words, these helicases of the invention do not necessarily have a substitution at positions corresponding to any of amino acid positions 40, 55, 114, 156, 177, 210, 221, 350 and 358 in Dda 1993 or any of amino acid positions 40, 114, 350, 177 and K358 in Dda 1993. Such modified helicases of the invention are preferably a variant of any of SEQ ID NOs: 118 to 133. The variant may have any % of the sequence homologies/identities to any of SEQ ID NOs: 118 to 133 set out below.

The use of Dda helicases in analyte characterisation are described in WO2015/055981, WO2015/166276 and WO2016/055777 (all incorporated by reference).

The modified helicases of the invention provide more consistent movement of the target analyte with respect to, such as through, the transmembrane pore leading to improved accuracy. The helicases preferably provide more consistent movement from one k-mer to another or from k-mer to k-mer as the target analyte, such as polynucleotide, moves with respect to, such as through, the pore. The helicases allow the target analyte, such as target polynucleotide, to move with respect to, such as through, the transmembrane pore more smoothly. The helicases preferably provide more regular or less irregular movement of the target analyte, such as target polynucleotide, with respect to, such as through, the transmembrane pore.

The modification(s) typically increase accuracy by at least 0.1%, at least 0.5%, at least 1%, at least 5%, at least 10%, at least 20%, at least 30%, at least 40%, at least 50%, at least 60%, at least 70%, at least 80% or at least 90% compared to a helicase without the modification. The ability of a helicase to control the movement of a polynucleotide can be determined as described in the Examples.

The modified helicase has the ability to control the movement of a polynucleotide. The ability of a helicase to control the movement of a polynucleotide can be assayed using any method known in the art. For instance, the helicase may be contacted with a polynucleotide and the position of the polynucleotide may be determined using standard methods. The ability of a modified helicase to control the movement of a polynucleotide is typically assayed in a nanopore system, such as the ones described below and, in particular, as described in the Examples.

A modified helicase of the invention may be isolated, substantially isolated, purified or substantially purified. A helicase is isolated or purified if it is completely free of any other components, such as lipids, polynucleotides, pore monomers or other proteins. A helicase is substantially isolated if it is mixed with carriers or diluents which will not interfere with its intended use. For instance, a helicase is substantially isolated or substantially purified if it is present in a form that comprises less than 10%, less than 5%, less than 2% or less than 1% of other components, such as lipids, polynucleotides, pore monomers or other proteins.

### Dda Helicases

Any Dda helicase may be modified in accordance with the invention. Preferred Dda helicases are discussed below and described in WO2015/055981, WO2015/166276 and WO2016/055777 (all incorporated by reference).

Dda helicases typically comprises the following five domains: 1A (RecA-like motor) domain, 2A (RecA-like motor) domain, tower domain, pin domain and hook domain (Xiaoping He et al., 2012, Structure; 20: 1189-1200). The domains may be identified using protein modelling, x-ray diffraction measurement of the protein in a crystalline state (Rupp B (2009). Biomolecular Crystallography: Principles, Practice and Application to Structural Biology. New York: Garland Science), nuclear magnetic resonance (NMR) spectroscopy of the protein in solution (Mark Rance; Cavanagh, John; Wayne J. Fairbrother; Arthur W. Hunt III; Skelton, Nicholas J. (2007). Protein NMR spectroscopy: principles and practice (2nd ed.). Boston: Academic Press) or cryo-electron microscopy of the protein in a frozen-hydrated state (van Heel M, Gowen B, Matadeen R, Orlova E V, Finn R, Pape T, Cohen D, Stark H, Schmidt R, Schatz M, Patwardhan A (2000). “Single-particle electron cryo-microscopy: towards atomic resolution.”. Q Rev Biophys. 33: 307-69). Structural information of proteins determined by above mentioned methods are publicly available from the protein bank (PDB) database.

In addition to a modification or substitution at one or more positions corresponding to amino acid positions 55, 114, 156, 177, 210, 221, 350 and 358 in Dda 1993, a modification or substitution at one or more positions corresponding to amino acid positions 114, 177, 350 and 358 in Dda 1993, a modification or substitution at one or more positions corresponding to amino acid positions 114, 177 and 350 in Dda 1993 and/or a modification or substitution at the position corresponding to position 40 in Dda 1993, the modified helicase of the invention preferably comprises any of the following additional modifications, substitutions, combinations of modifications or combination of substitutions. As explained above, the invention also provides a modified helicase having any of the modifications, substitutions, combinations of modifications or combinations of substitutions set out below in isolation (i.e., without necessarily having a substitution at the any of positions 40, 55, 114, 156, 177, 210, 221, 350 and 358 of Dda 1993 or the any of positions 40, 114, 177, 350 and 358 of Dda 1993).

### Modifications of the Invention

The helicase of the invention may be one in which at least one amino acid which interacts with a transmembrane pore is substituted. Any number of amino acids may substituted, such as 1 or more, 2 or more, 3 or more, 4 or more, 5 or more or 6 or more amino acids. 1, 2, 3, 4, 5, 6, 7, 8, 9, 10 or more amino acids may be substituted. The amino acids which interact with a transmembrane pore can be identified using protein modelling as discussed above.

Base and/or Sugar Interactions

The helicase of the invention is preferably one in which at least one amino acid which interacts with the sugar and/or base of one or more nucleotides in single stranded DNA (ssDNA) is substituted with an amino acid which comprises a larger side chain (R group). Any number of amino acids may substituted, such as 1 or more, 2 or more, 3 or more, 4 or more, 5 or more or 6 or more amino acids. Each amino acid may interact with the base, the sugar or the base and the sugar. The amino acids which interact with the sugar and/or base of one or more nucleotides in single stranded DNA can be identified using protein modelling as discussed above.

The helicase of the invention preferably comprises a variant of SEQ ID NO: 118 wherein the at least one amino acid which interacts with the sugar and/or base of one or more nucleotides in ssDNA is at least one of H82, N88, P89, F98, D121, V150, P152, F240, F276, S287, H396 and Y415. These numbers correspond to the relevant positions in SEQ ID NO: 118 and may need to be altered in the case of variants where one or more amino acids have been inserted or deleted compared with SEQ ID NO: 118. A skilled person can determine the corresponding positions in a variant as discussed above. The helicase of the invention preferably comprises a variant of SEQ ID NO: 118 wherein the at least one amino acid which interacts with the sugar and/or base of one or more nucleotides in ssDNA is F98 and one or more H82, N88, P89, D121, V150, P152, F240, F276, S287, H396 and Y415, such as F98/H82, F98/N88, F98/P89, F98/D121, F98/V150, F98/P152, F98/F240, F98/F276, F98/5287 or F98/H396.

The helicase of the invention is preferably a variant of SEQ ID NO: 119, 120, 121, 122, 123, 124, 125, 126, 127, 128, 129, 130, 131, 132 or 133 wherein the at least one amino acid which interacts with the sugar and/or base of one or more nucleotides in ssDNA is at least one of the amino acids which correspond to H82, N88, P89, F98, D121, V150, P152, F240, F276, S287, H396 and Y415 in SEQ ID NO: 118. The helicase of the invention preferably comprises a variant of SEQ ID NO: 119, 120, 121, 122, 123, 124, 125, 126, 127, 128, 129, 130, 131, 132 or 133 wherein the at least one amino acid which interacts with the sugar and/or base of one or more nucleotides in ssDNA is the amino acid which corresponds to F98 in SEQ ID NO: 118 and one or more of the amino acids which correspond to H82, N88, P89, D121, V150, P152, F240, F276, S287, H396 and Y415 in SEQ ID NO: 118, such as the amino acids which correspond to F98/H82, F98/N88, F98/P89, F98/D121, F98/V150, F98/P152, F98/F240, F98/F276, F98/S287 or F98/H396.

Table 6 shows the amino acids in SEQ ID NOs: 119 to 133 which correspond to H82, N88, P89, F98, D121, V150, P152, F240, F276, S287, H396 and Y415 in SEQ ID NO: 118.

| SEQ ID NO: | | | | | | | | | | | | |
|---|---|---|---|---|---|---|---|---|---|---|---|---|
| 118 | H82 | N88 | P89 | F98 | D121 | V150 | P152 | F240 | F276 | S287 | H396 | Y415 |
| 119 | H81 | F87 | D88 | S105 | S131 | V181 | Q183 | R274 | H313 | G314 | H428 | H447 |
| 120 | A144 | Q150 | P151 | G158 | N187 | V217 | K218 | W307 | F344 | K355 | H455 | H473 |
| 121 | H87 | R93 | L94 | G100 | G126 | V154 | N155 | Y240 | Y277 | G280 | H377 | R395 |
| 122 | H112 | K118 | P119 | E128 | G154 | I185 | N187 | Y273 | F309 | K310 | H414 | H433 |
| 123 | H93 | V99 | M100 | D106 | N132 | I159 | Q160 | Y267 | M302 | G303 | H400 | K419 |
| 124 | H74 | H80 | P81 | F90 | D114 | V143 | H145 | Y230 | M266 | P273 | H378 | Y397 |
| 125 | H78 | H84 | P85 | F94 | S117 | E147 | A149 | Y235 | M271 | I279 | H387 | Y406 |
| 126 | H65 | H71 | P72 | F81 | S104 | V133 | H135 | F222 | M258 | I266 | H373 | Y392 |
| 127 | H84 | S90 | P91 | F100 | D126 | V155 | A157 | Y243 | V279 | T290 | H399 | A418 |
| 128 | H84 | S90 | P91 | F100 | D126 | V155 | T157 | Y243 | V279 | V290 | H398 | A417 |
| 129 | Q82 | N88 | P89 | F98 | T121 | V150 | E152 | Y237 | M274 | K285 | H393 | Q412 |
| 130 | H88 | N94 | P95 | F104 | D127 | V156 | P158 | F246 | I282 | S293 | H399 | K418 |
| 131 | H84 | N90 | P91 | F100 | D123 | V152 | P154 | Y242 | M278 | S289 | H399 | M418 |
| 132 | H83 | N89 | P90 | F99 | D122 | V151 | P153 | Y241 | M277 | H288 | H396 | M415 |
| 133 | H83 | N89 | P90 | F99 | D122 | V151 | P153 | F241 | M277 | H288 | H397 | M416 |

The at east one amino acid which interacts with the sugar an or base of one or more nucleotides in ssDNA is preferably at least one amino acid which intercalates between the nucleotides in ssDNA. Amino acids which intercalate between nucleotides in ssDNA can be modeled as discussed above. The at least one amino acid which intercalates between the nucleotides in ssDNA is preferably at least one of P89, F98 and V150 in SEQ ID NO: 118, such as P89, F98, V150, P89/F98, P89/V150, F98/V150 or P89/F98/V150.

The at least one amino acid which intercalates between the nucleotides in ssDNA in SEQ ID NO: 119, 120, 121, 122, 123, 124, 125, 126, 127, 128, 129, 130, 131, 132 or 133 is preferably at least one of the amino acids which correspond to P89, F98 and V150 in SEQ ID NO: 118, such as P89, F98, V150, P89/F98, P89/V150, F98/V150 or P89/F98/V150. Corresponding amino acids are shown in Table 6 above.

### Larger R Groups

The larger side chain (R group) preferably (a) contains an increased number of carbon atoms, (b) has an increased length, (c) has an increased molecular volume and/or (d) has an increased van der Waals volume. The larger side chain (R group) preferably (a); (b); (c); (d); (a) and (b); (a) and (c); (a) and (d); (b) and (c); (b) and (d); (c) and (d); (a), (b) and (c); (a), (b) and (d); (a), (c) and (d); (b), (c) and (d); or (a), (b), (c) and (d). Each of (a) to (d) may be measured using standard methods in the art.

The larger side chain (R group) preferably increases the (i) electrostatic interactions (ii) (ii) hydrogen bonding and/or (iii) cation-pi (cation-π) interactions between the at least one amino acid and the one or more nucleotides in ssDNA, such as increases (i); (ii); (iii); (i) and (ii); (i) and (iii); (ii) and (iii); and (i), (ii) and (iii). A skilled person can determine if the R group increases any of these interactions. For instance in (i), positively charged amino acids, such as arginine (R), histidine (H) and lysine (K), have R groups which increase electrostatic interactions. For instance in (ii), amino acids such as asparagine (N), serine (S), glutamine (Q), threonine (T) and histidine (H) have R groups which increase hydrogen bonding. For instance in (iii), aromatic amino acids, such as phenylalanine (F), tryptophan (W), tyrosine (Y) or histidine (H), have R groups which increase cation-pi (cation-π) interactions. Specific substitutions below are labelled (i) to (iii) to reflect these changes. Other possible substitutions are labelled (iv).

These (iv) substitutions typically increase the length of the side chain (R group).

The amino acid which comprises a larger side chain (R) may be a non-natural amino acid. The non-natural amino acid may be any of those discussed below.

The amino acid which comprises a larger side chain (R group) is preferably not alanine (A), cysteine (C), glycine (G), selenocysteine (U), methionine (M), aspartic acid (D) or glutamic acid (E).

Histidine (H) is preferably substituted with (i) arginine (R) or lysine (K), (ii) glutamine (Q) or asparagine (N) or (iii) phenylalanine (F), tyrosine (Y) or tryptophan (W). Histidine (H) is more preferably substituted with (a) N, Q or W or (b) Y, F, Q or K.

Asparagine (N) is preferably substituted with (i) arginine (R) or lysine (K), (ii) glutamine (Q) or histidine (H) or (iii) phenylalanine (F), tyrosine (Y) or tryptophan (W). Asparagine (N) is more preferably substituted with R, H, W or Y.

Proline (P) is preferably substituted with (i) arginine (R) or lysine (K), (ii) glutamine (Q), asparagine (N), threonine (T) or histidine (H), (iii) tyrosine (Y), phenylalanine (F) or tryptophan (W) or (iv) leucine (L), valine (V) or isoleucine (I). Proline (P) is more preferably substituted with (i) arginine (R) or lysine (K), (ii) glutamine (Q), asparagine (N), threonine (T) or histidine (H), (iii) phenylalanine (F) or tryptophan (W) or (iv) leucine (L), valine (V) or isoleucine (I). Proline (P) is more preferably substituted with (a) F, (b) L, V, I, T or F or (c) W, F, Y, H, I, L or V.

Valine (V) is preferably substituted with (i) arginine (R) or lysine (K), (ii) glutamine (Q), asparagine (N) or histidine (H), (iii) phenylalanine (F), tyrosine (Y) or tryptophan (W) or (iv) isoleucine (I) or leucine (L). Valine (V) is more preferably substituted with (i) arginine (R) or lysine (K), (ii) glutamine (Q), asparagine (N) or histidine (H), (iii) tyrosine (Y) or tryptophan (W) or (iv) isoleucine (I) or leucine (L). Valine (V) is more preferably substituted with I or H or I, L, N, W or H.

Phenylalanine (F) is preferably substituted with (i) arginine (R) or lysine (K), (ii) histidine (H) or (iii) tyrosine (Y) or tryptophan (W). Phenylalanine (F) is more preferably substituted with (a) W, (b) W, Y or H, (c) W, R or K or (d) K, H, W or R.

Glutamine (Q) is preferably substituted with (i) arginine (R) or lysine (K) or (iii) phenylalanine (F), tyrosine (Y) or tryptophan (W).

Alanine (A) is preferably substituted with (i) arginine (R) or lysine (K), (ii) glutamine (Q), asparagine (N) or histidine (H), (iii) phenylalanine (F), tyrosine (Y) or tryptophan (W) or (iv) isoleucine (I) or leucine (L).

Serine (S) is preferably substituted with (i) arginine (R) or lysine (K), (ii) glutamine (Q), asparagine (N) or histidine (H), (iii) phenylalanine (F), tyrosine (Y) or tryptophan (W) or (iv) isoleucine (I) or leucine (L). Serine (S) is preferably substituted with K, R, W or F Lysine (K) is preferably substituted with (i) arginine (R) or (iii) tyrosine (Y) or tryptophan (W).

Arginine (R) is preferably substituted with (iii) tyrosine (Y) or tryptophan (W).

Methionine (M) is preferably substituted with (i) arginine (R) or lysine (K), (ii) glutamine (Q), asparagine (N) or histidine (H) or (iii) phenylalanine (F), tyrosine (Y) or tryptophan (W).

Leucine (L) is preferably substituted with (i) arginine (R) or lysine (K), (ii) glutamine (Q) or asparagine (N) or (iii) phenylalanine (F), tyrosine (Y) or tryptophan (W).

Aspartic acid (D) is preferably substituted with (i) arginine (R) or lysine (K), (ii) glutamine (Q), asparagine (N) or histidine (H) or (iii) phenylalanine (F), tyrosine (Y) or tryptophan (W). Aspartic acid (D) is more preferably substituted with H, Y or K.

Glutamic acid (E) is preferably substituted with (i) arginine (R) or lysine (K), (ii) glutamine (Q), asparagine (N) or histidine (H) or (iii) phenylalanine (F), tyrosine (Y) or tryptophan (W).

Isoleucine (I) is preferably substituted with (i) arginine (R) or lysine (K), (ii) glutamine (Q), asparagine (N) or histidine (H), (iii) phenylalanine (F), tyrosine (Y) or tryptophan (W) or (iv) leucine (L).

Tyrosine (Y) is preferably substituted with (i) arginine (R) or lysine (K) or (iii) tryptophan (W). Tyrosine (Y) is more preferably substituted with W or R.

The helicase more preferably comprises a variant of SEQ ID NO: 118 and comprises (a) P89F, (b) F98W, (c) V150I, (d) V150H, (e) P89F and F98W, (f) P89F and V150I, (g) P89F and V150H, (h) F98W and V150I, (i) F98W and V150H (j) P89F, F98W and V150I or (k) P89F, F98W and V150H.

The helicase more preferably comprises a variant of SEQ ID NO: 118 which comprises: H82N; H82Q; H82W; N88R; N88H; N88W; N88Y; P89L; P89V; P89I; P89E; P89T; P89F; D121H; D121Y; D121K; V150I; V150L; V150N; V150W; V150H; P152W; P152F; P152Y; P152H; P152I; P152L; P152V; F240W; F240Y; F240H; F276W; F276R; F276K; F276H; S287K; S287R; S287W; S287F; H396Y; H396F; H396Q; H396K; Y415W; Y415R; F98W/H82N; F98W/H82Q; F98W/H82W; F98W/N88R; F98W/N88H; F98W/N88W; F98W/N88Y; F98W/P89L; F98W/P89V; F98W/P89I; F98W/P89T; F98W/P89F; F98W/D121H; F98W/D121Y; F98W/D121K; F98W/V150I; F98W/V150L; F98W/V150N; F98W/V150W; F98W/V150H; F98W/P152W; F98W/P152F; F98W/P152Y; F98W/P152H; F98W/P152I; F98W/P152L; F98W/P152V; F98W/F240W; F98W/F240Y; F98W/F240H; F98W/F276W; F98W/F276R; F98W/F276K; F98W/F276H; F98W/S287K; F98W/S287R; F98W/S287W; F98W/S287F; F98W/H396Y; F98W/H396F; F98W/H396Q; F98W/Y415W; or F98W/Y415R.

### Phosphate Interactions

The helicase of the invention is preferably one in which at least one amino acid which interacts with one or more phosphate groups in one or more nucleotides in ssDNA is substituted. Any number of amino acids may be substituted, such as 1 or more, 2 or more, 3 or more, 4 or more, 5 or more or 6 or more amino acids. Nucleotides in ssDNA each comprise three phosphate groups. Each amino which is substituted may interact with any number of the phosphate groups at a time, such as one, two or three phosphate groups at a time. The amino acids which interact with one or more phosphate groups can be identified using protein modelling as discussed above.

The substitution preferably increases the (i) electrostatic interactions, (ii) hydrogen bonding and/or (iii) cation-pi (cation-π) interactions between the at least one amino acid and the one or more phosphate groups in ssDNA. Preferred substitutions which increase (i), (ii) and (iii) are discussed below using the labelling (i), (ii) and (iii).

The substitution preferably increases the net positive charge of the position. The net charge at any position can be measured using methods known in the art. For instance, the isoelectric point may be used to define the net charge of an amino acid. The net charge is typically measured at about 7.5. The substitution is preferably the substitution of a negatively charged amino acid with a positively charged, uncharged, non-polar or aromatic amino acid. A negatively charged amino acid is an amino acid with a net negative charge. Negatively charged amino acids include, but are not limited to, aspartic acid (D) and glutamic acid (E). A positively charged amino acid is an amino acid with a net positive charge. The positively charged amino acid can be naturally-occurring or non-naturally-occurring. The positively charged amino acid may be synthetic or modified. For instance, modified amino acids with a net positive charge may be specifically designed for use in the invention. A number of different types of modification to amino acids are well known in the art. Preferred naturally-occurring positively charged amino acids include, but are not limited to, histidine (H), lysine (K) and arginine (R).

The uncharged amino acid, non-polar amino acid or aromatic amino acid can be naturally occurring or non-naturally-occurring. It may be synthetic or modified. Uncharged amino acids have no net charge. Suitable uncharged amino acids include, but are not limited to, cysteine (C), serine (S), threonine (T), methionine (M), asparagines (N) and glutamine (Q). Non-polar amino acids have non-polar side chains. Suitable non-polar amino acids include, but are not limited to, glycine (G), alanine (A), proline (P), isoleucine (I), leucine (L) and valine (V). Aromatic amino acids have an aromatic side chain. Suitable aromatic amino acids include, but are not limited to, histidine (H), phenylalanine (F), tryptophan (W) and tyrosine (Y).

The helicase preferably comprises a variant of SEQ ID NO: 118 wherein the at least one amino acid which interacts with one or more phosphates in one or more nucleotides in ssDNA is at least one of H64, T80, S83, N242, K243, N293, T394 and K397. These numbers correspond to the relevant positions in SEQ ID NO: 89 and may need to be altered in the case of variants where one or more amino acids have been inserted or deleted compared with SEQ ID NO: 118. A skilled person can determine the corresponding positions in a variant as discussed above.

The helicase preferably comprises a variant of SEQ ID NO: 119, 120, 121, 122, 123, 124, 125, 126, 127, 128, 129, 130, 131, 132 or 133 and wherein the at least one amino acid which interacts with one or more phosphates in one or more nucleotides in ssDNA is at least one of the amino acids which correspond to H64, T80, S83, N242, K243, N293, T394 and K397 in SEQ ID NO: 118.

Table 7 shows the amino acids in SEQ ID NOs: 119 to 133 which correspond to H64, T80, S83, N242, K243, N293, T 394 and K397 in SEQ ID NO: 118.

| SEQ ID NO: | | | | | | | | |
|---|---|---|---|---|---|---|---|---|
| 118 | H64 | T80 | S83 | N242 | K243 | N293 | T394 | K397 |
| 119 | G63 | T79 | S82 | N276 | A277 | N317 | T426 | K429 |
| 120 | N121 | T142 | K145 | N309 | K310 | N361 | T453 | K456 |
| 121 | H66 | T85 | S88 | N242 | K243 | N286 | T375 | R378 |
| 122 | H89 | T110 | S113 | N275 | K276 | V315 | T412 | K415 |
| 123 | H68 | T91 | S94 | N269 | A270 | N308 | T398 | K401 |
| 124 | H56 | T72 | S75 | N232 | D233 | N279 | T376 | K379 |
| 125 | H60 | T76 | S79 | N237 | K238 | N285 | T385 | K388 |
| 126 | H47 | T63 | S66 | N224 | K225 | N272 | T371 | K374 |
| 127 | H66 | T82 | S85 | N245 | D246 | N296 | T397 | K400 |
| 128 | H66 | T82 | S85 | N245 | A246 | N296 | T396 | K399 |
| 129 | H64 | T80 | S83 | N239 | N240 | N291 | T391 | K394 |
| 130 | H70 | T86 | S89 | N248 | K249 | N299 | T397 | K400 |
| 131 | H66 | T82 | S85 | N244 | K245 | N295 | T397 | K400 |
| 132 | H65 | T81 | S84 | N243 | K244 | N294 | T394 | K397 |
| 133 | H65 | T81 | K84 | N243 | K244 | N294 | T395 | K398 |

Histidine (H) is preferably substituted with (i) arginine (R) or lysine (K), (ii) asparagine (N), serine (S), glutamine (Q) or threonine (T), (iii) phenylalanine (F), tryptophan (W) or tyrosine (Y). Histidine (H) is preferably substituted with (a) N, Q, K or F or (b) N, Q or W.

Threonine (T) is preferably substituted with (i) arginine (R), histidine (H) or lysine (K), (ii) asparagine (N), serine (S), glutamine (Q) or histidine (H) or (iii) phenylalanine (F), tryptophan (W), tyrosine (Y) or histidine (H). Threonine (T) is more preferably substituted with (a) K, Q or N or (b) K, H or N.

Serine (s) is preferably substituted with (i) arginine (R), histidine (H) or lysine (K), (ii) asparagine (N), glutamine (Q), threonine (T) or histidine (H) or (iii) phenylalanine (F), tryptophan (W), tyrosine (Y) or histidine (H). Serine (S) is more preferably substituted with H, N, K, T, R or Q.

Asparagine (N) is preferably substituted with (i) arginine (R), histidine (H) or lysine (K), (ii) serine (S), glutamine (Q), threonine (T) or histidine (H) or (iii) phenylalanine (F), tryptophan (W), tyrosine (Y) or histidine (H). Asparagine (N) is more preferably substituted with (a) H or Q or (b) Q, K or H.

Lysine (K) is preferably substituted with (i) arginine (R) or histidine (H), (ii) asparagine (N), serine (S), glutamine (Q), threonine (T) or histidine (H) or (iii) phenylalanine (F), tryptophan (W), tyrosine (Y) or histidine (H). Lysine (K) is more preferably substituted with (a) Q or H or (b) R, H or Y.

The helicase more preferably comprises a variant of SEQ ID NO: 118 and comprises one or more of, such as all of, (a) H64N, H64Q, H64K or H64F, (b) T80K, T80Q or T80N, (c) S83H, S83N, S83K, S83T, S83R, or S83Q (d) N242H or N242Q, (e) K243Q or K243H, (f) N293Q, N293K or N293H, (g) T394K, T394H or T394N or (h) K397R, K397H or K397Y.

### Combinations

The helicase is preferably a variant of SEQ ID NO: 118 which comprises substitutions at:
F98/H64, such as F98W/H64N, F98W/H64Q, F98W/H64K or F98W/H64F;
F98/T80, such as F98W/T80K, F98W/T80Q, F98W/T80N;
F98/H82, such as F98W/H82N, F98W/H82Q or F98W/H82W;
F98/583, such as F98W/S83H, F98W/S83N, F98W/S83K, F98W/S83T, F98W/S83R or F98W/S83Q;
F98/N242, such as F98W/N242H, F98W/N242Q, F98W/K243Q or F98W/K243H;
F98/N293, such as F98W/N293Q, F98W/N293K, F98W/N293H, F98W/T394K, F98W/T394H, F98W/T394N, F98W/H396Y, F98W/H396F, F98W/H396Q or F98W/H396K; or
F98/K397, such as F98W/K397R, F98W/K397H or F98W/K397Y.

Preferred combinations in SEQ ID NO: 119, 120, 121, 122, 123, 124, 125, 126, 127, 128, 129, 130, 131, 132 or 133 include the combinations of amino acids which correspond to the combinations in SEQ ID NO: 118 listed above.

### Pore Interaction

The helicase of the invention is further one in which the part of the helicase which interacts with a transmembrane pore comprises one or more modifications, preferably one or more substitutions. The part of the helicase which interacts with a transmembrane pore is typically the part of the helicase which interacts with a transmembrane pore when the helicase is used to control the movement of a polynucleotide through the pore, for instance as discussed in more detail below. The part typically comprises the amino acids that interact with or contact the pore when the helicase is used to control the movement of a polynucleotide through the pore, for instance as discussed in more detail below. The part typically comprises the amino acids that interact with or contact the pore when the helicase is bound to or attached to an analyte such as polynucleotide which is moving through the pore under an applied potential.

In SEQ ID NO: 118, the part which interacts with the transmembrane pore typically comprises the amino acids at positions 1, 2, 3, 4, 5, 6, 51, 176, 177, 178, 179, 180, 181, 185, 189, 191, 193, 194, 195, 197, 198, 199, 200, 201, 202, 203, 204, 207, 208, 209, 210, 211, 212, 213, 216, 219, 220, 221, 223, 224, 226, 227, 228, 229, 247, 254, 255, 256, 257, 258, 259, 260, 261, 298, 300, 304, 308, 318, 319, 321, 337, 347, 350, 351, 405, 415, 422, 434, 437, 438. These numbers correspond to the relevant positions in SEQ ID NO: 118 and may need to be altered in the case of variants where one or more amino acids have been inserted or deleted compared with SEQ ID NO: 118. A skilled person can determine the corresponding positions in a variant as discussed above. The part which interacts with the transmembrane pore preferably comprises the amino acids at
(a) positions 1, 2, 4, 51, 177, 178, 179, 180, 185, 193, 195, 197, 198, 199, 200, 202, 203, 204, 207, 208, 209, 210, 211, 212, 216, 221, 223, 224, 226, 227, 228, 229, 254, 255, 256, 257, 258, 260, 304, 318, 321, 347, 350, 351, 405, 415, 422, 434, 437 and 438 in SEQ ID NO: 118; or
(b) positions 1, 2, 178, 179, 180, 185, 195, 197, 198, 199, 200, 202, 203, 207, 209, 210, 212, 216, 221, 223, 226, 227, 255, 258, 260, 304, 350 and 438 in SEQ ID NO: 118.

The part which interacts with the transmembrane pore preferably comprises one or more of, such as 2, 3, 4 or 5 of, the amino acids at positions K194, W195, K198, K199 and E258 in SEQ ID NO: 118. The variant of SEQ ID NO: 118 preferably comprises a modification at one or more of (a), K194, (b) W195, (c) D198, (d) K199 and (d) E258. The variant of SEQ ID NO: 118 preferably comprises a substitution at one or more of (a) K194, such as K194L, (b) W195, such as W195A, (c) D198, such as D198V, (d) K199, such as K199L and (e) E258, such as E258L.

The variant may comprise {a}; {b}; {c}; {d}; {e}; {a,b}; {a,c}; {a,d}; {a,e}; {b,c}; {b,d}; {b,e}; {c,d}; {c,e}; {d,e}; {a,b,c}; {a,b,d}; {a,b,e}; {a,c,d}; {a,c,e}; {a,d,e}; {b,c,d}; {b,c,e}; {b,d,e}; {c,d,e}; {a,b,c,d}; {a,b,c,e}; {a,b,d,e}; {a,c,d,e}; {b,c,d,e}; or {a,b,c,d,e}. The modifications or substitutions set out in this paragraph are preferred when the modified polynucleotide binding protein interacts with a pore derived from MspA, particularly any of the modified pores discussed below.

The part of the polynucleotide binding protein which interacts with the transmembrane pore preferably comprises the amino acid at position 194 or 199 of SEQ ID NO: 118. The variant preferably comprises K194A, K194V, K194F, K194D, K194S, K194W or K194L and/or K199A, K199V, K199F, K199D, K199S, K199W or K199L. In SEQ ID NO: 119, 120, 121, 122, 123, 124, 125, 126, 127, 128, 129, 130, 131, 132 or 133, the part which interacts with the transmembrane pore typically comprises the amino acids at positions which correspond to those in SEQ ID NO: 118 listed above. Amino acids in SEQ ID NOs: 119, 120, 121, 122, 123, 124, 125, 126, 127, 128, 129, 130, 131, 132 and 133 which correspond to these positions in SEQ ID NO: 118 can be identified using the alignment in Table 8 below.

| | | | | | |
|---|---|---|---|---|---|
| SEQ ID NO: 118 | K194 | W195 | D198 | K199 | E258 |
| SEQ ID NO: 119 | L230 | E231 | H234 | Y235 | R293 |
| SEQ ID NO: 120 | W259 | N260 | T263 | Y264 | E326 |
| SEQ ID NO: 121 | A192 | D193 | F196 | G197 | A259 |
| SEQ ID NO: 122 | I224 | K225 | D228 | F229 | Q292 |
| SEQ ID NO: 123 | Q213 | D214 | Y217 | A218 | A286 |
| SEQ ID NO: 124 | Q185 | W186 | T189 | N190 | N248 |
| SEQ ID NO: 125 | G190 | W191 | P194 | N195 | K253 |
| SEQ ID NO: 126 | G177 | W178 | Q181 | N182 | K240 |
| SEQ ID NO: 127 | K200 | M201 | P204 | M205 | K261 |
| SEQ ID NO: 128 | K200 | P201 | P204 | L205 | K261 |
| SEQ ID NO: 129 | K193 | W194 | E197 | K198 | A256 |
| SEQ ID NO: 130 | N200 | W201 | E204 | N205 | N264 |
| SEQ ID NO: 131 | G196 | W197 | D200 | C201 | E260 |
| SEQ ID NO: 132 | G195 | W196 | E199 | N200 | E259 |
| SEQ ID NO: 133 | S195 | W196 | E199 | K200 | Q259 |

### Preferred Combinations

The helicase of the invention preferably comprises a variant of SEQ ID NO: 118 which comprises a substitution at F98, such as F98R, F98K, F98Q, F98N, F98H, F98Y, F98F or F98W, and a substitution at K194, such as K194A, K194V, K194F, K194D, K194S, K194W or K194L, and/or K199, such as K199A, K199V, K199F, K199D, K199S, K199W or K199L. The helicase of the invention preferably comprises a variant of SEQ ID NO: 119, 120, 121, 122, 123, 124, 125, 126, 127, 128, 129, 130, 131, 132 or 133 which comprises a substitution at the position which corresponds to F98 in SEQ ID NO: 118 and a substitution at the position(s) which correspond to K194 and/or K199 in SEQ ID NO: 118. These corresponding positions may be replaced with any of the amino acids listed above for F98, K194 and K119 in SEQ ID NO: 118. The helicase is preferably a variant of SEQ ID NO: 118 which comprises substitutions at:
F98/K194/H64, such as F98W/K194L/H64N, F98W/K194L/H64Q, F98W/K194L/H64K or F98W/K194L/H64F;
F98/K194/T80, such as F98W/K194L/T80K, F98W/K194L/T80Q or F98W/K194L/T80N;
F98/K194/H82, such as F98W/K194L/H82N, F98W/K194L/H82Q or F98W/K194L/H82W
F98/S83/K194, such as F98W/S83H/K194L, F98W/S83T/K194L, F98W/S83R/K194L, F98W/S83Q/K194L, F98W/S83N/K194L, F98W/S83K/K194L, F98W/N88R/K194L, F98W/N88H/K194L, F98W/N88W/K194L or F98W/N88Y/K194L;
F98/S83/K194/F276, such as F98W/S83H/K194L/F276K;
F98/P89/K194, such as F98W/P89L/K194L, F98W/P89V/K194L, F98W/P89I/K194L or F98W/P89T/K194L;
F98/D121/K194, such as F98W/D121H/K194L, F98W/D121Y/K194L or F98W/D121K/K194L;
F98/V150/K194, such as F98W/V150I/K194L, F98W/V150L/K194L, F98W/V150N/K194L, F98W/V150W/K194L or F98W/V150H/K194L;
F98/P152/K194, such as F98W/P152W/K194L, F98W/P152F/K194L, F98W/P152Y/K194L, F98W/P152H/K194L, F98W/P152I/K194L, F98W/P152L/K194L or F98W/P152V/K194L;
F98/F240/K194, such as F98W/F240W/K194L, F98W/F240Y/K194L or F98W/F240H/K194L;
F98/N242/K194, such as F98W/N242H/K194L or F98W/N242Q/K194L;
F98/K194/F276, such as F98W/K194L/F276K, F98W/K194L/F276H, F98W/K194L/F276W or F98W/K194L/F276R;
F98/K194/5287, such as F98W/K194L/S287K, F98W/K194L/S287R, F98W/K194L/S287W or F98W/K194L/S287F;
F98/N293/K194, such as F98W/N293Q/K194L, F98W/N293K/K194L or F98W/N293H/K194L;
F98/T394/K194, such as F98W/T394K/K194L, F98W/T394H/K194L or F98W/T394N/K194L;
F98/H396/K194, such as F98W/H396Y/K194L, F98W/H396F/K194L, F98W/H396Q/K194L or F98W/H396K/K194L;
F98/K397/K194, such as F98W/K397R/K194L, F98W/K397H/K194L or F98W/K397Y/K194L; or
F98/Y415/K194, such as F98W/Y415W/K194L or F98W/Y415R/K194L.

In any of the above combinations, K194 may be replaced with any of W195, D198, K199 and E258.

The modified helicase preferably comprises a modification or substitution at the position(s) corresponding to amino acid positions 98 and/or 194 in Dda 1993. This is preferably in addition to a modification or substitution at one or more of the positions corresponding to amino acid positions 55, 114, 156, 177, 210, 221, 350 and 358 in Dda 1993, a modification or substitution at one or more positions corresponding to amino acid positions 114, 177, 350 and 358 in Dda 1993 and/or a modification or substitution at the position corresponding to position 40 in Dda 1993. Position 98 or the corresponding position may be substituted with R, H, K, S, T, N, Q, A, V, I, L, M, Y or W. Position 98 or the corresponding position is preferably substituted with R, K, Q, N, H, Y or W. Position 194 or the corresponding position may be substituted with A, V, I, L, M, F, Y, W, D, E, S, T, N or Q. Position 194 or the corresponding position is preferably substituted with A, V, F, D, S, W or L. The helicase of the invention preferably comprises a variant of SEQ ID NO: 118 which comprises a substitution at F98, such as F98R, F98K, F98Q, F98N, F98H, F98Y or F98W, and/or a substitution at K194, such as K194A, K194V, K194F, K194D, K194S, K194W or K194L. The helicase of the invention preferably comprises a variant of SEQ ID NO: 118 which comprises F98W and K194L. The helicase of the invention preferably comprises a variant of SEQ ID NO: 119, 120, 121, 122, 123, 124, 125, 126, 127, 128, 129, 130, 131, 132 or 133 which comprises a substitution at the position which corresponds to F98 in SEQ ID NO: 118 and/or a substitution at the position which corresponds to K194 in SEQ ID NO: 118.

In any of the above combinations, K194 may be replaced with any of W195, D198, K199 and E258.

Modifications in the Tower Domain and/or Pin Domain and/or 1A Domain

The modified helicase preferably comprises a modification or substitution at the position corresponding to amino acid position 360 in Dda 1993. This may be in addition to a modification or substitution at one or more positions corresponding to amino acid positions 55, 114, 156, 177, 210, 221, 350 and 358 in Dda 1993, a modification or substitution at one or more positions corresponding to amino acid positions 114, 177, 350 and 358 in Dda 1993 and/or a modification or substitution at the position corresponding to position 40 in Dda 1993. A360 is in the tower domain of Dda 1993, like Y350 and K358. Position 360 or the corresponding position may be substituted with C, G, P, A, V, I, L, M, F, Y or W. Position 360 or the corresponding position is preferably substituted with C or Y. The helicase of the invention preferably comprises a variant of SEQ ID NO: 118 which comprises a substitution at A360, such as A360C or A360Y. The helicase of the invention preferably comprises a variant of SEQ ID NO: 118 which comprises K358I and A360C. The helicase of the invention preferably comprises a variant of SEQ ID NO: 119, 120, 121, 122, 123, 124, 125, 126, 127, 128, 129, 130, 131, 132 or 133 which comprises a substitution at the position which corresponds to A360 in SEQ ID NO: 118.

The modified helicase preferably comprises a modification or substitution at one or more of the positions corresponding to amino acid positions 94, 98 and 109 in Dda 1993, such as position(s) 94, 98, 109, 94 and 98, 94 and 109, 98 and 109 and 94, 98 and 109. This may be in addition to a modification or substitution at one or more positions corresponding to amino acid positions 55, 114, 156, 177, 210, 221, 350 and 358 in Dda 1993, a modification or substitution at one or more positions corresponding to amino acid positions 114, 177, 350 and 358 in Dda 1993 and/or a modification or substitution at the position corresponding to position 40 in Dda 1993. These positions are all in the pin domain. Position 94 or the corresponding position may be substituted with C, G, P, A, V, I, L, M, F, Y or W. Position 94 or the corresponding position is preferably substituted with C or Y. Position 98 or the corresponding position may be substituted with R, H, K, S, T, N, Q, A, V, I, L, MY or W. Position 98 or the corresponding position is preferably substituted with R, K, Q, N, H, Y or W. Position 109 or the corresponding position may be substituted with A, V, I, L, M, F, Y or W. Position 109 or the corresponding position is preferably substituted with A or V. The helicase of the invention preferably comprises a variant of SEQ ID NO: 118 which comprises a substitution at one or more of E94, F98 and C109 (including all the combinations set out above). Preferred variants comprise substitutions at:
E94 and F98, such as E94C or E94Y and F98R, F98K, F98Q, F98N, F98H, F98Y, F98F or F98W;
E94 and C109, such as E94C or E94Y and C109A or C109V;
F98 and C109, such as F98R, F98K, F98Q, F98N, F98H, F98Y, F98F or F98W and C109A or C109V; or
E94, F98 and C109, such as E94C or E94Y and F98R, F98K, F98Q, F98N, F98H, F98Y, F98F or F98W and C109A or C109V.

More preferred variants comprise: E94C and F98W; E94C and C109A; F98W and C109A; or E94C, F98W and C109A. The helicase of the invention preferably comprises a variant of SEQ ID NO: 119, 120, 121, 122, 123, 124, 125, 126, 127, 128, 129, 130, 131, 132 or 133 which comprises a substitution at the position(s) which corresponds to one or more of E94, F98 and C109 in SEQ ID NO: 118.

Table 9 includes information for E94, C109, C136 and A360 (with reference to modified helicases disclosed above and below).

| | | | | |
|---|---|---|---|---|
| SEQ ID NO: 118 | E94 | C109 | C136 | A360 |
| SEQ ID NO: 119 | E95 | V124 | T167 | I388 |
| SEQ ID NO: 120 | D156 | I180 | S204 | A417 |
| SEQ ID NO: 121 | E101 | V119 | V140 | E342 |
| SEQ ID NO: 122 | E128 | V147 | V172 | A377 |
| SEQ ID NO: 123 | E107 | I125 | S145 | A364 |
| SEQ ID NO: 124 | D86 | V107 | C129 | A342 |
| SEQ ID NO: 125 | D90 | V110 | C132 | A349 |
| SEQ ID NO: 126 | D77 | I89 | C119 | A347 |
| SEQ ID NO: 127 | E96 | V119 | A141 | A336 |
| SEQ ID NO: 128 | E96 | V119 | A141 | A360 |
| SEQ ID NO: 129 | E94 | C114 | C136 | A357 |
| SEQ ID NO: 130 | D100 | C120 | C142 | A363 |
| SEQ ID NO: 131 | D96 | C116 | C138 | A365 |
| SEQ ID NO: 132 | E95 | C115 | C137 | A360 |
| SEQ ID NO: 133 | E95 | C115 | C137 | A361 |

The helicase of the invention is preferably one in which at least one cysteine residue (i.e. one or more cysteine residues) and/or at least one non-natural amino acid (i.e. one or more non-natural amino acids) have been introduced into (i) the tower domain and/or (ii) the pin domain and/or the (iii) 1A (RecA-like motor) domain, wherein the helicase has the ability to control the movement of a polynucleotide. These types of modification are disclosed in WO 2015/055981 (incorporated herein by reference in its entirety). At least one cysteine residue and/or at least one non-natural amino acid may be introduced into the tower domain, the pin domain, the 1A domain, the tower domain and the pin domain, the tower domain and the 1A domain or the tower domain, the pin domain and the 1A domain.

The helicase of the invention is preferably one in which at least one cysteine residue and/or at least one non-natural amino acid have been introduced into each of (i) the tower domain and (ii) the pin domain and/or the 1A (RecA-like motor) domain, i.e. into the tower domain and the pin domain, the tower domain and the 1A domain or the tower domain, the pin domain and the 1A domain.

Any number of cysteine residues and/or non-natural amino acids may be introduced into each domain. For instance, 1, 2, 3, 4, 5, 6, 7, 8, 9, 10 or more cysteine residues may be introduced and/or 1, 2, 3, 4, 5, 6, 7, 8, 9, 10 or more non-natural amino acids may be introduced. Only one or more cysteine residues may be introduced. Only one or more non-natural amino acids may be introduced. A combination of one or more cysteine residues and one or more non-natural amino acids may be introduced.

The at least one cysteine residue and/or at least one non-natural amino acid are/is preferably introduced by substitution. Methods for doing this are known in the art.

These modifications do not prevent the helicase from binding to a polynucleotide. These modifications decrease the ability of the polynucleotide to unbind or disengage from the helicase. In other words, the one or more modifications increase the processivity of the helicase by preventing dissociation from the polynucleotide strand. The thermal stability of the enzyme is typically also increased by the one or more modifications giving it an improved structural stability that is beneficial in Strand Sequencing.

A non-natural amino acid is an amino that is not naturally found in a helicase. The non-natural amino acid is preferably not histidine, alanine, isoleucine, arginine, leucine, asparagine, lysine, aspartic acid, methionine, cysteine, phenylalanine, glutamic acid, threonine, glutamine, tryptophan, glycine, valine, proline, serine or tyrosine. The non-natural amino acid is more preferably not any of the twenty amino acids in the previous sentence or selenocysteine.

Preferred non-natural amino acids for use in the invention include, but are not limited, to 4-Azido-L-phenylalanine (Faz), 4-Acetyl-L-phenylalanine, 3-Acetyl-L-phenylalanine, 4-Acetoacetyl-L-phenylalanine, O-Allyl-L-tyrosine, 3-(Phenylselanyl)-L-alanine, O-2-Propyn-1-yl-L-tyrosine, 4-(Dihydroxyboryl)-L-phenylalanine, 4-[(Ethylsulfanyl)carbonyl]-L-phenylalanine, (2S)-2-amino-3-4-[(propan-2-ylsulfanyl)carbonyl]phenyl;propanoic acid, (2S)-2-amino-3-4-[(2-amino-3-sulfanylpropanoyl)amino]phenyl;propanoic acid, O-Methyl-L-tyrosine, 4-Amino-L-phenylalanine, 4-Cyano-L-phenylalanine, 3-Cyano-L-phenylalanine, 4-Fluoro-L-phenylalanine, 4-Iodo-L-phenylalanine, 4-Bromo-L-phenylalanine, O-(Trifluoromethyl)tyrosine, 4-Nitro-L-phenylalanine, 3-Hydroxy-L-tyrosine, 3-Amino-L-tyrosine, 3-Iodo-L-tyrosine, 4-Isopropyl-L-phenylalanine, 3-(2-Naphthyl)-L-alanine, 4-Phenyl-L-phenylalanine, (2S)-2-amino-3-(naphthalen-2-ylamino)propanoic acid, 6-(Methylsulfanyl)norleucine, 6-Oxo-L-lysine, D-tyrosine, (2R)-2-Hydroxy-3-(4-hydroxyphenyl)propanoic acid, (2R)-2-Ammoniooctanoate3-(2,2′-Bipyridin-5-yl)-D-alanine, 2-amino-3-(8-hydroxy-3-quinolyl)propanoic acid, 4-Benzoyl-L-phenylalanine, S-(2-Nitrobenzyl)cysteine, (2R)-2-amino-3-[(2-nitrobenzyl)sulfanyl]propanoic acid, (2S)-2-amino-3-[(2-nitrobenzyl)oxy]propanoic acid, 0-(4,5-Dimethoxy-2-nitrobenzyl)-L-serine, (2S)-2-amino-6-([(2-nitrobenzyl)oxy]carbonyl;amino)hexanoic acid, 0-(2-Nitrobenzyl)-L-tyrosine, 2-Nitrophenylalanine, 4-[(E)-Phenyldiazenyl]-L-phenylalanine, 4-[3-(Trifluoromethyl)-3H-diaziren-3-yl]-D-phenylalanine, 2-amino-3-[[5-(dimethylamino)-1-naphthyl]sulfonylamino]propanoic acid, (2S)-2-amino-4-(7-hydroxy-2-oxo-2H-chromen-4-yl)butanoic acid, (2S)-3-[(6-acetylnaphthalen-2-yl)amino]-2-aminopropanoic acid, 4-(Carboxymethyl)phenylalanine, 3-Nitro-L-tyrosine, O-Sulfo-L-tyrosine, (2R)-6-Acetamido-2-ammoniohexanoate, 1-Methylhistidine, 2-Aminononanoic acid, 2-Aminodecanoic acid, L-Homocysteine, 5-Sulfanylnorvaline, 6-Sulfanyl-L-norleucine, 5-(Methylsulfanyl)-L-norvaline, N⁶-[(2R,3R)-3-Methyl-3,4-dihydro-2H-pyrrol-2-yl]carbonyl; -L-lysine, N⁶-[(Benzyloxy)carbonyl]lysine, (2S)-2-amino-6-[(cyclopentylcarbonyl)amino]hexanoic acid, N⁶-[(Cyclopentyloxy)carbonyl]-L-lysine, (2S)-2-amino-6-[(2R)-tetrahydrofuran-2-ylcarbonyl]amino;hexanoic acid, (2S)-2-amino-8-[(2R,3S)-3-ethynyltetrahydrofuran-2-yl]-8-oxooctanoic acid, N⁶-(tert-Butoxycarbonyl)-L-lysine, (2S)-2-Hydroxy-6-([(2-methyl-2-propanyl)oxy]carbonyl;amino)hexanoic acid, N⁶-[(Allyloxy)carbonyl]lysine, (2S)-2-amino-6-([(2-azidobenzyl)oxy]carbonyl;amino)hexanoic acid, N⁶-L-Prolyl-L-lysine, (2S)-2-amino-6-[(prop-2-yn-1-yloxy)carbonyl]amino;hexanoic acid and N⁶-[(2-Azidoethoxy)carbonyl]-L-lysine. The most preferred non-natural amino acid is 4-azido-L-phenylalanine (Faz).

Table 10 below (which is separated in two parts) identifies the residues making up each domain in each Dda homologue (SEQ ID NOs: 118 to 133).

| Homologue | SEQ ID NO | 1A | 2A |
|---|---|---|---|
| Dda-Rma-DSM | 119 | M1-I84 + R113-Y211 | R212-E294 + G422-S678 |
| Dda-Csp | 120 | M1-L147 + S166-V240 | R241-N327 + A449-G496 |
| Dda-Sru | 121 | M1-L90 + E108-H173 | R174-D260 + A371-V421 |
| Dda-Sgo | 122 | M1-L115 + N136-V205 | R206-K293 + I408-L500 |
| Dda-Vph12B8 | 123 | M1-L96 + F114-V194 | R195-D287 + V394-Q450 |
| Dda-Vph | 124 | M1-L77 + V96-V166 | R167-T249 + L372-N421 |
| Dda-Aph65 | 125 | M1-M81 + L99-M171 | R172-T254 + L381-K434 |
| Dda-AphCC2 | 126 | M1-M68 + M86-M158 | R159-T241 + L367-K420 |
| Dda-Cph | 127 | M1-L87 + A108-M181 | R182-T262 + L393-V443 |
| Dda-Kph | 128 | M1-L87 + A108-M181 | R182-T262 + L392-V442 |
| Dda-SphIME13 | 129 | M1-L85 + T103-K176 | R177-N257 + L387-V438 |
| Dda-AphAc42 | 130 | M1-L91 + V109-M183 | R184-T265 + L393-1442 |
| Dda-SphSP18 | 131 | M1-L87 + M105-M179 | R180-T261 + L393-V442 |
| Dda-Yph | 132 | M1-L86 + V104-K178 | R179-T260 + L390-1439 |
| Dda-SphS16 | 133 | M1-L86 + V104-M178 | R179-T260 + L391-V441 |
| Dda-1993 | 118 | M1-L85 + V103-K177 | R178-T259 + L390-V439 |

| Homologue | SEQ ID NO: | tower | pin | hook |
|---|---|---|---|---|
| Dda-Rma-DSM | 119 | G295-N309 + F316-Y421 | Y85-L112 | A310-L315 |
| Dda-Csp | 120 | V328-P342 + N360-Y448 | K148-N165 | V343-L359 |
| Dda-Sru | 121 | A261-T275 + T285-Y370 | G91-E107 | W276-L284 |
| Dda-Sgo | 122 | G294-I307 + T314-Y407 | G116-T135 | R308-Y313 |
| Dda-Vph12B8 | 123 | V288-E301 + N307-N393 | G97-P113 | M302-W306 |
| Dda-Vph | 124 | S250-P264 + E278-S371 | K78-E95 | V265-I277 |
| Dda-Aph65 | 125 | K255-P269 + T284-S380 | K82-K98 | V270-F283 |
| Dda-AphCC2 | 126 | D242-P256 + T271-S366 | K69-K85 | V257-F270 |
| Dda-Cph | 127 | T263-P277 + N295-P392 | K88-K107 | L278-Y294 |
| Dda-Kph | 128 | D263-P277 + N295-A391 | K88-K107 | L278-Y294 |
| Dda-SphIME13 | 129 | A258-P272 + N290-P386 | K86-G102 | L273-F289 |
| Dda-AphAc42 | 130 | L266-P280 + N298-A392 | K92-D108 | L281-F297 |
| Dda-SphSP18 | 131 | D262-P276 + N294-A392 | K88-E104 | H277-F293 |
| Dda-Yph | 132 | D261-P275 + N293-A389 | K87-E103 | L276-F292 |
| Dda-SphS16 | 133 | E261-P275 + T293-A390 | K87-E103 | L276-F292 |
| Dda-1993 | 118 | D260-P274 + N292-A389 | K86-E102 | L275-F291 |

The helicase of the invention preferably comprises a variant of SEQ ID NO: 118 in which at least one cysteine residue and/or at least one non-natural amino acid have been introduced into (i) the tower domain (residues D260-P274 and N292-A389) and/or (ii) the pin domain (residues K86-E102) and/or the (iii) 1A domain (residues M1-L85 and V103-K177). The at least one cysteine residue and/or at least one non-natural amino acid are preferably introduced into residues N292-A389 of the tower domain.

The helicase of the invention preferably comprises a variant of SEQ ID NO: 119 in which at least one cysteine residue and/or at least one non-natural amino acid have been introduced into (i) the tower domain (residues G295-N309 and F316-Y421) and/or (ii) the pin domain (residues Y85-L112) and/or the (iii) 1A domain (residues M1-I84 and R113-Y211). The at least one cysteine residue and/or at least one non-natural amino acid are preferably introduced into residues F316-Y421 of the tower domain.

The helicase of the invention preferably comprises a variant of SEQ ID NO: 120 in which at least one cysteine residue and/or at least one non-natural amino acid have been introduced into (i) the tower domain (residues V328-P342 and N360-Y448) and/or (ii) the pin domain (residues K148-N165) and/or the (iii) 1A domain (residues M1-L147 and 5166-V240). The at least one cysteine residue and/or at least one non-natural amino acid are preferably introduced into residues N360-Y448 of the tower domain.

The helicase of the invention preferably comprises a variant of SEQ ID NO: 121 in which at least one cysteine residue and/or at least one non-natural amino acid have been introduced into (i) the tower domain (residues A261-T275 and T285-Y370) and/or (ii) the pin domain (residues G91-E107) and/or the (iii) 1A domain (residues M1-L90 and E108-H173). The at least one cysteine residue and/or at least one non-natural amino acid are preferably introduced into residues T285-Y370 of the tower domain.

The helicase of the invention preferably comprises a variant of SEQ ID NO: 122 in which at least one cysteine residue and/or at least one non-natural amino acid have been introduced into (i) the tower domain (residues G294-I307 and T314-Y407) and/or (ii) the pin domain (residues G116-T135) and/or the (iii) 1A domain (residues M1-L115 and N136-V205). The at least one cysteine residue and/or at least one non-natural amino acid are preferably introduced into residues T314-Y407 of the tower domain.

The helicase of the invention preferably comprises a variant of SEQ ID NO: 123 in which at least one cysteine residue and/or at least one non-natural amino acid have been introduced into (i) the tower domain (residues V288-E301 and N307-N393) and/or (ii) the pin domain (residues G97-P113) and/or the (iii) 1A domain (residues M1-L96 and F114-V194). The at least one cysteine residue and/or at least one non-natural amino acid are preferably introduced into residues N307-N393 of the tower domain.

The helicase of the invention preferably comprises a variant of SEQ ID NO: 124 in which at least one cysteine residue and/or at least one non-natural amino acid have been introduced into (i) the tower domain (residues 5250-P264 and E278-S371) and/or (ii) the pin domain (residues K78-E95) and/or the (iii) 1A domain (residues M1-L77 and V96-V166). The at least one cysteine residue and/or at least one non-natural amino acid are preferably introduced into residues E278-S371 of the tower domain.

The helicase of the invention preferably comprises a variant of SEQ ID NO: 125 in which at least one cysteine residue and/or at least one non-natural amino acid have been introduced into (i) the tower domain (residues K255-P269 and T284-S380) and/or (ii) the pin domain (residues K82-K98) and/or the (iii) 1A domain (residues M1-M81 and L99-M171). The at least one cysteine residue and/or at least one non-natural amino acid are preferably introduced into residues T284-S380 of the tower domain.

The helicase of the invention preferably comprises a variant of SEQ ID NO: 126 in which at least one cysteine residue and/or at least one non-natural amino acid have been introduced into (i) the tower domain (residues D242-P256 and T271-S366) and/or (ii) the pin domain (residues K69-K85) and/or the (iii) 1A domain (residues M1-M68 and M86-M158). The at least one cysteine residue and/or at least one non-natural amino acid are preferably introduced into residues T271-S366 of the tower domain.

The helicase of the invention preferably comprises a variant of SEQ ID NO: 127 in which at least one cysteine residue and/or at least one non-natural amino acid have been introduced into (i) the tower domain (residues T263-P277 and N295-P392) and/or (ii) the pin domain (residues K88-K107) and/or the (iii) 1A domain (residues M1-L87 and A108-M181). The at least one cysteine residue and/or at least one non-natural amino acid are preferably introduced into residues N295-P392 of the tower domain.

The helicase of the invention preferably comprises a variant of SEQ ID NO: 128 in which at least one cysteine residue and/or at least one non-natural amino acid have been introduced into (i) the tower domain (residues D263-P277 and N295-A391) and/or (ii) the pin domain (residues K88-K107) and/or the (iii) 1A domain (residues M1-L87 and A108-M181). The at least one cysteine residue and/or at least one non-natural amino acid are preferably introduced into residues N295-A391 of the tower domain.

The helicase of the invention preferably comprises a variant of SEQ ID NO: 129 in which at least one cysteine residue and/or at least one non-natural amino acid have been introduced into (i) the tower domain (residues A258-P272 and N290-P386) and/or (ii) the pin domain (residues K86-G102) and/or the (iii) 1A domain (residues M1-L85 and T103-K176). The at least one cysteine residue and/or at least one non-natural amino acid are preferably introduced into residues N290-P386 of the tower domain.

The helicase of the invention preferably comprises a variant of SEQ ID NO: 130 in which at least one cysteine residue and/or at least one non-natural amino acid have been introduced into (i) the tower domain (residues L266-P280 and N298-A392) and/or (ii) the pin domain (residues K92-D108) and/or the (iii) 1A domain (residues M1-L91 and V109-M183). The at least one cysteine residue and/or at least one non-natural amino acid are preferably introduced into residues N298-A392 of the tower domain.

The helicase of the invention preferably comprises a variant of SEQ ID NO: 131 in which at least one cysteine residue and/or at least one non-natural amino acid have been introduced into (i) the tower domain (residues D262-P276 and N294-A392) and/or (ii) the pin domain (residues K88-E104) and/or the (iii) 1A domain (residues M1-L87 and M105-M179). The at least one cysteine residue and/or at least one non-natural amino acid are preferably introduced into residues N294-A392 of the tower domain.

The helicase of the invention preferably comprises a variant of SEQ ID NO: 132 in which at least one cysteine residue and/or at least one non-natural amino acid have been introduced into (i) the tower domain (residues D261-P275 and N293-A389) and/or (ii) the pin domain (residues K87-E103) and/or the (iii) 1A domain (residues M1-L86 and V104-K178). The at least one cysteine residue and/or at least one non-natural amino acid are preferably introduced into residues N293-A389 of the tower domain.

The helicase of the invention preferably comprises a variant of SEQ ID NO: 133 in which at least one cysteine residue and/or at least one non-natural amino acid have been introduced into (i) the tower domain (residues E261-P275 and T293-A390) and/or (ii) the pin domain (residues K87-E103) and/or the (iii) 1A domain (residues M1-L86 and V104-M178). The at least one cysteine residue and/or at least one non-natural amino acid are preferably introduced into residues T293-A390 of the tower domain.

The helicase of the invention preferably comprises a variant of any one of SEQ ID NOs: 118 to 133 in which at least one cysteine residue and/or at least one non-natural amino acid have been introduced into each of (i) the tower domain and (ii) the pin domain and/or the 1A domain. The helicase of the invention more preferably comprises a variant of any one of SEQ ID NOs: 118 to 133 in which at least one cysteine residue and/or at least one non-natural amino acid have been introduced into each of (i) the tower domain, (ii) the pin domain and (iii) the 1A domain. Any number and combination of cysteine residues and non-natural amino acids may be introduced as discussed above.

The helicase of the invention preferably comprises a variant of SEQ ID NO: 118 which comprises (i) E94C and/or A360C; (ii) E93C and/or K358C; (iii) E93C and/or A360C; (iv) E93C and/or E361C; (v) E93C and/or K364C; (vi) E94C and/or L354C; (vii) E94C and/or K358C; (viii) E93C and/or L354C; (ix) E94C and/or E361C; (x) E94C and/or K364C; (xi) L97C and/or L354C; (xii) L97C and/or K358C; (xiii) L97C and/or A360C; (xiv) L97C and/or E361C; (xv) L97C and/or K364C; (xvi) K123C and/or L354C; (xvii) K123C and/or K358C; (xviii) K123C and/or A360C; (xix) K123C and/or E361C; (xx) K123C and/or K364C; (xxi) N155C and/or L354C; (xxii) N155C and/or K358C; (xxiii) N155C and/or A360C; (xxiv) N155C and/or E361C; (xxv) N155C and/or K364C; (xxvi) any of (i) to (xxv) and G357C; (xxvii) any of (i) to (xxv) and Q100C; (xxviii) any of (i) to (xxv) and I127C; (xxix) any of (i) to (xxv) and Q100C and I127C; (xxx) E94C and/or F377C; (xxxi) N95C; (xxxii) T91C; (xxxiii) Y92L, E94Y, Y350N, A360C and Y363N; (xxxiv) E94Y and A360C; (xxxv) A360C; (xxxvi) Y92L, E94C, Y350N, A360Y and Y363N; (xxxvii) Y92L, E94C and A360Y; (xxxviii) E94C and/or A360C and F276A; (xxxix) E94C and/or L356C; (xl) E93C and/or E356C; (xli) E93C and/or G357C; (xlii) E93C and/or A360C; (xliii) N95C and/or W378C; (xliv) T91C and/or S382C; (xlv) T91C and/or W378C; (xlvi) E93C and/or N353C; (xlvii) E93C and/or S382C; (xlviii) E93C and/or K381C; (xlix) E93C and/or D379C; (l) E93C and/or S375C; (li) E93C and/or W378C; (lii) E93C and/or W374C; (liii) E94C and/or N353C; (liv) E94C and/or S382C; (lv) E94C and/or K381C; (lvi) E94C and/or D379C; (lvii) E94C and/or S375C; (lviii) E94C and/or W378C; (lix) E94C and/or W374C; (lx) E94C and A360Y; (lxi) E94C, G357C and A360C or (lxii) T2C, E94C and A360C. In any one of (i) to (lxii), and/or is preferably and.

The helicase of the invention preferably comprises a variant of any one of SEQ ID NOs: 119 to 133 which comprises a cysteine residue at the positions which correspond to those in SEQ ID NO: 118 as defined in any of (i) to (lxii). Positions in any one of SEQ ID NOs: 119 to 133 which correspond to those in SEQ ID NO: 118 can be identified using the alignment of SEQ ID NOs: 118 to 133 below. The helicase of the invention preferably comprises a variant of SEQ ID NO: 92 which comprises (a) D99C and/or L341C, (b) Q98C and/or L341C or (d) Q98C and/or A340C. The helicase of the invention preferably comprises a variant of SEQ ID NO: 96 which comprises D90C and/or A349C. The helicase of the invention preferably comprises a variant of SEQ ID NO: 102 which comprises D96C and/or A362C.

The helicase of the invention preferably comprises a variant of any one of SEQ ID NOs: 118 to 133 as defined in any one of (i) to (lxii) in which Faz is introduced at one or more of the specific positions instead of cysteine. Faz may be introduced at each specific position instead of cysteine.

The helicase of the invention preferably comprises a variant of SEQ ID NO: 118 which comprises (i) E94Faz and/or A360C; (ii) E94C and/or A360Faz; (iii) E94Faz and/or A360Faz; (iv) Y92L, E94Y, Y350N, A360Faz and Y363N; (v) A360Faz; (vi) E94Y and A360Faz; (vii) Y92L, E94Faz, Y350N, A360Y and Y363N; (viii) Y92L, E94Faz and A360Y; (ix) E94Faz and A360Y; and (x) E94C, G357Faz and A360C.

The helicase of the invention preferably further comprises one or more single amino acid deletions from the pin domain. Any number of single amino acid deletions may be made, such as 1, 2, 3, 4, 5, 6, 7, 8, 9, 10 or more. The helicase more preferably comprises a variant of SEQ ID NO: 118 which comprises deletion of E93, deletion of E95 or deletion of E93 and E95. The helicase more preferably comprises a variant of SEQ ID NO: 118 which comprises (a) E94C, deletion of N95 and A360C; (b) deletion of E93, deletion of E94, deletion of N95 and A360C; (c) deletion of E93, E94C, deletion of N95 and A360C or (d) E93C, deletion of N95 and A360C.

The helicase of the invention preferably comprises a variant of any one of SEQ ID NOs: 119 to 133 which comprises deletion of the position corresponding to E93 in SEQ ID NO: 118, deletion of the position corresponding to E95 in SEQ ID NO: 118 or deletion of the positions corresponding to E93 and E95 in SEQ ID NO: 118.

The helicase of the invention preferably further comprises one or more single amino acid deletions from the hook domain. Any number of single amino acid deletions may be made, such as 1, 2, 3, 4, 5, 6, 7, 8, 9, 10 or more. The helicase more preferably comprises a variant of SEQ ID NO: 118 which comprises deletion of any number of positions T278 to S287. The helicase more preferably comprises a variant of SEQ ID NO: 118 which comprises (a) E94C, deletion of Y279 to K284 and A360C, (b) E94C, deletion of T278, Y279, V286 and S287 and A360C, (c) E94C, deletion of I281 and K284 and replacement with a single G and A360C, (d) E94C, deletion of K280 and P2845 and replacement with a single G and A360C, or (e) deletion of Y279 to K284, E94C, F276A and A230C. The helicase of the invention preferably comprises a variant of any one of SEQ ID NOs: 119 to 133 which comprises deletion of any number of the positions corresponding to 278 to 287 in SEQ ID NO: 118.

The helicase of the invention preferably further comprises one or more single amino acid deletions from the pin domain and one or more single amino acid deletions from the hook domain.

The helicase of the invention is preferably one in which at least one cysteine residue and/or at least one non-natural amino acid have been further introduced into the hook domain and/or the 2A (RecA-like) domain. Any number and combination of cysteine residues and non-natural amino acids may be introduced as discussed above for the tower, pin and 1A domains.

The helicase of the invention preferably comprises a variant of SEQ ID NO: 118 in which at least one cysteine residue and/or at least one non-natural amino acid have further been introduced into the hook domain (residues L275-F291) and/or the 2A (RecA-like) domain (residues R178-T259 and L390-V439).

The helicase of the invention preferably comprises a variant of SEQ ID NO: 119 in which at least one cysteine residue and/or at least one non-natural amino acid have further been introduced into the hook domain (residues A310-L315) and/or the 2A (RecA-like) domain (residues R212-E294 and G422-S678).

The helicase of the invention preferably comprises a variant of SEQ ID NO: 120 in which at least one cysteine residue and/or at least one non-natural amino acid have further been introduced into the hook domain (residues V343-L359) and/or the 2A (RecA-like) domain (residues R241-N327 and A449-G496).

The helicase of the invention preferably comprises a variant of SEQ ID NO: 121 in which at least one cysteine residue and/or at least one non-natural amino acid have further been introduced into the hook domain (residues W276-L284) and/or the 2A (RecA-like) domain (residues R174-D260 and A371-V421).

The helicase of the invention preferably comprises a variant of SEQ ID NO: 122 in which at least one cysteine residue and/or at least one non-natural amino acid have further been introduced into the hook domain (residues R308-Y313) and/or the 2A (RecA-like) domain (residues R206-K293 and I408-L500).

The helicase of the invention preferably comprises a variant of SEQ ID NO: 123 in which at least one cysteine residue and/or at least one non-natural amino acid have further been introduced into the hook domain (residues M302-W306) and/or the 2A (RecA-like) domain (residues R195-D287 and V394-Q450).

The helicase of the invention preferably comprises a variant of SEQ ID NO: 124 in which at least one cysteine residue and/or at least one non-natural amino acid have further been introduced into the hook domain (residues V265-I277) and/or the 2A (RecA-like) domain (residues R167-T249 and L372-N421).

The helicase of the invention preferably comprises a variant of SEQ ID NO: 125 in which at least one cysteine residue and/or at least one non-natural amino acid have further been introduced into the hook domain (residues V270-F283) and/or the 2A (RecA-like) domain (residues R172-T254 and L381-K434).

The helicase of the invention preferably comprises a variant of SEQ ID NO: 126 in which at least one cysteine residue and/or at least one non-natural amino acid have further been introduced into the hook domain (residues V257-F270) and/or the 2A (RecA-like) domain (residues R159-T241 and L367-K420).

The helicase of the invention preferably comprises a variant of SEQ ID NO: 127 in which at least one cysteine residue and/or at least one non-natural amino acid have further been introduced into the hook domain (residues L278-Y294) and/or the 2A (RecA-like) domain (residues R182-T262 and L393-V443).

The helicase of the invention preferably comprises a variant of SEQ ID NO: 128 in which at least one cysteine residue and/or at least one non-natural amino acid have further been introduced into the hook domain (residues L278-Y294) and/or the 2A (RecA-like) domain (residues R182-T262 and L392-V442).

The helicase of the invention preferably comprises a variant of SEQ ID NO: 129 in which at least one cysteine residue and/or at least one non-natural amino acid have further been introduced into the hook domain (residues L273-F289) and/or the 2A (RecA-like) domain (residues R177-N257 and L387-V438).

The helicase of the invention preferably comprises a variant of SEQ ID NO: 130 in which at least one cysteine residue and/or at least one non-natural amino acid have further been introduced into the hook domain (residues L281-F297) and/or the 2A (RecA-like) domain (residues R184-T265 and L393-I442).

The helicase of the invention preferably comprises a variant of SEQ ID NO: 131 in which at least one cysteine residue and/or at least one non-natural amino acid have further been introduced into the hook domain (residues H277-F293) and/or the 2A (RecA-like) domain (residues R180-T261 and L393-V442).

The helicase of the invention preferably comprises a variant of SEQ ID NO: 132 in which at least one cysteine residue and/or at least one non-natural amino acid have further been introduced into the hook domain (residues L276-F292) and/or the 2A (RecA-like) domain (residues R179-T260 and L390-I439).

The helicase of the invention preferably comprises a variant of SEQ ID NO: 133 in which at least one cysteine residue and/or at least one non-natural amino acid have further been introduced into the hook domain (residues L276-F292) and/or the 2A (RecA-like) domain (residues R179-T260 and L391-V441).

The helicase of the invention preferably comprises a variant of SEQ ID NO: 118 which comprises one or more of (i) I181C; (ii) Y279C; (iii) I281C; and (iv) E288C. The helicase may comprise any combination of (i) to (iv), such as (i); (ii); (iii); (iv); (i) and (ii); (i) and (iii); (i) and (iv); (ii) and (iii); (ii) and (iv); (iii) and (iv); or (i), (ii), (iii) and (iv). The helicase more preferably comprises a variant of SEQ ID NO: 118 which comprises (a) E94C, I281C and A360C or (b) E94C, I281C, G357C and A360C. The helicase of the invention preferably comprises a variant of any one of SEQ ID NOs: 119 to 133 which comprises a cysteine residue at one or more of the position(s) which correspond to those in SEQ ID NO: 118 as defined in (i) to (iv), (a) and (b). The helicase may comprise any of these variants in which Faz is introduced at one or more of the specific positions (or each specific position) instead of cysteine.

The helicase of the invention is further modified to reduce its surface negative charge. Surface residues can be identified in the same way as the Dda domains disclosed above. Surface negative charges are typically surface negatively-charged amino acids, such as aspartic acid (D) and glutamic acid (E).

The helicase is preferably modified to neutralise one or more surface negative charges by substituting one or more negatively charged amino acids with one or more positively charged amino acids, uncharged amino acids, non-polar amino acids and/or aromatic amino acids or by introducing one or more positively charged amino acids, preferably adjacent to one or more negatively charged amino acids. Suitable positively charged amino acids include, but are not limited to, histidine (H), lysine (K) and arginine (R). Uncharged amino acids have no net charge. Suitable uncharged amino acids include, but are not limited to, cysteine (C), serine (S), threonine (T), methionine (M), asparagine (N) and glutamine (Q). Non-polar amino acids have non-polar side chains. Suitable non-polar amino acids include, but are not limited to, glycine (G), alanine (A), proline (P), isoleucine (I), leucine (L) and valine (V). Aromatic amino acids have an aromatic side chain. Suitable aromatic amino acids include, but are not limited to, histidine (H), phenylalanine (F), tryptophan (W) and tyrosine (Y).

Preferred substitutions include, but are not limited to, substitution of E with R, substitution of E with K, substitution of E with N, substitution of D with K and substitution of D with R.

The helicase of the invention preferably comprises a variant of SEQ ID NO: 118 and the one or more negatively charged amino acids are one or more of D5, E8, E23, E47, D167, E172, D202, D212 and E273. Any number of these amino acids may be neutralised, such as 1, 2, 3, 4, 5, 6, 7 or 8 of them. Any combination may be neutralised. The helicase of the invention preferably comprises a variant of any one of SEQ ID NOs: 119 to 133 and the one or more negatively charged amino acids correspond to one or more of D5, E8, E23, E47, D167, E172, D202, D212 and E273 in SEQ ID NO: 118. Amino acids in SEQ ID NOs: 119 to 133 which correspond to D5, E8, E23, E47, D167, E172, D202, D212 and E273 in SEQ ID NO: 118 can be determined using the alignment in WO2015/055981. The helicase of the invention preferably comprises a variant of SEQ ID NO: 118 which comprises (a) E94C, E273G and A360C or (b) E94C, E273G, N292G and A360C.

The helicase of the invention is preferably further modified by the removal of one or more native cysteine residues. Any number of native cysteine residues may be removed. The one or more cysteine residues are preferably removed by substitution. The one or more cysteine residues are preferably substituted with alanine (A), serine (S) or valine (V). The helicase of the invention preferably comprises a variant of SEQ ID NO: 118 and the one or more native cysteine residues are one or more of C109, C114, C136, C171 and C412. Any number and combination of these cysteine residues may be removed. For instance, the variant of SEQ ID NO: 118 may comprise C109; C114; C136; C171; C412; C109 and C114; C109 and C136; C109 and C171; C109 and C412; C114 and C136; C114 and C171; C114 and C412; C136 and C171; C136 and C412; C171 and C412; C109, C114 and C136; C109, C114 and C171; C109, C114 and C412; C109, C136 and C171; C109, C136 and C412; C109, C171 and C412; C114, C136 and C171; C114, C136 and C412; C114, C171 and C412; C136, C171 and C412; C109, C114, C136 and C171; C109, C114, C136 and C412; C109, C114, C171 and C412; C109, C136, C171 and C412; C114, C136, C171 and C412; or C109, C114, C136, C171 and C412.

The modified helicase preferably comprises a modification or substitution at the position(s) corresponding to amino acid position(s) 109 and/or 136 in Dda 1993. This removes one or two cysteine residues. This may be in addition to a modification or substitution at one or more positions corresponding to amino acid positions 55, 114, 156, 177, 210, 221, 350 and 358 in Dda 1993, a modification or substitution at one or more positions corresponding to amino acid positions 114, 177, 350 and 358 in Dda 1993 and/or a modification or substitution at the position corresponding to position 40 in Dda 1993. Position 109 or the corresponding position may be substituted with A, V, I, L, M, F, Y or W. Position 109 or the corresponding position is preferably substituted with A or V. Position 136 or the corresponding position may be substituted with A, V, I, L, M, F, Y or W. Position 136 or the corresponding position is preferably substituted with A or V. The helicase of the invention preferably comprises a variant of SEQ ID NO: 118 which comprises a substitution at C109, such as C109A, C109V, C109I, C109L, C109M, C109F, C109Y or C109W and/or at C136, such as C136A, C136V, C136I, C136L, C136M, C136F, C136Y or C136W. The helicase of the invention preferably comprises a variant of SEQ ID NO: 118 which comprises C109A and/or C136A. The helicase of the invention preferably comprises a variant of SEQ ID NO: 119, 120, 121, 122, 123, 124, 125, 126, 127, 128, 129, 130, 131, 132 or 133 which comprises a substitution at the position(s) which correspond(s) to C109 and/or C136 in SEQ ID NO: 118. The helicase of the invention is preferably one in which at least one cysteine residue (i.e. one or more cysteine residues) and/or at least one non-natural amino acid (i.e. one or more non-natural amino acids) have been introduced into the tower domain only. Suitable modifications are discussed above.

The helicase of the invention preferably comprises a variant of SEQ ID NO: 118 comprising the following mutations: E93C and K364C; E94C and K364C; E94C and A360C; L97C and E361C; L97C and E361C and C412A; K123C and E361C; K123C, E361C and C412A; N155C and K358C; N155C, K358C and C412A; N155C and L354C; N155C, L354C and C412A; deltaE93, E94C, deltaN95 and A360C; E94C, deltaN95 and A360C; E94C, Q100C, I127C and A360C; L354C; G357C; E94C, G357C and A360C; E94C, Y279C and A360C; E94C, I281C and A360C; E94C, Y279Faz and A360C; Y279C and G357C; I281C and G357C; E94C, Y279C, G357C and A360C; E94C, I281C, G357C and A360C; E8R, E47K, E94C, D202K and A360C; DSK, E23N, E94C, D167K, E172R, D212R and A360C; DSK, E8R, E23N, E47K, E94C, D167K, E172R, D202K, D212R and A360C; E94C, C114A, C171A, A360C and C412D; E94C, C114A, C171A, A360C and C412S; E94C, C109A, C136A and A360C; E94C, C109A, C114A, C136A, C171A, A360C and C412S; E94C, C109V, C114V, C171A, A360C and C412S; C109A, C114A, C136A, G153C, C171A, E361C and C412A; C109A, C114A, C136A, G153C, C171A, E361C and C412D; C109A, C114A, C136A, G153C, C171A, E361C and C412S; C109A, C114A, C136A, G153C, C171A, K358C and C412A; C109A, C114A, C136A, G153C, C171A, K358C and C412D; C109A, C114A, C136A, G153C, C171A, K358C and C412S; C109A, C114A, C136A, N155C, C171A, K358C and C412A; C109A, C114A, C136A, N155C, C171A, K358C and C412D; C109A, C114A, C136A, N155C, C171A, K358C and C412S; C109A, C114A, C136A, N155C, C171A, L354C and C412A; C109A, C114A, C136A, N155C, C171A, L354C and C412D; C109A, C114A, C136A, N155C, C171A, L354C and C412S; C109A, C114A, K123C, C136A, C171A, E361C and C412A; C109A, C114A, K123C, C136A, C171A, E361C and C412D; C109A, C114A, K123C, C136A, C171A, E361C and C412S; C109A, C114A, K123C, C136A, C171A, K358C and C412A; C109A, C114A, K123C, C136A, C171A, K358C and C412D; C109A, C114A, K123C, C136A, C171A, K358C and C412S; C109A, C114A, C136A, G153C, C171A, E361C and C412A; E94C, C109A, C114A, C136A, C171A, A360C and C412D; E94C, C109A, C114V, C136A, C171A, A360C and C412D; E94C, C109V, C114A, C136A, C171A, A360C and C412D; L97C, C109A, C114A, C136A, C171A, E361C and C412A; L97C, C109A, C114A, C136A, C171A, E361C and C412D; or L97C, C109A, C114A, C136A, C171A, E361C and C412S.

Modifications in the Hook Domain and/or 2A Domain

In one embodiment, the helicase of the invention is one in which at least one cysteine residue and/or at least one non-natural amino acid have been introduced into the hook domain and/or the 2A (RecA-like motor) domain, wherein the helicase has the ability to control the movement of a polynucleotide. At least one cysteine residue and/or at least one non-natural amino acid is preferably introduced into the hook domain and the 2A (RecA-like motor) domain.

Any number of cysteine residues and/or non-natural amino acids may be introduced into each domain. For instance, 1, 2, 3, 4, 5, 6, 7, 8, 9, 10 or more cysteine residues may be introduced and/or 1, 2, 3, 4, 5, 6, 7, 8, 9, 10 or more non-natural amino acids may be introduced. Only one or more cysteine residues may be introduced. Only one or more non-natural amino acids may be introduced. A combination of one or more cysteine residues and one or more non-natural amino acids may be introduced.

The at least one cysteine residue and/or at least one non-natural amino acid are preferably introduced by substitution. Methods for doing this are known in the art. Suitable modifications of the hook domain and/or the 2A (RecA-like motor) domain are discussed above.

The helicase of the invention is preferably a variant of SEQ ID NO: 118 comprising (a) Y279C, I181C, E288C, Y279C and I181C, (b) Y279C and E288C, (c) I181C and E288C or (d) Y279C, I181C and E288C. The helicase of the invention preferably comprises a variant of any one of SEQ ID NOs: 199 to 133 which comprises a mutation at one or more of the position(s) which correspond to those in SEQ ID NO: 118 as defined in (a) to (d).

### Surface Modification

In one embodiment, the helicase is modified to reduce its surface negative charge, wherein the helicase has the ability to control the movement of a polynucleotide. Suitable modifications are discussed above. Any number of surface negative charges may be neutralised.

The helicase of the invention preferably comprises a variant of SEQ ID NO: 118 comprising the following mutations: E273G; E8R, E47K and D202K; D5K, E23N, D167K, E172R and D212R; or D5K, E8R, E23N, E47K, D167K, E172R, D202K and D212R.

### Other Modified Helicases

In one embodiment, the helicase of the invention comprises a variant of SEQ ID NO: 118 comprising: A360K; Y92L and/or A360Y; Y92L, Y350N and Y363N; Y92L and/or Y363N; or Y92L.

### Other Modifications

In addition to the specific mutations disclosed above, a variant of SEQ ID NO: 118 may comprise one or more of the following mutations: K38A; T91F; T91N; T91Q; T91W; V96E; V96F; V96L; V96Q; V96R; V96W; V96Y; P274G; V286F; V286W; V286Y; F291G; N292F; N292G; N292P; N292Y; G294Y; G294F; K364A; and W378A.

In addition to the specific mutations disclosed above, a variant of SEQ ID NO: 118 may comprise: K38A, E94C and A360C; H64K; E94C and A360C; H64N; E94C and A360C; H64Q; E94C and A360C; H64S; E94C and A360C; H64W, E94C and A360C; T80K, E94C and A360C; T80K, S83K, E94C, N242K, N293K and A360C; T80K, S83K, E94C, N242K, N293K, A360C and T394K; T80K, S83K, E94C, N293K and A360C; T80K, S83K, E94C, A360C and T394K; T80K, S83K, E94C, A360C and T394N; T80K, E94C, N242K and A360C; T80K, E94C, N242K, N293K and A360C; T80K, E94C, N293K and A360C; T80N, E94C and A360C; H82A, E94C and A360C; H82A, P89A, E94C, F98A and A360C; H82F, E94C and A360C; H82Q, E94C, A360C; H82R, E94C and A360C; H82W, E94C and A360C; H82W, P89W, E94C, F98W and A360C; H82Y, E94C and A360C; S83K, E94C and A360C; S83K, T80K, E94C, A360C and T394K; S83N, E94C and A360C; S83T, E94C and A360C; N88H, E94C and A360C; N88Q, E94C and A360C; P89A, E94C and A360C; P89A, F98W, E94C and A360C; P89A, E94C, F98Y and A360C; P89A, E94C, F98A and A360C; P89F, E94C and A360C; P89S, E94C and A360C; P89T, E94C and A360C; P89W, E94C, F98W and A360C; P89Y, E94C and A360C; T91F, E94C and A360C; T91N, E94C and A360C; T91Q, E94C and A360C; T91W, E94C and A360C; E94C, V96E and A360C; E94C, V96F and A360C; E94C, V96L and A360C; E94C, V96Q and A360C; E94C, V96R and A360C; E94C, V96W and A360C; E94C, V96Y and A360C; E94C, F98A and A360C; E94C, F98L and A360C; E94C, F98V and A360C; E94C, F98Y and A360C; E94C; F98W and A360C; E94C, V150A and A360C; E94C, V150F and A360C; E94C, V150I and A360C; E94C, V150K and A360C; E94C, V150L and A360C; E94C, V150S and A360C; E94C, V150T and A360C; E94C, V150W and A360C; E94C, V150Y and A360C; E94C, F240Y and A360C; E94C, F240W and A360C; E94C, N242K and A360C; E94C, N242K, N293K and A360C; E94C, P274G and A360C; E94C, L275G and A360C; E94C, F276A and A360C; E94C, F276I and A360C; E94C, F276M and A360C; E94C, F276V and A360C; E94C, F276W and A360C; E94C, F276Y and A360C; E94C, V286F and A360C; E94C, V286W and A360C; E94C, V286Y and A360C; E94C, S287F and A360C; E94C, S287W and A360C; E94C, S287Y and A360C; E94C, F291G and A360C; E94C, N292F and A360C; E94C, N292G and A360C; E94C, N292P and A360C; E94C, N292Y and A360C; E94C, N293F and A360C; E94C, N293K and A360C; E94C, N293Q and A360C; E94C, N293Y and A360C; E94C, G294F and A360C; E94C, G294Y and A360C; E94C, A36C and K364A; E94C, A360C, W378A; E94C, A360C and T394K; E94C, A360C and H396Q; E94C, A360C and H396S; E94C, A360C and H396W; E94C, A360C and Y415F; E94C, A360C and Y415K; E94C, A360C and Y415M; or E94C, A360C and Y415W.

The helicase of the invention preferably comprises a variant of SEQ ID NO: 118 which comprises (a) E94C/A360C/W378A or (b) E94C/A360C/C109A/C136A/W378A or (d) E94C/A360C/C109A/C136A/W378A and then (ΔM1)G1G2 (i.e. deletion of M1 and then addition G1 and G2).

Preferred variants of any one of SEQ ID NOs: 118 to 133 have (in addition to the modifications of the invention) the N-terminal methionine (M) replaced with one glycine residue (G). In the examples this is shown as (ΔM1)G1. It may also be termed M1G. Any of the variants discussed above may further comprise M1G.

The most preferred helicases of the invention comprise a variant of SEQ ID NO: 118 which comprises (a) E94C/F98W/A360C/C109A/C136A/K194L, (b) M1G/E94C/F98W/A360C/C109A/C136A/K194L; (c) E94C/F98W/A360C/C109A/C136A/K199L; or (d) M1G/E94C/F98W/A360C/C109A/C136A/K199L.

### Other Preferred Helicases

Other preferred helicases of the invention comprise a variant of SEQ ID NO: 118 which comprises substitutions at:
T40/E94/F98, such as T40Y/E94C/F98W;
T40/E94/F98/C114, such as T40Y/E94C/F98W/C114I;
T40/E94/F98/K177, such as T40Y/E94C/F98W/K177M;
T40/E94/F98/Y350, such as T40Y/E94C/F98W/Y350, T40Y/E94C/F98W/Y350I or T40Y/E94C/F98W/Y350E;
T40/E94/F98/K358, such as T40Y/E94C/F98W/K358I;
E94/F98/C114, such as E94C/F98W/C114I;
E94/F98/K177, such as E94C/F98W/K177M;
E94/F98/Y350, such as E94C/F98W/Y350I or E94C/F98W/Y350E;
E94/F98/K358, such as E94C/F98W/K358I;
T40/E94/F98/C114/K177, such as T40Y/E94C/F98W/C114I/K177M;
T40/E94/F98/C114/Y350, such as T40Y/E94C/F98W/C114I/Y350I or T40Y/E94C/F98W/C114I/Y350E;
T40/E94/F98/C114/K358, such as T40Y/E94C/F98W/C114I/K358I;
T40/E94/F98/K177/Y350, such as T40Y/E94C/F98W/K177M/Y350I or T40Y/E94C/F98W/K177M/Y350E;
T40/E94/F98/K177/K358, such as T40Y/E94C/F98W/K177M/K358I;
T40/E94/F98/Y350/K358, such as T40Y/E94C/F98W/Y350I/K358I or T40Y/E94C/F98W/Y350E/K358I;
E94/F98/C114/K177, such as E94C/F98W/C114I/K177M;
E94/F98/C114/Y350, such as E94C/F98W/C114I/Y350I or E94C/F98W/C114I/Y350E;
E94/F98/C114/K358, such as E94C/F98W/C114I/K358I;
E94/F98/K177/Y350, such as E94C/F98W/K177M/Y350I or E94C/F98W/K177M/Y350E;
E94/F98/K177/K358, such as E94C/F98W/K177M/K358I;
E94/F98/Y350/K358, such as E94C/F98W/Y350I/K358I or E94C/F98W/Y350E/K358I;
T40/E94/F98/C114/K177/Y350, such as T40Y/E94C/F98W/C114I/K177M/Y350I or T40Y/E94C/F98W/C114I/K177M/Y350E;
T40/E94/F98/C114/K177/K358, such as T40Y/E94C/F98W/C114I/K177M/K358I;
T40/E94/F98/C114/Y350/K358, such as T40Y/E94C/F98W/C114I/Y350I/K358I or T40Y/E94C/F98W/C114I/Y350E/K358I;
T40/E94/F98/K177/Y350/K358, such as T40Y/E94C/F98W/K177M/Y350I/K358I or T40Y/E94C/F98W/K177M/Y350E/K358I;
E94/F98/C114/K177/Y350, such as E94C/F98W/C114I/K177M/Y350I or E94C/F98W/C114I/K177M/Y350E;
E94/F98/C114/K177/K358, such as E94C/F98W/C114I/K177M/K358I;
E94/F98/C114/Y350/K358, such as E94C/F98W/C114I/Y350I/K358I or E94C/F98W/C114I/Y350E/K358I;
E94/F98/K177/Y350/K358, such as E94C/F98W/K177M/Y350I/K358I or E94C/F98W/K177M/Y350E/K358I;
T40/E94/F98/C114/K177/Y350/K358, such as T40Y/E94C/F98W/C114I/K177M/Y350I/K358I or T40Y/E94C/F98W/C114I/K177M/Y350E/K358I;
E94/F98/C114/K177/Y350/K358, such as E94C/F98W/C114I/K177M/Y350I/K358I or E94C/F98W/C114I/K177M/Y350E/K358I;
T40/F98/K194, such as T40Y/E94C/F98W/K194L;
T40/F98/C114/K194, such as T40Y/E94C/F98W/C114I K194L;
T40/F98/K177/K194, such as T40Y/E94C/F98W/K177M K194L;
T40/F98/K194/Y350, such as T40Y/E94C/F98W/K194L/Y350I or T40Y/E94C/F98W/K194L/Y350E;
T40/F98/K194/K358, such as T40Y/E94C/F98W/K194L/K358I;
F98/C114/K194, such as E94C/F98W/C114I/K194L;
F98/K177/K194, such as E94C/F98W/K177M/K194L;
F98/K194/Y350, such as E94C/F98W/K194L/Y350I or E94C/F98W/K194L/Y350E;
F98/K194/K358, such as E94C/F98W/K194L/K358I;
T40/F98/C114/K177/K194, such as T40Y/E94C/F98W/C114I/K177M/K194L;
T40/F98/C114/K194/Y350, such as T40Y/E94C/F98W/C114I/K194L/Y350I or T40Y/E94C/F98W/C114I/K194L/Y350E;
T40/F98/C114/K194/K358, such as T40Y/E94C/F98W/C114I/K194L/K358I;
T40/F98/K177/K194/Y350, such as T40Y/E94C/F98W/K177M/K194L/Y350I or T40Y/E94C/F98W/K177M/K194L/Y350E;
T40/F98/K177/K194/K358, such as T40Y/E94C/F98W/K177M/K194L/K358I;
T40/F98/K194/Y350/K358, such as T40Y/E94C/F98W/K194L/Y350I/K358I or T40Y/E94C/F98W/K194L/Y350E/K358I;
F98/C114/K177/K194, such as E94C/F98W/C114I/K177M/K194L;
F98/C114/K194/Y350, such as E94C/F98W/C114I/K194L/Y350I or E94C/F98W/C114I/K194L/Y350E;
F98/C114/K194/K358, such as E94C/F98W/C114I/K194L/K358I;
F98/K177/K194/Y350, such as E94C/F98W/K177M/K194L/Y350I or E94C/F98W/K177M/K194L/Y350E;
F98/K177/K194/K358, such as E94C/F98W/K177M/K194L/K358I;
F98/K194/Y350/K358, such as E94C/F98W/K194L/Y350I/K358I or E94C/F98W/K194L/Y350E/K358I;
T40/F98/C114/K177/K194/Y350, such as T40Y/E94C/F98W/C114I/K177M/K194L/Y350I or T40Y/E94C/F98W/C114I/K177M/K194L/Y350E;
T40/F98/C114/K177/K194/K358, such as T40Y/E94C/F98W/C114I/K177M/K194L/K358I;
T40/F98/C114/K194/Y350/K358, such as T40Y/E94C/F98W/C114I/K194L/Y350I/K358I or T40Y/E94C/F98W/C114I/K194L/Y350E/K358I;
T40/F98/K177/K194/Y350/K358, such as T40Y/E94C/F98W/K177M/K194L/Y350I/K358I or T40Y/E94C/F98W/K177M/K194L/Y350E/K358I;
F98/C114/K177/K194/Y350, such as E94C/F98W/C114I/K177M/K194L/Y350I or E94C/F98W/C114I/K177M/K194L/Y350E;
F98/C114/K177/K194/K358, such as E94C/F98W/C114I/K177M/K194L/K358I;
F98/C114/K194/Y350/K358, such as E94C/F98W/C114I/K194L/Y350I/K358I or E94C/F98W/C114I/K194L/Y350E/K358I;
F98/K177/K194/Y350/K358, such as E94C/F98W/K177M/K194L/Y350I/K358I or E94C/F98W/K177M/K194L/Y350E/K358I;
T40/F98/C114/K177/K194/Y350/K358, such as
T40Y/E94C/F98W/C114I/K177M/K194L/Y350I/K358I or T40Y/E94C/F98W/C114I/K177M/K194L/Y350E/K358I;
F98/C114/K177/K194/Y350/K358, such as E94C/F98W/C114I/K177M/K194L/Y350I/K358I or E94C/F98W/C114I/K177M/K194L/Y350E/K358I;
T40/A360, such as T40Y/A360C;
T40/C114/A360, such as T40Y/C114I/A360C;
T40/K177/A360, such as T40Y/K177M/A360C;
T40/Y350/A360, such as T40Y/Y350I/A360C or T40Y/Y350E/A360C;
T40/K358/A360, such as T40Y/K358I/A360C;
C114/A360, such as C114I/A360C;
K177/A360, such as K177M/A360C;
Y350/A360, such as Y350I/A360C or Y350E/A360C;
K358/A360, such as K358I/A360C;
T40/C114/K177/A360, such as T40Y/C114I/K177M/A360C;
T40/C114/Y350/A360, such as T40Y/C114I/Y350I/A360C or T40Y/C114I/Y350E/A360C;
T40/C114/K358/A360, such as T40Y/C114I/K358I/A360C;
T40/K177/Y350/A360, such as T40Y/K177M/Y350I/A360C or T40Y/K177M/Y350E/A360C;
T40/K177/K358/A360, such as T40Y/K177M/K358I/A360C;
T40/Y350/K358/A360, such as T40Y/Y350I/K358I/A360C or T40Y/Y350E/K358I/A360C;
C114/K177/A360, such as C114I/K177M/A360C;
C114/Y350/A360, such as C114I/Y350I/A360C or C114I/Y350E/A360C;
C114/K358/A360, such as C114I/K358I/A360C;
K177/Y350/A360, such as K177M/Y350I/A360C or K177M/Y350E/A360C;
K177/K358/A360, such as K177M/K358I/A360C;
Y350/K358/A360, such as Y350I/K358I/A360C or Y350E/K358I/A360C;
T40/C114/K177/Y350/A360, such as T40Y/C114I/K177M/Y350I/A360C or T40Y/C114I/K177M/Y350E/A360C;
T40/C114/K177/K358/A360, such as T40Y/C114I/K177M/K358I/A360C;
T40/C114/Y350/K358/A360, such as T40Y/C114I/Y350I/K358I/A360C or T40Y/C114I/Y350E/K358I/A360C;
T40/K177/Y350/K358/A360, such as T40Y/K177M/Y350I/K358I/A360C or T40Y/K177M/Y350E/K358I/A360C;
C114/K177/Y350/A360, such as C114I/K177M/Y350I/A360C or C114I/K177M/Y350E/A360C;
C114/K177/K358/A360, such as C114I/K177M/K358I/A360C;
C114/Y350/K358/A360, such as C114I/Y350I/K358I/A360C or C114I/Y350E/K358I/A360C;
K177/Y350/K358/A360, such as K177M/Y350I/K358I/A360C or K177M/Y350E/K358I/A360C;
T40/C114/K177/Y350/K358/A360, such as T40Y/C114I/K177M/Y350I/K358I/A360C or K177M/Y350E/K358I/A360C;
C114/K177/Y350/K358/A360, such as C114I/K177M/Y350I/K358I/A360C or C114I/K177M/Y350E/K358I/A360C;
T40/E94/F98/C109, such as T40Y/E94C/F98W/C109A;
T40/E94/F98/C109/C114, such as T40Y/E94C/F98W/C109A/C114I;
T40/E94/F98/C109/K177, such as T40Y/E94C/F98W/C109A/K177M;
T40/E94/F98/C109/Y350, such as T40Y/E94C/F98W/C109A/Y350I or T40Y/E94C/F98W/C109A/Y350E;
T40/E94/F98/C109/K358, such as T40Y/E94C/F98W/C109A/K358I;
E94/F98/C109/C114, such as E94C/F98W/C109A/C114I;
E94/F98/C109/K177, such as E94C/F98W/C109A/K177M;
E94/F98/C109/Y350, such as E94C/F98W/C109A/Y350I or E94C/F98W/C109A/Y350E;
E94/F98/C109/K358, such as E94C/F98W/C109A/K358I;
T40/E94/F98/C109/C114/K177, such as T40Y/E94C/F98W/C109A/C114I/K177M;
T40/E94/F98/C109/C114/Y350, such as T40Y/E94C/F98W/C109A/C114I/Y350I or T40Y/E94C/F98W/C109A/C114I/Y350E;
T40/E94/F98/C109/C114/K358, such as T40Y/E94C/F98W/C109A/C114I/K358I;
T40/E94/F98/C109/K177/Y350, such as T40Y/E94C/F98W/C109A/K177M/Y350I oe T40Y/E94C/F98W/C109A/K177M/Y350E;
T40/E94/F98/C109/K177/K358, such as T40Y/E94C/F98W/C109A/K177M/K358I;
T40/E94/F98/C109/Y350/K358, such as T40Y/E94C/F98W/C109A/Y350I/K358I or T40Y/E94C/F98W/C109A/Y350E/K358I;
E94/F98/C109/C114/K177, such as E94C/F98W/C109A/C114I/K177M;
E94/F98/C109/C114/Y350, such as E94C/F98W/C109A/C114I/Y350I or E94C/F98W/C109A/C114I/Y350E;
E94/F98/C109/C114/K358, such as E94C/F98W/C109A/C114I/K358I;
E94/F98/C109/K177/Y350, such as E94C/F98W/C109A/K177M/Y350I or E94C/F98W/C109A/K177M/Y350E;
E94/F98/C109/K177/K358, such as E94C/F98W/C109A/K177M/K358I;
E94/F98/C109/Y350/K358, such as E94C/F98W/C109A/Y350I/K358I or E94C/F98W/C109A/Y350E/K358I;
T40/E94/F98/C109/C114/K177/Y350, such as T40Y/E94C/F98W/C109A/C114I/K177M/Y350I or T40Y/E94C/F98W/C109A/C114I/K177M/Y350E;
T40/E94/F98/C109/C114/K177/K358, such as T40Y/E94C/F98W/C109A/C114I/K177M/K358I;
T40/E94/F98/C109/C114/Y350/K358, such as T40Y/E94C/F98W/C109A/C114I/Y350I/K358I or T40Y/E94C/F98W/C109A/C114I/Y350E/K358I;
T40/E94/F98/C109/K177/Y350/K358, such as T40Y/E94C/F98W/C109A/K177M/Y350I/K358I or T40Y/E94C/F98W/C109A/K177M/Y350E/K358I;
E94/F98/C109/C114/K177/Y350, such as E94C/F98W/C109A/C114I/K177M/Y350I or E94C/F98W/C109A/C114I/K177M/Y350E;
E94/F98/C109/C114/K177/K358, such as E94C/F98W/C109A/C114I/K177M/K358I;
E94/F98/C109/C114/Y350/K358, such as E94C/F98W/C109A/C114I/Y350I/K358I or E94C/F98W/C109A/C114I/Y350E/K358I;
E94/F98/C109/K177/Y350/K358, such as E94C/F98W/C109A/K177M/Y350I/K358I or E94C/F98W/C109A/K177M/Y350E/K358I;
T40/E94/F98/C109/C114/K177/Y350/K358, such as T40Y/E94C/F98W/C109A/C114I/K177M/Y350I/K358I or T40Y/E94C/F98W/C109A/C114I/K177M/Y350E/K358I;
E94/F98/C109/C114/K177/Y350/K358, such as E94C/F98W/C109A/C114I/K177M/Y350I/K358I or E94C/F98W/C109A/C114I/K177M/Y350E/K358I;
T40/C109/C136, such as T40Y/C109A/C136A;
T40/C109/C114/C136, such as T40Y/C109A/C114I/C136A;
T40/C109/C136/K177, such as T40Y/C109A/C136A/K177M;
T40/C109/C136/Y350, such as T40Y/C109A/C136A/Y350I or T40Y/C109A/C136A/Y350E;
T40/C109/C136/K358, such as T40Y/C109A/C136A/K358I;
C109/C114/C136, such as C109A/C136A/C114I;
C109/C136/K177, such as C109A/C136A/K177M;
C109/C136/Y350, such as C109A/C136A/Y350I or C109A/C136A/Y350E;
C109/C136/K358, such as C109A/C136A/K358I;
T40/C109/C114/C136/K177, such as T40Y/C109A/C114I/C136A/K177M;
T40/C109/C114/C136/Y350, such as T40Y/C109A/C114I/C136A/Y350 or T40Y/C109A/C114I/C136A/Y350I I;
T40/C109/C114/C136/K358, such as T40Y/C109A/C114I/C136A/K358I;
T40/C109/C136/K177/Y350, such as T40Y/C109A/C136A/K177M/Y350E or T40Y/C109A/C136A/K177M/Y350I;
T40/C109/C136/K177/K358, such as T40Y/C109A/C136A/K177M/K358I;
T40/C109/C136/Y350/K358, such as T40Y/C109A/C136A/Y350I/K358I or T40Y/C109A/C136A/Y350E/K358I;
C109/C114/C136/K177, such as C109A/C114I/C136A/K177M;
C109/C114/C136/Y350, such as C109A/C114I/C136A/Y350I or C109A/C114I/C136A/Y350E;
C109/C114/C136/K358, such as C109A/C114I/C136A/K358I;
C109/C136/K177/Y350, such as C109A/C136A/K177M/Y350I or C109A/C136A/K177M/Y350E;
C109/C136/K177/K358, such as C109A/C136A/K177M/K358I;
C109/C136/Y350/K358, such as C109A/C136A/Y350I/K358I or C109A/C136A/Y350E/K358I;
T40/C109/C114/C136/K177/Y350, such as T40Y/C109A/C114I/C136A/K177M/Y350I or T40Y/C109A/C114I/C136A/K177M/Y350E;
T40/C109/C114/C136/K177/K358, such as T40Y/C109A/C114I/C136A/K177M/K358I;
T40/C109/C114/C136/Y350/K358, such as T40Y/C109A/C114I/C136AY350I/K358I or T40Y/C109A/C114I/C136AY350E/K358I;
T40/C109/C136/K177/Y350/K358, such as T40Y/C109A/C136A/K177M/Y350I/K358I or T40Y/C109A/C136A/K177M/Y350E/K358I;
C109/C114/C136/K177/Y350, such as C109A/C114I/C136A/K177M/Y350I or C109A/C114I/C136A/K177M/Y350E;
C109/C114/C136/K177/K358, such as C109A/C114I/C136A/K177M/K358I;
C109/C114/C136/Y350/K358, such as C109A/C114I/C136AY350I/K358I or C109A/C114I/C136AY350E/K358I;
C109/C136/K177/Y350/K358, such as C109A/C136A/K177M/Y350I/K358I or C109A/C136A/K177M/Y350E/K358I;
T40/C109/C114/C136/K177/Y350/K358, such as T40Y/C109A/C114I/C136A/K177M/Y350I/K358I or T40Y/C109A/C114I/C136A/K177M/Y350E/K358I;
C109/C114/C136/K177/Y350/K358, such as C109A/C114I/C136A/K177M/Y350I/K358I C109A/C114I/C136A/K177M/Y350E/K358I;
T40/E94/F98/C109/K194, such as T40Y/E94C/F98W/C109A/K194L;
T40/E94/F98/C109/C114/K194, such as T40Y/E94C/F98W/C109A/C114I/K194L;
T40/E94/F98/C109/K177/K194, such as T40Y/E94C/F98W/C109A/K177M/K194L;
T40/E94/F98/C109/K194/Y350, such as T40Y/E94C/F98W/C109A/K194L/Y350I or T40Y/E94C/F98W/C109A/K194L/Y350E;
T40/E94/F98/C109/K194/K358, such as T40Y/E94C/F98W/C109A/K194L/K358I;
E94/F98/C109/C114/K194, such as E94C/F98W/C109A/C114I/K194L;
E94/F98/C109/K177/K194, such as E94C/F98W/C109A/K177M/K194L;
E94/F98/C109/K194/Y350, such as E94C/F98W/C109A/K194L/Y350I or E94C/F98W/C109A/K194L/Y350E;
E94/F98/C109/K194/K358, such as E94C/F98W/C109A/K194L/K358I;
T40/E94/F98/C109/C114/K177/K194, such as T40Y/E94C/F98W/C109A/C114I/K194L/K177M;
T40/E94/F98/C109/C114/K194/Y350, such as T40Y/E94C/F98W/C109A/C114I/K194L/Y350I or T40Y/E94C/F98W/C109A/C114I/K194L/Y350E;
T40/E94/F98/C109/C114/K194/K358, such as T40Y/E94C/F98W/C109A/C114I/K194L/K358I;
T40/E94/F98/C109/K177/K194/Y350, such as T40Y/E94C/F98W/C109A/K177M/K194L/Y350I or T40Y/E94C/F98W/C109A/K177M/K194L/Y350E;
T40/E94/F98/C109/K177/K194/K358, such as T40Y/E94C/F98W/C109A/K177M/K194L/K358I;
T40/E94/F98/C109/K194/Y350/K358, such as T40Y/E94C/F98W/C109A/K194L/Y350I/K358I or T40Y/E94C/F98W/C109A/K194L/Y350E/K358I;
E94/F98/C109/C114/K177/K194, such as E94C/F98W/C109A/C114I/K194L/K177M;
E94/F98/C109/C114/K194/Y350, such as E94C/F98W/C109A/C114I/K194L/Y350I or E94C/F98W/C109A/C114I/K194L/Y350E;
E94/F98/C109/C114/K194/K358, such as E94C/F98W/C109A/C114I/K194L/K358I;
E94/F98/C109/K177/K194/Y350, such as E94C/F98W/C109A/K177M/K194L/Y350I o E94C/F98W/C109A/K177M/K194L/Y350E;
E94/F98/C109/K177/K194/K358, such as E94C/F98W/C109A/K177M/K194L/K358I;
E94/F98/C109/K194/Y350/K358, such as E94C/F98W/C109A/K194L/Y350I/K358I or E94C/F98W/C109A/K194L/Y350E/K358I;
T40/E94/F98/C109/C114/K177/K194/Y350, such as T40Y/E94C/F98W/C109A/C114I/K177M/K194L/Y350I or T40Y/E94C/F98W/C109A/C114I/K177M/K194L/Y350E;
T40/E94/F98/C109/C114/K177/K194/K358, such as T40Y/E94C/F98W/C109A/C114I/K177M/K194L/K358I;
T40/E94/F98/C109/C114/K194/Y350/K358, such as T40Y/E94C/F98W/C109A/C114I/K194L/Y350I/K358I or T40Y/E94C/F98W/C109A/C114I/K194L/Y350E/K358I;
T40/E94/F98/C109/K177/K194/Y350/K358, such as T40Y/E94C/F98W/C109A/K177M/K194L/Y350I/K358I or T40Y/E94C/F98W/C109A/K177M/K194L/Y350E/K358I;
E94/F98/C109/C114/K177/K194/Y350, such as E94C/F98W/C109A/C114I/K177M/K194L/Y350I or E94C/F98W/C109A/C114I/K177M/K194L/Y350E;
E94/F98/C109/C114/K177/K194/K358, such as E94C/F98W/C109A/C114I/K177M/K194L/K358I;
E94/F98/C109/C114/K194/Y350/K358, such as E94C/F98W/C109A/C114I/K194L/Y350I/K358I or E94C/F98W/C109A/C114I/K194L/Y350E/K358I;
E94/F98/C109/K177/K194/Y350/K358, such as E94C/F98W/C109A/K177M/K194L/Y350I/K358I or E94C/F98W/C109A/K177M/K194L/Y350E/K358I;
T40/E94/F98/C109/C114/K177/K194/Y350/K358, such as T40Y/E94C/F98W/C109A/C114I/K177M/K194L/Y350I/K358I or T40Y/E94C/F98W/C109A/C114I/K177M/K194L/Y350E/K358I;
E94/F98/C109/C114/K177/K194/Y350/K358, such as E94C/F98W/C109A/C114I/K177M/K194L/Y350I/K358I or E94C/F98W/C109A/C114I/K177M/K194L/Y350E/K358I;
T40/E94/F98/C109/C136, such as T40Y/E94C/F98W/C109A/C136A;
T40/E94/F98/C109/C114/C136, such as T40Y/E94C/F98W/C109A/C114I/C136A;
T40/E94/F98/C109/C136/K177, such as T40Y/E94C/F98W/C109A/C136A/K177M;
T40/E94/F98/C109/C136/Y350, such as T40Y/E94C/F98W/C109A/C136A/Y350I or T40Y/E94C/F98W/C109A/C136A/Y350E;
T40/E94/F98/C109/C136/K358, such as T40Y/E94C/F98W/C109A/C136A/K358I;
E94/F98/C109/C114/C136, such as E94C/F98W/C109A/C114I/C136A;
E94/F98/C109/C136/K177, such as E94C/F98W/C109A/C136A/K177M;
E94/F98/C109/C136/Y350, such as E94C/F98W/C109A/C136A/Y350I or T40Y/E94C/F98W/C109A/C136A/Y350E;
E94/F98/C109/C136/K358, such as E94C/F98W/C109A/C136A/K358I;
T40/E94/F98/C109/C114/C136/K177, such as T40Y/E94C/F98W/C109A/C114I/C136A/K177M;
T40/E94/F98/C109/C114/C136/Y350, such as T40Y/E94C/F98W/C109A/C114I/C136A/Y350I or T40Y/E94C/F98W/C109A/C114I/C136A/Y350E;
T40/E94/F98/C109/C114/C136/K358, such as T40Y/E94C/F98W/C109A/C114I/C136A/K358I;
T40/E94/F98/C109/C136/K177/Y350, such as T40Y/E94C/F98W/C109A/C136AK177M/Y350I or T40Y/E94C/F98W/C109A/C136AK177M/Y350E;
T40/E94/F98/C109/C136/K177/K358, such as T40Y/E94C/F98W/C109A/C136AK177M/K358I;
T40/E94/F98/C109/C136/Y350/K358, such as T40Y/E94C/F98W/C109A/C136AY350I/K358I or T40Y/E94C/F98W/C109A/C136AY350E/K358I;
E94/F98/C109/C114/C136/K177, such as E94C/F98W/C109A/C114I/C136A/K177M;
E94/F98/C109/C114/C136/Y350, such as E94C/F98W/C109A/C114I/C136A/Y350I or E94C/F98W/C109A/C114I/C136A/Y350E;
E94/F98/C109/C114/C136/K358, such as E94C/F98W/C109A/C114I/C136A/K358I;
E94/F98/C109/C136/K177/Y350, such as E94C/F98W/C109A/C136AK177M/Y350I or E94C/F98W/C109A/C136AK177M/Y350E;
E94/F98/C109/C136/K177/K358, such as E94C/F98W/C109A/C136AK177M/K358I;
E94/F98/C109/C136/Y350/K358, such as E94C/F98W/C109A/C136AY350I/K358I or E94C/F98W/C109A/C136AY350E/K358I;
T40/E94/F98/C109/C114/C136/K177/Y350, such as T40Y/E94C/F98W/C109A/C114I/C136A/K177M/Y350I or T40Y/E94C/F98W/C109A/C114I/C136A/K177M/Y350E;
T40/E94/F98/C109/C114/C136/K177/K358, such as T40Y/E94C/F98W/C109A/C114I/C136A/K177M/K358I;
T40/E94/F98/C109/C114/C136/Y350/K358, such as T40Y/E94C/F98W/C109A/C114I/C136A/Y350I/K358I or T40Y/E94C/F98W/C109A/C114I/C136A/Y350E/K358I;
T40/E94/F98/C109/C136/K177/Y350/K358, such as T40Y/E94C/F98W/C109A/C136A/K177M/Y350I/K358I or T40Y/E94C/F98W/C109A/C136A/K177M/Y350E/K358I;
E94/F98/C109/C114/C136/K177/Y350, such as E94C/F98W/C109A/C114I/C136A/K177M/Y350I or E94C/F98W/C109A/C114I/C136A/K177M/Y350E;
E94/F98/C109/C114/C136/K177/K358, such as E94C/F98W/C109A/C114I/C136A/K177M/K358I;
E94/F98/C109/C114/C136/Y350/K358, such as E94C/F98W/C109A/C114I/C136A/Y350I/K358I or E94C/F98W/C109A/C114I/C136A/Y350E/K358I;
E94/F98/C109/C136/K177/Y350/K358, such as E94C/F98W/C109A/C136A/K177M/Y350I/K358I or E94C/F98W/C109A/C136A/K177M/Y350E/K358I;
T40/E94/F98/C109/C114/C136/K177/Y350/K358, such as T40Y/E94C/F98W/C109A/C114I/C136A/K177M/Y350I/K358I or T40Y/E94C/F98W/C109A/C114I/C136A/K177M/Y350E/K358I;
E94/F98/C109/C114/C136/K177/Y350/K358, such as E94C/F98W/C109A/C114I/C136A/K177M/Y350I/K358I or E94C/F98W/C109A/C114I/C136A/K177M/Y350E/K358I;
T40/E94/F98/C109/A360, such as T40Y/E94C/F98W/C109A/A360C;
T40/E94/F98/C109/C114/A360, such as T40Y/E94C/F98W/C109A/C114I/A360C;
T40/E94/F98/C109/K177/A360, such as T40Y/E94C/F98W/C109A/K177M/A360C;
T40/E94/F98/C109/Y350/A360, such as T40Y/E94C/F98W/C109A/Y350I/A360C or T40Y/E94C/F98W/C109A/Y350E/A360C;
T40/E94/F98/C109/K358/A360, such as T40Y/E94C/F98W/C109A/K358I/A360C;
E94/F98/C109/C114/A360, such as E94C/F98W/C109A/C114I/A360C;
E94/F98/C109/K177/A360, such as E94C/F98W/C109A/K177M/A360C;
E94/F98/C109/Y350/A360, such as E94C/F98W/C109A/Y350I/A360C or E94C/F98W/C109A/Y350E/A360C;
E94/F98/C109/K358/A360, such as E94C/F98W/C109A/K358I/A360C;
T40/E94/F98/C109/C114/K177/A360, such as T40Y/E94C/F98W/C109A/C114I/K177M/A360C;
T40/E94/F98/C109/C114/Y350/A360, such as T40Y/E94C/F98W/C109A/C114I/Y350I/A360C or T40Y/E94C/F98W/C109A/C114I/Y350E/A360C;
T40/E94/F98/C109/C114/K358/A360, such as T40Y/E94C/F98W/C109A/C114I/K358I/A360C;
T40/E94/F98/C109/K177/Y350/A360, such as T40Y/E94C/F98W/C109A/K177M/Y350I/A360C or T40Y/E94C/F98W/C109A/K177M/Y350E/A360C;
/A360C T40/E94/F98/C109/Y350/K358/A360, such as T40Y/E94C/F98W/C109A/Y350I/K358I/A360C or T40Y/E94C/F98W/C109A/Y350E/K358I/A360C;
E94/F98/C109/C114/K177/A360, such as E94C/F98W/C109A/C114I/K177M/A360C;
E94/F98/C109/C114/Y350/A360, such as E94C/F98W/C109A/C114I/Y350I/A360C or E94C/F98W/C109A/C114I/Y350E/A360C;
E94/F98/C109/C114/K358/A360, such as E94C/F98W/C109A/C114I/K358I/A360C;
E94/F98/C109/K177/Y350/A360, such as E94C/F98W/C109A/K177M/Y350I/A360C or E94C/F98W/C109A/K177M/Y350E/A360C;
E94/F98/C109/Y350/K358/A360, such as E94C/F98W/C109A/Y350I/K358I/A360C or E94C/F98W/C109A/Y350E/K358I/A360C;
T40/E94/F98/C109/C114/K177/Y350/A360, such as T40Y/E94C/F98W/C109A/C114I/K177M/Y350I/A360C or T40Y/E94C/F98W/C109A/C114I/K177M/Y350E/A360C;
T40/E94/F98/C109/C114/K177/K358/A360, such as T40Y/E94C/F98W/C109A/C114I/K177M/K358I/A360C;
T40/E94/F98/C109/C114/Y350/K358/A360, such as T40Y/E94C/F98W/C109A/C114I/Y350I/K358I/A360C or T40Y/E94C/F98W/C109A/C114I/Y350E/K358I/A360C;
T40/E94/F98/C109/K177/Y350/K358/A360, such as T40Y/E94C/F98W/C109A/K177M/Y350I/K358I/A360C or T40Y/E94C/F98W/C109A/K177M/Y350E/K358I/A360C;
E94/F98/C109/C114/K177/Y350/A360, such as E94C/F98W/C109A/C114I/K177M/Y350I/A360C or E94C/F98W/C109A/C114I/K177M/Y350E/A360C;
E94/F98/C109/C114/K177/K358/A360, such as E94C/F98W/C109A/C114I/K177M/K358I/A360C;
E94/F98/C109/C114/Y350/K358/A360, such as E94C/F98W/C109A/C114I/Y350I/K358I/A360C or E94C/F98W/C109A/C114I/Y350E/K358I/A360C;
E94/F98/C109/K177/Y350/K358/A360, such as E94C/F98W/C109A/K177M/Y350I/K358I/A360C or E94C/F98W/C109A/K177M/Y350E/K358I/A360C;
T40/E94/F98/C109/C114/K177/Y350/K358/A360, such as T40Y/E94C/F98W/C109A/C114I/K177M/Y350I/K358I/A360C or T40Y/E94C/F98W/C109A/C114I/K177M/Y350E/K358I/A360C; or
E94/F98/C109/C114/K177/Y350/K358/A360, such as E94C/F98W/C109A/C114I/K177M/Y350I/K358I/A360C or E94C/F98W/C109A/C114I/K177M/Y350E/K358I/A360C.

Any of these variants of SEQ ID NO: 118 may further comprise a modification or substitution at any number and combination of positions (a) 55, (b) 156, (c) 210 and (d) 221, including at (a); (b); (c); (d); (a) and (b); (a) and (c); (a) and (d); (b) and (c); (b) and (d); (c) and (d); (a), (b) and (c); (a), (b) and (d); (a), (c) and (d); (b), (c) and (d); or (a), (b), (c) and (d). Any of these variants of SEQ ID NO: 118 may further comprise any number and combination of (a) T55K, (b) T156F, (c) T210K and (d) N221E, including at (a); (b); (c); (d); (a) and (b); (a) and (c); (a) and (d); (b) and (c); (b) and (d); (c) and (d); (a), (b) and (c); (a), (b) and (d); (a), (c) and (d); (b), (c) and (d); or (a), (b), (c) and (d).

### Additional Modifications/Substitutions

The invention also provides a modified DNA dependent ATPase (Dda) helicase in which one or more of the positions corresponding to the following amino acid positions in Dda 1993 are modified or substituted: 86, 90, 92, 97, 101, 102, 273, 293, 300, 301, 303, 305, 308, 310, 312 317, 323, 328, 332, 334, 335, 336, 337, 339, 351, 354, 359, 361, 364, 366, 368, 371, 374, 376, 377, 379 and 388. The invention also provides a modified DNA dependent ATPase (Dda) helicase in which one or more of the positions corresponding to the following amino acid positions in Dda 1993 are modified or substituted: 351, 354 and 361. Any number and combination of these modifications/substitutions may be made, including at 351, 354, 361, 351 and 354, 351 and 361, 354 and 361 or 351, 354 and 361. These positions may be modified or substituted in isolation or in combination with any of the modifications or substitutions of the invention above.

The helicase of the invention preferably comprises a variant of SEQ ID NO: 118 comprising one or more of the following substitutions: K86A, V90T, Y92A, Y92D, Y92F, Y92G, Y92H, Y92N, Y92Q, Y92S, Y92T, Y92V, Y92W, L97H, K101A, E102G, E273A, N293H, I200A, I300F, E301I, E303I, Y305L, F308I, F308L, K310A, K310I, K310L, R312I, R312L, R312M, E317I, E317Y, W323H, E328I, D332A, D332L, E334A, E334I, E334Y, Y335A, Y335I, Y335L, Y336L, R337I, R337L, R337M, K339A, K339I, K339L, K351I, K351Q, L354Q, L354A, T359L, E361T, E361I, E361Q, K364A, K364R, W366L, K368A, K368I, K368L, K371I, K371L, K371M, W374A, W374L, W374L, D376S, F377A, D379A, D379L or K388R. The helicase of the invention preferably comprises a variant of SEQ ID NO: 118 comprising one or more of the following substitutions: (a) K351I or K351Q, (b) L354A or L354Q and (c) E361I or E361Q. The variant may comprise (a), (b), (c), (a) and (b), (a) and (c), (b) and (c) or (a), (b) and (c). These substitutions may be made in isolation or in combination with any of the modifications or substitutions of the invention above.

### Variants

A variant of a helicase is an enzyme that has an amino acid sequence which varies from that of the wild-type helicase and which has polynucleotide binding activity. In particular, a variant of any one of SEQ ID NOs: 118 to 133 is an enzyme that has an amino acid sequence which varies from that of any one of SEQ ID NOs: 118 to 133 and which has polynucleotide binding activity. Polynucleotide binding activity can be determined using methods known in the art. Suitable methods include, but are not limited to, fluorescence anisotropy, tryptophan fluorescence and electrophoretic mobility shift assay (EMSA). For instance, the ability of a variant to bind a single stranded polynucleotide can be determined as described in the Examples.

The variant has helicase activity. This can be measured in various ways. For instance, the ability of the variant to translocate along a polynucleotide can be measured using electrophysiology, a fluorescence assay or ATP hydrolysis.

The variant may include modifications that facilitate handling of the polynucleotide encoding the helicase and/or facilitate its activity at high salt concentrations and/or room temperature.

Over the entire length of the amino acid sequence of any one of SEQ ID NOs: 118 to 133, a variant will preferably be at least 20% homologous to that sequence based on amino acid similarity or identity. More preferably, the variant polypeptide may be at least 30%, at least 40%, at least 45%, at least 50%, at least 55%, at least 60%, at least 65%, at least 70%, at least 75%, at least 80%, at least 85%, at least 90% and more preferably at least 95%, 97% or 99% homologous based on amino acid identity to the amino acid sequence of any one of SEQ ID NOs: 118 to 133 over the entire sequence. More preferably, the variant polypeptide may be at least 30%, at least 40%, at least 45%, at least 50%, at least 55%, at least 60%, at least 65%, at least 70%, at least 75%, at least 80%, at least 85%, at least 90% and more preferably at least 95%, 97% or 99% identical to the amino acid sequence of any one of SEQ ID NOs: 118 to 133 over the entire sequence. There may be at least 70%, for example at least 80%, at least 85%, at least 90% or at least 95%, amino acid identity over a stretch of 100 or more, for example 150, 200, 300, 400 or 500 or more, contiguous amino acids (“hard homology”). Homology is determined as described below. In particular, in addition to the specific modifications discussed above, the variant of any one of SEQ ID NOs: 118 to 133 may comprise one or more substitutions, one or more deletions and/or one or more additions as discussed below.

Preferred variants of any one of SEQ ID NOs: 118 to 133 have a non-natural amino acid, such as Faz, at the amino- (N-) terminus and/or carboxy (C-) terminus. Preferred variants of any one of SEQ ID NOs: 118 to 133 have a cysteine residue at the amino- (N-) terminus and/or carboxy (C-) terminus. Preferred variants of any one of SEQ ID NOs: 118 to 133 have a cysteine residue at the amino- (N-) terminus and a non-natural amino acid, such as Faz, at the carboxy (C-) terminus or vice versa.

Preferred variants of SEQ ID NO: 118 contain one or more of, such as all of, the following modifications E54G, D151E, I196N and G357A.

### No Connection

In one preferred embodiment, none of the introduced cysteines and/or non-natural amino acids in a modified helicase of the invention are connected to one another.

Connecting Two More of the Introduced Cysteines and/or Non-Natural Amino Acids

In another preferred embodiment, two more of the introduced cysteines and/or non-natural amino acids in a modified helicase of the invention are connected to one another. This typically reduces the ability of the helicase of the invention to unbind from a polynucleotide.

Any number and combination of two more of the introduced cysteines and/or non-natural amino acids may be connected to one another. For instance, 3, 4, 5, 6, 7, 8 or more cysteines and/or non-natural amino acids may be connected to one another. One or more cysteines may be connected to one or more cysteines. One or more cysteines may be connected to one or more non-natural amino acids, such as Faz. One or more non-natural amino acids, such as Faz, may be connected to one or more non-natural amino acids, such as Faz.

The two or more cysteines and/or non-natural amino acids may be connected in any way. The connection can be transient, for example non-covalent. Even transient connection will reduce unbinding of the polynucleotide from the helicase.

The two or more cysteines and/or non-natural amino acids are preferably connected by affinity molecules. Suitable affinity molecules are known in the art. The affinity molecules are preferably (a) complementary polynucleotides (WO 2010/086602 incorporated herein by reference in its entirety), (b) an antibody or a fragment thereof and the complementary epitope (Biochemistry 6th Ed, W.H. Freeman and co (2007) pp 953-954), (c) peptide zippers (O'Shea et al., Science 254 (5031): 539-544), (d) capable of interacting by 3-sheet augmentation (Remaut and Waksman Trends Biochem. Sci. (2006) 31 436-444), (e) capable of hydrogen bonding, pi-stacking or forming a salt bridge, (f) rotaxanes (Xiang Ma and He Tian Chem. Soc. Rev., 2010, 39, 70-80), (g) an aptamer and the complementary protein (James, W. in Encyclopedia of Analytical Chemistry, R. A. Meyers (Ed.) pp. 4848-4871 John Wiley & Sons Ltd, Chichester, 2000) or (h) half-chelators (Hammerstein et al. J Biol Chem. 2011 Apr. 22; 286(16): 14324-14334). For (e), hydrogen bonding occurs between a proton bound to an electronegative atom and another electronegative atom. Pi-stacking requires two aromatic rings that can stack together where the planes of the rings are parallel. Salt bridges are between groups that can delocalize their electrons over several atoms, e. g. between aspartate and arginine.

The two or more parts may be transiently connected by a hexa-his tag or Ni-NTA.

The two or more cysteines and/or non-natural amino acids are preferably permanently connected.

In the context of the invention, a connection is permanent if is not broken while the helicase is used or cannot be broken without intervention on the part of the user, such as using reduction to open —S—S— bonds.

The two or more cysteines and/or non-natural amino acids are preferably covalently-attached.

The two or more cysteines and/or non-natural amino acids may be covalently attached using any method known in the art.

The two or more cysteines and/or non-natural amino acids may be covalently attached via their naturally occurring amino acids, such as cysteines, threonines, serines, aspartates, asparagines, glutamates and glutamines. Naturally occurring amino acids may be modified to facilitate attachment. For instance, the naturally occurring amino acids may be modified by acylation, phosphorylation, glycosylation or farnesylation. Other suitable modifications are known in the art. Modifications to naturally occurring amino acids may be post-translation modifications. The two or more cysteines and/or non-natural amino acids may be attached via amino acids that have been introduced into their sequences. Such amino acids are preferably introduced by substitution. The introduced amino acid may be cysteine or a non-natural amino acid that facilitates attachment. Suitable non-natural amino acids include, but are not limited to, 4-azido-L-phenylalanine (Faz), any one of the amino acids numbered 1-71 included in FIG. 1 of Liu C. C. and Schultz P. G., Annu. Rev. Biochem., 2010, 79, 413-444 or any one of the amino acids listed below. The introduced amino acids may be modified as discussed above.

In a preferred embodiment, the two or more cysteines and/or non-natural amino acids are connected using linkers. Linker molecules are discussed in more detail below. One suitable method of connection is cysteine linkage. This is discussed in more detail below. The two or more cysteines and/or non-natural amino acids are preferably connected using one or more, such as two or three, linkers. The one or more linkers may be designed to reduce the size of, or close, the opening as discussed above. If one or more linkers are being used to close the opening as discussed above, at least a part of the one or more linkers is preferably oriented such that it is not parallel to the polynucleotide when it is bound by the helicase. More preferably, all of the linkers are oriented in this manner. If one or more linkers are being used to close the opening as discussed above, at least a part of the one or more linkers preferably crosses the opening in an orientation that is not parallel to the polynucleotide when it bound by the helicase. More preferably, all of the linkers cross the opening in this manner. In these embodiments, at least a part of the one or more linkers may be perpendicular to the polynucleotide. Such orientations effectively close the opening such that the polynucleotide cannot unbind from the helicase through the opening.

Each linker may have two or more functional ends, such as two, three or four functional ends. Suitable configurations of ends in linkers are well known in the art.

One or more ends of the one or more linkers are preferably covalently attached to the helicase. If one end is covalently attached, the one or more linkers may transiently connect the two or more cysteines and/or non-natural amino acids as discussed above. If both or all ends are covalently attached, the one or more linkers permanently connect the two or more cysteines and/or non-natural amino acids.

The one or more linkers are preferably amino acid sequences and/or chemical crosslinkers. Suitable amino acid linkers, such as peptide linkers, are known in the art. The length, flexibility and hydrophilicity of the amino acid or peptide linker are typically designed such that it reduces the size of the opening, but does not to disturb the functions of the helicase. Preferred flexible peptide linkers are stretches of 2 to 20, such as 4, 6, 8, 10 or 16, serine and/or glycine amino acids. More preferred flexible linkers include (SG)₁, (SG)₂, (SG)₃, (SG)₄, (SG)₅, (SG)₅, (SG)₁₀, (SG)₁₅ or (SG)₂₀ wherein S is serine and G is glycine. Preferred rigid linkers are stretches of 2 to 30, such as 4, 6, 8, 16 or 24, proline amino acids. More preferred rigid linkers include (P)₁₂ wherein P is proline. The amino acid sequence of a linker preferably comprises a polynucleotide binding moiety. Such moieties and the advantages associated with their use are discussed below. Suitable chemical crosslinkers are well-known in the art. Suitable chemical crosslinkers include, but are not limited to, those including the following functional groups: maleimide, active esters, succinimide, azide, alkyne (such as dibenzocyclooctynol (DIBO or DBCO), difluoro cycloalkynes and linear alkynes), phosphine (such as those used in traceless and non-traceless Staudinger ligations), haloacetyl (such as iodoacetamide), phosgene type reagents, sulfonyl chloride reagents, isothiocyanates, acyl halides, hydrazines, disulphides, vinyl sulfones, aziridines and photoreactive reagents (such as aryl azides, diaziridines).

Reactions between amino acids and functional groups may be spontaneous, such as cysteine/maleimide, or may require external reagents, such as Cu(I) for linking azide and linear alkynes.

Linkers can comprise any molecule that stretches across the distance required. Linkers can vary in length from one carbon (phosgene-type linkers) to many Angstroms. Examples of linear molecules, include but are not limited to, are polyethyleneglycols (PEGs), polypeptides, polysaccharides, deoxyribonucleic acid (DNA), peptide nucleic acid (PNA), threose nucleic acid (TNA), glycerol nucleic acid (GNA), saturated and unsaturated hydrocarbons, polyamides.

These linkers may be inert or reactive, in particular they may be chemically cleavable at a defined position, or may be themselves modified with a fluorophore or ligand. The linker is preferably resistant to dithiothreitol (DTT).

Preferred crosslinkers include 2,5-dioxopyrrolidin-1-yl 3-(pyridin-2-yldisulfanyl)propanoate, 2,5-dioxopyrrolidin-1-yl 4-(pyridin-2-yldisulfanyl)butanoate and 2,5-dioxopyrrolidin-1-yl 8-(pyridin-2-yldisulfanyl)octananoate, di-maleimide PEG 1k, di-maleimide PEG 3.4k, di-maleimide PEG 5k, di-maleimide PEG 10k, bis(maleimido)ethane (BMOE), bis-maleimidohexane (BMH), 1,4-bis-maleimidobutane (BMB), 1,4 bis-maleimidyl-2,3-dihydroxybutane (BMDB), BM[PEO]2 (1,8-bis-maleimidodiethyleneglycol), BM[PEO]3 (1,11-bis-maleimidotriethylene glycol), tris[2-maleimidoethyl]amine (TMEA), DTME dithiobismaleimidoethane, bis-maleimide PEG3, bis-maleimide PEG11, DBCO-maleimide, DBCO-PEG4-maleimide, DBCO-PEG4-NH2, DBCO-PEG4-NHS, DBCO-NHS, DBCO-PEG-DBCO 2.8 kDa, DBCO-PEG-DBCO 4.0 kDa, DBCO-15 atoms-DBCO, DBCO-26 atoms-DBCO, DBCO-35 atoms-DBCO, DBCO-PEG4-S-S-PEG3-biotin, DBCO-S-S-PEG3-biotin, DBCO-S-S-PEG11-biotin, (succinimidyl 3-(2-pyridyldithio)propionate (SPDP) and maleimide-PEG(2 kDa)-maleimide (ALPHA,OMEGA-BIS-MALEIMIDO POLY(ETHYLENE GLYCOL)). The most preferred crosslinker is maleimide-propyl-SRDFWRS-(1,2-diaminoethane)-propyl-maleimide.

The one or more linkers may be cleavable. This is discussed in more detail below.

The two or more cysteines and/or non-natural amino acids may be connected using two different linkers that are specific for each other. One of the linkers is attached to one part and the other is attached to another part. The linkers should react to form a modified helicase of the invention.

The two or more cysteines and/or non-natural amino acids may be connected using the hybridization linkers described in WO 2010/086602 (incorporated herein by reference in its entirety). In particular, the two or more cysteines and/or non-natural amino acids may be connected using two or more linkers each comprising a hybridizable region and a group capable of forming a covalent bond. The hybridizable regions in the linkers hybridize and link the two or more cysteines and/or non-natural amino acids. The linked cysteines and/or non-natural amino acids are then coupled via the formation of covalent bonds between the groups. Any of the specific linkers disclosed in WO 2010/086602 (incorporated herein by reference in its entirety) may be used in accordance with the invention.

The two or more cysteines and/or non-natural amino acids may be modified and then attached using a chemical crosslinker that is specific for the two modifications. Any of the crosslinkers discussed above may be used.

The linkers may be labeled. Suitable labels include, but are not limited to, fluorescent molecules (such as Cy3 or AlexaFluor®555), radioisotopes, e.g. ¹²⁵I, ³⁵S, enzymes, antibodies, antigens, polynucleotides and ligands such as biotin. Such labels allow the amount of linker to be quantified. The label could also be a cleavable purification tag, such as biotin, or a specific sequence to show up in an identification method, such as a peptide that is not present in the protein itself, but that is released by trypsin digestion.

A preferred method of connecting two or more cysteines is via cysteine linkage. This can be mediated by a bi-functional chemical crosslinker or by an amino acid linker with a terminal presented cysteine residue.

The length, reactivity, specificity, rigidity and solubility of any bi-functional linker may be designed to ensure that the size of the opening is reduced sufficiently and the function of the helicase is retained. Suitable linkers include bismaleimide crosslinkers, such as 1,4-bis(maleimido)butane (BMB) or bis(maleimido)hexane. One drawback of bi-functional linkers is the requirement of the helicase to contain no further surface accessible cysteine residues if attachment at specific sites is preferred, as binding of the bi-functional linker to surface accessible cysteine residues may be difficult to control and may affect substrate binding or activity. If the helicase does contain several accessible cysteine residues, modification of the helicase may be required to remove them while ensuring the modifications do not affect the folding or activity of the helicase. This is discussed in WO 2010/086603 (incorporated herein by reference in its entirety). The reactivity of cysteine residues may be enhanced by modification of the adjacent residues, for example on a peptide linker. For instance, the basic groups of flanking arginine, histidine or lysine residues will change the pKa of the cysteines thiol group to that of the more reactive S-group. The reactivity of cysteine residues may be protected by thiol protective groups such as 5,5′-dithiobis-(2-nitrobenzoic acid) (dTNB). These may be reacted with one or more cysteine residues of the helicase before a linker is attached. Selective deprotection of surface accessible cysteines may be possible using reducing reagents immobilized on beads (for example immobilized tris(2-carboxyethyl) phosphine, TCEP). Cysteine linkage is discussed in more detail below.

Another preferred method of attachment via Faz linkage. This can be mediated by a bi-functional chemical linker or by a polypeptide linker with a terminal presented Faz residue.

### Other Modifications

The helicase of the invention may also be modified to increase the attraction between (i) the tower domain and (ii) the pin domain and/or the 1A domain. Any known chemical modifications can be made in accordance with the invention. These types of modification are disclosed in WO 2015/055981 (incorporated herein by reference in its entirety).

In particular, the invention provides a helicase of the invention in which at least one charged amino acid has been introduced into (i) the tower domain and/or (ii) the pin domain and/or (iii) the 1A (RecA-like motor) domain, wherein the helicase has the ability to control the movement of a polynucleotide. The ability of the helicase to control the movement of a polynucleotide may be measured as discussed above. The invention preferably provides a helicase of the invention in which at least one charged amino acid has been introduced into (i) the tower domain and (ii) the pin domain and/or the 1A domain.

The at least one charged amino acid may be negatively charged or positively charged. The at least one charged amino acid is preferably oppositely charged to any amino acid(s) with which it interacts in the helicase. For instance, at least one positively charged amino acid may be introduced into the tower domain at a position which interacts with a negatively charged amino acid in the pin domain. The at least one charged amino acid is typically introduced at a position which is not charged in the wild-type (i.e. unmodified) helicase. The at least one charged amino acid may be used to replace at least one oppositely charged amino acid in the helicase. For instance, a positively charged amino acid may be used to replace a negatively charged amino acid.

Suitable charged amino acids are discussed above. The at least one charged amino acid may be natural, such as arginine (R), histidine (H), lysine (K), aspartic acid (D) or glutamic acid (D). Alternatively, the at least one charged amino acid may be artificial or non-natural. Any number of charged amino acids may be introduced into each domain. For instance, 1, 2, 3, 4, 5, 6, 7, 8, 9, 10 or more charged amino acids may be introduced into each domain.

The helicase preferably comprises a variant of SEQ ID NO: 118 which comprises a positively charged amino acid at one or more of the following positions: (i) 93; (ii) 354; (iii) 360; (iv) 361; (v) 94; (vi) 97; (vii) 155; (viii) 357; (ix) 100; and (x) 127. The helicase preferably comprises a variant of SEQ ID NO: 118 which comprises a negatively charged amino acid at one or more of the following positions: (i) 354; (ii) 358; (iii) 360; (iv) 364; (v) 97; (vi) 123; (vii) 155; (viii); 357; (ix) 100; and (x) 127. The helicase preferably comprises a variant of any one of SEQ ID NOs: 119 to 133 which comprises a positively charged amino acid or negatively charged amino acid at the positions which correspond to those in SEQ ID NO: 118 as defined in any of (i) to (x). Positions in any one of SEQ ID NOs: 119 to 133 which correspond to those in SEQ ID NO: 118 can be identified using the alignment of SEQ ID NOs: 118 to 133 below.

The helicase preferably comprises a variant of SEQ ID NO: 118 which is modified by the introduction of at least one charged amino acid such that it comprises oppositely charged amino acid at the following positions: (i) 93 and 354; (ii) 93 and 358; (iii) 93 and 360; (iv) 93 and 361; (v) 93 and 364; (vi) 94 and 354; (vii) 94 and 358; (viii) 94 and 360; (ix) 94 and 361; (x) 94 and 364; (xi) 97 and 354; (xii) 97 and 358; (xiii) 97 and 360; (xiv) 97 and 361; (xv) 97 and 364; (xvi) 123 and 354; (xvii) 123 and 358; (xviii) 123 and 360; (xix) 123 and 361; (xx) 123 and 364; (xxi) 155 and 354; (xxii) 155 and 358; (xxiii) 155 and 360; (xxiv) 155 and 361; (xxv) 155 and 364. The helicase of the invention preferably comprises a variant of any one of SEQ ID NOs: 119 to 133 which comprises oppositely charged amino acids at the positions which correspond to those in SEQ ID NO: 118 as defined in any of (i) to (xxv).

The invention also provides a helicase in which (i) at least one charged amino acid has been introduced into the tower domain and (ii) at least one oppositely charged amino acid has been introduced into the pin domain and/or the 1A (RecA-like motor) domain, wherein the helicase has the ability to control the movement of a polynucleotide. The at least one charged amino acid may be negatively charged and the at least one oppositely charged amino acid may be positively charged or vice versa. Suitable charged amino acids are discussed above. Any number of charged amino acids and any number of oppositely charged amino acids may be introduced. For instance, 1, 2, 3, 4, 5, 6, 7, 8, 9, 10 or more charged amino acids may be introduced and/or 1, 2, 3, 4, 5, 6, 7, 8, 9, 10 or more oppositely charged amino acids may be introduced.

The charged amino acids are typically introduced at positions which are not charged in the wild-type helicase. One or both of the charged amino acids may be used to replace charged amino acids in the helicase. For instance, a positively charged amino acid may be used to replace a negatively charged amino acid. The charged amino acids may be introduced at any of the positions in the (i) tower domain and (ii) pin domain and/or 1A domain discussed above. The oppositely charged amino acids are typically introduced such that they will interact in the resulting helicase. The helicase preferably comprises a variant of SEQ ID NO: 118 in which oppositely charged amino acids have been introduced at the following positions: (i) 97 and 354; (ii) 97 and 360; (iii) 155 and 354; or (iv) 155 and 360. The helicase of the invention preferably comprises a variant of any one of SEQ ID NOs: 119 to 133 which comprises oppositely charged amino acids at the positions which correspond to those in SEQ ID NO: 118 as defined in any of (i) to (iv).

### Construct

The invention also provides a construct comprising a modified helicase of the invention and an additional polynucleotide binding moiety, wherein the helicase is attached to the polynucleotide binding moiety and the construct has the ability to control the movement of a polynucleotide. The construct is artificial or non-natural.

A construct of the invention is a useful tool for controlling the movement of a polynucleotide during Strand Sequencing. A construct of the invention is even less likely than a modified helicase of the invention to disengage from the polynucleotide being sequenced. The construct can provide even greater read lengths of the polynucleotide as it controls the translocation of the polynucleotide through a nanopore.

A targeted construct that binds to a specific polynucleotide sequence can also be designed. As discussed in more detail below, the polynucleotide binding moiety may bind to a specific polynucleotide sequence and thereby target the helicase portion of the construct to the specific sequence.

The construct has the ability to control the movement of a polynucleotide. This can be determined as discussed above.

A construct of the invention may be isolated, substantially isolated, purified or substantially purified. A construct is isolated or purified if it is completely free of any other components, such as lipids, polynucleotides or pore monomers. A construct is substantially isolated if it is mixed with carriers or diluents which will not interfere with its intended use. For instance, a construct is substantially isolated or substantially purified if it is present in a form that comprises less than 10%, less than 5%, less than 2% or less than 1% of other components, such as lipids, polynucleotides or pore monomers.

The helicase may be any of the helicases of the invention discussed above.

The helicase is preferably covalently attached to the additional polynucleotide binding moiety.

The helicase may be attached to the moiety at more than one, such as two or three, points.

The helicase can be covalently attached to the moiety using any method known in the art.

Suitable methods are discussed above with reference to connecting the two or more parts.

The helicase and moiety may be produced separately and then attached together. The two components may be attached in any configuration. For instance, they may be attached via their terminal (i.e. amino or carboxy terminal) amino acids. Suitable configurations include, but are not limited to, the amino terminus of the moiety being attached to the carboxy terminus of the helicase and vice versa. Alternatively, the two components may be attached via amino acids within their sequences. For instance, the moiety may be attached to one or more amino acids in a loop region of the helicase. In a preferred embodiment, terminal amino acids of the moiety are attached to one or more amino acids in the loop region of a helicase.

In a preferred embodiment, the helicase is chemically attached to the moiety, for instance via one or more linker molecules as discussed above. In another preferred embodiment, the helicase is genetically fused to the moiety. A helicase is genetically fused to a moiety if the whole construct is expressed from a single polynucleotide sequence. The coding sequences of the helicase and moiety may be combined in any way to form a single polynucleotide sequence encoding the construct. Genetic fusion of a pore to a nucleic acid binding protein is discussed in WO 2010/004265 (incorporated herein by reference in its entirety).

The helicase and moiety may be genetically fused in any configuration. The helicase and moiety may be fused via their terminal amino acids. For instance, the amino terminus of the moiety may be fused to the carboxy terminus of the helicase and vice versa. The amino acid sequence of the moiety is preferably added in frame into the amino acid sequence of the helicase. In other words, the moiety is preferably inserted within the sequence of the helicase. In such embodiments, the helicase and moiety are typically attached at two points, i.e. via the amino and carboxy terminal amino acids of the moiety. If the moiety is inserted within the sequence of the helicase, it is preferred that the amino and carboxy terminal amino acids of the moiety are in close proximity and are each attached to adjacent amino acids in the sequence of the helicase or variant thereof. In a preferred embodiment, the moiety is inserted into a loop region of the helicase.

The helicase may be attached directly to the moiety. The helicase is preferably attached to the moiety using one or more, such as two or three, linkers as discussed above. The one or more linkers may be designed to constrain the mobility of the moiety. The helicase and/or the moiety may be modified to facilitate attachment of the one or more linker as discussed above. Cleavable linkers can be used as an aid to separation of constructs from non-attached components and can be used to further control the synthesis reaction. For example, a hetero-bifunctional linker may react with the helicase, but not the moiety. If the free end of the linker can be used to bind the helicase protein to a surface, the unreacted helicases from the first reaction can be removed from the mixture. Subsequently, the linker can be cleaved to expose a group that reacts with the moiety. In addition, by following this sequence of linkage reactions, conditions may be optimised first for the reaction to the helicase, then for the reaction to the moiety after cleavage of the linker. The second reaction would also be much more directed towards the correct site of reaction with the moiety because the linker would be confined to the region to which it is already attached.

The helicase may be covalently attached to the bifunctional crosslinker before the helicase/crosslinker complex is covalently attached to the moiety. Alternatively, the moiety may be covalently attached to the bifunctional crosslinker before the bifunctional crosslinker/moiety complex is attached to the helicase. The helicase and moiety may be covalently attached to the chemical crosslinker at the same time.

Preferred methods of attaching the helicase to the moiety are cysteine linkage and Faz linkage as described above. In a preferred embodiment, a reactive cysteine is presented on a peptide linker that is genetically attached to the moiety. This means that additional modifications will not necessarily be needed to remove other accessible cysteine residues from the moiety.

Cross-linkage of helicases or moieties to themselves may be prevented by keeping the concentration of linker in a vast excess of the helicase and/or moiety. Alternatively, a “lock and key” arrangement may be used in which two linkers are used. Only one end of each linker may react together to form a longer linker and the other ends of the linker each react with a different part of the construct (i.e. helicase or moiety). This is discussed in more detail below.

The site of attachment is selected such that, when the construct is contacted with a polynucleotide, both the helicase and the moiety can bind to the polynucleotide and control its movement.

### Embodiments Involving the Modified Helicases and Constructs of the Invention

The invention provides polynucleotides, vectors and host cells. These are discussed above.

The invention provides various methods using the Dda helicases of the invention in particular for controlling the movement of an analyte and characterising a target analyte. Such methods are described above and in WO2016/034591, WO2017/149316, WO2017/149317, WO2017/149318, WO2018/211241, WO2019/002893, WO2015/055981, WO2015/166276 and WO2016/055777 (all incorporated by reference herein).

The modified Dda helicases and constructs of the invention may be used to form sensors for characterising target analytes. These sensors may be formed by contacting the pore and the helicase or construct in the presence of the target analyte and applying a potential across the pore. The helicase or the construct may be covalently attached to the pore, for instance as described above.

In these methods and sensors, the modified Dda helicases and constructs of the invention may be combined/used with the pores of the invention. Alternatively, they may be used with a solid state pore or a transmembrane protein pore is derived from a hemolysin, leukocidin, Mycobacterium smegmatis porin A (MspA), MspB, MspC, MspD, lysenin, outer membrane porin F (OmpF), outer membrane porin G (OmpG), outer membrane phospholipase A, Neisseria autotransporter lipoprotein (NalP) and WZA, CsgG, CsgG/CsgF, lysenin, ClyA, SpI or haemolytic protein fragaceatoxin C (FraC). Preferred combinations of pores and helicases are discussed in more detail below.

### Kits

The invention also provides a kit for characterising a target analyte comprising (a) a pore and a helicase or a construct of the invention or (b) a helicase or construct of the invention and one or more loading moieties. The pore may be any of those discussed above, including the pore of the invention. Preferred combinations of pores and helicases are discussed in more detail below.

The invention also provides a kit for characterising a target analyte, preferably a target polynucleotide, comprising (a) a helicase or construct of the invention and (b) an isolated CsgG pore or isolated pore complex of the invention. Preferred combinations of pores and helicases are discussed in more detail below.

The kit preferably further comprises the components of a membrane. The kit may comprise components of any type of membranes, such as an amphiphilic layer or a triblock copolymer membrane. The kit may further comprise one or more anchors, such as cholesterol, for coupling the target analyte to the membrane. The kit may further comprise one or more polynucleotide adaptors that can be attached to a target polynucleotide to facilitate characterisation of the polynucleotide. In one embodiment, the anchor, such as cholesterol, is attached to the polynucleotide adaptor.

The invention provides a kit for characterising a target analyte comprising (a) an isolated pore or an isolated pore complex of the invention and one or both of (b) the components of a membrane and (c) a polynucleotide binding protein.

Preferred polynucleotide binding proteins are polymerases, exonucleases, helicases and topoisomerases, such as gyrases. Suitable enzymes include, but are not limited to, exonuclease I from E. coli, exonuclease III enzyme from E. coli, RecJ from T. thermophilus and bacteriophage lambda exonuclease, TatD exonuclease and variants thereof. Three subunits comprising the RecJ sequence from T. thermophilus or a variant thereof interact to form a trimer exonuclease. The polymerase may be PyroPhage® 3173 DNA Polymerase (which is commercially available from Lucigen® Corporation), SD Polymerase (commercially available from Bioron®) or variants thereof. The enzyme may be Phi29 DNA polymerase (SEQ ID NO: 7) or a variant thereof. The topoisomerase is preferably a member of any of the Moiety Classification (EC) groups 5.99.1.2 and 5.99.1.3.

The enzyme is most preferably derived from a helicase, such as Hel308 Mbu, Hel308 Csy, Hel308 Tga, Hel308 Mhu, TraI Eco, XPD Mbu or a variant thereof. Any helicase may be used in the invention. The helicase may be or be derived from a Hel308 helicase, a RecD helicase, such as TraI helicase or a TrwC helicase, a XPD helicase or a Dda helicase. The helicase may be any of the helicases, modified helicases or helicase constructs disclosed in WO 2013/057495; WO 2013/098562; WO2013098561; WO 2014/013260; WO 2014/013259; WO 2014/013262 and WO 2015/055981. All of these are incorporated by reference in their entirety.

The kit may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotides or voltage or patch clamp apparatus. Reagents may be present in the kit in a dry state such that a fluid sample resuspends the reagents. The kit may also, optionally, comprise instructions to enable the kit to be used in the method of the invention or details regarding for which organism the method may be used. Finally, the kit may also comprise additional components useful in analyte characterization.

### Apparatus

The invention also provides an apparatus for characterising target analytes in a sample, comprising (a) a plurality of pores and (b) a plurality of helicases or a plurality of constructs of the invention. The plurality of pores may be any of those discussed above, including the pores of the invention. Preferred combinations of pores and helicases are discussed in more detail below.

The invention also provides an apparatus comprising a transmembrane protein pore or pore complex of the invention inserted into an in vitro membrane.

The invention also provides an apparatus produced by a method comprising: (i) obtaining an isolated pore or an isolated pore complex of the invention and (ii) contacting the isolated pore or isolated pore complex with an in vitro membrane such that the pore is inserted in the in vitro membrane.

Any of the specific embodiments discussed above, especially in relation to the pores, pore complexes, helicases and constructs of the invention, are equally applicable to the apparatuses of the invention.

### Membranes of the Invention

The invention also provides a membrane comprising a pore or a pore complex of the invention.

The pore is preferably present in the membrane, together forming a transmembrane pore. The membrane may comprise components of any type of membranes, such as an amphiphilic layer or a triblock copolymer membrane. The membrane may further comprise a polynucleotide binding protein or a polypeptide handling enzyme attached to the pore. The membrane may further comprise one or more anchors for coupling the polynucleotide or polypeptide to the membrane. The pore may be any of those discussed above.

### Arrays

The invention also provides an array comprising a plurality of membranes of the invention. Any of the embodiments discussed above with respect to the membrane of the invention equally apply the array of the invention. The array may be set up to perform any of the methods described below.

In a preferred embodiment, each membrane in the array comprises one pore. Due to the manner in which the array is formed, for example, the array may comprise one or more membranes that do not comprise a pore, and/or one or more membranes that comprise two or more pores. The array may comprise from about 2 to about 1000, such as from about 10 to about 800, from about 20 to about 600 or from about 30 to about 500 membranes.

### System

The invention provides a system comprising (a) a membrane of the invention or an array of the invention, (b) means for applying a potential across the membrane(s) and (c) means for detecting electrical or optical signals across the membrane(s).

The pores and membranes may be any as described above and below.

In one embodiment, the system further comprises a first chamber and a second chamber, wherein the first and second chambers are separated by the membrane(s). When used to characterise a target analyte, the system may further comprise a target analyte, wherein the target analyte is transiently located within the continuous channel and wherein one end of the target analyte is located in the first chamber and one end of the target analyte is located in the second chamber. The target analyte is preferably a target polypeptide or a target polynucleotide.

In one embodiment, the system further comprises an electrically conductive solution in contact with the pore(s), electrodes providing a voltage potential across the membrane(s), and a measurement system for measuring the current through the pore(s). In one embodiment, the voltage applied across the membranes and pore is from +5 V to −5 V, such as −600 mV to +600 mV or −400 mV to +400 mV. The voltage used is preferably in the range 100 mV to 240 mV and more preferably in the range of 120 mV to 220 mV. It is possible to increase discrimination between different amino acids or nucleotides by a pore by using an increased applied potential. Any suitable electrically conductive solution may be used. For example, the solution may comprise charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In an exemplary system, salt is present in the aqueous solution in the chamber. Potassium chloride (KCl), sodium chloride (NaCl), caesium chloride (CsCl) or a mixture of potassium ferrocyanide and potassium ferricyanide is typically used. KCl, NaCl and a mixture of potassium ferrocyanide and potassium ferricyanide are preferred. The charge carriers may be asymmetric across the membrane. For instance, the type and/or concentration of the charge carriers may be different on each side of the membrane, e.g., in each chamber.

The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of an amino acid or nucleotide to be identified against the background of normal current fluctuations.

A buffer may be present in the electrically conductive solution. Typically, the buffer is phosphate buffer. Other suitable buffers are HEPES and Tris-HCl buffer. The pH of the electrically conductive solution may be from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5. The system may be comprised in an apparatus. The apparatus may be any conventional apparatus for analyte analysis, such as an array or a chip. The apparatus is preferably set up to carry out the disclosed method. For example, the apparatus may comprise a chamber comprising an aqueous solution and a barrier that separates the chamber into two sections. The barrier typically has an aperture in which the membrane(s) containing the pore(s) are formed. Alternatively, the barrier forms the membrane in which the pore is present.

The apparatus may also comprise an electrical circuit capable of applying a potential and measuring an electrical signal across the membrane and pore.

The apparatus may be any of those described in WO 2008/102120, WO 2009/077734, WO 2010/122293, WO 2011/067559, or WO 00/28312 (all incorporated herein by reference in their entirety).

### Membrane

Any suitable membrane may be used in the system. The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e., lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units) but may also be constructed from more than two monomer sub-units to form more complex arrangements that behave as amphiphiles. The copolymer may be a triblock, tetrablock or pentablock copolymer. The membrane is preferably a triblock copolymer membrane.

The membrane is most preferably one of the membranes disclosed in International Application No. WO2014/064443 or WO2014/064444.

The amphiphilic molecules may be chemically modified or functionalised to facilitate coupling of the polynucleotide. The amphiphilic layer may be a monolayer or a bilayer. The amphiphilic layer is typically planar. The amphiphilic layer may be curved. The amphiphilic layer may be supported.

Amphiphilic membranes are typically naturally mobile, essentially acting as two-dimensional fluids with lipid diffusion rates of approximately 10⁻⁸ cm s⁻¹. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer, or a liposome. The lipid bilayer is preferably a planar lipid bilayer. Suitable lipid bilayers are disclosed in WO 2008/102121, WO 2009/077734, and WO 2006/100484 (all incorporated herein by reference in their entirety).

In another preferred embodiment, the membrane comprises a solid-state layer. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid-state layer may be formed from graphene. Suitable graphene layers are disclosed in WO 2009/035647 (incorporated herein by reference in its entirety). If the membrane comprises a solid-state layer, the pore is typically present in an amphiphilic membrane or layer contained within the solid-state layer, for instance within a hole, well, gap, channel, trench or slit within the solid-state layer. The skilled person can prepare suitable solid state/amphiphilic hybrid systems. Suitable systems are disclosed in WO 2009/020682 and WO 2012/005857 (both incorporated herein by reference in their entirety). Any of the amphiphilic membranes or layers discussed above may be used.

The method is typically carried out using (i) an artificial amphiphilic layer comprising a pore, (ii) an isolated, naturally occurring lipid bilayer comprising a pore, or (iii) a cell having a pore inserted therein. The method is typically carried out using an artificial amphiphilic layer, such as an artificial triblock copolymer layer. The layer may comprise other transmembrane and/or intramembrane proteins as well as other molecules in addition to the pore. Suitable apparatus and conditions are discussed below. The method of the invention is typically carried out in vitro.

### Preferred Combinations of Pores and Helicases

In several embodiments above, the modified helicase of the invention may be used with a pore or a transmembrane pore. In several embodiments of the invention, the isolated pore of the invention or the isolated pore complex of the invention may be used in combination with a modified helicase of the invention. The following combinations are preferred in the invention. The modified helicase of the invention may be used in combination with any of the pores described in WO2016/034591, WO2017/149316, WO2017/149317 and, WO2017/149318, WO2018/211241, and WO2019/002893 (all incorporated by reference herein in their entirety). The modified helicase of the invention is preferably used with a CsgG pore as described in WO2016/034591 (incorporated herein by reference) as in Example 2 or a CsgG:CsgF pore complex as described in WO2019/002893 (incorporated herein by reference).

The isolated pore of the invention or the isolated pore complex of the invention may be used in combination with any of the helicases disclosed in WO2015/055981, WO2015/166276 and WO2016/055777 (all incorporated herein by reference in their entirety).

The helicase of the invention is preferably used with a CsgG pore or a CsgG:CsgF pore complex of the invention and vice versa as in Example 4. The CsgG pore or a CsgG:CsgF pore complex of the invention preferably comprises Q100A and/or N102A or N102S and the helicases preferably comprises any of the substitutions in Example 4, preferably Y350I and/or K358I.

It is to be understood that although particular embodiments, specific configurations as well as materials and/or molecules, have been discussed herein for engineered cells and methods according to the present invention, various changes or modifications in form and detail may be made without departing from the scope and spirit of this invention. The following examples are provided to better illustrate particular embodiments, and they should not be considered limiting the application. The application is limited only by the claims.

### Sequences

SEQ ID NO:1 shows polynucleotide sequence of wild-type E. coli CsgG from strain K12, including signal sequence (Gene ID: 945619).
SEQ ID NO:2 shows amino acid sequence of wild-type E. coli CsgG including signal sequence (Uniprot accession number P0AEA2).
SEQ ID NO:3 shows amino acid sequence of wild-type E. coli CsgG as mature protein (Uniprot accession number P0AEA2).
SEQ ID NO:4 shows polynucleotide sequence of wild-type E. coli CsgF from strain K12, including signal sequence (Gene ID: 945622).
SEQ ID NO:5 shows amino acid sequence of wild-type E. coli CsgF including signal sequence (Uniprot accession number P0AE98).
SEQ ID NO:6 shows amino acid sequence of wild-type E. coli CsgF as mature protein (Uniprot accession number P0AE98).
SEQ ID NO:7 shows polynucleotide sequence of a fragment of wild-type E. coli CsgF encoding amino acids 1 to 27 and a C-terminal 6 His tag.
SEQ ID NO:8 shows amino acid sequence of a fragment of wild-type E. coli CsgF encompassing amino acids 1 to 27 and a C-terminal 6 His tag.
SEQ ID NO:9 shows polynucleotide sequence of a fragment of wild-type E. coli CsgF encoding amino acids 1 to 38 and a C-terminal 6 His tag.
SEQ ID NO:10 shows amino acid sequence of a fragment of wild-type E. coli CsgF encompassing amino acids 1 to 38 and a C-terminal 6 His tag.
SEQ ID NO:11 shows polynucleotide sequence of a fragment of wild-type E. coli CsgF encoding amino acids 1 to 48 and a C-terminal 6 His tag.
SEQ ID NO:12 shows amino acid sequence of a fragment of wild-type E. coli CsgF encompassing amino acids 1 to 48 and a C-terminal 6 His tag.
SEQ ID NO:13 shows polynucleotide sequence of a fragment of wild-type E. coli CsgF encoding amino acids 1 to 64 and a C-terminal 6 His tag.
SEQ ID NO:14 shows amino acid sequence of a fragment of wild-type E. coli CsgF encompassing amino acids 1 to 64 and a C-terminal 6 His tag.
SEQ ID NO:15 shows amino acid sequence of a peptide corresponding to residues 20 to 53 of E. coli CsgF
SEQ ID NO:16 shows amino acid sequence of a peptide corresponding to residues 20 to 42 of E. coli CsgF, including KD at its C-terminus
SEQ ID NO:17 shows amino acid sequence of a peptide corresponding to residues 23 to 55 of CsgF homologue Q88H88
SEQ ID NO:18 shows amino acid sequence of a peptide corresponding to residues 25 to 57 of CsgF homologue A0A143HJA0
SEQ ID NO:19 shows amino acid sequence of a peptide corresponding to residues 21 to 53 of CsgF homologue Q5E245
SEQ ID NO:20 shows amino acid sequence of a peptide corresponding to residues 19 to 51 of CsgF homologue Q084E5
SEQ ID NO:21 shows amino acid sequence of a peptide corresponding to residues 15 to 47 of CsgF homologue F0LZU2
SEQ ID NO:22 shows amino acid sequence of a peptide corresponding to residues 26 to 58 of CsgF homologue A0A136HQR0
SEQ ID NO:23 shows amino acid sequence of a peptide corresponding to residues 21 to 53 of CsgF homologue A0A0W1SRL3
SEQ ID NO:24 shows amino acid sequence of a peptide corresponding to residues 26 to 59 of CsgF homologue B0UH01
SEQ ID NO:25 shows amino acid sequence of a peptide corresponding to residues 22 to 53 of CsgF homologue Q6NAU5
SEQ ID NO:26 shows amino acid sequence of a peptide corresponding to residues 7 to 38 of CsgF homologue G8PUY5
SEQ ID NO:27 shows amino acid sequence of a peptide corresponding to residues 25 to 57 of CsgF homologue A0A0S2ETP7
SEQ ID NO:28 shows amino acid sequence of a peptide corresponding to residues 19 to 51 of CsgF homologue E3I1Z1
SEQ ID NO:29 shows amino acid sequence of a peptide corresponding to residues 24 to 55 of CsgF homologue F3Z094
SEQ ID NO:30 shows amino acid sequence of a peptide corresponding to residues 21 to 53 of CsgF homologue A0A176T7M2
SEQ ID NO:31 shows amino acid sequence of a peptide corresponding to residues 14 to 45 of CsgF homologue D2QPP8
SEQ ID NO:32 shows amino acid sequence of a peptide corresponding to residues 28 to 58 of CsgF homologue N2IYT1
SEQ ID NO:33 shows amino acid sequence of a peptide corresponding to residues 26 to 58 of CsgF homologue W7QHV5
SEQ ID NO:34 shows amino acid sequence of a peptide corresponding to residues 23 to 55 of CsgF homologue D4ZLW2
SEQ ID NO:35 shows amino acid sequence of a peptide corresponding to residues 21 to 53 of CsgF homologue D2QT92
SEQ ID NO:36 shows amino acid sequence of a peptide corresponding to residues 20 to 51 of CsgF homologue AOA167UJA2
SEQ ID NO:37 shows amino acid sequence of a fragment of wild-type E. coli CsgF encompassing amino acids 20 to 27.
SEQ ID NO:38 shows amino acid sequence of a fragment of wild-type E. coli CsgF encompassing amino acids 20 to 38.
SEQ ID NO:39: shows amino acid sequence of a fragment of wild-type E. coli CsgF encompassing amino acids 20 to 48.
SEQ ID NO:40 shows amino acid sequence of a fragment of wild-type E. coli CsgF encompassing amino acids 20 to 64.
SEQ ID NO:41 shows the nucleotide sequence of primer CsgF_d27_end
SEQ ID NO:42 shows the nucleotide sequence of primer CsgF_d38_end
SEQ ID NO:43 shows the nucleotide sequence of primer CsgF_d48_end
SEQ ID NO:44 shows the nucleotide sequence of primer CsgF_d64_end
SEQ ID NO:45 shows the nucleotide sequence of primer pNa62_CsgF_histag_Fw
SEQ ID NO:46 shows the nucleotide sequence of primer CsgF-His_pET22b_FW
SEQ ID NO:47 shows the nucleotide sequence of primer CsgF-His_pET22b_Rev
SEQ ID NO:48 shows the nucleotide sequence of primer csgEFG_pDONR221_FW
SEQ ID NO:49 shows the nucleotide sequence of primer csgEFG_pDONR221_Rev
SEQ ID NO:50 shows the nucleotide sequence of primer Mut_csgF_His_FW
SEQ ID NO:51 shows the nucleotide sequence of primer Mut_csgF_His_Rev
SEQ ID NO:52 shows the nucleotide sequence of primer DelCsgE_Rev
SEQ ID NO:53 shows the nucleotide sequence of primer DelCsgE FW
SEQ ID NO: 54 shows the amino acid sequence of residues 1 to 30 of mature E. coli CsgF
SEQ ID NO: 55 shows the amino acid sequence of residues 1 to 35 of mature E. coli CsgF
SEQ ID NO: 56 shows the amino acid sequence of a mutated (T4C/N17S) CsgF sequence with a signal sequence, and a TEV protease cleavage site (ENLYFQS) inserted between residues 35 and 36 of sequence of the mature protein.
SEQ ID NO: 57 shows the amino acid sequence of a mutated (N17S-Del) CsgF sequence with a signal sequence, and a TEV protease cleavage site (ENLYFQS) inserted between residues 35 and 36 of sequence of the mature protein.
SEQ ID NO: 58 shows the amino acid sequence of a mutated (G1C/N17S) CsgF sequence with a signal sequence, and a TEV protease cleavage site (ENLYFQS) inserted between residues 35 and 36 of sequence of the mature protein.
SEQ ID NO: 59 shows the amino acid sequence of a mutated (G1C) CsgF sequence with a signal sequence, and a TEV protease cleavage site (ENLYFQS) inserted between residues 35 and 36 of sequence of the mature protein.
SEQ ID NO: 60 shows the amino acid sequence of a CsgF sequence with a signal sequence, a TEV protease cleavage site (ENLYFQS) inserted between residues 45 and 46 of sequence of the mature protein, and a His₁₀ tag at the C-terminus.
SEQ ID NO: 61 shows the amino acid sequence of a CsgF sequence with a signal sequence, a TEV protease cleavage site (ENLYFQS) inserted between residues 35 and 36 of sequence of the mature protein, and a His₁₀ tag at the C-terminus.
SEQ ID NO: 62 shows the amino acid sequence of a CsgF sequence with a signal sequence, a TEV protease cleavage site (ENLYFQS) inserted between residues 30 and 31 of sequence of the mature protein, and a His₁₀ tag at the C-terminus.
SEQ ID NO: 63 shows the amino acid sequence of a CsgF sequence with a signal sequence, a TEV protease cleavage site (ENLYFQS) inserted between residues 45 and 51 of sequence of the mature protein, and a His₁₀ tag at the C-terminus.
SEQ ID NO: 64 shows the amino acid sequence of a CsgF sequence with a signal sequence, a TEV protease cleavage site (ENLYFQS) inserted between residues 30 and 37 of sequence of the mature protein, and a His₁₀ tag at the C-terminus.
SEQ ID NO: 65 shows the amino acid sequence of a CsgF sequence with a signal sequence, a HCV C3 protease cleavage site (LEVLFQGP) inserted between residues 34 and 36 of sequence of the mature protein, and a His₁₀ tag at the C-terminus.
SEQ ID NO: 66 shows the amino acid sequence of a CsgF sequence with a signal sequence, a HCV C3 protease cleavage site (LEVLFQGP) inserted between residues 42 and 43 of sequence of the mature protein, and a His₁₀ tag at the C-terminus.
SEQ ID NO: 67 shows the amino acid sequence of a CsgF sequence with a signal sequence, a HCV C3 protease cleavage site (LEVLFQGP) inserted between residues 38 and 47 of sequence of the mature protein, and a His₁₀ tag at the C-terminus.
SEQ ID NO: 68 shows the amino acid sequence of YP_001453594.1: 1-248 of hypothetical protein CKO_02032 [Citrobacter koseri ATCC BAA-895], which is 99% identical to SEQ ID NO: 3.
SEQ ID NO: 69 shows the amino acid sequence of WP_001787128.1: 16-238 of curli production assembly/transport component CsgG, partial [Salmonella enterica], which is 98% to SEQ ID NO: 3.
SEQ ID NO: 70 shows the amino acid sequence of KEY44978.1|: 16-277 of curli production assembly/transport protein CsgG [Citrobacter amalonaticus], which is 98% identical to SEQ ID NO: 3.
SEQ ID NO: 71 shows the amino acid sequence of YP_003364699.1: 16-277 of curli production assembly/transport component [Citrobacter rodentium ICC168], which is 97% identical to SEQ ID NO: 3.
SEQ ID NO: 72 shows the amino acid sequence of YP_004828099.1: 16-277 of curli production assembly/transport component CsgG [Enterobacter asburiae LF7a], which is 94% identical to SEQ ID NO: 3.
SEQ ID NO: 73 shows the amino acid sequence of WP_006819418.1: 19-280 of transporter [Yokenella regensburgei], which is 91% identical to SEQ ID NO: 3.
SEQ ID NO: 74 shows the amino acid sequence of WP_024556654.1: 16-277 of curli production assembly/transport protein CsgG [Cronobacter pulveris], which is 89% identical to SEQ ID NO: 3.
SEQ ID NO: 75 shows the amino acid sequence of YP_005400916.1:16-277 of curli production assembly/transport protein CsgG [Rahnella aquatilis HX2], which is 84% identical to SEQ ID NO: 3.
SEQ ID NO: 76 shows the amino acid sequence of KFC99297.1: 20-278 of CsgG family curli production assembly/transport component [Kluyvera ascorbata ATCC 33433], which is 82% identical to SEQ ID NO: 3.
SEQ ID NO: 77 shows the amino acid sequence of KFC86716.1|:16-274 of CsgG family curli production assembly/transport component [Hafnia alvei ATCC 13337], which is 81% identical to SEQ ID NO: 3.
SEQ ID NO: 78 shows the amino acid sequence of YP_007340845.1|:16-270 of uncharacterised protein involved in formation of curli polymers [Enterobacteriaceae bacterium strain FGI 57], which is 76% identical to SEQ ID NO: 3.
SEQ ID NO: 79 shows the amino acid sequence of WP_010861740.1: 17-274 of curli production assembly/transport protein CsgG [Plesiomonas shigelloides], which is 70% identical to SEQ ID NO: 3.
SEQ ID NO: 80 shows the amino acid sequence of YP_205788.1: 23-270 of curli production assembly/transport outer membrane lipoprotein component CsgG [Vibrio fischeri ES114], which is 60% identical to SEQ ID NO: 3.
SEQ ID NO: 81 shows the amino acid sequence of WP_017023479.1: 23-270 of curli production assembly protein CsgG [Aliivibrio logei], which is 59% identical to SEQ ID NO: 3.
SEQ ID NO: 82 shows the amino acid sequence of WP_007470398.1: 22-275 of Curli production assembly/transport component CsgG [Photobacterium sp. AK15], which is 57% identical to SEQ ID NO: 3.
SEQ ID NO: 83 shows the amino acid sequence of WP_021231638.1: 17-277 of curli production assembly protein CsgG [Aeromonas veronii], which is 56% identical to SEQ ID NO: 3.
SEQ ID NO: 84 shows the amino acid sequence of WP_033538267.1: 27-265 of curli production assembly/transport protein CsgG [Shewanella sp. ECSMB14101], which is 56% identical to SEQ ID NO: 3.
SEQ ID NO: 85 shows the amino acid sequence of WP_003247972.1: 30-262 of curli production assembly protein CsgG [Pseudomonas putida], which is 54% identical to SEQ ID NO: 3.
SEQ ID NO: 86 shows the amino acid sequence of YP_003557438.1: 1-234 of curli production assembly/transport component CsgG [Shewanella violacea DSS12], which is 53% identical to SEQ ID NO: 3.
SEQ ID NO: 87 shows the amino acid sequence of WP_027859066.1: 36-280 of curli production assembly/transport protein CsgG [Marinobacterium jannaschii], which is 53% identical to SEQ ID NO: 3.
SEQ ID NO: 88 shows the amino acid sequence of CEJ70222.1: 29-262 of Curli production assembly/transport component CsgG [Chryseobacterium oranimense G311], which is 50% identical to SEQ ID NO: 3.
SEQ ID NOs: 89 to 104 show the sequences in Table 4.

SEQ ID NO: 1 (>P0AEA2; coding sequence for WT CsgG from
E. coli K12)
ATGCAGCGCTTATTTCTTTTGGTTGCCGTCATGTTACTGAGCGGATGCTTAACCGCCC
CGCCTAAAGAAGCCGCCAGACCGACATTAATGCCTCGTGCTCAGAGCTACAAAGAT
TTGACCCATCTGCCAGCGCCGACGGGTAAAATCTTTGTTTCGGTATACAACATTCAG
GACGAAACCGGGCAATTTAAACCCTACCCGGCAAGTAACTTCTCCACTGCTGTTCCG
CAAAGCGCCACGGCAATGCTGGTCACGGCACTGAAAGATTCTCGCTGGTTTATACCG
CTGGAGCGCCAGGGCTTACAAAACCTGCTTAACGAGCGCAAGATTATTCGTGCGGC
ACAAGAAAACGGCACGGTTGCCATTAATAACCGAATCCCGCTGCAATCTTTAACGG
CGGCAAATATCATGGTTGAAGGTTCGATTATCGGTTATGAAAGCAACGTCAAATCTG
GCGGGGTTGGGGCAAGATATTTTGGCATCGGTGCCGACACGCAATACCAGCTCGAT
CAGATTGCCGTGAACCTGCGCGTCGTCAATGTGAGTACCGGCGAGATCCTTTCTTCG
GTGAACACCAGTAAGACGATACTTTCCTATGAAGTTCAGGCCGGGGTTTTCCGCTTT
ATTGACTACCAGCGCTTGCTTGAAGGGGAAGTGGGTTACACCTCGAACGAACCTGTT
ATGCTGTGCCTGATGTCGGCTATCGAAACAGGGGTCATTTTCCTGATTAATGATGGT
ATCGACCGTGGTCTGTGGGATTTGCAAAATAAAGCAGAACGGCAGAATGACATTCT
GGTGAAATACCGCCATATGTCGGTTCCACCGGAATCCTGA
SEQ ID NO: 2 (>P0AEA2 (1:277); WT prepro CsgG from
E. coli K12)
MQRLFLLVAVMLLSGCLTAPPKEAARPTLMPRAQSYKDLTHLPAPTGKIFVSVYNIQDE
TGQFKPYPASNFSTAVPQSATAMLVTALKDSRWFIPLERQGLQNLLNERKIIRAAQENGT
VAINNRIPLQSLTAANIMVEGSIIGYESNVKSGGVGARYFGIGADTQYQLDQIAVNLRVV
NVSTGEILSSVNTSKTILSYEVQAGVFRFIDYQRLLEGEVGYTSNEPVMLCLMSAIETGVI
FLINDGIDRGLWDLQNKAERQNDILVKYRHMSVPPES
SEQ ID NO: 3 (>P0AEA2 (16:277); mature CsgG from E. coli K12)
CLTAPPKEAARPTLMPRAQSYKDLTHLPAPTGKIFVSVYNIQDETGQFKPYPASNFSTAV
PQSATAMLVTALKDSRWFIPLERQGLQNLLNERKIIRAAQENGTVAINNRIPLQSLTAAN
IMVEGSIIGYESNVKSGGVGARYFGIGADTQYQLDQIAVNLRVVNVSTGEILSSVNTSKTI
LSYEVQAGVFRFIDYQRLLEGEVGYTSNEPVMLCLMSAIETGVIFLINDGIDRGLWDLQN
KAERQNDILVKYRHMSVPPES
SEQ ID NO: 4 (>P0AE98; coding sequence for WT CsgF from
E. coli K12)
ATGCGTGTCAAACATGCAGTAGTTCTACTCATGCTTATTTCGCCATTAAGTTGGGCT
GGAACCATGACTTTCCAGTTCCGTAATCCAAACTTTGGTGGTAACCCAAATAATGGC
GCTTTTTTATTAAATAGCGCTCAGGCCCAAAACTCTTATAAAGATCCGAGCTATAAC
GATGACTTTGGTATTGAAACACCCTCAGCGTTAGATAACTTTACTCAGGCCATCCAG
TCACAAATTTTAGGTGGGCTACTGTCGAATATTAATACCGGTAAACCGGGCCGCATG
GTGACCAACGATTATATTGTCGATATTGCCAACCGCGATGGTCAATTGCAGTTGAAC
GTGACAGATCGTAAAACCGGACAAACCTCGACCATCCAGGTTTCGGGTTTACAAAA
TAACTCAACCGATTTT
SEQ ID NO: 5 (>P0AE98 (1:138); WT pre CsgF from E. coli K12)
MRVKHAVVLLMLISPLSWAGTMTFQFRNPNFGGNPNNGAFLLNSAQAQNSYKDPSYN
DDFGIETPSALDNFTQAIQSQILGGLLSNINTGKPGRMVTNDYIVDIANRDGQLQLNVTD
RKTGQTSTIQVSGLQNNSTDF
SEQ ID NO: 6 (>P0AE98 (20:138); WT mature CsgF from E. coli
K12)
GTMTFQFRNPNFGGNPNNGAFLLNSAQAQNSYKDPSYNDDFGIETPSALDNFTQAIQSQI
LGGLLSNINTGKPGRMVTNDYIVDIANRDGQLQLNVTDRKTGQTSTIQVSGLQNNSTDF
SEQ ID NO: 7 (>P0AE98; coding sequence for CsgF 1:27_6His)
ATGCGTGTCAAACATGCAGTAGTTCTACTCATGCTTATTTCGCCATTAAGTTGGGCT
GGAACCATGACTTTCCAGTTCCGTCATCACCATCACCATCACTAAGCCC
SEQ ID NO: 8 (>P0AE98 (1:28); preprotein of CsgF 20:27_6His)
MRVKHAVVLLMLISPLSWA GTMTFQFR HHHHHH
SEQ ID NO: 9 (>P0AE98; coding sequence for CsgF 1:38_6His)
ATGCGTGTCAAACATGCAGTAGTTCTACTCATGCTTATTTCGCCATTAAGTTGGGCT
GGAACCATGACTTTCCAGTTCCGTAATCCAAACTTTGGTGGTAACCCAAATAATGGC
CATCACCATCACCATCACTAAGCCC
SEQ ID NO: 10 (>P0AE98 (1:39); preprotein of CsgF 20:38_6His)
MRVKHAVVLLMLISPLSWAGTMTFQFRNPNFGGNPNNG HHHHHH
SEQ ID NO: 11 (>P0AE98; coding sequence for CsgF 1:48_6His)
ATGCGTGTCAAACATGCAGTAGTTCTACTCATGCTTATTTCGCCATTAAGTTGGGCT
GGAACCATGACTTTCCAGTTCCGTAATCCAAACTTTGGTGGTAACCCAAATAATGGC
GCTTTTTTATTAAATAGCGCTCAGGCCCAACATCACCATCACCATCACTAAGCCC
SEQ ID NO: 12 (>P0AE98 (1:49); preprotein of CsgF 20:48_6His)
MRVKHAVVLLMLISPLSWAGTMTFQFRNPNFGGNPNNGAFLLNSAQAQ HHHHHH
SEQ ID NO: 13 (>P0AE98; coding sequence for CsgF 1:64_6His)
ATGCGTGTCAAACATGCAGTAGTTCTACTCATGCTTATTTCGCCATTAAGTTGGGCT
GGAACCATGACTTTCCAGTTCCGTAATCCAAACTTTGGTGGTAACCCAAATAATGGC
GCTTTTTTATTAAATAGCGCTCAGGCCCAAAACTCTTATAAAGATCCGAGCTATAAC
GATGACTTTGGTATTGAAACA CATCACCATCACCATCACTAAGCCC
SEQ ID NO: 14 (>P0AE98 (1:65); preprotein of CsgF 20:64_6His)
MRVKHAVVLLMLISPLSWAGTMTFQFRNPNFGGNPNNGAFLLNSAQAQNSYKDPSYN
DDFGIETHHHHHH
SEQ ID NO: 15 (>P0AE98 (20:53); mature peptide of CsgF 20:53)
GTMTFQFRNPNFGGNPNNGAFLLNSAQAQNSYKD
SEQ ID NO: 16 (>P0AE98 (20:42); mature peptide of CsgF
20:42 + KD)
GTMTFQFRNPNFGGNPNNGAFLLKD
SEQ ID NO: 17 (>Q88H88_PSEPK (23:55))
TELVYTPVNPAFGGNPLNGTWLLNNAQAQNDY
SEQ ID NO: 18 (>A0A143HJA0_9GAMM (25:57))
TELIYEPVNPNFGGNPLNGSYLLNNAQAQDRH
SEQ ID NO: 19 (>Q5E245_VIBF1 (21:53))
SELVYTPVNPNFGGNPLNTSHLFGGANAINDY
SEQ ID NO: 20 (>Q084E5_SHEFN (19:51))
TQLVYTPVNPAFGGSYLNGSYLLANASAQNEH
SEQ ID NO: 21 (>F0LZU2_VIBFN (15:47))
SSLVYEPVNPTFGGNPLNTTHLFSRAEAINDY
SEQ ID NO: 22 (>A0A136HQR0_9ALTE (26:58))
TELVYEPINPSFGGNPLNGSFLLSKANSQNAH
SEQ ID NO: 23 (>A0A0W1SRL3_9GAMM (21:53))
TEIVYQPINPSFGGNPMNGSFLLQKAQSQNAH
SEQ ID NO: 24 (>B0UH01_METS4 (26:59))
SSLVYQPVNPAFGGPQLNGSWLQAEANAQNIPQ
SEQ ID NO: 25 (>Q6NAU5_RHOPA (22:53))
GSLVYTPTNPAFGGSPLNGSWQMQQATAGNH
SEQ ID NO: 26 (>G8PUY5_PSEUV (7:38))
QQLIYQPTNPSFGGYAANTTHLFATANAQKTA
SEQ ID NO: 27 (>A0A0S2ETP7_9RHIZ (25:57))
GDLVYTPVNPSFGGSPLNSAHLLSIAGAQKNA
SEQ ID NO: 28 (>E3I1Z1_RHOVT (19:51))
AELGYTPVNPSFGGSPLNGSTLLSEASAQKPN
SEQ ID NO: 29 (>F3Z094_DESAF (24:55))
TELVFSFTNPSFGGDPMIGNFLLNKADSQKR
SEQ ID NO: 30 (>A0A176T7M2_9FLAO (21:53))
QQLVYKSINPFFGGGDSFAYQQLLASANAQND
SEQ ID NO: 31 (>D2QPP8_SPILD (14:45))
QALVYHPNNPAFGGNTFNYQWMLSSAQAQDR
SEQ ID NO: 32 (>N2IYT1_9PSED (26:58))
TELVYTPKNPAFGGSPLNGSYLLGNAQAQNDY
SEQ ID NO: 33 (>W7QHV5_9GAMM (26:58))
GQLIYQPINPSFGGDPLLGNHLLNKAQAQDTK
SEQ ID NO: 34 (>D4ZLW2_SHEVD (23:55))
TQLIYTPVNPNFGGSYLNGSYLLANASVQNDH
SEQ ID NO: 35 (>D2QT92_SPILD (21:53))
QAFVYHPNNPNFGGNTFNYSWMLSSAQAQDRT
SEQ ID NO: 36 (>A0A167UJA2_9FLAO (20:51))
QGLIYKPKNPAFGGDTFNYQWLASSAESQNK
SEQ ID NO: 37 (>P0AE98 (20:28); mature peptide of CsgF 20:27)
GTMTFQFR
SEQ ID NO: 38 (>P0AE98 (20:39); mature peptide of CsgF 20:38)
GTMTFQFRNPNFGGNPNNG
SEQ ID NO: 39 (>P0AE98 (20:49); mature peptide of CsgF 20:48)
GTMTFQFRNPNFGGNPNNGAFLLNSAQAQ
SEQ ID NO: 40 (>P0AE98 (20:65); mature peptide of CsgF 20:64)
GTMTFQFRNPNFGGNPNNGAFLLNSAQAQNSYKDPSYNDDFGIET
SEQ ID NO: 41 (CsgF_d27_end)
ACGGAACTGGAAAGTCATGGTTCC
SEQ ID NO: 42 (CsgF_d38_end)
GCCATTATTTGGGTTACCACCAAAGTTTGG
SEQ ID NO: 43 (CsgF_d48_end)
TTGGGCCTGAGCGCTATTTAATAAAAAAGC
SEQ ID NO: 44 (CsgF_d64_end)
TGTTTCAATACCAAAGTCATCGTTATAGCTCGG
SEQ ID NO: 45 (pNa62_CsgF_histag_Fw)
CATCACCATCACCATCACTAAGCCC
SEQ ID NO: 46 (CsgF-His_pET22b_FW)
cccccatatgGGAACCATGACTTTCCAGTTCC
SEQ ID NO: 47: (CsgF-His_pET22b_Rev)
ccccGAATTCCTAatggtgatggtgatggtgGTAAAAATCGGTTGAGTTATTTTG
SEQ ID NO: 48: (csgEFG_pDONR221_FW)
GGGGACAAGTTTGTACAAAAAAGCAGGCTACCTCAGGCGATAAAGCCATGAAACGT
TA
SEQ ID NO: 49: (csgEFG_pDONR221_Rev)
GGGGACCACTTTGTACAAGAAAGCTGGGTGTTTAAACTCATTTTTCGAACTGCGGGT
GGCTCCAAGCGCTGG
SEQ ID NO: 50: (Mut_csgF_His_FW)
CAAAATAACTCAACCGATTTTcatcaccatcaccatcacTAAGCCCCAGCTTCATAAGG
SEQ ID NO: 51: (Mut_csgF_His_Rev)
CCTTATGAAGCTGGGGCTTAgtgatggtgatggtgatgAAAATCGGTTGAGTTATTTTG
SEQ ID NO: 52: (DelCsgE_Rev)
AGCCTGCTTTTTTGTACAAAC
SEQ ID NO: 53: (DelCsgE FW)
ATAAAAAATTGTTCGGAGGCTGC
SEQ ID NO: 54 (>P0AE98 (20:50); mature peptide of CsgF 1:30)
GTMTFQFRNPNFGGNPNNGAFLLNSAQAQN
SEQ ID NO: 55 (>P0AE98 (20:54); mature peptide of CsgF 1:35)
GTMTFQFRNPNFGGNPNNGAFLLNSAQAQNSYKDP

Examples of CsgF sequences with protease cleavage sites made into proteins. Signal peptide is shown in bold TEV protease cleavage site in bold and underline and HCV C3 protease cleavage site in underline. StrepII indicate the Strep tag at the C terminus, H10 indicates the 10×Histidine tag at the C terminus and ** indicates STOP codons.

SEQ ID NO: 56 Pro-CsgF-Eco-(WT-T4C/N17S/P35-TEV-S36)-StrepII
MRVKHAVVLLMLISPLSWAGTMCFQFRNPNFGGNPSNGAFLLNSAQAQNSYKDPENL
YFQSSYNDDFGIETPSALDNFTQAIQSQILGGLLSNINTGKPGRMVTNDYIVDIANRDGQ
LQLNVTDRKTGQTSTIQVSGLQNNSTDFSAWSHPQFEK**
SEQ ID NO: 57 Pro-CsgF-Eco-(WT-N17S-Del(P35-[TEV]-S36)-StrepII
MRVKHAVVLLMLISPLSWAGTMTFQFRNPNFGGNPSNGAFLLNSAQAQNSYKDPENL
YFQSSYNDDFGIETPSALDNFTQAIQSQILGGLLSNINTGKPGRMVTNDYIVDIANRDGQ
LQLNVTDRKTGQTSTIQVSGLQNNSTDFSAWSHPQFEK**
SEQ ID NO: 58 Pro-CsgF-Eco-(WT-G1C/N17S/P35-[TEV]-S36)-StrepII
MRVKHAVVLLMLISPLSWACTMTFQFRNPNFGGNPSNGAFLLNSAQAQNSYKDPENL
YFQSSYNDDFGIETPSALDNFTQAIQSQILGGLLSNINTGKPGRMVTNDYIVDIANRDGQ
LQLNVTDRKTGQTSTIQVSGLQNNSTDFSAWSHPQFEK**
SEQ ID NO: 59 Pro-CsgF-Eco-(WT-G1C/P35-[TEV]-S36)-StrepII
MRVKHAVVLLMLISPLSWACTMTFQFRNPNFGGNPNNGAFLLNSAQAQNSYKDPEN
LYFQSSYNDDFGIETPSALDNFTQAIQSQILGGLLSNINTGKPGRMVTNDYIVDIANRDG
QLQLNVTDRKTGQTSTIQVSGLQNNSTDFSAWSHPQFEK**
SEQ ID NO: 60 Pro-CsgF-Eco-(WT-T45-TEV-P46)-H10
MRVKHAVVLLMLISPLSWAGTMTFQFRNPNFGGNPNNGAFLLNSAQAQNSYKDPSY
NDDFGIETENLYFQSPSALDNFTQAIQSQILGGLLSNINTGKPGRMVTNDYIVDIANRDG
QLQLNVTDRKTGQTSTIQVSGLQNNSTDFHHHHHHHHHH**
SEQ ID NO: 61 Pro-CsgF-Eco-(WT-P35-TEV-S36)-H10
MRVKHAVVLLMLISPLSWAGTMTFQFRNPNFGGNPNNGAFLLNSAQAQNSYKDPEN
LYFQSSYNDDFGIETPSALDNFTQAIQSQILGGLLSNINTGKPGRMVTNDYIVDIANRDG
QLQLNVTDRKTGQTSTIQVSGLQNNSTDFHHHHHHHHHH**
SEQ ID NO: 62 Pro-CsgF-Eco-(WT-N30-TEV-S31)-H10
MRVKHAVVLLMLISPLSWAGTMTFQFRNPNFGGNPNNGAFLLNSAQAQNENLYFQS
SYKDPSYNDDFGIETPSALDNFTQAIQSQILGGLLSNINTGKPGRMVTNDYIVDIANRDG
QLQLNVTDRKTGQTSTIQVSGLQNNSTDFHHHHHHHHHH**
SEQ ID NO: 63 Pro-CsgF-Eco-(WT-T45-TEV-F51)-H10
MRVKHAVVLLMLISPLSWAGTMTFQFRNPNFGGNPNNGAFLLNSAQAQNSYKDPSY
NDDFGIETENLYFQSFTQAIQSQILGGLLSNINTGKPGRMVTNDYIVDIANRDGQLQLNV
TDRKTGQTSTIQVSGLQNNSTDFHHHHHHHHHH**
SEQ ID NO: 64 Pro-CsgF-Eco-(WT-N30-TEV-Y37)-H10
MRVKHAVVLLMLISPLSWAGTMTFQFRNPNFGGNPNNGAFLLNSAQAQNENLYFQS
YNDDFGIETPSALDNFTQAIQSQILGGLLSNINTGKPGRMVTNDYIVDIANRDGQLQLNV
TDRKTGQTSTIQVSGLQNNSTDFHHHHHHHHHH**
SEQ ID NO: 65 Pro-CsgF-Eco-(WT-D34-[C3]-S36)
MRVKHAVVLLMLISPLSWACTMTFQFRNPNFGGNPNNGAFLLNSAQAQNSYKDLEVL
FQGPSYNDDFGIETPSALDNFTQAIQSQILGGLLSNINTGKPGRMVTNDYIVDIANRDGQ
LQLNVTDRKTGQTSTIQVSGLQNNSTDFSAWSHPQFEK**
SEQ ID NO: 66 Pro-CsgF-Eco-(WT-I42-[C3]-E43)
MRVKHAVVLLMLISPLSWACTMTFQFRNPNFGGNPNNGAFLLNSAQAQNSYKDPSYN
DDFGILEVLFQGPETPSALDNFTQAIQSQILGGLLSNINTGKPGRMVTNDYIVDIANRDGQ
LQLNVTDRKTGQTSTIQVSGLQNNSTDFSAWSHPQFEK**
SEQ ID NO: 67 Pro-CsgF-Eco-(WT-N38-[C3]-S47)
MRVKHAVVLLMLISPLSWACTMTFQFRNPNFGGNPNNGAFLLNSAQAQNSYKDPSYN
LEVLFQGPSALDNFTQAIQSQILGGLLSNINTGKPGRMVTNDYIVDIANRDGQLQLNVTD
RKTGQTSTIQVSGLQNNSTDFSAWSHPQFEK**
SEQ ID NO: 68
MPRAQSYKDLTHLPMPTGKIFVSVYNIQDETGQFKPYPASNFSTAVPQSATAMLVTALK
DSRWFIPLERQGLQNLLNERKIIRAAQENGTVAINNRIPLQSLTAANIMVEGSIIGYESNV
KSGGVGARYFGIGADTQYQLDQIAVNLRVVNVSTGEILSSVNTSKTILSYEVQAGVFRFI
DYQRLLEGEIGYTSNEPVMLCLMSAIETGVIFLINDGIDRGLWDLQNKAERQNDILVKYR
HMSVPPES
SEQ ID NO: 69
CLTAPPKQAAKPTLMPRAQSYKDLTHLPAPTGKIFVSVYNIQDETGQFKPYPASNFSTAV
PQSATAMLVTALKDSRWFIPLERQGLQNLLNERKIIRAAQENGTVAMNNRIPLQSLTAA
NIMVEGSIIGYESNVKSGGVGARYFGIGADTQYQLDQIAVNLRVVNVSTGEILSSVNTSK
TILSYEVQAGVFRFIDYQRLLEGEIGYTSNEPVMLCLMSAIETG
SEQ ID NO: 70
CLTAPPKEAAKPTLMPRAQSYKDLTHLPIPTGKIFVSVYNIQDETGQFKPYPASNFSTAVP
QSATAMLVTALKDSRWFVPLERQGLQNLLNERKIIRAAQENGTVAINNRIPLQSLTAANI
MVEGSIIGYESNVKSGGVGARYFGIGADTQYQLDQIAVNLRVVNVSTGEILSSVNTSKTI
LSYEVQAGVFRFIDYQRLLEGEIGYTSNEPVMLCLMSAIETGVIFLINDGIDRGLWDLQN
KADRQNDILVKYRHMSVPPES
SEQ ID NO: 71
CLTTPPKEAAKPTLMPRAQSYKDLTHLPVPTGKIFVSVYNIQDETGQFKPYPASNFSTAV
PQSATAMLVTALKDSRWFIPLERQGLQNLLNERKIIRAAQENGTVAINNRIPLPSLTAANI
MVEGSIIGYESNVKSGGAGARYFGIGADTQYQLDQIAVNLRVVNVSTGEILSSVNTSKTI
LSYEVQAGVFRFIDYQRLLEGEIGYTSNEPVMLCLMSAIETGVIFLINDGIDRGLWDLQN
KADRQNDILVKYRQMSVPPES
SEQ ID NO: 72
CLTAPPKEAAKPTLMPRAQSYRDLTHLPAPTGKIFVSVYNIQDETGQFKPYPASNFSTAV
PQSATAMLVTALKDSHWFIPLERQGLQNLLNERKIIRAAQENGTVANNNRMPLQSLAA
ANVMIEGSIIGYESNVKSGGVGARYFGIGADTQYQLDQIAVNLRVVNVSTGEVLSSVNT
SKTILSYEVQAGVFRFIDYQRLLEGEIGYTSNEPVMMCLMSAIETGVIFLINDGIDRGLWD
LONKADAQNPVLVKYRDMSVPPES
SEQ ID NO: 73
CLTAPPKEAAKPTLMPRAQSYRDLTHLPLPSGKVFVSVYNIQDETGQFKPYPASNFSTAV
PQSATAMLVTALKDSRWFVPLERQGLQNLLNERKIIRAAQENGTVADNNRIPLQSLTAA
NVMIEGSIIGYESNVKSGGVGARYFGIGADTQYQLDQIAVNLRVVNVSTGEVLSSVNTS
KTILSYEVQAGVFRFVDYQRLLEGEIGYTSNEPVMLCLMSAIETGVIYLINDGIERGLWD
LQQKADVDNPILARYRNMSAPPES
SEQ ID NO: 74
CLTAPPKEAAKPTLMPRAQSYRDLTNLPDPKGKLFVSVYNIQDETGQFKPYPASNFSTA
VPQSATSMLVTALKDSRWFIPLERQGLQNLLNERKIIRAAQENGTVAENNRMPLQSLVA
ANVMIEGSIIGYESNVKSGGVGARYFGIGGDTQYQLDQIAVNLRVVNVSTGEVLSSVNT
SKTILSYEVQAGVFRFIDYQRLLEGEIGYTANEPVMLCLMSAIETGVIHLINDGINRGLWE
LKNKGDAKNTILAKYRSMAVPPES
SEQ ID NO: 75
CLTAAPKEAARPTLLPRAPSYTDLTHLPSPQGRIFVSVYNIQDETGQFKPYPACNFSTAVP
QSATAMLVSALKDSKWFIPLERQGLQNLLNERKIIRAAQENGSVAINNQRPLSSLVAANI
LIEGSIIGYESNVKSGGVGARYFGIGASTQYQLDQIAVNLRAVDVNTGEVLSSVNTSKTIL
SYEVQAGVFRFIDYQRLLEGELGYTTNEPVMLCLMSAIESGVIYLVNDGIERNLWQLQN
PSEINSPILQRYKNNIVPAES
SEQ ID NO: 76
CITSPPKQAAKPTLLPRSQSYQDLTHLPEPQGRLFVSVYNISDETGQFKPYPASNFSTSVP
QSATAMLVSALKDSNWFIPLERQGLQNLLNERKIIRAAQENGTVAVNNRTQLPSLVAAN
ILIEGSIIGYESNVKSGGAGARYFGIGASTQYQLDQIAVNLRVVNVSTGEVLSSVNTSKTI
LSYEFQAGVFRYIDYQRLLEGEVGYTVNEPVMLCLMSAIETGVIYLVNDGISRNLWQLK
NASDINSPVLEKYKSIIVP
SEQ ID NO: 77
CLTAPPKQAAKPTLMPRAQSYQDLTHLPEPAGKLFVSVYNIQDETGQFKPYPASNFSTA
VPQSATAMLVSALKDSGWFIPLERQGLQNLLNERKIIRAAQENGTAAVNNQHQLSSLVA
ANVLVEGSIIGYESNVKSGGAGARFFGIGASTQYQLDQIAVNLRVVDVNTGQVLSSVNT
SKTILSYEVQAGVFRYIDYQRLLEGEIGYTTNEPVMLCVMSAIETGVIYLVNDGINRNLW
TLKNPQDAKSSVLERYKSTIVP
SEQ ID NO: 78
CITTPPQEAAKPTLLPRDATYKDLVSLPQPRGKIYVAVYNIQDETGQFQPYPASNFSTSVP
QSATAMLVSSLKDSRWFVPLERQGLNNLLNERKIIRAAQQNGTVGDNNASPLPSLYSAN
VIVEGSIIGYASNVKTGGFGARYFGIGGSTQYQLDQVAVNLRIVNVHTGEVLSSVNTSKT
ILSYEIQAGVFRFIDYQRLLEGEAGFTTNEPVMTCLMSAIEEGVIHLINDGINKKLWALSN
AADINSEVLTRYRK
SEQ ID NO: 79
ITEVPKEAAKPTLMPRASTYKDLVALPKPNGKIIVSVYSVQDETGQFKPLPASNFSTAVP
QSGNAMLTSALKDSGWFVPLEREGLQNLLNERKIIRAAQENGTVAANNQQPLPSLLSAN
VVIEGAIIGYDSDIKTGGAGARYFGIGADGKYRVDQVAVNLRAVDVRTGEVLLSVNTSK
TILSSELSAGVFRFIEYQRLLELEAGYTTNEPVMMCMMSALEAGVAHLIVEGIRQNLWS
LQNPSDINNPIIQRYMKEDVP
SEQ ID NO: 80
PETSESPTLMQRGANYIDLISLPKPQGKIFVSVYDFRDQTGQYKPQPNSNFSTAVPQGGT
ALLTMALLDSEWFYPLERQGLQNLLTERKIIRAAQKKQESISNHGSTLPSLLSANVMIEG
GIVAYDSNIKTGGAGARYLGIGGSGQYRADQVTVNIRAVDVRSGKILTSVTTSKTILSYE
VSAGAFRFVDYKELLEVELGYTNNEPVNIALMSAIDSAVIHLIVKGVQQGLWRPANLDT
RNNPIFKKY
SEQ ID NO: 81
PDASESPTLMQRGATYLDLISLPKPQGKIYVSVYDFRDQTGQYKPQPNSNFSTAVPQGG
TALLTMALLDSEWFYPLERQGLQNLLTERKIIRAAQKKQESISNHGSTLPSLLSANVMIE
GGIVAYDSNIKTGGAGARYLGIGGSGQYRADQVTVNIRAVDVRSGKILTSVTTSKTILSY
ELSAGAFRFVDYKELLEVELGYTNNEPVNIALMSAIDSAVIHLIVKGIEEGLWRPENQNG
KENPIFRKY
SEQ ID NO: 82
PETSKEPTLMARGTAYQDLVSLPLPKGKVYVSVYDFRDQTGQYKPQPNSNFSTAVPQG
GAALLTTALLDSRWFMPLEREGLQNLLTERKIIRAAQKKDEIPTNHGVHLPSLASANIMV
EGGIVAYDTNIQTGGAGARYLGVGASGQYRTDQVTVNIRAVDVRTGRILLSVTTSKTIL
SKELQTGVFKFVDYKDLLEAELGYTTNEPVNLAVMSAIDAAVVHVIVDGIKTGLWEPL
RGEDLQHPIIQEYMNRSKP
SEQ ID NO: 83
CATHIGSPVADEKATLMPRSVSYKELISLPKPKGKIVAAVYDFRDQTGQYLPAPASNFST
AVTQGGVAMLSTALWDSQWFVPLEREGLQNLLTERKIVRAAQNKPNVPGNNANQLPS
LVAANILIEGGIVAYDSNVRTGGAGAKYFGIGASGEYRVDQVTVNLRAVDIRSGRILNS
VTTSKTVMSQQVQAGVFRFVEYKRLLEAEAGFSTNEPVQMCVMSAIESGVIRLIANGVR
DNLWQLADQRDIDNPILQEYLQDNAP
SEQ ID NO: 84
ASSSLMPKGESYYDLINLPAPQGVMLAAVYDFRDQTGQYKPIPSSNFSTAVPQSGTAFL
AQALNDSSWFIPVEREGLQNLLTERKIVRAGLKGDANKLPQLNSAQILMEGGIVAYDTN
VRTGGAGARYLGIGAATQFRVDTVTVNLRAVDIRTGRLLSSVTTTKSILSKEITAGVFKFI
DAQELLESELGYTSNEPVSLCVASAIESAVVHMIADGIWKGAWNLADQASGLRSPVLQ
KY
SEQ ID NO: 85
QDSETPTLTPRASTYYDLINMPRPKGRLMAVVYGFRDQTGQYKPTPASSFSTSVTQGAA
SMLMDALSASGWFVVLEREGLQNLLTERKIIRASQKKPDVAENIMGELPPLQAANLMLE
GGIIAYDTNVRSGGEGARYLGIDISREYRVDQVTVNLRAVDVRTGQVLANVMTSKTIYS
VGRSAGVFKFIEFKKLLEAEVGYTTNEPAQLCVLSAIESAVGHLLAQGIEQRLWQV
SEQ ID NO: 86
MPKSDTYYDLIGLPHPQGSMLAAVYDFRDQTGQYKAIPSSNFSTAVPQSGTAFLAQALN
DSSWFVPVEREGLQNLLTERKIVRAGLKGEANQLPQLSSAQILMEGGIVAYDTNIKTGG
AGARYLGIGVNSKFRVDTVTVNLRAVDIRTGRLLSSVTTTKSILSKEVSAGVFKFIDAQD
LLESELGYTSNEPVSLCVAQAIESAVVHMIADGIWKRAWNLADTASGLNNPVLQKY
SEQ ID NO: 87
LTRRMSTYQDLIDMPAPRGKIVTAVYSFRDQSGQYKPAPSSSFSTAVTQGAAAMLVNVL
NDSGWFIPLEREGLQNILTERKIIRAALKKDNVPVNNSAGLPSLLAANIMLEGGIVGYDS
NIHTGGAGARYFGIGASEKYRVDEVTVNLRAIDIRTGRILHSVLTSKKILSREIRSDVYRFI
EFKHLLEMEAGITTNDPAQLCVLSAIESAVAHLIVDGVIKKSWSLADPNELNSPVIQAYQ
QQRI
SEQ ID NO: 88
PSDPERSTMGELTPSTAELRNLPLPNEKIVIGVYKFRDQTGQYKPSENGNNWSTAVPQGT
TTILIKALEDSRWFIPIERENIANLLNERQIIRSTRQEYMKDADKNSQSLPPLLYAGILLEG
GVISYDSNTMTGGFGARYFGIGASTQYRQDRITIYLRAVSTLNGEILKTVYTSKTILSTSV
NGSFFRYIDTERLLEAEVGLTQNEPVQLAVTEAIEKAVRSLIIEGTRDKIW
SEQ ID NOs: 89 to 116 are CsgF peptides shown in Table 3.
SEQ ID NO: 117 is SEQ ID NO: 3 with a W at position 97.
CLTAPPKEAARPTLMPRAQSYKDLTHLPAPTGKIFVSVYNIQDETGQFKPYPASNFSTAV
PQSATAMLVTALKDSRWFIPLERQGLQNLLNERKIIWAAQENGTVAINNRIPLQSLTAAN
IMVEGSIIGYESNVKSGGVGARYFGIGADTQYQLDQIAVNLRVVNVSTGEILSSVNTSKTI
LSYEVQAGVFRFIDYQRLLEGEVGYTSNEPVMLCLMSAIETGVIFLINDGIDRGLWDLQN
KAERQNDILVKYRHMSVPPES
SEQ ID NO: 118 (>P32270; Enterobacteria phage T4)
MTFDDLTEGQKNAFNIVMKAIKEKKHHVTINGPAGTGKTTLTKFIIEALISTGGTGIILA
APTHAAKKILSKLSGKEASTIHSILKINPVTYEENVLFEQKEVPDLAKCRVLICDEVSMY
DRKLFKILLSTIPPWCTIIGIGDNKQIRPVEPGENTAYISPFFTHKDFYQCELTEVKRSN
APIIDVATDVRNGKWNYDKVVDGHGVRGFTGDTALRDFMVNYFSIVKSLDDLFENRVMAF
TNKSVDKLNSIIRKKIFETDKDFIVGEIIVMQEPLFKTYKIDGKPVSEIIFNNGQLVRII
EAEYTSTFVKARGVPGEYLIRHWDLTVETYGDDEYYREKIKIISSDEELYKFNLFLAKTA
ETYKNWNKGGKAPWSDFWDAKSQFSKVKALPASTFHKAQGMSVDRAFIYTPCIHYADVEL
AQQLLYVGVTRGRYDVFYV
SEQ ID NO: 119 (>D0MKQ2; Rhodothermus marinus)
MEELSNEQQRVLDHVLAWLERNDAPPIFILTGSAGTGKTLLIRHLVRALQDRRIHYALAA
PTGRAARILSERTGDHARTLHSLIYIFDRYQLVEEADRQTDEPLSLQLHFALRSAEHDAR
LIIVDEASMVSDTAGEEELYRFGSGRLLNDLLTFARLIPKRDRPPTTRLLFVGDPAQLPP
VGQSVSPALSAQYLRDTFGLSAETAHLRSVYRQRKGHPILETATALRNALEKGHYHTFRL
PEQPPDLRPVGLEEAIETTATDFRRQNPSVLLCRTNALARKLNAAVRARLWGREGLPPQP
GDLLLVNRNAPLHGLFNGDLVLVETVGPLEHRRVGRRGRPPVDLYFRDVELLYPHEKPRN
RIRCKLLENLLESPDGQLSPDIIQALLIDFYRRHPSLKHGSSEFRLMLANDAYFNALHVR
YGYAMTVHKAQGGEWKRATVVFNDWRHFRHAEFFRWAYTAITRAREELLTIGAPSFEALS
DMRWQPAPSVPAPEQAAENATRFPLKALETYHQRLSEALTAAGIETTGVELLQYAVRYHL
ARADRTTRIQYYYRGDGQISRIVTLGGADDPELTQQAYALFERILSEPPADSGELPENPL
LREFLERAHLRLEGSGIRIVHWKEMPYALRLYFSADGENVTIDFYYNRRGVWTHAQEVGR
SSSGALFARIQSLLQADS
SEQ ID NO: 120 (>B1X365; Cyanothece ATCC51142)
MSQSVVVPDELGEIITAVIEFYQDAVDKIEPKIVFLELRKNVVDWVSRTQLKIEEKEIQA
TGLTRQQQTAYKEMINFIENSSEQYFRLSGYAGTGKSFLMAKVIEWLKQEDYKYSVAAPT
NKAAKNLTQIARSQGIKIEATTVAKLLKLQPTIDVDTGQQSFEFNSEKELELKDYDVIII
DEYSMLNKDNFRDLQQAVKGGESKFIFVGDSSQLPPVKEKEPIVANHPDIRKSANLTQIV
RYDGEIVKVAESIRRNPRWNHQTYPFETVADGTIIKLNTEDWLQQALSHFEKEDWLSNPD
YVRMITWRNKTADKYNQAIREALYGENVEQLVVGDRLIAKKPVFRSLPGGKKKEKKIILN
NSEECKVIETPKINYNEKYKWEFYQVKVRTDEGGMIELRILTSESEEKRQKKLKELAKRA
REEENYSEKKKQWAIYYELDELFDNMAYAYALTCHKAQGSSIDNVFLLVSDMHYCRDKTK
MIYTGLTRAKKCCYVG
SEQ ID NO: 121 (>Q2S429; Salinibacter ruber)
MSTFADAPFTEDQEEAYDHVYDRLAQGERFTGLRGYAGTGKTYLVSRLVEQLLDEDCTVT
VCAPTHKAVQVLSDELGDAPVQMQTLHSFLGLRLQPKQDGEYELVAEEERNFAEGVVIVD
EASMIGREEWSHIQDAPFWVQWLFVGDPAQLPPVNEDPSPALDVPGPTLETIHRQAADNP
ILELATKIRTGADGRFGSTFEDGKGVAVTRNREEFLDSILRAFDADAFAEDATHARVLAY
RNKTVRRYNREIRAERYGADADRFVEGEWLVGTETWYYDGVQRLTNSEEVRVKKAQVETF
EADDQSEWTVWELKIRTPGRGLTRTIHVLHEEERERYENALERRRGKAEDDPSKWDRFFE
LRERFARVDYAYATTVHRAQGSTYDTVFVDHRDLRVCRGEERGALLYVAVTRPSRRLALL
V
SEQ ID NO: 122 (>B6BJ43 (UPI000183B2F5); Sullfurimonas
gotlandica GD1)
MKILNKETYKLSLHQEEVFTQIVSQLDTKVSSILKSTNIEDYLLSLTGPAGTGKTFLTTQ
IAKYLVEKRKESEYPMSSDFDFTITAPTHKAVGVLSKLLRENNIQSSCKTIHSFLGIKPF
IDYTTGEEKFVVDKTNKRKDRTSILIVDESSMIGNTLYEYILEAIEDKRVNVVLFIGDPY
QLLPIENSKNEIYDLPNRFFLSEVVRQAENSYIIRVATKLRERIKNQDFISLQQFFQENM
EDEITFFHNKEAFLEDFYKEEEWYKENKILATYKNKDVDAFNKIIRNKFWEQKGNTTPST
LLAGDMIRFKDAYTVGDITIYHNGQELQLGSTEVKYHDSLHIEYWECKSIYALEQQVFRV
VNPDSEAVFNQKLQSLATKAKQAKFPDNKKLWKLYYETRNMFANVQYIHASTIHKLQGST
YDVSYIDIFSLVHNHYMSDEEKYRLLYVAITRASKDIKIFMSAFDRTSDEKVIINNQNSE
TMNTLKQLHDIDIILKDLDL
SEQ ID NO: 123 (>M4MBC3; Vibrio phage henriette 12B8)
MADFELTLGQKTVLGEVISTILKPVNLNDTSRFHTMHGPAGSGKTTVLQRIISQIPAYKT
IGFCSPTHKSVKVIRRMAREAGISHRVDIRTIHSALGLVMKPVRGDEVLVKEPFAEERIY
DVLIIDEAGMLNDELIMYILESQSSKVIFVGDMCQIGPIQSNLPEEDGYTPTSTDDVSKV
FTEVEMMSALTEVVRQAEGSPIIQLATEFRLAQDDIYADLPRIVTNTTPDGNGIITMPNG
NWVDSAVARFQSDQFKEDPDHCRIVCYTNAMVDLCNDLVRKRLFGADVPEWLEDEILVAQ
EMGSTWNNADELRIVSIDDHFDQQYEVPCWRMQLESVEDHKLHNALVVKGDYIEDFKFRL
NAIAERANTDKNMSGMHWKEFWGMRKKFNTFKNVYAGTAHKSQGSTFDYTYVFTPDFYKF
GATMTIKRLLYTAITRSRYTTYFAMNTGAQ
SEQ ID NO: 124 (>I6XGX8; Vibrio phage phi-pp2)
MGLTNCQQGAMDAFLESDGHMTISGPAGSGKTFLMKSILEALESKGKNVTMVTPTHQAKN
VLHKATGQEVSTIHSLLKIHPDTYEDQKHFTQSGEVEGLDEIDVLVVEEASMVDEELFQI
TGRTMPRKCRILAVGDKYQLQPVKHDPGVISPFFTKFTTFEMNEVVRQAKDNPLIQVATE
VRNGQWLRTNWSKERRQGVLHVPNVNKMLDTYLSKVNSPEDLLDYRILAYTNDCVDTFNG
IIREHVYNTSEPFIPGEYLVTQMPVMVSNGKYPVCVIENGEVVKILDVRQKTIDGMLPKV
DNEAFDVAVLTVEKEDGNVYEFTVLWDDLQKERFARYLSVAAGTYKSMRGNTKRYWRAFW
GLKEQMIETKSLGASTVHKSQGTTVKGVCLYTQDMGYAEPEILQQLVYVGLTRPTDWALY
N
SEQ ID NO: 125 (>E5DRP6; Aeromonas phage 65)
MSESEITLTPSQNMAVNEVKNGTGHITISGPPGSGKTFLVKYLIKMLGDELGTVLAAPTH
QAKIVLTEMSGIEACTIHSLMKIHPETLEDIQIFDQSKLPDLSNIRYLIVEEASMHSKTL
FKITMKSIPPTCRIIAIGDKDQIQPEEHAQGELSPYFTDPRFSQIRLTDIMRQSLDNPII
QVATKIREGGWIEPNWNRDTKTGVYKVSGITDLVNSYLRAVKTPEDLTKYRFLAYTNKVV
NKVNSIVREHVYKTKLPFIEGEKIVLQEPVMVEHEDDTIETIFTNGEVVTINEIEVFDRT
IRIDGSPEFKVNAAKLSVSSDYSGIEHDFCVLYGSESRLEFEYQLSESAGNIKQMGKGGN
QRSAWKSFWAAKKMFIETKSLGASTIHKSQGSTVKGVWLALHDIHYADEELKQQLVYVGV
TRPTDFCLYFDGTK
SEQ ID NO: 126 (>I6XH64; Aeromonas phage CC2)
MAVDAVQSGTGHITISGPPGSGKTFLVKYIIKMLGDELGTVLAAPTHQAKIVLTEMSGIE
ACTIHSLMKIHPETLEDIQIFDQSKMPDLSTVRYLIIEEASMHSKALFNITMKSIPPTCR
IIAIGDKDQIQPVDHAPGELSPYFTDSRFTQIRMTDIMRQSLDNPIIQVATTIREGGWIY
QNWNKEKKSGVYKVKSITDLINSYLRVVKTPEDLTKYRFLAFTNKVVDKVNSIVRKHVYK
TDLPFIEGEKLVLQEPVMVEYDDDTIETIFTNGEVVTVDEIEVSDMNIRIDGSPAFSISV
AKLKVTSDFSGVTHDIMSVYGEDSKAEFNYQLSEAAAVIKQMQRGQTKAAWASFWDAKKT
FTETKSLGACTIHKSQGSTVKGVWLGLHDISYADTDLQQQLVYVGVTRPTDFCLYFDGSK
SEQ ID NO: 127 (>K4FBD0; Cronobacter phage vB CsaM GAP161)
MSELTFDDLSDDQKSAHDRVIHNIQNAIHTTITGGPGVGKTTLVKFVFNTLKGLGISGIW
LTAPTHQAKNVLAAATGMDATTIHSALKISPVTNEELRVFEQQKGKKAPDLSTCRVFVVE
EVSMVDMDLFRIIRRSIPSNAVILGLGDKDQIRPVNADGRVELSPFFDEEIFDVIRMDKI
MRQAEGNPIIQVSRAVRDGKMLKPMSVGDLGVFQHANAVDFLRQYFRRVKTPDDLIENRM
FAYTNDNVDKLNATIRKHLYKTTEPFILDEVIVMQEPLVQEMRLNGQIFTEIVYNNNEKI
RVLEIIPRREVIKAEKCDEKIEIEFYLLKTVSLEEETEAQIQVVVDPVMKDRLGNYLAYV
ASTYKRIKQQTGYKAPWHSFWAIKNKFQDVKPLPVCTYHKSQGSTYDHAYMYTRDAYAFA
SEQ ID NO: 128 (>D5JF67; Klebsiella phage KP15)
MSELTFDDLSEDQKNAHDRVIKNIRNKIHTTITGGPGVGKTTLVKFVFETLKKLGISGIW
LTAPTHQAKNVLSEAVGMDATTIHSALKISPVTNEELRVFEQQKGKKAADLSECRVFVVE
EVSMVDKELFRIIKRTIPSCAVILGLGDKDQIRPVNTEGITELSPFFDEEIFDVIRMDKI
MRQAEGNPIIQVSRAIRDGKPLMPLMNGELGVMKHENASDFLRRYFSRVKTPDDLNNNRM
FAYTNANVDKLNAVIRKHLYKTDQPFIVGEVVVMQEPLVTEGRVNGVSFVEVIYNNNEQI
KILEIIPRSDTIKADRCDPVQIDYFLMKTESMFEDTKADIQVIADPVMQERLGDYLNYVA
FQYKKMKQETGYKAPWYSFWQIKNKFQTVKALPVCTYHKGQGSTYDHSYMYTRDAYAYAD
YELCKQLLYVGTTRARFTVDYV
SEQ ID NO: 129 (>J7HXT5; Stenotrophomonas phage IME13)
MVTYDDLTVGQKDAIEKALQAMRTKRHITIRGPAGSGKTTMTRFLLERLFQTGQQGIVLT
APTHQAKKELSKHALRKSYTIQSVLKINPSTLEENQIFEQKGTPDFSKTRVLICDEVSFY
TRKLFDILMRNVPSHCVVIGIGDKAQIRGVSEDDTHELSPFFTDNRFEQVELTEVKRHQG
PIIEVATDIRNGKWIYEKLDDSGNGVKQFHTVKDFLSKYFERTKTPNDLLENRIMAYTNN
SVDKLNSVIRKQLYGANAAPFLPDEILVMQEPLMFDIDIGGQTLKEVIFNNGQNVRVINV
KPSRKTLKAKGVGEIEVECTMLECESYEEDEDDYRRAWFTVVHDQNTQYAINEFLSIIAE
KYRSREVFPNWKDFWAIRNTFVKVRPLGAMTFHKSQGSTFDNAYLFTPCLHQYCRDPDVA
QELIYVGNTRARKNVCFV
SEQ ID NO: 130 (>E5EYE6; Acinetobacter phage Ac42)
MNFEDLTEGQKNAYTAAIKAIETVPSSSAEKRHLTINGPAGTGKTTLTKFLIAELIRRGE
RGVYLAAPTHQAKKVLSQHAGMEASTIHSLLKINPTTYEDSTTFEQKDVPDMSECRVLIC
DEASMYDLKLFQILMSSIPLCCTVIALGDIAQIRPVEPGAFEGQVSPFFTYEKFEQVSLT
EVMRSNAPIIDVATSIRTGNWIYENVIDGAGVHNLTSERSVKSFMEKYFSIVKTPEDLFE
NRLLAFTNKSVDDLNKIVRKKIYNTLEPFIDGEVLVMQEPLIKSYTYEGKKVSEIVFNNG
EMVKVLCCSQTSDEISVRGCSTKYMVRYWQLDLQSLDDPDLTGSINVIVDEAEINKLNLV
LGKSAEQFKSGAVKAAWADWWKLKRNFHKVKALPCSTIHKSQGTSVDNVFLYTPCIHKAD
SQLAQQLLYVGATRARHNVYYI
SEQ ID NO: 131 (>E3SFA5; Shigella phage SP18)
MIKFEDLNTGQKEAFDYITEAIQRRSGECITLNGPAGTGKTTLTKFVIDHLVRNGVMGIV
LAAPTHQAKKVLSKLSGQTANTIHSILKINPTTYEDQNIFEQREMPDMSKCNVLVCDEAS
MYDGSLFKIICNSVPEWCTILGIGDMHQLQPVDPGSTQQKISPFFTHPKFKQIHLTEVMR
SNAPIIEVATEIRNGGWFRDCMYDGHGVQGFTSQTALKDFMVNYFGIVKDADMLMENRMY
AYTNKSVEKLNNIIRRKLYETDKAFLPYEVLVMQEPHMKELEFEGKKFSETIFNNGQLVR
IKDCKYTSTILRCKGESHQLVINYWDLEVESIDEDEEYQVDRIKVLPEDQQPKFQAYLAK
VADTYKQMKAAGKRPEWKDFWKARRTFLKVRALPVSTIHKAQGVSVDKAFIYTPCIHMAE
ASLASQLAYVGITRARYDAYYV
SEQ ID NO: 132 (>I7J3V8; Yersinia phage phiR1-RT)
MITYDDLTDGQKSAFDNTMEAIKNKKGHITINGPAGTGKTTLTKFIIDHLIKTGEAGIIL
CAPTHQAKKVLSKLSGMDASTIHSVLKINPTTYEENQIFEQREVPDLAACRVLICDEASF
YDRKLFGIILATVPSWCTVIALGDKDQLRPVTPGESEQQLSPFFSHAKFKQVHLTEIKRS
NGPIIQVATDIRNGGWLSENIVDGEGVHAFNSNTALKDFMIRYFDVVKTADDLIESRMLA
YTNKSVDKLNGIIRRKLYETDKPFINGEVLVMQEPLMKELEFDGKKFHEIVFNNGQLVKI
LYASETSTFISARNVPGEYMIRYWNLEVETADSDDDYATSQIQVICDPAEMTKFQMFLAK
TADTYKNSGVKAYWKDFWSVKNKFKKVKALPVSTIHKSQGCTVNNTFLYTPCIHMADAQL
AKQLLYVGATRARTNLYYI
SEQ ID NO: 133 (>M1EA88; Salmonella phage S16)
MITFEQLTSGQKLAFDETIRAIKEKKNHVTINGPAGTGKTTLTKFIMEHLVSTGETGIIL
TAPTHAAKKVLTKLSGMEANTIHKILKINPTTYEESMLFEQKEVPDLASCRVLICDEASM
WDRKLFKILMASIPKWCTIVAIGDVAQIRPVDPGETEAHISPFFIHKDFKQLNLTEVMRS
NAPIIDVATDIRNGSWIYEKTVDGHGVHGFTSTTALKDFMMQYFSIVKSPEDLFENRMLA
FTNKSVDKLNSIIRRRLYQTEEAFVVGEVIVMQEPLMRELVFEGKKFHETLFTNGQYVRI
LSADYTSSFLGAKGVSGEHLIRHWVLDVETYDDEEYAREKINVISDEQEMNKFQFFLAKT
ADTYKNWNKGGKAPWSEFWDAKRKFHKVKALPCSTFHKAQGISVDSSFIYTPCIHVSSDN
KFKLELLYVGATRGRHDVFFV

The following Examples illustrate the invention.

### EXAMPLES

Detailed methods for making and testing mutant CsG pores and mutant CsgG/CsGF complexes are described in WO2016/034591, WO2017/149316, WO2017/149317, WO2017/149318, WO2018/211241 and WO2019/002893 (all incorporated by reference herein in their entirety). Detailed methods for making modified Dda helicases are described in WO2015/055981, WO2015/166276 and WO2016/055777 (all incorporated herein by reference in their entirety).

### Example 1

This example describes a method of comparing speed properties of a variant nanopore against a control variant nanopore using a polynucleotide binding protein controlling the movement of a polynucleotide.

### 3.6 kb Analyte Preparation

A double stranded 3.6 kb DNA analyte was prepared using specific primers and PCR. The PCR product was subjected to NEBNext end repair and NEBNext dA-tailing modules (New England Biolabs (NEB)), to generate 3′ dA overhangs.

### Variant and Control Nanopore Preparation

Recombinant expression vectors encoding the variant nanopores or control nanopore as described in WO 2019/002893 (incorporated herein by reference in its entirety) were transformed into chemically competent E. coli cells. The constructs comprised a C-terminal Strep affinity tag. The cells were plated onto an LB Agar plate containing appropriate antibiotics for selection. Colonies from agar plate were inoculated in LB Media with antibiotics and grown overnight before diluting into autoinduction media and incubated at 18 C for 68 hrs. Cells were harvested through centrifugation before being lysed and extracted in bugbuster (Merck 70921) and 0.1% DDM. Supernatant was purified using affinity chromatography, heat treatment at 60° C. for 30 mins and size exclusion chromatography selecting for oligomeric nanopores as judged by SDS-PAGE.

CsgG-CsgF complexes were prepared from nanopores purified as above and chemically synthesised CsgF peptides. Nanopores were buffer exchanged into a buffer comprising 50 mM Tris, 150 mM NaCl, 2 mM EDTA, 0.1% SDS, 0.1% Brij58, pH7.0 and then incubated in a 4× molar excess of peptide to CsgG monomer for 1 hr at 25° C. Reactions were stopped with heating at 60° C. for 15 mins followed by centrifugation to remove any precipitate.

### Sequencing Adaptor Preparation

Recombinant expression vectors encoding the variants of polynucleotide binding protein described in WO2016/055777, with an N-terminal affinity and solubility tag were transformed into chemically competent E. coli cells. The cells were plated onto an LB agar plate containing antibiotics. Colonies from the agar plate were inoculated into LB growth media, grown to OD 0.400-0.800 and induced, then grown for a further 16 hours at 18° C. The cells were lysed by sonication in the presence of benzonase and protease inhibitors. The supernatant was further purified using affinity chromatography. The purified control polynucleotide binding proteins were bound to sequencing Y adaptors described in WO 2015/110813A1 (herein incorporated by reference in its entirety) in 50 mM Hepes pH8.0, 100 mM Potassium acetate, 1 mM EDTA for 10 minutes at ambient temperature. TMAD (SIGMA) was added to a final concentration of 100 μM and incubated for an hour at 34° C. 10 mM ATP, 10 mM MgCl₂ and 0.5 M NaCl were added and incubated for 10 minutes at ambient temperature. The polynucleotide binding protein bound sequencing adaptors were purified by anion exchange.

### Library Preparation

The sequencing adaptors were ligated to the 3.6 kb analyte. The library was prepared for sequencing and run on a MinION flow cell following the manufacturer's guidelines (Oxford Nanopore Technologies).

### Electrical Measurements

Electrical measurements were acquired from nanopores that were inserted into MinION flow cells. After a single pore inserted into the block co-polymer membrane within each channel, 1 mL of a buffer comprising 25 mM Potassium Phosphate, 150 mM Potassium Ferrocyanide (II), 150 mM Potassium Ferricyanide (III), pH 8.0 was flowed through the system to remove any excess protein nanopores.

A standard sequencing script was run and raw data collected in a bulk FAST5 file using MinKNOW software (Oxford Nanopore Technologies).

### Data Analysis

The DNA library was sequenced using a standard basecalling algorithm from Guppy (Oxford Nanopore Technologies). Greater than or equal to 145 reads for each nanopore variant were mapped to the 3.6 kb analyte reference sequence.

The speed of an individual DNA strand as it translocates through the pore was calculated by dividing the number of bases mapped to the reference by the duration of the read (measured in bases per second). The median speed (bases per second) was the median speed of multiple individual DNA strands as they translocated through the pore. The median speed of the control nanopore flow cell was subtracted from the median speed of the variant nanopore flow cell. This was the Speed delta. Hence, a positive Speed delta indicated that DNA translocated more quickly through the variant nanopore than the control nanopore.

The variation of speed of multiple individual DNA strands as they translocated through the pore was measured by calculating the interquartile range. Hence, a small interquartile range implied a narrow distribution of speeds, and a large interquartile range implied a broad distribution of speeds. The normalised speed distribution was calculated by dividing the interquartile range of the speed by the median speed and multiplying by 100. The normalised speed distribution of the control nanopore flow cell was subtracted from the normalised speed distribution of the variant nanopore flow cell, this was the normalised speed distribution delta. Hence, a negative normalised speed distribution delta indicated that the variant nanopore has a narrower distribution of speeds than the control nanopore.

TABLE 11
The Speed Δ is the difference in median speed (bps) between
the variant nanopore flow cell and the control nanopore flow cell.

| Variant Position | Variant AA | Speed Δ |
|---|---|---|
| 97 | D | 58.23 |
| 97 | G | 28.47 |
| 97 | N | 23.22 |
| 97 | R | 44.64 |
| 97 | S | 64.24 |
| 100 | A | 77.22 |
| 100 | K | 76.49 |
| 100 | S | 33.38 |
| 101 | A | −3.93 |
| 101 | deletion | 43.00 |
| 101 | G | 58.77 |
| 101 | S | 44.82 |
| 102 | A | 33.83 |
| 102 | D | 21.27 |
| 102 | R | 35.98 |
| 102 | S | 14.59 |
| 102 | W | 44.70 |

TABLE 12
The Normalised Speed Distribution Δ is the difference
in the normalised speed distribution between the variant
nanopore flow cell and the control nanopore flow cell.

| | | Normalised |
|---|---|---|
| | | Speed |
| | | Distribution |
| Variant Position | Variant AA | Δ |
| 97 | D | 3.72 |
| 97 | G | −0.06 |
| 97 | N | −0.33 |
| 97 | R | 0.56 |
| 97 | S | −3.18 |
| 100 | A | −1.04 |
| 100 | K | 0.03 |
| 100 | S | −0.66 |
| 101 | A | −0.44 |
| 101 | deletion | 3.99 |
| 101 | G | −0.13 |
| 101 | S | 1.13 |
| 102 | A | −0.64 |
| 102 | D | 11.75 |
| 102 | R | −1.37 |
| 102 | S | −2.37 |
| 102 | W | 27.66 |

### Example 2

This example describes a method of comparing speed and accuracy properties of a variant polynucleotide binding protein controlling the movement of a polynucleotide against a control variant polynucleotide binding protein controlling the movement of a polynucleotide using a nanopore described in WO2017/149316.

### Barcoded 3.6 kb Analyte Preparation

A double stranded 3.6 kb DNA analyte was prepared using specific primers and PCR. The PCR product was subjected to NEBNext end repair and NEBNext dA-tailing modules (New England Biolabs (NEB)), to generate 3′ dA overhangs. Barcodes were introduced into the analyte using EXP-PBC0001 (Oxford Nanopore Technologies), following the manufacturer's guidelines.

### Variant Sequencing Adaptor Preparation

Recombinant expression vectors encoding the variants of polynucleotide binding protein described in WO2016/055777, with an N-terminal affinity and solubility tag were transformed into chemically competent E. coli cells. The cells were plated onto an LB agar plate containing antibiotics. Colonies from the agar plate were inoculated into LB growth media, grown to OD 0.400-0.800 and induced, then grown for a further 16 hours at 18° C. The cells were either lysed with Bugbuster extraction reagent (Merck 70921) in the presence of lysozyme, benzonase and protease inhibitors or lysed by sonication in the presence of benzonase and protease inhibitors. The supernatant was further purified using affinity chromatography. A molar excess of purified variant polynucleotide binding protein was bound to sequencing Y adaptors described in WO 2015/110813A1 (herein incorporated by reference in its entirety) in 50 mM Hepes pH8.0, 100 mM Potassium acetate, 1 mM EDTA for 10 minutes at ambient temperature. TMAD (SIGMA) was added to a final concentration of 100 μM and incubated for an hour at 34° C. 10 mM ATP, 10 mM MgCl₂ and 0.5 M NaCl were added and incubated for 10 minutes at ambient temperature. The variant polynucleotide binding protein bound sequencing adaptors were purified using Sera-Mag SpeedBeads (Thermo Scientific). These were the variant sequencing adaptors.

### Control Sequencing Adaptor Preparation

A variant of the polynucleotide binding protein described in WO2016/055777 was used as a control for each of the variant positions. The control polynucleotide binding proteins were purified and loaded onto sequencing adaptors as described above. The control polynucleotide binding protein bound sequencing adaptors were purified on an anion exchange column or using Sera-Mag SpeedBeads (Thermo Scientific). These were the control sequencing adaptors.

### Library Preparation

The variant sequencing adaptors and control sequencing adaptors were ligated to barcoded 3.6 kb analytes, each variant was ligated to a different barcode, and each control was ligated to a different barcode. The variant libraries and control libraries were pooled. The pooled library was prepared for sequencing and run on a MinION flow cell following the manufacturer's guidelines (Oxford Nanopore Technologies). Up to 6 variants were run on a single MinION flow cell with their control, this control was the internal flow cell control.

### Electrical Measurements

Electrical measurements were acquired on a FLO-MIN106 MinION flow cell and GridION (Oxford Nanopore Technologies). A standard sequencing script was run and raw data collected in a bulk FAST5 file using MinKNOW software (Oxford Nanopore Technologies).

### Data Analysis

The DNA library was sequenced using a standard basecalling algorithm from Guppy (Oxford Nanopore Technologies). The sequenced reads were de-multiplexed using Guppy (Oxford Nanopore Technologies). Greater than or equal to 500 reads for each variant and control were mapped to the 3.6 kb analyte reference sequence. Speed and Normalised Speed Distribution delta were calculated as described in Example 1.

TABLE 13
The Speed Δ is the difference in median speed (bps) between
the variant at position 177 and the internal flow cell control.

| Variant Position | Variant AA | Speed Δ |
|---|---|---|
| 177 | D | 3.07 |
| 177 | E | 58.70 |
| 177 | F | −33.41 |
| 177 | G | −27.62 |
| 177 | H | 10.64 |
| 177 | I | 42.98 |
| 177 | L | 51.39 |
| 177 | M | 48.31 |
| 177 | N | 2.40 |
| 177 | Q | 59.68 |
| 177 | R | 21.69 |
| 177 | S | −31.10 |
| 177 | T | 22.62 |
| 177 | V | −16.00 |
| 177 | W | −23.42 |
| 177 | Y | −43.93 |

TABLE 14
The Speed Δ is the difference in median speed (bps) between
the variant at position 114 and the internal flow cell control.

| Variant Position | Variant AA | Speed Δ |
|---|---|---|
| 114 | A | 13.80 |
| 114 | G | −11.30 |
| 114 | I | 93.69 |
| 114 | L | −42.01 |
| 114 | M | 81.76 |
| 114 | P | 84.49 |
| 114 | S | −7.28 |
| 114 | T | −35.67 |
| 114 | V | 26.04 |

TABLE 15
The Speed Δ is the difference in median speed (bps) between
the variant at position 358 and the internal flow cell control.

| Variant Position | Variant AA | Speed Δ |
|---|---|---|
| 358 | E | −53.89 |
| 358 | I | −40.19 |
| 358 | L | −42.66 |
| 358 | M | −48.58 |

TABLE 16
The Accuracy Δ is the difference in median accuracy (%) between
the variant at the 177 position and the internal flow cell control.

| Variant Position | Variant AA | Accuracy Δ |
|---|---|---|
| 177 | F | −0.08 |
| 177 | H | −0.2 |
| 177 | I | −0.14 |
| 177 | L | −0.08 |
| 177 | M | −0.09 |
| 177 | N | −0.66 |
| 177 | W | −0.46 |

TABLE 17
The Accuracy Δ is the difference in median accuracy (%) between
the variant at the 114 position and the internal flow cell control.

| Variant Position | Variant AA | Accuracy Δ |
|---|---|---|
| 114 | G | 0.13 |
| 114 | I | −0.76 |
| 114 | P | −0.70 |

TABLE 18
The Accuracy Δ is the difference in median accuracy (%) between
the variant at the 358 position and the internal flow cell control.

| Variant Position | Variant AA | Accuracy Δ |
|---|---|---|
| 358 | I | 0.96 |
| 358 | M | 0.94 |

TABLE 19
The Normalised Speed Distribution Δ is the difference
in the normalised speed distribution between the variant
at the 177 position and the internal flow cell control.

| Variant | Variant | Normalised Speed |
|---|---|---|
| Position | AA | Distribution Δ |
| 177 | F | −8.19 |
| 177 | H | −5.38 |
| 177 | I | −4.87 |
| 177 | L | −4.44 |
| 177 | M | −1.06 |
| 177 | N | 0.85 |
| 177 | W | −4.42 |

TABLE 20
The Normalised Speed Distribution Δ is the difference
in normalised speed distribution between the variant
at the 114 position and the internal flow cell control.

| Variant | Variant | Normalised Speed |
|---|---|---|
| Position | AA | Distribution Δ |
| 114 | G | −4.99 |
| 114 | I | −8.23 |
| 114 | P | −7.45 |

TABLE 21
The Normalised Speed Distribution Δ is the difference
in the normalised speed distribution between the variant
at the 358 position and the internal flow cell control.

| Variant | Variant | Normalised Speed |
|---|---|---|
| Position | AA | Distribution Δ |
| 358 | I | −0.86 |
| 358 | M | 0.45 |

### Example 3

This example describes a method of comparing speed and accuracy properties of a variant polynucleotide binding protein controlling the movement of a polynucleotide against a control variant polynucleotide binding protein controlling the movement of a polynucleotide using a nanopore described in WO2019/002893.

### Barcoded 3.6 kb Analyte Preparation

A double stranded 3.6 kb DNA analyte was prepared using specific primers and PCR. The PCR product was subjected to NEBNext end repair and NEBNext dA-tailing modules (New England Biolabs (NEB)), to generate 3′ dA overhangs. Barcodes were introduced into the analyte using EXP-PBC0001 (Oxford Nanopore Technologies), following the manufacturer's guidelines.

### Variant Sequencing Adaptor Preparation

Recombinant expression vectors encoding the variants of polynucleotide binding protein described in WO2016/055777, with an N-terminal affinity and solubility tag were transformed into chemically competent E. coli cells. The cells were plated onto an LB agar plate containing antibiotics. Colonies from the agar plate were inoculated into LB growth media, grown to OD 0.400-0.800 and induced, then grown for a further 16 hours at 18° C. The cells were either lysed with Bugbuster extraction reagent (Merck 70921) in the presence of lysozyme, benzonase and protease inhibitors or lysed by sonication in the presence of benzonase and protease inhibitors. The supernatant was further purified using affinity chromatography. A molar excess of purified variant polynucleotide binding protein was bound to sequencing Y adaptors described in WO 2015/110813A1 (herein incorporated by reference in its entirety) in 50 mM Hepes pH8.0, 100 mM Potassium acetate, 1 mM EDTA for 10 minutes at ambient temperature. TMAD (SIGMA) was added to a final concentration of 100 μM and incubated for an hour at 34° C. 10 mM ATP, 10 mM MgCl₂ and 0.5 M NaCl were added and incubated for 10 minutes at ambient temperature. The variant polynucleotide binding protein bound sequencing adaptors were purified using Sera-Mag SpeedBeads (Thermo Scientific). These were the variant sequencing adaptors.

### Control Sequencing Adaptor Preparation

A variant of the polynucleotide binding protein described in WO2016/055777 was used as a control for each of the variant positions. The control polynucleotide binding proteins were purified and loaded onto sequencing adaptors as described above. These were the control sequencing adaptors.

### Library Preparation

The variant sequencing adaptors and control sequencing adaptors were ligated to barcoded 3.6 kb analytes, each variant was ligated to a different barcode, and each control was ligated to a different barcode. The variant libraries and control libraries were pooled. The pooled library was prepared for sequencing and run on a MinION flow cell following the manufacturer's guidelines (Oxford Nanopore Technologies). Up to 6 variants were run on a single MinION flow cell with their control, this control was the internal flow cell control.

### Electrical Measurements

Electrical measurements were acquired on a FLO-MIN112 MinION flow cell and GridION (Oxford Nanopore Technologies). A standard sequencing script was run and raw data collected in a bulk FAST5 file using MinKNOW software (Oxford Nanopore Technologies).

### Data Analysis

The DNA library was sequenced using a standard basecalling algorithm from Guppy (Oxford Nanopore Technologies). The sequenced reads were de-multiplexed using Guppy (Oxford Nanopore Technologies). Greater than or equal to 500 reads for each variant and control were mapped to the 3.6 kb analyte reference sequence. Speed and Normalised Speed Distribution delta were calculated as described in Example 1.

TABLE 22
The Speed Δ is the difference in median speed (bps) between
the variant positions and the internal flow cell control.

| Variant Position | Variant AA | Speed Δ |
|---|---|---|
| 350 | I | 2.22 |
| 351 | I | −9.76 |
| 351 | Q | −5.61 |
| 354 | A | 11.26 |
| 354 | Q | 0.09 |
| 358 | A | −0.99 |
| 358 | E | −7.28 |
| 358 | F | −10.93 |
| 358 | I | −5.51 |
| 358 | M | −11.75 |
| 358 | S | 1.60 |
| 361 | I | 21.60 |
| 361 | Q | 13.79 |

TABLE 23
The Accuracy Δ is the difference in median accuracy (%)
between the variant positions and the internal flow cell control.

| Variant Position | Variant AA | Accuracy Δ |
|---|---|---|
| 350 | I | −0.50 |
| 351 | I | −3.36 |
| 351 | Q | −0.89 |
| 354 | A | −1.96 |
| 354 | Q | −1.43 |
| 358 | A | 1.07 |
| 358 | E | 0.84 |
| 358 | F | 1.36 |
| 358 | I | 1.05 |
| 358 | M | 0.81 |
| 358 | S | 0.86 |
| 361 | I | −4.62 |
| 361 | Q | −1.81 |

TABLE 24
The Normalised Speed Distribution Δ is the difference
in the normalised speed distribution between the variant
positions and the internal flow cell control.

| Variant | Variant | Normalised Speed |
|---|---|---|
| Position | AA | Distribution Δ |
| 350 | I | −0.52 |
| 351 | I | 2.25 |
| 351 | Q | 0.54 |
| 354 | A | −0.44 |
| 354 | Q | −0.53 |
| 358 | A | −29.70 |
| 358 | E | −26.60 |
| 358 | F | 0.73 |
| 358 | I | −5.31 |
| 358 | M | −0.56 |
| 358 | S | −1.56 |
| 361 | I | 0.51 |
| 361 | Q | 0.07 |

### Example 4

This example describes a method of comparing speed and accuracy properties of a variant polynucleotide binding protein controlling the movement of a polynucleotide against a control variant polynucleotide binding protein controlling the movement of a polynucleotide using a variant nanopore.

### Barcoded 3.6 kb Analyte Preparation

A double stranded 3.6 kb DNA analyte was prepared using specific primers and PCR. The PCR product was subjected to NEBNext end repair and NEBNext dA-tailing modules (New England Biolabs (NEB)), to generate 3′ dA overhangs. Barcodes were introduced into the analyte using EXP-PBC001 (Oxford Nanopore Technologies), following the manufacturer's guidelines.

### Variant Nanopore Preparation

Recombinant expression vectors encoding the variant nanopores or control nanopore as described in WO2019002893 were transformed into chemically competent E. coli cells. The constructs comprised a C-terminal Strep affinity tag. The cells were plated onto an LB Agar plate containing appropriate antibiotics for selection. Colonies from agar plate were inoculated in LB Media with antibiotics and grown overnight before diluting into autoinduction media and incubated at 18 C for 68 hrs. Cells were harvested through centrifugation before being lysed and extracted in bugbuster (Merck 70921) and 0.1% DDM. Supernatant was purified using affinity chromatography, heat treatment at 60° C. for 30 mins and size exclusion chromatography selecting for oligomeric nanopores as judged by SDS-PAGE.

CsgG-CsgF complexes were prepared from nanopores purified as above and chemically synthesised CsgF peptides. Nanopores were buffer exchanged into a buffer comprising 50 mM Tris, 150 mM NaCl, 2 mM EDTA, 0.1% SDS, 0.1% Brij58, pH7.0 and then incubated in a 4× molar excess of peptide to CsgG monomer for 1 hr at 25° C., reactions were stopped with heating at 60° C. for 15 mins followed by centrifugation to remove any precipitate.

### Variant Sequencing Adaptor Preparation

Recombinant expression vectors encoding the variants of polynucleotide binding protein described in WO2016/055777, with an N-terminal affinity and solubility tag were transformed into chemically competent E. coli cells. The cells were plated onto an LB agar plate containing antibiotics. Colonies from the agar plate were inoculated into LB growth media, grown to OD 0.400-0.800 and induced, then grown for a further 16 hours at 18° C. The cells were either lysed with Bugbuster extraction reagent (Merck 70921) in the presence of lysozyme, benzonase and protease inhibitors or lysed by sonication in the presence of benzonase and protease inhibitors. The supernatant was further purified using affinity chromatography. A molar excess of purified variant polynucleotide binding protein was bound to a sequencing Y adaptor as disclosed in WO 2015/110813 (herein incorporated by reference in its entirety) in 50 mM Hepes pH8.0, 100 mM Potassium acetate, 1 mM EDTA for 10 minutes at ambient temperature. TMAD (SIGMA) was added to a final concentration of 100 μM and incubated for an hour at 34° C. 10 mM ATP, 10 mM MgCl₂ and 0.5 M NaCl were added and incubated for 10 minutes at ambient temperature. The variant polynucleotide binding protein bound sequencing adaptors were purified using Sera-Mag SpeedBeads (Thermo Scientific). These were the variant sequencing adaptors.

### Control Sequencing Adaptor Preparation

A variant of the polynucleotide binding protein described in WO2016/055777 was used as a control for each of the variant positions. The control polynucleotide binding proteins were purified and loaded onto sequencing adaptors as described above. The control polynucleotide binding protein bound sequencing adaptors were purified on an anion exchange column or using Sera-Mag SpeedBeads (Thermo Scientific). These were the control sequencing adaptors.

### Library Preparation

The variant sequencing adaptors and control sequencing adaptors were ligated to barcoded 3.6 kb analytes, each variant was ligated to a different barcode, and each control was ligated to a different barcode. The variant libraries and control libraries were pooled. The pooled library was prepared for sequencing and run on a MinION flow cell following the manufacturer's guidelines (Oxford Nanopore Technologies). Up to 6 variants were run on a single MinION flow cell with their control, this control was the internal flow cell control.

### Electrical Measurements

Electrical measurements were acquired from nanopores that were inserted into MinION flow cells. After a single pore inserted into the block co-polymer membrane within each channel, 1 mL of a buffer comprising 25 mM Potassium Phosphate, 150 mM Potassium Ferrocyanide (II), 150 mM Potassium Ferricyanide (III), pH 8.0 was flowed through the system to remove any excess protein nanopores.

A standard sequencing script was run and raw data collected in a bulk FAST5 file using MinKNOW software (Oxford Nanopore Technologies).

### Data Analysis

The DNA library was sequenced using a standard basecalling algorithm from Guppy (Oxford Nanopore Technologies). Greater than or equal to 145 reads for each nanopore variant were mapped to the 3.6 kb analyte reference sequence. Speed and Normalised Speed Distribution delta were calculated as described in Example 1.

TABLE 25
The Speed Δ is the difference in median speed (bps)
between the variant polynucleotide binding protein and
the internal flow cell control on the variant nanopore.

| | | Variant | Variant | |
|---|---|---|---|---|
| | | Polynucleotide | Polynucleotide | |
| Nanopore | Nanopore | Binding | Binding | |
| Variant | Variant | Protein | Protein | Speed |
| Position | AA | Position | Variant AA | Δ |
| 100 | A | 350 | I | −0.75 |
| 100 | A | 351 | I | −11.90 |
| 100 | A | 351 | Q | −3.05 |
| 100 | A | 354 | A | −3.11 |
| 100 | A | 354 | Q | −4.27 |
| 100 | A | 358 | I | −9.09 |
| 100 | A | 361 | I | 8.41 |
| 100 | A | 361 | Q | 1.36 |
| 102 | A | 350 | I | −7.29 |
| 102 | A | 350 | S | 1.75 |
| 102 | A | 351 | I | −12.19 |
| 102 | A | 351 | Q | −8.71 |
| 102 | A | 354 | A | 7.82 |
| 102 | A | 354 | Q | 1.54 |
| 102 | A | 358 | I | −8.99 |
| 102 | A | 358 | L | −5.86 |
| 102 | A | 358 | Q | −1.89 |
| 102 | A | 361 | I | 14.99 |
| 102 | A | 361 | Q | 8.76 |
| 102 | S | 350 | I | 4.49 |
| 102 | S | 351 | I | −9.90 |
| 102 | S | 351 | Q | −6.19 |
| 102 | S | 354 | A | 7.74 |
| 102 | S | 354 | Q | 3.20 |
| 102 | S | 358 | I | −12.91 |
| 102 | S | 361 | I | 13.93 |
| 102 | S | 361 | Q | 7.43 |

TABLE 26
The Accuracy Δ is the difference in median accuracy
(%) between the variant polynucleotide binding protein
and the internal flow cell control on the variant nanopore.

| | | Variant | Variant | |
|---|---|---|---|---|
| | | Polynucleotide | Polynucleotide | |
| Nanopore | Nanopore | Binding | Binding | |
| Variant | Variant | Protein | Protein | Accuracy |
| Position | AA | Position | Variant AA | Δ |
| 100 | A | 350 | I | −0.03 |
| 100 | A | 351 | I | −2.65 |
| 100 | A | 351 | Q | −1.32 |
| 100 | A | 354 | A | −1.06 |
| 100 | A | 354 | Q | −1.24 |
| 100 | A | 358 | I | 0.89 |
| 100 | A | 361 | I | −2.99 |
| 100 | A | 361 | Q | −1.73 |
| 102 | A | 350 | I | −0.81 |
| 102 | A | 350 | S | −0.34 |
| 102 | A | 351 | I | −4.42 |
| 102 | A | 351 | Q | −1.60 |
| 102 | A | 354 | A | −3.22 |
| 102 | A | 354 | Q | −1.59 |
| 102 | A | 358 | I | 1.00 |
| 102 | A | 358 | L | 0.97 |
| 102 | A | 358 | Q | 0.94 |
| 102 | A | 361 | I | −4.12 |
| 102 | A | 361 | Q | −2.47 |
| 102 | S | 350 | I | −0.36 |
| 102 | S | 351 | I | −4.40 |
| 102 | S | 351 | Q | −1.24 |
| 102 | S | 354 | A | −2.33 |
| 102 | S | 354 | Q | −1.43 |
| 102 | S | 358 | I | 1.17 |
| 102 | S | 361 | I | −4.00 |
| 102 | S | 361 | Q | −1.93 |

TABLE 27
The Normalised Speed Distribution Δ is the difference in the normalised
speed distribution between the variant polynucleotide binding protein
and the internal flow cell control on the variant nanopore.

| | | Variant | Variant | |
|---|---|---|---|---|
| | | Polynucleotide | Polynucleotide | Normalised |
| Nanopore | Nanopore | Binding | Binding | Speed |
| Variant | Variant | Protein | Protein | Distribution |
| Position | AA | Position | Variant AA | Δ |
| 100 | A | 350 | I | 0.27 |
| 100 | A | 351 | I | 1.96 |
| 100 | A | 351 | Q | 0.57 |
| 100 | A | 354 | A | 0.54 |
| 100 | A | 354 | Q | 0.75 |
| 100 | A | 358 | I | 0.30 |
| 100 | A | 361 | I | 0.32 |
| 100 | A | 361 | Q | 0.35 |
| 102 | A | 350 | I | 3.65 |
| 102 | A | 350 | S | 1.25 |
| 102 | A | 351 | I | 2.25 |
| 102 | A | 351 | Q | 1.29 |
| 102 | A | 354 | A | 0.17 |
| 102 | A | 354 | Q | −0.04 |
| 102 | A | 358 | I | 0.67 |
| 102 | A | 358 | L | 0.82 |
| 102 | A | 358 | Q | 1.35 |
| 102 | A | 361 | I | 1.04 |
| 102 | A | 361 | Q | 0.75 |
| 102 | S | 350 | I | −0.05 |
| 102 | S | 351 | I | 2.78 |
| 102 | S | 351 | Q | 1.41 |
| 102 | S | 354 | A | −0.11 |
| 102 | S | 354 | Q | 0.04 |
| 102 | S | 358 | I | −0.55 |
| 102 | S | 361 | I | 1.05 |
| 102 | S | 361 | Q | 0.96 |

### Example 5

This example describes a method of comparing speed and accuracy properties of a variant polynucleotide binding protein controlling the movement of a polynucleotide against a control variant polynucleotide binding protein controlling the movement of a polynucleotide using a nanopore.

### Barcoded 3.6 kb Analyte Preparation

A double stranded 3.6 kb DNA analyte was prepared using specific primers and PCR. The PCR product was subjected to NEBNext end repair and NEBNext dA-tailing modules (New England Biolabs (NEB)), to generate 3′ dA overhangs. Barcodes were introduced into the analyte using EXP-PBC0001 (Oxford Nanopore Technologies), following the manufacturer's guidelines.

### Nanopore Preparation

The nanopore was prepared as described in Example 4.

### Variant Sequencing Adaptor Preparation

Recombinant expression vectors encoding the variants of polynucleotide binding protein described in WO2016/055777, with an N-terminal affinity and solubility tag were transformed into chemically competent E. coli cells. The cells were plated onto an LB agar plate containing antibiotics. Colonies from the agar plate were inoculated into LB growth media, grown to OD 0.400-0.800 and induced, then grown for a further 16 hours at 18° C. The cells were lysed with Bugbuster extraction reagent (Merck 70921) in the presence of lysozyme, benzonase and protease inhibitors. The supernatant was further purified using affinity chromatography. Variant polynucleotide binding protein was bound to sequencing Y adaptors described in WO 2015/110813A1 (herein incorporated by reference in its entirety) in 1× Buffer BXT (IBA Lifesciences GmbH) for 10 minutes at ambient temperature. TMAD (SIGMA) was added to a final concentration of 100 μM and incubated for an hour at 34° C. 10 mM ATP, 10 mM MgCl₂ and 0.5 M NaCl were added and incubated for 10 minutes at ambient temperature. The variant polynucleotide binding protein bound sequencing adaptors were purified using Sera-Mag SpeedBeads (Thermo Scientific). These were the variant sequencing adaptors.

### Control Sequencing Adaptor Preparation

A variant of the polynucleotide binding protein described in WO2016/055777 was used as a control for each of the variant positions. The control polynucleotide binding proteins were purified and loaded onto sequencing adaptors as described above. These were the control sequencing adaptors

### Library Preparation

The variant sequencing adaptors and control sequencing adaptors were ligated to barcoded 3.6 kb analytes, each variant was ligated to a different barcode, and each control was ligated to a different barcode. The variant libraries and control libraries were pooled. The pooled library was prepared for sequencing and run on a MinION flow cell following the manufacturer's guidelines (Oxford Nanopore Technologies). Up to 46 variants were run on a single PromethION flow cell with their control, this control is the internal flow cell control. At least two flowcells were run per library.

### Electrical Measurements

Electrical measurements were acquired from nanopores that were inserted into PromethION flow cells. After a single pore inserted into the block co-polymer membrane within each channel, 1 mL of a buffer comprising 25 mM Potassium Phosphate, 150 mM Potassium Ferrocyanide (II), 150 mM Potassium Ferricyanide (III), pH 8.0 was flowed through the system to remove any excess protein nanopores.

A standard sequencing script was run and raw data collected in a bulk FAST5 file using MinKNOW software (Oxford Nanopore Technologies).

### Data Analysis

The DNA library was sequenced using a standard basecalling algorithm from Guppy (Oxford Nanopore Technologies). The sequenced reads were de-multiplexed using Guppy (Oxford Nanopore Technologies). Greater than or equal to 20,000 reads for each variant and control were mapped to the 3.6 kb analyte reference sequence. Speed and Normalised Speed Distribution delta were calculated as described in Example 1.

TABLE 28
The Speed Δ is the difference in median speed (bps) between
the variant positions and the internal flow cell control.

| Variant Position | Variant AA | Speed Δ |
|---|---|---|
| 55 | K | 8.29 |
| 156 | F | 13.84 |
| 210 | K | 0.52 |
| 221 | E | 4.37 |
| 350 | E | 6.06 |

TABLE 29
The Accuracy Δ is the difference in median accuracy (%)
between the variant positions and the internal flow cell control.

| Variant Position | Variant AA | Accuracy Δ |
|---|---|---|
| 55 | K | 0.15 |
| 156 | F | 0.26 |
| 210 | K | 0.45 |
| 221 | E | 0.23 |
| 350 | E | 0.48 |

TABLE 30
The Normalised Speed Distribution Δ is the difference
in the normalised speed distribution between the variant
positions and the internal flow cell control.

| Variant | Variant | Normalised Speed |
|---|---|---|
| Position | AA | Distribution Δ |
| 55 | K | −0.20 |
| 156 | F | −0.67 |
| 210 | K | −0.85 |
| 221 | E | −0.94 |
| 350 | E | −1.02 |

1. A modified DNA dependent ATPase (Dda) helicase, wherein the helicase comprises a modification or substitution at one or more of the positions corresponding to amino acid positions 55, 114, 156, 177, 210, 221, 350 and 358 in Dda 1993.

2. The modified DNA dependent ATPase (Dda) helicase according to claim 1, wherein (a) the amino acid corresponding to position 55 is substituted with D, E, K, N or S, (b) the amino acid corresponding to position 114 is substituted with A, V, I, L, M, F, Y, W, G, P, S, T, N or Q, (c) the amino acid corresponding to position 156 is substituted with A, E, F, G, I, L, M, P, S, V, Y, D, K or N, (d) the amino acid corresponding to position 177 is substituted with D, E, F, G, H, I, L, M, N, Q, R, S, T, V, W or Y, (e), the amino acid corresponding to position 210 is substituted with D, E, K, S, N, R, H or Y (f), the amino acid at position 221 is substituted with D, K, E, Q, R, A, H, L, T or Y, (g) the amino acid corresponding to position 350 is substituted with A, D, E, G, K, L, N, Q, R, T, V, H or M or with D, E, A, V, I, L, M, F, W, R, H, K, L, S, T, N or Q and/or (h) the amino acid corresponding to position 358 is substituted with D, E, A, V, I, L, M, F, Y, W, R, H, L, S, T, N or Q.

3. The modified Dda helicase according to claim 1, wherein the helicase further comprises a modification or substitution at the position corresponding to amino acid position 40 in Dda 1993, optionally wherein the amino acid corresponding to position 40 is substituted with A, V, I, L, M, F, Y or W.

4. (canceled)

5. A construct comprising a helicase according to claim 1 and an additional polynucleotide binding moiety, wherein the helicase is attached to the polynucleotide binding moiety and the construct has the ability to control the movement of an analyte.

6. (canceled)

7. A polynucleotide which comprises a sequence which encodes a helicase according to claim 1.

8. A vector which comprises a polynucleotide according to claim 7 operably linked to a promoter.

9. A host cell comprising a vector according to claim 8.

10. A method of making a helicase, the method comprising expressing a polynucleotide according to claim 7.

11. A method of controlling the movement of an analyte, comprising contacting the analyte with a helicase according to claim 1 and thereby controlling the movement of the analyte.

12. (canceled)

13. A method of characterising a target analyte, comprising:
(a) contacting the target analyte with a transmembrane pore and a helicase according to claim 1 such that the helicase or construct controls the movement of the target analyte through the pore; and
(b) taking one or more measurements as the polynucleotide moves with respect to the pore wherein the measurements are indicative of one or more characteristics of the target analyte and thereby characterising the target analyte.

14. A method according to claim 13, wherein the one or more characteristics are selected from (i) the length of the target analyte, (ii) the identity of the target analyte, (iii) the sequence of the target analyte, (iv) the secondary structure of the target analyte and (v) whether or not the target analyte is modified.

15.-23. (canceled)

24. A method of forming a sensor for characterising a target analyte, comprising forming a complex between (a) a pore and (b) a helicase according to claim 1 and thereby forming a sensor for characterising the target analyte.

25.-27. (canceled)

28. A sensor for characterising a target analyte, comprising a complex between (a) a pore and (b) a helicase according to claim 1.

29. (canceled)

30. A kit for characterising a target analyte comprising
(a) a pore and a helicase according to claim 1; or
(b) a helicase according to claim 1 and one or more loading moieties.

31. An apparatus for characterising target analytes in a sample, comprising (a) a plurality of pores and (b) a plurality of helicases according to claim 1.

32. A method of producing a helicase according to claim 1, comprising:
(a) providing a helicase; and
(b) modifying the helicase to produce a helicase according to claim 1.

33.-35. (canceled)

36. A series of two or more helicases attached to a polynucleotide, wherein at least one of the two or more helicases is a helicase according to claim 1.

37. A method of improving the movement of a target analyte with respect to a transmembrane pore when the movement is controlled by a DNA dependent ATPase (Dda) helicase, wherein the DNA dependent ATPase (Dda) helicase is modified to comprise a substitution at one or more of the positions corresponding to amino acid positions 55, 114, 156, 177, 210, 221, 350 and 358 in Dda 1993 and/or the position corresponding to amino acid position 40 in Dda 1993 which improves the movement of the target analyte with respect to the transmembrane pore.

38. An isolated CsgG pore or a homologue or mutant thereof, or an isolated pore complex comprising a CsgG pore, or a homologue or mutant thereof, and a modified CsgF peptide, or a homologue or mutant thereof, wherein the CsgG pore comprises at least one monomer comprising a modification at one or more of positions W97, Q100, E101, N102, and T104 in SEQ ID NO: 117.

39.-47. (canceled)

48. A method for determining the presence, absence or one or more characteristics of a target analyte, comprising the steps of:
(a) contacting the target analyte with an isolated pore or an isolated pore complex according to claim 38 such that the target analyte moves relative to or into the pore complex; and
(b) taking one or more measurements as the analyte moves through the pore complex and thereby determining the presence, absence or one or more characteristics of the analyte, optionally wherein the movement of the target analyte with respect to the pore complex is controlled by a polynucleotide binding protein, further optionally wherein the polynucleotide binding protein is a Dda helicase that comprises a modification or substitution at one or more of the positions corresponding to amino acid positions 55, 114, 156, 177, 210, 221, 350 and 358 in Dda 1993.

49.-63. (canceled)
